(19) United States
(12) Patent Application Publication
(10) Pub. No.: US 2024/0376052 A1
(43) Pub. Date: Nov. 14, 2024

(54) N-SUBSTITUTED INDOLE DERIVATIVES AS SEROTONERGIC AGENTS USEFUL FOR THE TREATMENT OF DISORDERS RELATED THERETO

(71) Applicant: Mindset Pharma Inc., St-Laurent (CA)

(72) Inventors: Abdelmalik Slassi, Mississauga (CA); Joseph A. Araujo, Grimsby (CA); Guy Higgins, Elora (CA)

(73) Assignee: Mindset Pharma Inc., St-Laurent, QC (CA)

(21) Appl. No.: 18/683,846

(22) PCT Filed: Aug. 19, 2022
(86) PCT No.: PCT/CA2022/051263

§ 371 Date: Feb. 15, 2024

Related U.S. Application Data
(60) Provisional application No. 63/260,470, filed on Aug. 20, 2021.
(60) Provisional application No. 63/326,406, filed on Apr. 1, 2022.
(60) Provisional application No. 63/347,845, filed on Jun. 1, 2022.

(51) Int. Cl. C07D 209/12; A61K 31/404; A61K 31/4439; C07B 59/00; C07D 401/04; C07D 403/06

(52) U.S. Cl. CPC C07D 209/12; A61K 31/404; A61K 31/4439; C07B 59/004; C07D 401/04; C07D 403/06; C07B 2200/05

(57) ABSTRACT

The present application relates to indole derivatives of general Formula I, to processes for their preparation, to compositions comprising them and to their use in activation of a serotonin receptors in a cell, as well as to treating diseases, disorders or conditions by activation of a serotonin receptors in a cell. The diseases, disorders or conditions include, for example, psychosis, mental illnesses and CNS disorders. Formula I wherein Q is selected from (Q1), (Q2), (Q2′), (Q3), (Q4) and (Q5). The treatment methods of the application have a reduced risk for or association with serotonin syndrome.

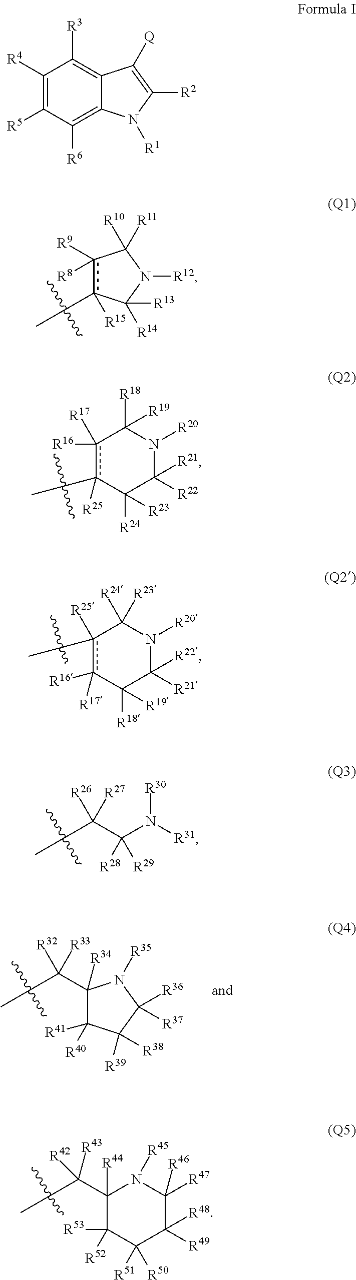

### CROSS-REFERENCE TO RELATED APPLICATIONS

The present application claims the benefit of priority to co-pending U.S. provisional patent application Ser. No. 63/260,470 filed on Aug. 20, 2021, to co-pending U.S. provisional patent application Ser. No. 63/326,406 filed on Apr. 1, 2022, and to co-pending U.S. provisional patent application Ser. No. 63/347,845 filed on Jun. 1, 2022, the contents of each of which are incorporated herein by reference in their entirety.

### FIELD

The application relates to N-substituted-indole compounds for the treatment of different conditions that are treated by activation of serotonin receptors, for example, mental illnesses and neurological disease, in the fields of psychiatry, neurobiology and pharmacotherapy.

### BACKGROUND OF THE APPLICATION

Serotonin syndrome (SS), also referred to as serotonin toxicity, is a potentially life-threatening drug-induced condition associated with increased serotonergic activity in both the peripheral and central nervous systems. It is characterized by a dose-relevant spectrum of clinical findings related to the level of free serotonin (5-hydroxytrypatmine or 5-HT) or 5-HT receptor activation. The predominant 5-HT receptors involved in SS are the 5-HT_{1A} and 5-HT_{2A} subtypes. Some of the main clinical symptoms include neuromuscular abnormalities, autonomic hyperactivity and mental state changes. Severe SS is usually caused by the simultaneous introduction of 2 or more serotonergic drugs, but the syndrome can also occur after the introduction of only one serotonergic drug in susceptible individuals, the addition of one or more therapeutics to a subject on long-term serotonergic drug therapy (such as a monoamine oxidase inhibitor), or after an overdose. SS has been observed across all age groups and is increasing in incidence, likely due to the increasing therapeutic use of serotonergic drugs. Several review articles have been published recently on this syndrome highlighting its significance in current healthcare (see for example: Francescangeli, J. et al., Int. J. Mol. Sci. 2019, 20:2288; Birmes, P. et al., Can. Med. Assoc. J. 2003, 168(11), 1439-1442; Koleva, K. and Nikolov, R., Pharmacia, 2018, 65(1), 64-70; Scotten, W. J. et al., Int. J. Tryptophan Res. 2019, 12, 1-14).

Conjugation of active pharmaceutical agents with fatty acids has been used, for example, to increase half-life, to enhance cellular uptake and retention, for targeted tumor delivery, to reduce chemoresistance in cancer and to improve blood brain barrier (BBB) penetration (Bhat, M. et al., Chem. Phys. Lipids, 2021, 236, 105053). This approach has been applied to many classes of drugs, including for example, non-steroid anti-inflammatory drugs (NSAIDs), angiotensin-converting enzyme inhibitors, angiotensin, nucleosides and testosterone.

It has been reported that fatty acids may enhance the activity of certain antidepressant drugs at low non-antidepressant doses (Laino, C. H. et al., Europ. J. Pharmacol. (2010), 648:117-126; Carlezon, W. A. Jr. et al. Biol. Psychiatry (2005), 57:343-350). Some reports show that omega-3 fatty acids have antidepressant activity when administered on a chronic basis (Lakhwani, L. et al., Acta Poloniae Pharmaceutica—Drug Res. (2007), 64:271-276). Docosahexanaenoic acid (DHA) has been reported to provide a synergistic effect in enhancing the absorption of carotenoids, such as lutein (US patent application no. US2006/0270739). Moreover, DHA has been implicated in augmented brain and cognitive development and maintenance in aging and neuropsychiatric disorders in both humans and animals (Ciappolino, V. et al., Nutrients (2019), 11:769 doi 10.3990/nu11040769; Lauritzen, L. et al., Nutrients (2016), 8:6 doi 10.3390/nu8010006; Weiser, M. J. et al., Nutrients (2016), 8:99 doi 10.3990/nu8020099).

The present applicant has several co-pending applications disclosing novel indole derivatives as serotonergic psychedelic agents for the treatment of various CNS disorders, including PCT patent application publication nos. WO2021/155470, WO2021/155468 and WO2021/155467, and PCT application S.N. PCT/CA2022/-50295 and U.S. provisional application Ser. No. 63/260,470. These applications describe the various pharmaceutical applications for such agents, including in the treatment of mental illnesses and neurological disease, and use in the fields of psychiatry, neurobiology and pharmacotherapy.

### SUMMARY OF THE APPLICATION

Compounds of the present application modulate the activity of serotonin receptors, in particular 5-HT_{2A}, by direct binding to these receptors and/or via the corresponding N-unsubstituted analogs which are produced in vivo via hydrolysis. Accordingly, in some embodiments, compounds of the present application act as prodrugs. However, in contrast to their corresponding N-unsubstituted analogs, compounds of the present application unexpectedly did not induce any significant signs of 5-HT syndrome (e.g. whole body twitches, forepaw treading (FPT), Straub tail) up to 60 mg/kg SC administration to mice.

Accordingly, the present application includes compounds of Formula I:

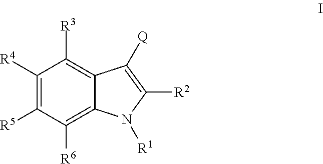

or a pharmaceutically acceptable salt and/or solvate thereof,
wherein:
R¹ is selected from C(O)R⁷, CO₂R⁷, C(O)N(R⁷)(R^{7′}), S(O)R⁷, SO₂R⁷, CO₂C₁₋₄alkyleneOC(O)R⁷, C(O)C₁₋₄alkyleneOC(O)R⁷ and CO₂C₁₋₄alkyleneC(O)R⁷; Q is selected from Q1, Q2, Q2′, Q3, Q4, Q5, and Q6:

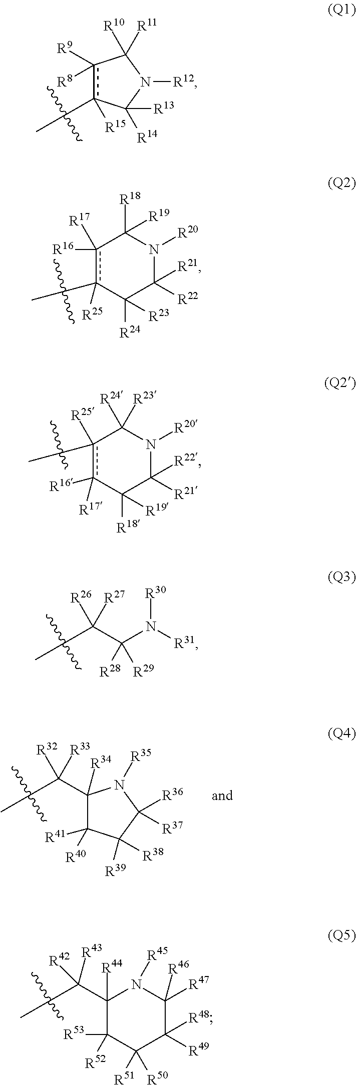

[symbol]

is a single bond or a double bond provided that when [symbol] in Q1 is a double bond then R⁹ and R¹⁵ are not present, when [symbol] in Q2 is a double bond then R¹⁷ and R²⁵ are not present and when [symbol] in Q2′ is a double bond then R^{17′} and R^{25′} are not present; R², R⁵ and R⁶ are independently selected from H, halo, CN, C₁₋₆alkyl, C₂₋₆alkenyl, C₂₋₆alkynyl and C₁₋₆alkoxy;
one of R³ and R⁴ is selected from H, halo, C₁₋₆alkyl, C₂₋₆alkenyl, C₂₋₆alkynyl and C₁₋₆alkoxy and the other of R³ and R⁴ is selected from A, H, halo, C₁₋₆alkyl, C₂₋₆alkenyl, C₂₋₆alkynyl and C₁₋₆alkoxy;
A is selected from OR⁵⁴, OP(O)(OR⁵⁴)(OR⁵⁵), N(R⁵⁴)(R⁵⁵), SR⁵⁴, S(O)R⁵⁴, SO₂R⁵⁴, C(O)R⁵⁴, CO₂R⁵⁴, C(O)N(R⁵⁴)(R⁵⁵), C(NR⁵⁶)R⁵⁴, C(NR⁵⁶)NR⁵⁴R⁵⁵, C(NR⁵⁶)OR⁵⁴, aryl, C₃₋₁₀cycloalkyl, 3- to 10-membered heterocycloalkyl comprising 1 to 4 heteromoeities independently selected from O, S, S(O), SO₂, N and NR⁵⁴ and 5- to 10-membered heteroaryl comprising 1 to 4 heteromoeities independently selected from O, S, S(O), SO₂, N and NR⁵⁴, wherein the C₁₋₆alkyl, C₂₋₆alkenyl, C₂₋₆alkynyl, C₃₋₇cycloalkyl, aryl, 3- to 10-membered heterocycloalkyl and 5- to 10-membered heteroaryl are optionally substituted with one or more substituents independently selected from halo, CN, OR⁵⁷, CO₂R⁵⁷, N(R⁵⁷)(R⁵⁸) and SR⁵⁷, and wherein the C₃₋₁₀cycloalkyl, aryl, 3- to 10-membered heterocycloalkyl and 5- to 10-membered heteroaryl are each further optionally substituted with a substituent selected from CO₂R⁵⁹, C(O)N(R⁵⁹)(R⁶⁰), S(O)R⁵⁹, SO₂R⁵⁹, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁰ and 5- to 6-membered heteroaryl comprising 1 to 2 ring heteromoieties independently selected from O, S, S(O), SO₂, N, and NR⁶⁰;
R⁷ is selected from C₇₋₃₀alkyl, C₇₋₃₀alkenyl and C₇₋₃₀alkynyl, wherein the C₇₋₃₀alkyl, C₇₋₃₀alkenyl and C₇₋₃₀alkynyl, are optionally substituted with one or more substituents independently selected from halo, OR⁶¹, N(R⁶¹)(R⁶²) and SR⁶¹ and/or are optionally interrupted by one to six heteromoieties independently selected from O, C(O), CO₂ and NR⁶³;
R^{7′} is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R^{16′}, R^{17′}, R^{18′}, R^{19′}, R²¹, R^{22′}, R^{23′}, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, halo and C₁₋₆alkyl;
R⁵⁴ is selected from H, C₁₋₆alkyl, C₂₋₆alkenyl, C₂₋₆alkynyl, C₃₋₁₀cycloalkyl, aryl, 3- to 10-membered heterocycloalkyl comprising 1 to 4 heteromoeities independently selected from O, S, S(O), SO₂, N and NR⁶⁴ and 5- to 10-membered heteroaryl comprising 1 to 4 heteromoeities independently selected from O, S, S(O), SO₂, N and NR⁶⁴, wherein said C₁₋₆alkyl, C₂₋₆alkenyl, C₂₋₆alkynyl, C₃₋₁₀cycloalkyl, 3- to 10-membered heterocycloalkyl and 5- to 10-membered heteroaryl are optionally substituted by one or more substituents independently selected from CN, OR⁶⁵, CO₂R⁶⁵, N(R⁶⁵)(R⁶⁶) and SR⁶⁵, and wherein the C₃₋₁₀cycloalkyl, aryl, 3- to 10-membered heterocycloalkyl and 5- to 10-membered heteroaryl are each further optionally substituted with a substituent selected from CO₂R⁶⁷, C(O)N(R⁶⁷)(R⁶⁸), S(O)R⁶⁷, SO₂R⁶⁷, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁹ and 5- to 6-membered heteroaryl comprising 1 to 2 ring heteromoieties independently selected from O, S, S(O), SO₂, N, and NR⁶⁹;
R¹², R²⁰, R^{20′}, R³⁵ and R⁴⁵ are independently selected from H, C₁₋₆alkyl and C(O)C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 8-membered heterocyclic ring which optionally comprises one or two additional heteromoieties independently selected from O, S, S(O), SO₂, N, and NR⁷⁰;
R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H and C₁₋₆alkyl; and wherein all available hydrogen atoms are optionally and independently substituted with a fluorine atom or chlorine atom and all available atoms are optionally substituted with alternate isotope thereof.

In a further embodiment, the compounds of the application are used as medicaments. Accordingly, the application also includes a compound of the application for use as a medicament.

The present application also includes a method of treating psychosis or psychotic symptoms comprising administering a therapeutically effective amount of one or more compounds of the application to a subject in need thereof.

The present application also includes a method of treating a mental illness comprising administering a therapeutically effective amount of one or more compounds of the application to a subject in need thereof.

The present application also includes a method of treating a neurological disease, disorder or condition comprising administering a therapeutically effective amount of one or more compounds of the application to a subject in need thereof.

In some embodiments, in the method of treatment of the application, the compounds of the application treat the specified disease, disorder or condition with a decreased or lower risk of the subject experiencing or having serotonin syndrome.

The application additionally provides a process for the preparation of compounds of the application. General and specific processes are discussed in more detail below and set forth in the examples below.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### BRIEF DESCRIPTION OF THE DRAWINGS

The present application will be described in greater detail with reference to the attached drawings and Tables in which:

FIG. 1 shows results comparing the scores for exemplary compound 1-1 and comparative compound 5-methoxy-dimethoxytryptamine (5-MeO-DMT, Cpd. 32) in the mouse head twitch and 5-HT syndrome assays at doses of up to 60 mg/kg.

FIG. 2 shows results comparing the scores for exemplary compound 1-1 and comparative compound 5-MeO-DMT (Cpd. 32) in the mouse locomotor activity and rearing assay at doses of up to 60 mg/kg.

FIG. 3 shows that the effect of exemplary compound 1-1 in the mouse head twitch assay is completely blocked by the 5-HT_{2A} antagonist M100907 (structure shown).

FIG. 4 shows results comparing the scores for exemplary compound 1-2 and comparative compound 9 (corresponding parent compound for 1-2) in the mouse head twitch and 5-HT syndrome assays at doses of up to 30 mg/kg

FIG. 5 shows results comparing the scores for exemplary compound 1-2 and comparative dimethoxytryptamine (DMT) in the mouse head twitch and 5-HT syndrome assays at doses of up to 30 mg/kg.

FIG. 6 shows results comparing the scores for exemplary compound 1-3 and comparative compound 32 (5-MeO-DMT) in the mouse head twitch and 5-HT syndrome assays at doses of up to 30 mg/kg.

FIG. 7 shows results from drug discrimination assay for exemplary compound 1-2 and comparative DMT.

FIG. 8 shows results from drug discrimination assay for exemplary compound 1-3 and comparative 5-MeO DMT for both sub-cutaneous and oral administration routes.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

The term “compound(s) of the application” or “compound(s) of the present application” and the like as used herein refers to a compound of Formula I and includes pharmaceutically acceptable salts and/or solvates thereof.

The term “composition(s) of the application” or “composition(s) of the present application” and the like as used herein refers to a composition, such a pharmaceutical composition, comprising one or more compounds of the application.

The term “and/or” as used herein means that the listed items are present, or used, individually or in combination. In effect, this term means that “at least one of” or “one or more” of the listed items is used or present. The term “and/or” with respect to pharmaceutically acceptable salts and/or solvates thereof means that the compounds of the application exist as individual salts and solvates, as well as a combination of, for example, a salt of a solvate of a compound of the application.

As used in the present application, the singular forms “a”, “an” and “the” include plural references unless the content clearly dictates otherwise. For example, an embodiment including “a compound” should be understood to present certain embodiments with one compound, or two or more additional compounds.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers and/or steps and also exclude the presence of other unstated features, elements, components, groups, integers and/or steps.

The term “consisting essentially of”, as used herein, is intended to specify the presence of the stated features, elements, components, groups, integers and/or steps as well as those that do not materially affect the basic and novel characteristic(s) of these features, elements, components, groups, integers and/or steps.

In embodiments comprising an “additional” or “second” component, such as an additional or second compound, the second component as used herein is chemically different from the other components or first component. A “third” component is different from the other, first and second components and further enumerated or “additional” components are similarly different.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The present description refers to a number of chemical terms and abbreviations used by those skilled in the art. Nevertheless, definitions of selected terms are provided for clarity and consistency.

The term “solvate” as used herein means a compound, or a salt and/or solvate of a compound, wherein molecules of a suitable solvent are incorporated in the crystal.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or 6 carbon atoms.

As used herein, the term “alkoxy” as used herein, alone or in combination, includes an alkyl group connected to an oxygen-connecting atom.

The term “alkenyl” as used herein, whether it is used alone or as part of another group, means straight or branched chain, unsaturated alkyl groups, containing one or more double bonds. The number of carbon atoms that are possible in the referenced alkenyl group are indicated by the prefix “Cₙ₁₋ₙ₂”. Thus, for example, the term “C₂₋₆alkenyl” means an alkenyl group having 2, 3, 4, 5 or 6 carbon atoms.

The term “alkynyl” as used herein, whether it is used alone or as part of another group, means straight or branched chain, unsaturated alkyl groups, containing one or more triple bonds. The number of carbon atoms that are possible in the referenced alkynyl group are indicated by the prefix “Cₙ₁₋ₙ₂”. Thus, for example, the term “C₂₋₆alkynyl” means an alkenyl group having 2, 3, 4, 5 or 6 carbon atoms.

The term “cycloalkyl,” as used herein, whether it is used alone or as part of another group, means a saturated carbocyclic group containing from 3 to 10 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains either 6 to 10 carbon atoms.

The term “heterocycloalkyl” as used herein, whether it is used alone or as part of another group, refers to cyclic groups containing at least one non-aromatic ring containing from 3 to 10 atoms in which one or more of the atoms are a heteromoiety selected from O, S, S(O), SO₂, N, NH and NC₁₋₆alkyl and the remaining atoms are C. Heterocycloalkyl groups are either saturated or unsaturated (i.e. contain one or more double bonds). When a heterocycloalkyl group contains the prefix Cₙ₁₋ₙ₂ or “n1 to n2” this prefix indicates the number of carbon atoms in the corresponding carbocyclic group, in which one or more, suitably 1 to 4, of the ring atoms is replaced with a heteromoeity as selected from O, S, S(O), SO₂, N, NH and NC₁₋₆alkyl and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-10 atoms in which one or more of the atoms are a heteroatom selected from O, S and N and the remaining atoms are C. When a heteroaryl group contains the prefix Cn1-n₂ this prefix indicates the number of carbon atoms in the corresponding carbocyclic group, in which one or more, suitably 1 to 4, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The term “halogen” (or “halo”) whether it is used alone or as part of another group, refers to a halogen atom and includes fluoro, chloro, bromo and iodo.

The term “haloalkyl” as used herein refers to an alkyl group as defined above in which one or more of the available hydrogen atoms have been replaced with a halogen. Thus, for example, “C₁₋₆haloalkyl” refers to a C, to C₆ linear or branched alkyl group as defined above with one or more halogen substituents.

As used herein, the term “haloalkenyl” refers to an alkenyl group as defined above in which one or more of the available hydrogen atoms have been replaced with a halogen. Thus, for example, “C₁₋₆haloalkenyl” refers to a C, to C₆ linear or branched alkenyl group as defined above with one or more halogen substituents.

The term “deuteroalkyl” as used herein refers to an alkyl group as defined above in which one or more of the available hydrogen atoms have been replaced with a deuterium. Thus, for example, “C₁₋₆deuteroalkyl” refers to a C₁ to C₆ linear or branched alkyl group as defined above with one or more dueterium substituents.

As used herein, the term “deuteroalkenyl” refers to an alkenyl group as defined above in which one or more of the available hydrogen atoms have been replaced with a deuterium. Thus, for example, “C₁₋₆deuteroalkenyl” refers to a C, to C₆ linear or branched alkenyl group as defined above with one or more deuterium substituents.

The term “available”, as in “available hydrogen atoms” or “available atoms” refers to atoms that would be known to a person skilled in the art to be capable of replacement by a substituent.

As used herein, the term “fatty acid” refers to carboxylic acids with either saturated or unsaturated aliphatic chains and are derived from the hydrolysis of fats or oils.

As used herein, the term “one or more” item includes a single item selected from the list as well as mixtures of two or more items selected from the list.

The term “alternate isotope thereof” as used herein refers to an isotope of an element that is other than the isotope that is most abundant in nature.

In the compounds of general Formula I and pharmaceutically acceptable salts and/or solvates thereof, the atoms may exhibit their natural isotopic abundances, or one or more of the atoms may be artificially enriched in a particular isotope having the same atomic number, but an atomic mass or mass number different from the atomic mass or mass number predominantly found in nature. The present disclosure is meant to include all suitable isotopic variations of the compounds of general Formula I and pharmaceutically acceptable salts and/or solvates thereof. For example, different isotopic forms of hydrogen (H) include protium (¹H), deuterium (²H) and tritium (³H). Protium is the predominant hydrogen isotope found in nature.

The term “compound” refers to the compound and, in certain embodiments, to the extent they are stable, any hydrate or solvate thereof. A hydrate is the compound complexed with water and a solvate is the compound complexed with a solvent, which may be an organic solvent or an inorganic solvent. A “stable” compound is a compound that can be prepared and isolated and whose structure and properties remain or can be caused to remain essentially unchanged for a period of time sufficient to allow use of the compound for the purposes described herein (e.g., therapeutic administration to a subject). The compounds of the present application are limited to stable compounds embraced by general Formula I, or pharmaceutically acceptable salts and/or solvates thereof.

The term “pharmaceutically acceptable” means compatible with the treatment of subjects.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with, the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

The term “subject” as used herein includes all members of the animal kingdom including mammals, and suitably refers to humans. Thus the methods of the present application are applicable to both human therapy and veterinary applications.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early neurological disease can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alliteratively comprise a series of administrations.

As used herein, the term “effective amount” or “therapeutically effective amount” means an amount of one or more compounds of the application that is effective, at dosages and for periods of time necessary to achieve the desired result. For example, in the context of treating a disease, disorder or condition mediated or treated by agonism or activation of serotonergic receptors and downstream second messengers, an effective amount is an amount that, for example, increases said activation compared to the activation without administration of the one or more compounds.

“Palliating” a disease, disorder or condition means that the extent and/or undesirable clinical manifestations of a disease, disorder or condition are lessened and/or time course of the progression is slowed or lengthened, as compared to not treating the disorder.

The term “administered” as used herein means administration of a therapeutically effective amount of one or more compounds or compositions of the application to a cell, tissue, organ or subject.

The term “prevention” or “prophylaxis”, or synonym thereto, as used herein refers to a reduction in the risk or probability of a patient becoming afflicted with a disease, disorder or condition or manifesting a symptom associated with a disease, disorder or condition.

The “disease, disorder or condition” as used herein refers to a disease, disorder or condition treated or treatable by activation a serotonin receptor, for example 5-HT_{2A} and particularly using a serotonin receptor agonist, such as one or more compounds of the application herein described. The disease, disorder or condition may also be treated or treatable via alternative mechanisms, for example by modulation, deactivation, antagonism or reverse agonism of a serotonin receptor, including 5-HT_{2A} and/or 5-HT_{1A}.

The term “treating a disease, disorder or condition by activation of a serotonin receptor” as used herein means that the disease, disorder or condition to be treated is affected by, modulated by and/or has some biological basis, either direct or indirect, that includes serotonergic activity, in particular increases in serotonergic activity. These diseases respond favourably when serotonergic activity associated with the disease, disorder or condition is agonized by one or more of the compounds or compositions of the application.

The term “activation” as used herein includes agonism, partial agonism and positive allosteric modulation of a serotonin receptor.

The terms “5-HT_{1A}” and “5-HT_{2A}” are used herein mean the 5-HT_{1A} and 5-HT_{2A} receptor subtypes of the 5-HT₂ serotonin receptor, respectively.

The term “therapeutic agent” as used herein refers to any drug or active agent that has a pharmacological effect when administered to a subject.

### II. Compounds

The present application includes compounds of Formula I:

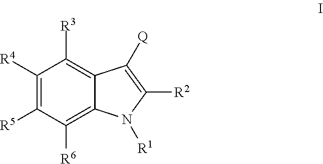

or a pharmaceutically acceptable salt and/or solvate thereof,
wherein:
R¹ is selected from C(O)R⁷, CO₂R⁷, C(O)N(R⁷)(R^{7′}), S(O)R⁷, SO₂R⁷, CO₂C₁₋₄alkyleneOC(O)R⁷, C(O)C₁₋₄alkyleneOC(O)R⁷ and CO₂C₁₋₄alkyleneC(O)R⁷;
Q is selected from Q1, Q2, Q2′, Q3, Q4, Q5, and Q6:

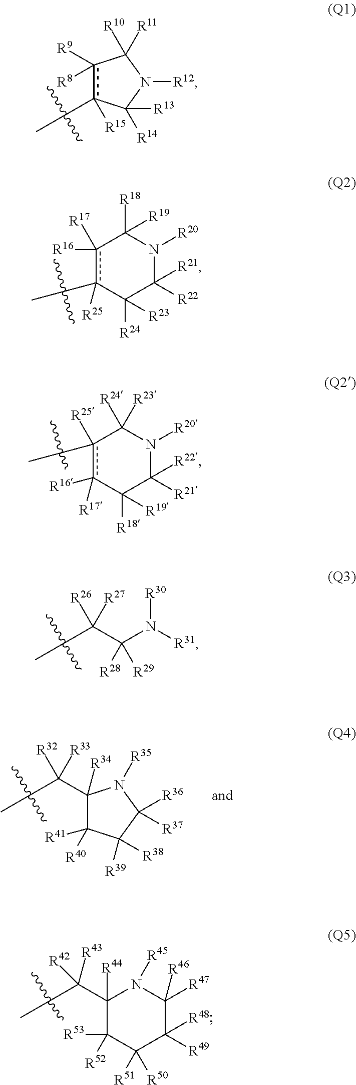

[symbol]

is a single bond or a double bond provided that when [symbol] in Q1 is a double bond then R⁹ and R¹⁵ are not present, when [symbol] in Q2 is a double bond then R¹⁷ and R²⁵ are not present and when [symbol] in Q2′ is a double bond then R^{17′} and R^{25′} are not present;
R², R⁵ and R⁶ are independently selected from H, halo, CN, C₁₋₆alkyl, C₂₋₆alkenyl, C₂₋₆alkynyl and C₁₋₆alkoxy;
one of R³ and R⁴ is selected from H, halo, C₁₋₆alkyl, C₂₋₆alkenyl, C₂₋₆alkynyl and C₁₋₆alkoxy and the other of R³ and R⁴ is selected from A, H, halo, C₁₋₆alkyl, C₂₋₆alkenyl, C₂₋₆alkynyl and C₁₋₆alkoxy;
A is selected from OR⁵⁴, OP(O)(OR⁵⁴)(OR⁵⁵), N(R⁵⁴)(R⁵⁵), SR⁵⁴, S(O)R⁵⁴, SO₂R⁵⁴, C(O)R⁵⁴, CO₂R⁵⁴, C(O)N(R⁵⁴)(R⁵⁵), C(NR⁵⁶)R⁵⁴, C(NR⁵⁶)NR⁵⁴R⁵⁵, C(NR⁵⁶)OR⁵⁴, aryl, C₃₋₁₀cycloalkyl, 3- to 10-membered heterocycloalkyl comprising 1 to 4 heteromoeities independently selected from O, S, S(O), SO₂, N and NR⁵⁴ and 5- to 10-membered heteroaryl comprising 1 to 4 heteromoeities independently selected from O, S, S(O), SO₂, N and NR⁵⁴, wherein the C₁₋₆alkyl, C₂₋₆alkenyl, C₂₋₆alkynyl, C₃₋₇cycloalkyl, aryl, 3- to 10-membered heterocycloalkyl and 5- to 10-membered heteroaryl are optionally substituted with one or more substituents independently selected from halo, CN, OR⁵⁷, CO₂R⁵⁷, N(R⁵⁷)(R⁵⁸) and SR⁵⁷, and wherein the C₃₋₁₀cycloalkyl, aryl, 3- to 10-membered heterocycloalkyl and 5- to 10-membered heteroaryl are each further optionally substituted with a substituent selected from CO₂R⁵⁹, C(O)N(R⁵⁹)(R⁶⁰), S(O)R⁵⁹, SO₂R⁵⁹, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁰ and 5- to 6-membered heteroaryl comprising 1 to 2 ring heteromoieties independently selected from O, S, S(O), SO₂, N, and NR⁶⁰;
R⁷ is selected from C₇₋₃₀alkyl, C₇₋₃₀alkenyl and C₇₋₃₀alkynyl, wherein the C₇₋₃₀alkyl, C₇₋₃₀alkenyl and C₇₋₃₀alkynyl, are optionally substituted with one or more substituents independently selected from halo, OR⁶¹, N(R⁶¹)(R⁶²) and SR⁶¹ and/or are optionally interrupted by one to six heteromoieties independently selected from O, C(O), CO₂ and NR⁶³;
R^{7′} is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R^{16′}, R^{17′}, R^{18′}, R^{19′}, R^{21′} R²², R^{23′}, R^{24′}, R^{25′}, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, halo and C₁₋₆alkyl;
R⁵⁴ is selected from H, C₁₋₆alkyl, C₂₋₆alkenyl, C₂₋₆alkynyl, C₃₋₁₀cycloalkyl, aryl, 3- to 10-membered heterocycloalkyl comprising 1 to 4 heteromoeities independently selected from O, S, S(O), SO₂, N and NR⁶⁴ and 5- to 10-membered heteroaryl comprising 1 to 4 heteromoeities independently selected from O, S, S(O), SO₂, N and NR⁶⁴, wherein said C₁₋₆alkyl, C₂₋₆alkenyl, C₂₋₆alkynyl, C₃₋₁₀cycloalkyl, 3- to 10-membered heterocycloalkyl and 5- to 10-membered heteroaryl are optionally substituted by one or more substituents independently selected from CN, OR⁶⁵, CO₂R⁶⁵, N(R⁶⁵)(R⁶⁶) and SR⁶⁵, and wherein the C₃₋₁₀cycloalkyl, aryl, 3- to 10-membered heterocycloalkyl and 5- to 10-membered heteroaryl are each further optionally substituted with a substituent selected from CO₂R⁶⁷, C(O)N(R⁶⁷)(R⁶⁸), S(O)R⁶⁷, SO₂R⁶⁷, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁹ and 5- to 6-membered heteroaryl comprising 1 to 2 ring heteromoieties independently selected from O, S, S(O), SO₂, N, and NR⁶⁹;
R¹², R²⁰, R^{20′}, R³⁵ and R⁴⁵ are independently selected from H, C₁₋₆alkyl and C(O)C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 8-membered heterocyclic ring which optionally comprises one or two additional heteromoieties independently selected from O, S, S(O), SO₂, N, and NR⁷⁰;
R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H and C₁₋₆alkyl; and
wherein all available hydrogen atoms are optionally and independently substituted with a fluorine atom or chlorine atom and all available atoms are optionally substituted with alternate isotope thereof.

The present application also includes compounds of Formula I:

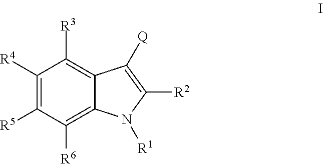

or a pharmaceutically acceptable salt and/or solvate thereof,
wherein:
R¹ is selected from C(O)R⁷, CO₂R, C(O)N(R⁷)(R^{7′}), S(O)R⁷ and SO₂R⁷;
Q is selected from Q1, Q2, Q3, Q4 and Q5:

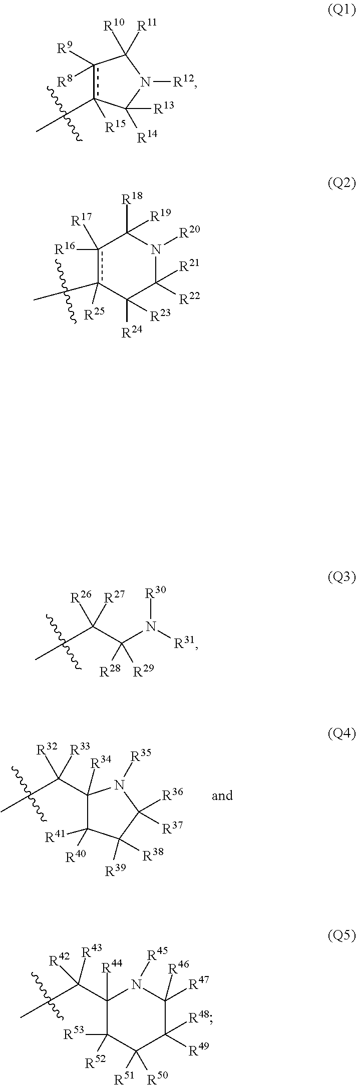

[symbol]

is a single bond or a double bond provided that when [symbol] in Q1 is a double bond then R⁹ and R¹⁵ are not present, and when [symbol] in Q2 is a double bond then R¹⁷ and R²⁵ are not present;
R², R⁵ and R⁶ are independently selected from H, halo, CN, C₁₋₆alkyl, C₂₋₆alkenyl, C₂₋₆alkynyl and C₁₋₆alkoxy;
one of R³ and R⁴ is selected from H, halo, C₁₋₆alkyl, C₂₋₆alkenyl, C₂₋₆alkynyl and C₁₋₆alkoxy and the other of R³ and R⁴ is selected from A, H, halo, C₁₋₆alkyl, C₂₋₆alkenyl, C₂₋₆alkynyl and C₁₋₆alkoxy;
A is selected from OR⁵⁴, OP(O)(OR⁵⁴)(OR⁵⁵), N(R⁵⁴)(R⁵⁵), SR⁵⁴, S(O)R⁵⁴, SO₂R⁵⁴, C(O)R⁵⁴, CO₂R⁵⁴, C(O)N(R⁵⁴)(R⁵⁵), C(NR⁵⁶)R⁵⁴, C(NR⁵⁶)NR⁵⁴R⁵⁵, C(NR⁵⁶)OR⁵⁴, aryl, C₃₋₁₀cycloalkyl, 3- to 10-membered heterocycloalkyl comprising 1 to 4 heteromoeities independently selected from O, S, S(O), SO₂, N and NR⁵⁴ and 5- to 10-membered heteroaryl comprising 1 to 4 heteromoeities independently selected from O, S, S(O), SO₂, N and NR⁵⁴, wherein the C₁₋₆alkyl, C₂₋₆alkenyl, C₂₋₆alkynyl, C₃₋₇cycloalkyl, aryl, 3- to 10-membered heterocycloalkyl and 5- to 10-membered heteroaryl are optionally substituted with one or more substituents independently selected from halo, CN, OR⁵⁷, CO₂R⁵⁷, N(R⁵⁷)(R⁵⁸) and SR⁵⁷, and wherein the C₃₋₁₀cycloalkyl, aryl, 3- to 10-membered heterocycloalkyl and 5- to 10-membered heteroaryl are each further optionally substituted with a substituent selected from CO₂R⁵⁹, C(O)N(R⁵⁹)(R⁶⁰), S(O)R⁵⁹, SO₂R⁵⁹, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁰ and 5- to 6-membered heteroaryl comprising 1 to 2 ring heteromoieties independently selected from O, S, S(O), SO₂, N, and NR⁶⁰;
R⁷ is selected from C₇₋₃₀alkyl, C₇₋₃₀alkenyl and C₇₋₃₀alkynyl, wherein the C₇₋₃₀alkyl, C₇₋₃₀alkenyl and C₇₋₃₀alkynyl, are optionally substituted with one or more substituents independently selected from halo, OR⁶¹, N(R⁶¹)(R⁶²) and SR⁶¹ and/or are optionally interrupted by one to six heteromoieties independently selected from O, C(O), CO₂ and NR⁶³;
R^{7′} is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, halo and C₁₋₆alkyl;
R⁵⁴ is selected from H, C₁₋₆alkyl, C₂₋₆alkenyl, C₂₋₆alkynyl, C₃₋₁₀cycloalkyl, aryl, 3- to 10-membered heterocycloalkyl comprising 1 to 4 heteromoeities independently selected from O, S, S(O), SO₂, N and NR⁶⁴ and 5- to 10-membered heteroaryl comprising 1 to 4 heteromoeities independently selected from O, S, S(O), SO₂, N and NR⁶⁴, wherein said C₁₋₆alkyl, C₂₋₆alkenyl, C₂₋₆alkynyl, C₃₋₁₀cycloalkyl, 3- to 10-membered heterocycloalkyl and 5- to 10-membered heteroaryl are optionally substituted by one or more substituents independently selected from CN, OR⁶⁵, CO₂R⁶⁵, N(R⁶⁵)(R⁶⁶) and SR⁶⁵, and wherein the C₃₋₁₀cycloalkyl, aryl, 3- to 10-membered heterocycloalkyl and 5- to 10-membered heteroaryl are each further optionally substituted with a substituent selected from CO₂R⁶⁷, C(O)N(R⁶⁷)(R⁶⁸), S(O)R⁶⁷, SO₂R⁶⁷, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁹ and 5- to 6-membered heteroaryl comprising 1 to 2 ring heteromoieties independently selected from O, S, S(O), SO₂, N, and NR⁶⁹;
R¹², R²⁰, R³⁵ and R⁴⁵ are independently selected from H, C₁₋₆alkyl and C(O)C₁₋₆alkyl;
R³⁰ and R³¹ are independently selected from H, C₁₋₆alkyl and C(O)C₁₋₆alkyl, or
R³⁰ and R³¹, together with the N atom to which they are bound, form a 3- to 8-membered heterocyclic ring which optionally comprises one or two additional heteromoieties independently selected from O, S, S(O), SO₂, N, and NR⁷⁰;
R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H and C₁₋₆alkyl; and
wherein all available hydrogen atoms are optionally and independently substituted with a fluorine atom or chlorine atom and all available atoms are optionally substituted with alternate isotope thereof.

In some embodiments, all available hydrogen atoms are optionally replaced with F.

In some embodiments, Q is Q1,

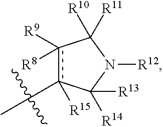

and [symbol] is a single bond and the compound of Formula I has the following structure:

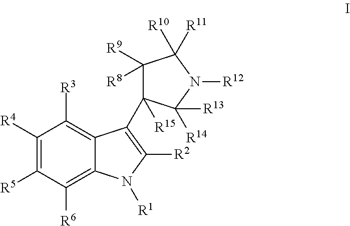

or a pharmaceutically acceptable salt and/or solvate thereof,
wherein:
R¹, R², R³, R⁴, R⁵, R⁶, R⁸, R⁹, R¹⁰, R¹¹, R¹², R¹³, R¹⁴ and R¹⁵ are as defined for Formula 1.

In some embodiments, Q is Q1,

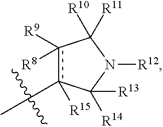

and [symbol] is a double bond and the compound of Formula I has the following structure:

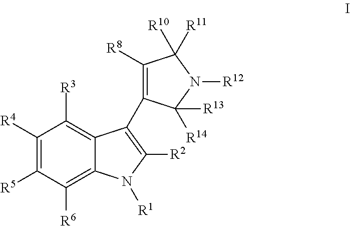

or a pharmaceutically acceptable salt and/or solvate thereof,
wherein:
R¹, R², R³, R⁴, R⁵, R⁶, R⁸, R¹⁰, R¹¹, R¹², R¹³ and R¹⁴ are as defined for Formula 1.

In some embodiments, Q is Q2,

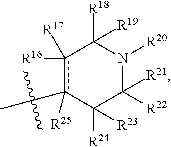

and [symbol] is a single bond and the compound of Formula I has the following structure:

[symbol]

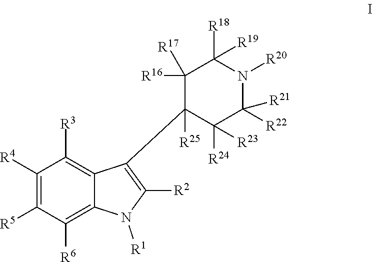

or a pharmaceutically acceptable salt and/or solvate thereof,
wherein:
R¹, R², R³, R⁴, R⁵, R⁶, R¹⁶, R¹⁷, R¹⁸, R¹⁹, R²⁰, R²¹, R²², R²³, R²⁴ and R²⁵ are as defined for Formula 1.

In some embodiments, Q is Q2,

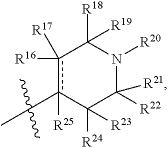

and [symbol] is a double bond and the compound of Formula I has the following structure:

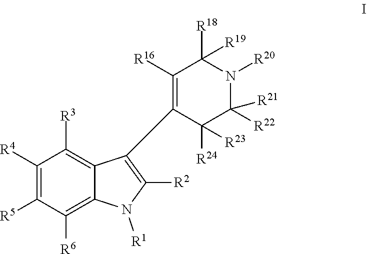

or a pharmaceutically acceptable salt and/or solvate thereof,
wherein:
R¹, R², R³, R⁴, R⁵, R⁶, R¹⁶, R¹⁸, R¹⁹, R²⁰, R²¹, R²², R²³ and R²⁴ are as defined for Formula I.

In some embodiments, Q is Q2′,

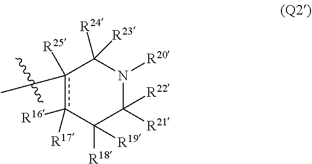

and [symbol] is a single bond and the compound of Formula I has the following structure:

[symbol]

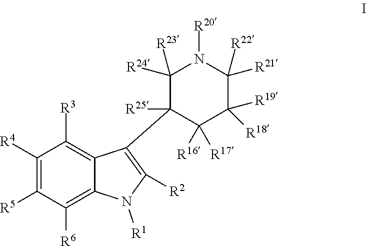

or a pharmaceutically acceptable salt and/or solvate thereof,
wherein:
R¹, R², R³, R⁴, R⁵, R⁶, R¹⁶, R¹⁷, R¹⁸, R¹⁹, R²⁰, R²¹, R²², R²³, R^{24′} and R^{25′} are as defined for Formula I.

In some embodiments, Q is Q2′,

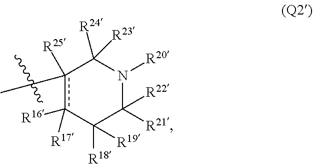

and [symbol] is a double bond and the compound of Formula I has the following structure:

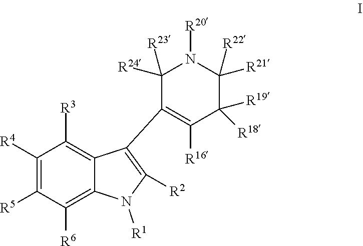

or a pharmaceutically acceptable salt and/or solvate thereof,
wherein:
R¹, R², R³, R⁴, R⁵, R⁶, R^{16′}, R^{18′}, R^{19′}, R^{20′}, R^{21′}, R^{22′}, R^{23′} and R^{24′} are as defined for Formula I.

In some embodiments, Q is Q3,

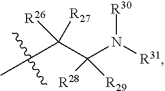

and the compound of Formula I has the following structure:

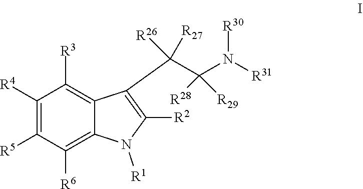

or a pharmaceutically acceptable salt and/or solvate thereof,
wherein:
R¹, R², R³, R⁴, R⁵, R⁶, R²⁶, R²⁷, R²⁸, R²⁹, R³⁰ and R³¹ are as defined for Formula I.

In some embodiments, Q is Q4,

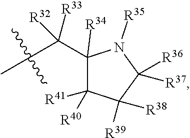

and the compound of Formula I has the following structure:

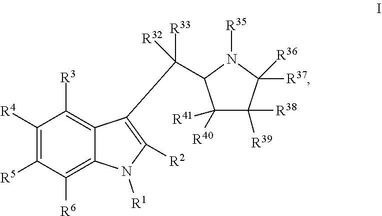

or a pharmaceutically acceptable salt and/or solvate thereof,
wherein:
R¹, R², R³, R⁴, R⁵, R⁶, R³², R³³, R³⁴, R³⁵, R³⁶, R³⁷, R³⁸, R³⁹, R⁴⁰ and R⁴¹ are as defined for Formula I.

In some embodiments, Q is Q5

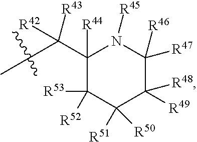

and the compound of Formula I has the following structure:

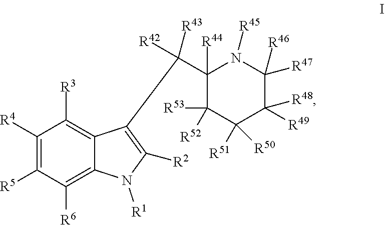

or a pharmaceutically acceptable salt and/or solvate thereof,
wherein:
R¹, R², R³, R⁴, R⁵, R⁶, R⁴², R⁴³, R⁴⁴, R⁴⁵, R⁴⁶, R⁴⁷, R⁴⁸, R⁴⁹, R⁵⁰, R⁵¹, R⁵² and R⁵³ are as defined for Formula I.

In some embodiments, R², R³, R⁵ and R⁶ are all H and R⁴ is A and the compound Formula I has the following structure:

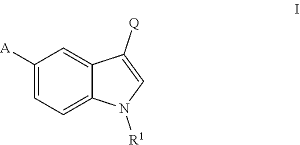

or a pharmaceutically acceptable salt and/or solvate thereof,
wherein:
A, R¹ and Q are defined for Formula 1.

In some embodiments, R², R⁴, R⁵ and R⁶ are all H and R³ is A and the compound Formula I has the following structure:

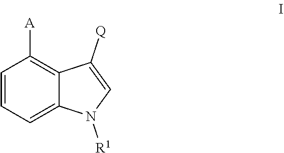

or a pharmaceutically acceptable salt and/or solvate thereof,
wherein:
A, R¹ and Q are defined for Formula 1.

In some embodiments, R², R³, R⁵ and R⁶ are all D and R⁴ is A and the compound of Formula I has the following structure:

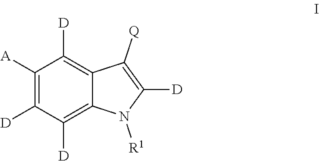

or a pharmaceutically acceptable salt and/or solvate thereof,
wherein:
A, Q and R¹ are as defined for Formula 1.

In some embodiments, R², R⁴, R⁵ and R⁶ are all D and R³ is A and the compound of Formula I has the following structure:

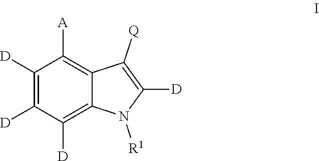

or a pharmaceutically acceptable salt and/or solvate thereof,
wherein:
A, Q and R¹ are as defined for Formula 1.

In some embodiments, R², R³, R⁴, R⁵ and R⁶ are all H and the compound Formula I has the following structure:

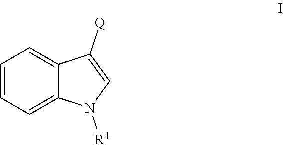

or a pharmaceutically acceptable salt and/or solvate thereof,
wherein:
A, R¹ and Q are defined for Formula 1.

In some embodiments, R², R³, R⁴, R⁵ and R⁶ are all D and the compound of Formula I has the following structure:

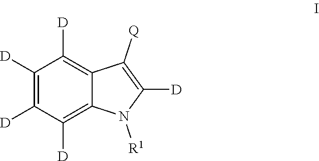

or a pharmaceutically acceptable salt and/or solvate thereof,
wherein:
A, Q and R¹ are as defined for Formula I.

In some embodiments Q is selected from one of the following groups:

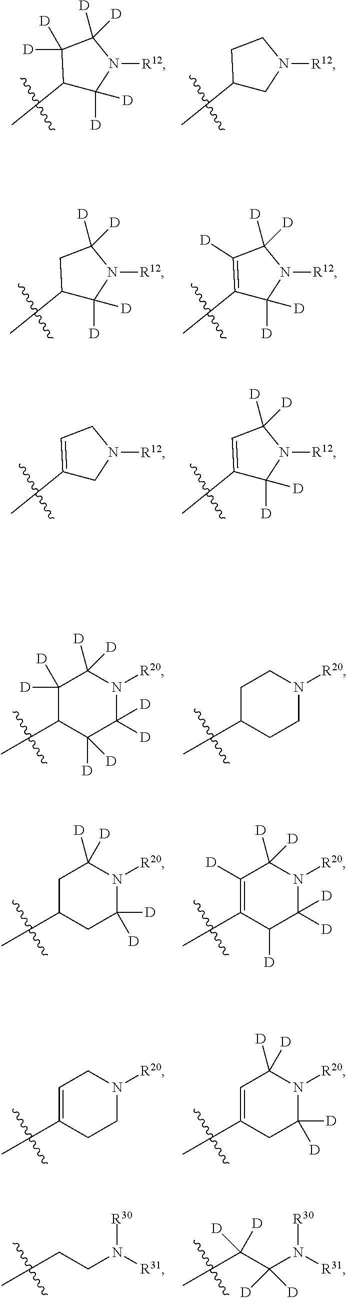

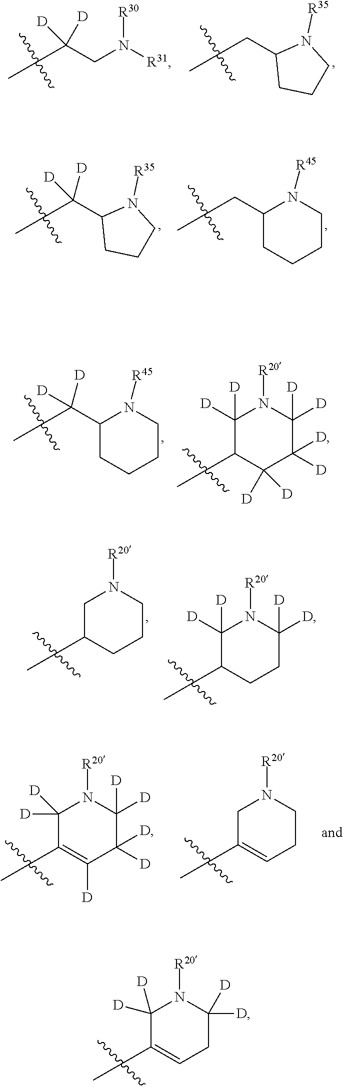

wherein R¹², R²⁰, R²⁰, R³⁰, R³¹, R³⁵ and R⁴⁵ are independently selected from H, D, C₁₋₆alkyl, C₁₋₆fluoroalkyl and C₁₋₆deuteroalkyl, or R³⁰ and R³¹, together with the N atom to which they are bound, form a 3- to 6-membered heterocyclic ring which optionally comprises one or two additional heteromoieties independently selected from O, N and NR⁷⁰. In some embodiments, R¹², R²⁰, R²⁰, R³⁰, R³¹, R³⁵ and R⁴⁵ are independently selected from H, C₁₋₄alkyl, C₁₋₄fluoroalkyl and C₁₋₄deuteroalkyl. In some embodiments, R¹², R²⁰, R²⁰, R³⁰, R³¹, R³⁵ and R⁴⁵ are independently selected from H, CH₃, CD₃, CF₂H and CF₃. In some embodiments, R³⁰ and R³¹, together with the N atom to which they are bound, form a 5- or 6-membered heterocyclic ring which optionally comprises one additional heteromoiety selected from O and NR⁷⁰.

In some embodiments Q is selected from one of the following groups:

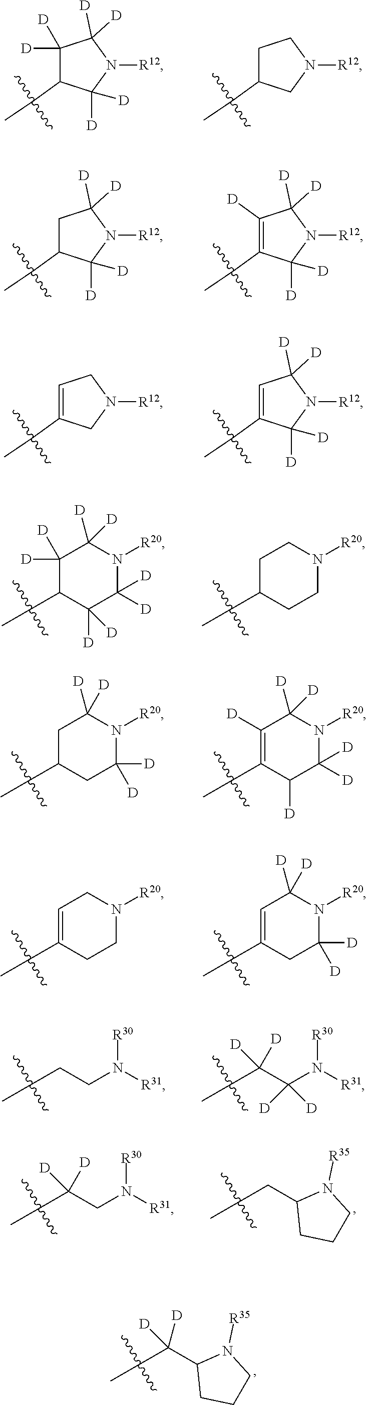

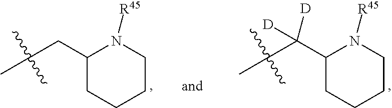

wherein R¹², R²⁰, R³⁰, R³¹, R³⁵ and R⁴⁵ are independently selected from H, D, C₁₋₆alkyl, C₁₋₆fluoroalkyl and C₁₋₆deuteroalkyl. In some embodiments, R¹², R²⁰, R³⁰, R³¹, R³⁵ and R⁴⁵ are independently selected from H, C₁₋₄alkyl, C₁₋₄fluoroalkyl and C₁₋₄deuteroalkyl. In some embodiments, R¹², R²⁰, R³⁰, R³¹, R³⁵ and R⁴⁵ are independently selected from H, CH₃, CD₃, CF₂H and CF₃.

In some embodiments, when Q is

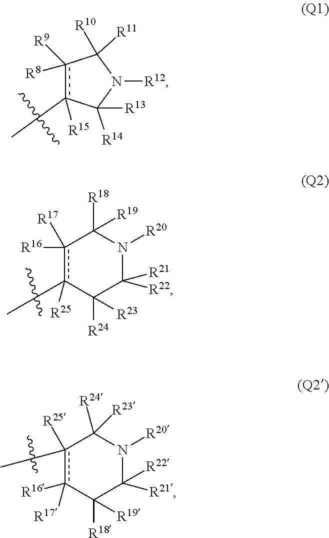

and [symbol] is a single bond, the stereochemistry at the carbon to which R¹⁵, R²⁵ or R^{25′}, respectively, is bonded is either R or S. Therefore in some embodiments, Q1 is

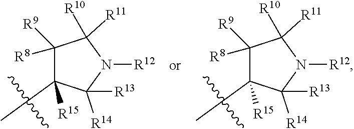

### Q2 is

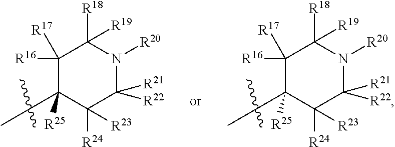

### and Q2′ is

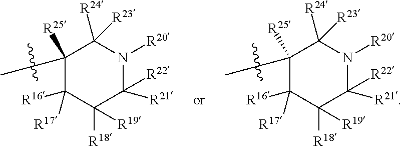

In some embodiments, when Q is

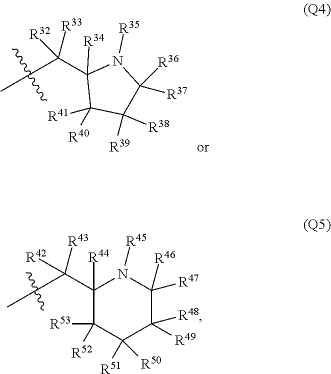

the stereochemistry at the carbon to which R³⁴ or R⁴⁵, respectively, is bonded is either R or S. Therefore, in some embodiments Q4 is

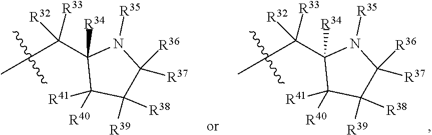

### and Q5 is

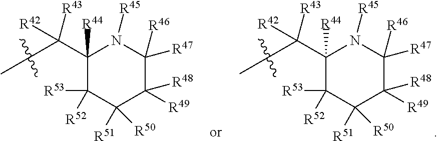

In some embodiment, R³⁰ and R³¹, together with the N atom to which they are bound, form a 3- to 6-membered heterocyclic ring which optionally comprises one or two additional heteromoieties independently selected from O, N and NR⁷⁰.

In some embodiments, R², R⁵ and R⁶ are independently selected from H, D, Cl, F, C₁₋₄alkyl and C₁₋₄deuteroalkyl. In some embodiments, R², R⁵ and R⁶ are independently selected from H, D, Cl, F, C₁₋₂alkyl and C₁₋₂deuteroalkyl. In some embodiments, R², R⁵ and R⁶ are independently selected from H, D, F, CH₃ and CD₃. In some embodiments, R², R⁵ and R⁶ are independently selected from H and D. In some embodiments, at least one of R², R⁵ and R⁶ is D. In some embodiments, all of R², R⁵ and R⁶ are D. In some embodiments, all of R², R⁵ and R⁶ are H. In some embodiments, R², R⁵ and R⁶ are independently selected from H, D, F, CH₃, CD₃

In some embodiments, one of R³ and R⁴ is selected from H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy and the other of R³ and R⁴ is selected from A, H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, one of R³ and R⁴ is selected from H, D, F, Cl, CH₃, CF₃, CF₂H, CD₃, CH₃O, CF₃O, CHF₂O, and CD₃O and the other of R³ and R⁴ is selected from A H, D, F, Cl, CH₃, CF₃, CF₂H, CD₃, CH₃O, CF₃O, CHF₂O, and CD₃O. In some embodiments, both R³ and R⁴ are H. In some embodiments, both R³ and R⁴ are D. In some embodiments R³ is A and R⁴ is H or D. In some embodiments R³ is H or D and R⁴ is A.

In some embodiments, one or both of R³ and R⁴ is independently selected from H, D, F, C, CH₃, CH₂CH₃, CH₂CH₂CH₃, CH(CH₃)₂, CF₃, CF₂H, CH₂CF₂H, CH₂CF₃, CH₂CFH₂, CH(CF₃)₂, CD₃, CH(CH₃)₂O, CH₃CH₂CH₂O, CH₃CH₂O, CH₃O, CF₃O, CHF₂O, CF₂HCH₂O, CF₃CH₂O, (CF₃)₂CHO, and CD₃O. In some embodiments, one or both of R³ and R⁴ is independently selected from H, D, F, C, CH₃, CH(CH₃)₂, CF₃, CF₂H, CD₃, CH(CH₃)₂O, CH₃O, CF₃O, CHF₂O, and CD₃O.

In some embodiments, both of R³ and R⁴ are independently selected from D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments both of R³ and R⁴ are independently selected from D, F, C, CH₃, CH(CH₃)₂, CF₃, CF₂H, CD₃, CH(CH₃)₂O, CH₃O, CF₃O, CHF₂O, and CD₃O.

In some embodiments, R³ is H or D and R⁴ is selected from H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R³ is H or D and R⁴ is selected from D, F, Cl, CH₃, CH(CH₃)₂, CF₃, CF₂H, CD₃, CH(CH₃)₂O, CH₃O, CF₃O, CHF₂O, and CD₃O.

In some embodiments, R⁴ is H or D and R³ is selected from H, D, F, Cl, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy. In some embodiments, R⁴ is H or D and R³ is selected from D, F, Cl, CH₃, CH(CH₃)₂, CF₃, CF₂H, CD₃, CH(CH₃)₂O, CH₃O, CF₃O, CHF₂O, and CD₃O.

In some embodiments, A is selected from OR⁵⁴, OP(O)(OR⁵⁴)(OR⁵⁵), N(R⁵⁴)(R⁵⁵), SR⁵⁴, S(O)R⁵⁴, SO₂R⁵⁴, C(O)R⁵⁴, CO₂R⁵⁴, C(O)N(R⁵⁴)(R⁵⁵), aryl, C₃₋₁₀cycloalkyl, 3- to 6-membered heterocycloalkyl comprising 1 to 3 heteromoeities independently selected from O, S, S(O), SO₂, N and NR⁵⁴ and 5- to 6-membered heteroaryl comprising 1 to 3 heteromoeities independently selected from O, S, S(O), SO₂, N and NR⁵⁴, wherein the C₃₋₇cycloalkyl, aryl, 3- to 6-membered heterocycloalkyl and 5- to 6-membered heteroaryl are optionally substituted with one or two substituents independently selected from Cl, F, CN, OR⁵⁷, CO₂R⁵⁷, N(R⁵⁷)(R⁵⁸) and SR⁵⁷, and wherein the C₃₋₁₀cycloalkyl, aryl, 3- to 6-membered heterocycloalkyl and 5- to 6-membered heteroaryl are each further optionally substituted with a substituent selected from CO₂R⁵⁹, C(O)N(R⁵⁹)(R⁶⁰), S(O)R⁵⁹, SO₂R⁵⁹, C₁₋₆alkyl, C₃₋₆cycloalkyl, phenyl, 3- to 6-membered heterocycloalkyl comprising 1 to 2 ring heteromoieties independently selected from O, S, S(O), SO₂, N, and NR⁶⁰ and 5- to 6-membered heteroaryl comprising 1 to 2 ring heteromoieties independently selected from O, S, S(O), SO₂, N, and NR⁶⁰, wherein all available hydrogen atoms are optionally and independently substituted with a fluorine atom and all available atoms are optionally substituted with alternate isotope thereof.

In some embodiments, A is selected from OR⁵⁴, OP(O)(OR⁵⁴)(OR⁵⁵), N(R⁵⁴)(R⁵⁵), C(O)R⁵⁴, CO₂R⁵⁴ and C(O)N(R⁵⁴)(R⁵⁵), wherein R⁵⁴ is selected from H, C₁₋₆alkyl, C₁₋₆fluoroalkyl, C₁₋₆deuteroalkyl, C₃₋₁₀cycloalkyl, aryl, 3- to 6-membered heterocycloalkyl comprising 1 to 3 heteromoeities independently selected from O, N and NR⁶⁴ and 5- to 6-membered heteroaryl comprising 1 to 3 heteromoeities independently selected from O, N and NR⁶⁴, wherein said C₁₋₆alkyl, C₁₋₆fluoroalkyl, C₁₋₆deuteroalkyl, C₃₋₆cycloalkyl, 3- to 6-membered heterocycloalkyl and 5- to 6-membered heteroaryl are optionally substituted by one or two substituents independently selected from CN, OR⁶⁵, CO₂R⁶⁵ and N(R⁶⁵)(R⁶⁶), and wherein the C₃₋₆cycloalkyl, aryl, 3- to 6-membered heterocycloalkyl and 5- to 6-membered heteroaryl are each further optionally substituted with a substituent selected from CO₂R⁶⁷, C(O)N(R⁶⁷)(R⁶⁸), C₁₋₆alkyl, C₁₋₆fluoroalkyl, C₁₋₆deuteroalkyl, C₃₋₆cycloalkyl, phenyl, 3- to 6-membered heterocycloalkyl comprising 1 to 2 ring heteromoieties independently selected from 0, N, and NR⁶⁹ and 5- to 6-membered heteroaryl comprising 1 to 2 ring heteromoieties independently selected from O, N and NR⁶⁹; and R⁵⁵ is selected from H, C₁₋₆alkyl, C₁₋₆fluoroalkyl and C₁₋₆deuteroalkyl.

In some embodiments, A is selected from OR⁵⁴, OP(O)(OR⁵⁴)(OR⁵⁵), N(R⁵⁴)(R⁵⁵), C(O)R⁵⁴, CO₂R⁵⁴ and C(O)N(R⁵⁴)(R⁵⁵), wherein R⁵⁴ and R⁵⁵ are independently selected from H, C₁₋₆alkyl, C₁₋₆fluoroalkyl and C₁₋₆deuteroalkyl. In some embodiments, A is selected from OR⁵⁴ and OP(O)(OR⁵⁴)(OR⁵⁵), wherein R⁵⁴ and R⁵⁵ are independently selected from H, CH₃, CF₃, CF₂H and CD₃.

In some embodiments, R¹ is selected from C(O)R⁷, CO₂R⁷ and C(O)N(R⁷)(R⁷). In some embodiments, R¹ is selected from C(O)R⁷ and CO₂R⁷.

In some embodiments, R¹ is selected from CO₂C₁₋₄alkyleneOC(O)R⁷, C(O)C₁₋₄alkyleneOC(O)R⁷ and CO₂C₁₋₄alkyleneC(O)R⁷. In some embodiments, R1 is selected from CO₂C₁₋₃alkyleneOC(O)R⁷, C(O)C₁₋₃alkyleneOC(O)R⁷ and CO₂C₁₋₃alkyleneC(O)R⁷. In some embodiments, R¹ is selected from CO₂CH₂OC(O)R⁷, CO₂CH(CH₃)OC(O)R⁷, C(O)CH₂C(O)R⁷, C(O)CH(CH₃)C(O)R⁷, CO₂CH₂C(O)R⁷ and CO₂CH(CH₃)C(O)R⁷. In some embodiments, R¹ is selected from CO₂CH₂OC(O)R⁷ and CO₂CH(CH₃)OC(O)R⁷.

In some embodiments, R⁷ is selected from C₇₋₃₀alkyl and C₇₋₃₀alkenyl, wherein the C₇₋₃₀alkyl and C₇₋₃₀alkenyl are optionally substituted with one or more substituents independently selected from D, Cl, F and OR⁶¹, and/or are optionally interrupted by one to four O and/or CO₂. In some embodiments, R⁷ is selected from C₇₋₃₀alkyl and C₇₋₃₀alkenyl, wherein the C₇₋₃₀alkyl and C₇₋₃₀alkenyl are optionally substituted with one or more substituents independently selected from F and D, and/or are optionally interrupted by one to four O and/or CO₂. In some embodiments, R⁷ is selected from C₇₋₃₀alkyl and C₇₋₃₀alkenyl, wherein the C₇₋₃₀alkyl and C₇₋₃₀alkenyl are optionally substituted with one or more substituents independently selected from F and D, and/or are optionally interrupted by one to two O atoms and/or CO₂.

In some embodiments, the alkyl or alkene group of R⁷ is an alkyl or alkenyl group present in a fatty acid, wherein all available H atoms are optionally substituted with deuterium. In some embodiments, R⁷ is an alkenyl group present in a fatty acid, wherein all available H atoms are optionally substituted with deuterium. In some embodiments, the fatty acid is an omega-6 fatty acid (i.e. an unsaturated or polyunsaturated fatty acid wherein the double bond that is closest to the methyl end of the molecule is located at carbon numbered 6 starting from the end methyl group) or an omega-3 fatty acid (i.e. an unsaturated or polyunsaturated fatty acid wherein the double bond that is closest to the methyl end of the molecule is located at carbon numbered 3 starting from the end methyl group), wherein all available H atoms are optionally substituted with deuterium. In some embodiments, R⁷ is an alkyl group present in a fatty acid wherein all available H atoms are optionally substituted with deuterium. In some embodiments, the alkyl or alkene group of R⁷ is an alkyl or alkene group present in a fatty acid selected from:

| | Lipid | Chemical |
|---|---|---|
| Common Name | Number | Name |
| linoleic acid (LA) | 18:2 (n-6) | all-cis-9,12- |
| | | octadecadienoic acid, |
| rumenic acid | 18:2 (n-6) | 9Z,11E- |
| (conjugated | | octadecadienoic acid, |
| linoleic acid) | | |
| conjugated | 18:2 (n-6) | 10E,12Z- |
| linoleic acid | | octadecadienoic acid, |
| conjugated | 18:2 (n-6) | 9Z,12E- |
| linoleic acid | | octadecadienoic acid, |
| gamma-linolenic | 18:3 (n-6) | all-cis-6,9,12- |
| acid (GLA) | | octadecatrienoic acid, |
| calendic acid | 18:3 (n-6) | 8E,10E,12Z- |
| | | octadecatrienoic acid, |
| eicosadienoic acid | 20:2 (n-6) | all-cis-11,14- |
| | | eicosadienoic acid, |
| dihomo-gamma-linolenic | 20:3 (n-6) | all-cis-8,11,14- |
| acid (DGLA) | | eicosatrienoic acid, |
| arachidonic | 20:4 (n-6) | all-cis-5,8,11,14- |
| acid (AA, ARA) | | eicosatetraenoic acid |
| docosadienoic acid | 22:2 (n-6) | all-cis-13,16- |
| | | docosadienoic acid, |
| adrenic acid | 22:4 (n-6) | all-cis-7,10,13,16- |
| | | docosatetraenoic acid, |
| osbond acid | 22:5 (n-6) | all-cis-4,7,10,13,16- |
| | | docosapentaenoic acid, |
| tetracosatetraenoic | 24:4 (n-6) | all-cis-9,12,15,18- |
| acid | | tetracosatetraenoic acid, |
| tetracosapentaenoic | 24:5 (n-6) | all-cis-6,9,12,15,18- |
| acid | | tetracosapentaenoic acid, |
| α-linolenic | 18:3 (n-3) | all-cis-9,12,15- |
| acid (ALA) | | octadecatrienoic acid, |
| stearidonic | 18:4 (n-3) | all-cis-6,9,12,15- |
| acid (SDA) | | octadecatetraenoic acid, |
| hexadecatrienoic | 16:3 (n-3) | all-cis-7,10,13- |
| acid (HTA) | | hexadecatrienoic acid, |
| eicosatrienoic | 20:3 (n-3) | all-cis-11,14,17- |
| acid (ETE) | | eicosatrienoic acid, |
| eicosatetraenoic | 20:4 (n-3) | all-cis-8,11,14,17- |
| acid (ETA) | | eicosatetraenoic acid, |
| eicosapentaenoic | 20:5 (n-3) | all-cis-5,8,11,14,17- |
| acid (EPA) | | eicosapentaenoic acid, |
| heneicosapentaenoic | 21:5 (n-3) | all-cis-6,9,12,15,18- |
| acid (HPA) | | heneicosapentaenoic acid, |
| docosapentaenoic | 22:5 (n-3) | all-cis-7,10,13,16,19- |
| acid (DPA) | | docosapentaenoic acid, |
| docosahexaenoic | 22:6 (n-3) | all-cis-4,7,10,13,16,19- |
| acid (DHA) | | docosahexaenoic acid, |
| tetracosapentaenoic | 24:5 (n-3) | all-cis-9,12,15,18,21- |
| acid | | tetracosapentaenoic acid, |
| tetracosahexaenoic | 24:6 (n-3) | all-cis-6,9,12,15,18,21- |
| acid (Nisinic acid) | | tetracosahexaenoic acid, |
| myristoleic acid | 14:1 (n-5) | 9Z-tetradecenoic acid, |
| palmitoleic acid | 16:1 (n-7) | (9Z)-hexadecenoic acid, |
| sapienic acid | 16:1 (n-10) | (6Z)-hexadecenoic acid, |
| oleic acid | 18-1 (n-9) | (9Z)-octadecenoic acid, |
| elaidic acid | 18:1 (n-9) | (E)-octadecenoic acid, |
| vaccenic acid | 18:1 (n-7) | (11E)-octadecenoic acid, |
| eruric acid | 22-1 (n-9) | (13Z)-Docosenoic acid, |
| caprylic acid | 8:0 | octanoic acid, |
| capric acid | 10:0 | decanoic acid, |
| lauric acid | 12:0 | dodecanoic acid, |
| myristic acid | 14:0 | tetradecanoic acid, |
| palmitic acid | 16:0 | hexadecenoic acid, |
| stearic acid | 18:0 | octadecanoic acid, |
| arachidic acid | 20:0 | Icosanoic acid, |
| behenic acid | 22:0 | docosanoic acid, |
| lignoceric acid | 24:0 | tetracosanoic acid, and |
| cerotic acid | 26:0 | hexacosanoic acid, |

wherein all available H atoms are optionally substituted with deuterium.

In some embodiments, alkene group of R⁷ is an alkyl or alkenyl group present in linoleic acid, docosadienoic acid or eicosadienoic acid. In some embodiments, alkene group of R⁷ is an alkyl or alkenyl group present in linoleic acid.

In some embodiments, when R⁷ is the alkyl or alkenyl group of a fatty acid wherein 1-10, 2-8, 2-6 or 2-4H atoms are substituted with deuterium.

In some embodiments, R^{7′} is selected from H, C₁₋₆alkyl, C₁₋₆fluoroalkyl and C₁₋₆deuteroalkyl. In some embodiments, R^{7′} is selected from H, C₁₋₄alkyl, C₁₋₄fluoroalkyl and C₁₋₄deuteroalkyl. In some embodiments, R^{7′} is selected from OH₃, OF₃, CFzH and 00₃.

In some embodiments, R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, O, F, Cl, C₁₋₆alkyl, C₁₋₆fluoroalkyl and C₁₋₆deuteroalkyl. In some embodiments, R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, F, D, CH₃, CD₂H, CDH₂, CD₃, CF₃, CHF₂, CH₂CH₃, CH₂CH₂D, CH₂CD₂H and CD₂CD₃. In some embodiments, R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H and D.

In some embodiments, R^{16′}, R^{17′}, R^{18′}, R^{19′}, R^{21′}, R²², R^{23′}, R^{24′} and R^{25′} are independently selected from H, D, F, Cl, C₁₋₆alkyl, C₁₋₆fluoroalkyl and C₁₋₆deuteroalkyl. In some embodiments, R^{16′}, R^{17′}, R^{18′}, R^{19′}, R^{21′}, R^{22′}, R^{23′}, R^{24′} and R^{25′} are independently selected from H, F, D, CH₃, CD₂H, CDH₂, CD₃, CF₃, CHF₂, CH₂CH₃, CH₂CH₂D, CH₂CD₂H and CD₂CD₃. In some embodiments, R^{16′}, R^{17′}, R^{18′}, R^{19′}, R^{21′}, R^{22′}, R^{23′}, R^{24′} and R^{25′} are independently selected from H and D.

In some embodiments, R¹², R²⁰, R²⁰, R³⁰, R³¹, R³⁵ and R⁴⁵ are independently selected from H, C₁₋₄alkyl, C₁₋₄fluoroalkyl and C₁₋₄deuteroalkyl. In some embodiments, R¹², R²⁰, R²⁰, R³⁰, R³¹, R³⁵ and R⁴⁵ are independently selected from H, CH₃, CD₃, CF₂H and CF₃.

In some embodiments, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H, C₁₋₆alkyl, C₁₋₆fluoroalkyl and C₁₋₆deuteroalkyl. In some embodiments, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸ and R⁶⁹ are independently selected from are independently selected from H, D, CH₃, CD₂H, CDH₂, CD₃, CF₃, CHF₂, CH₂CH₃, CH₂CH₂D, CH₂CD₂H and CD₂CD₃.

In some embodiments, R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H, D, CH₃, CD₂H, CDH₂, CD₃, CF₃, CHF₂, CH₂CH₃, CH₂CH₂D, CH₂CD₂H, CH(CH₃)₂ and CD₂CD₃.

In some embodiments, the compounds of Formula I are selected from the compounds listed below, or a pharmaceutically acceptable salt and/or solvate thereof:

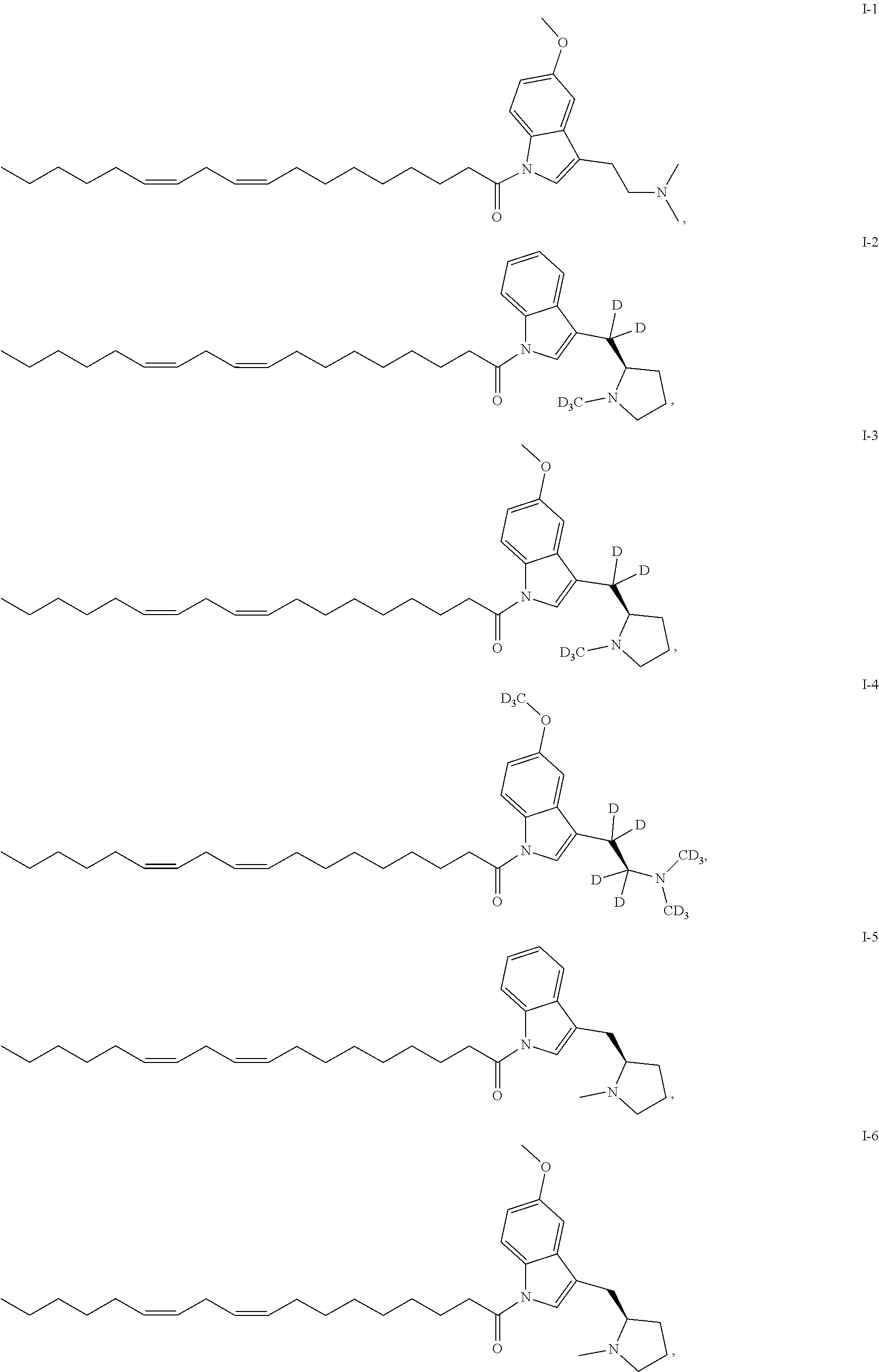

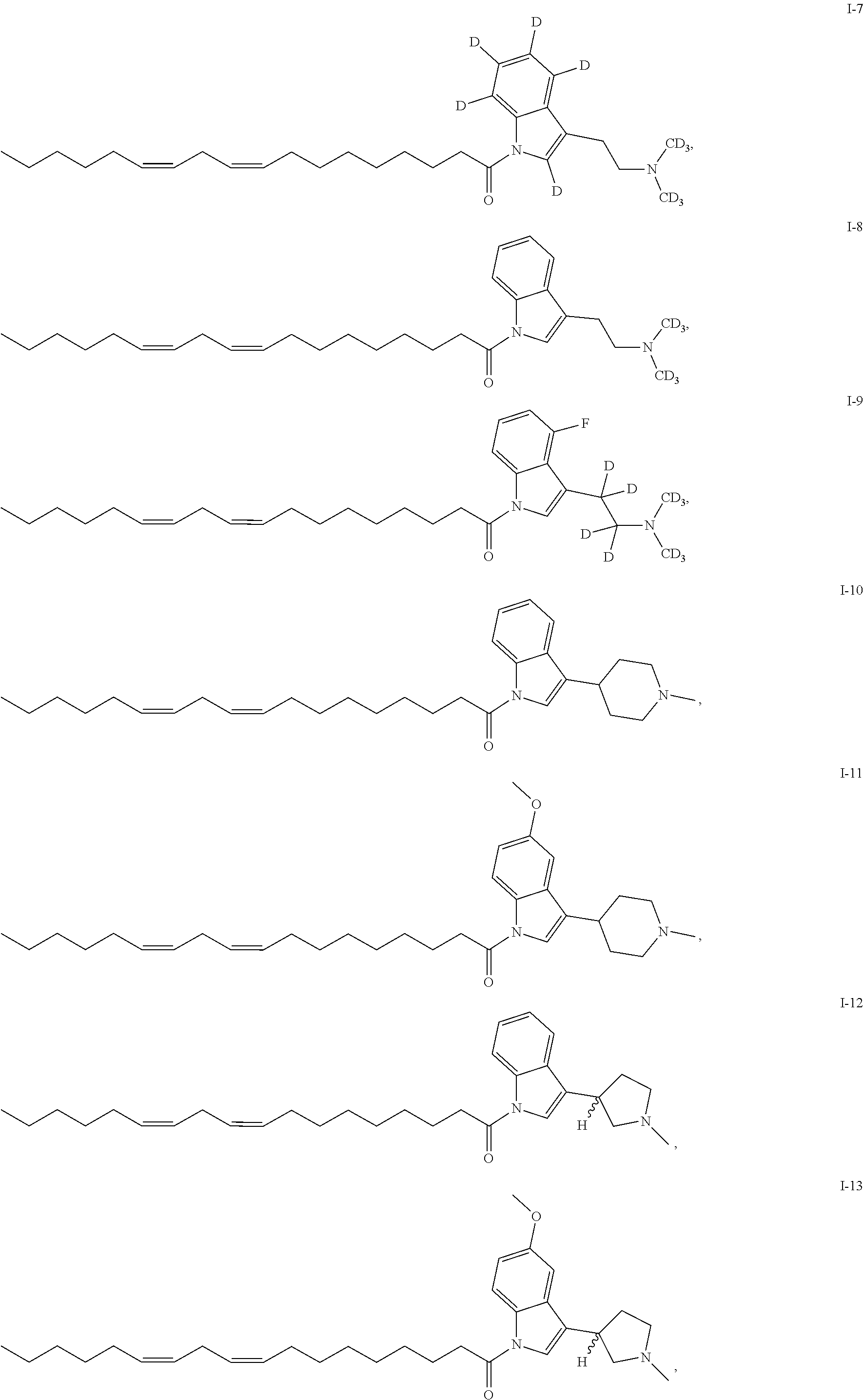

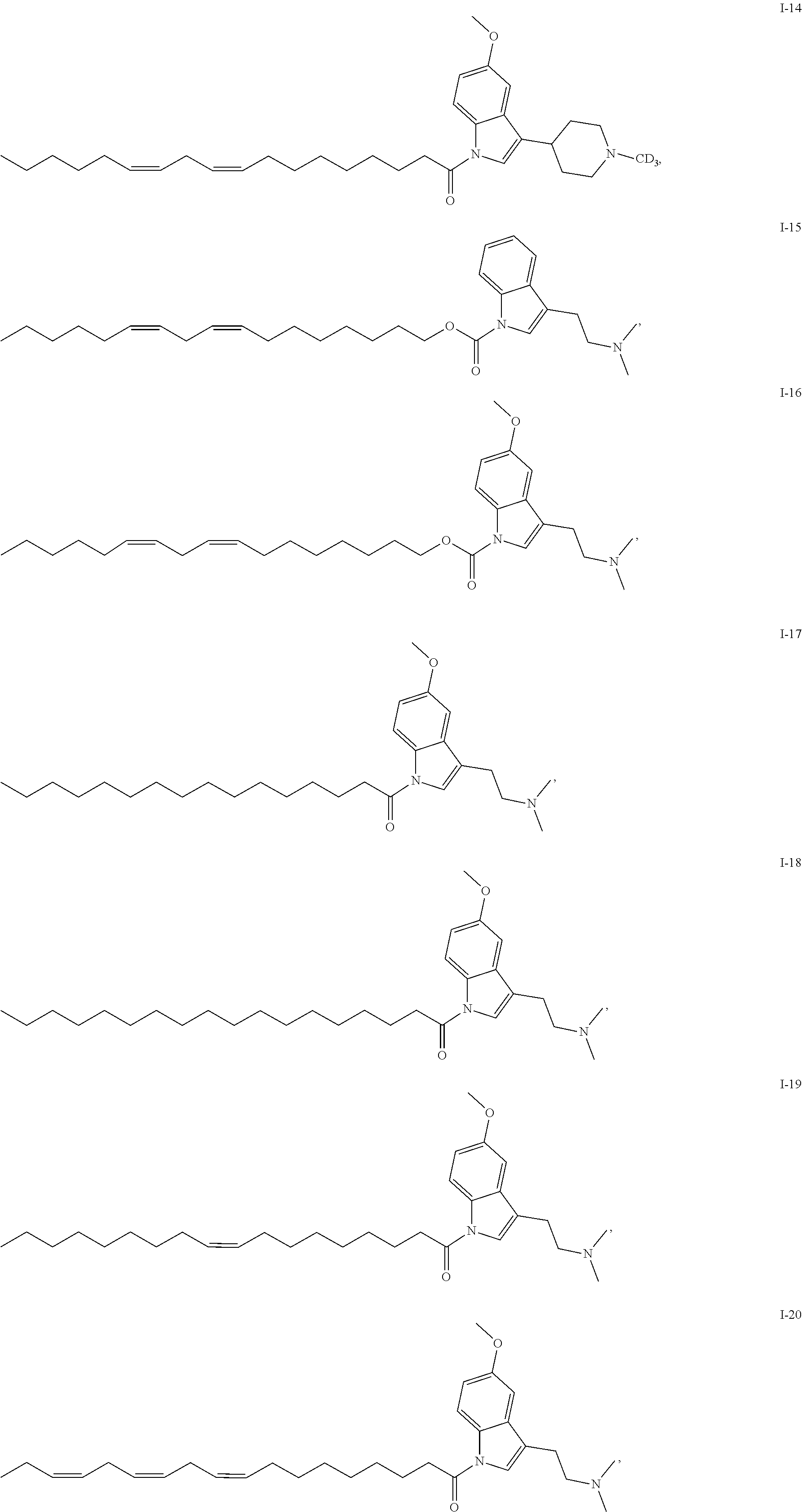

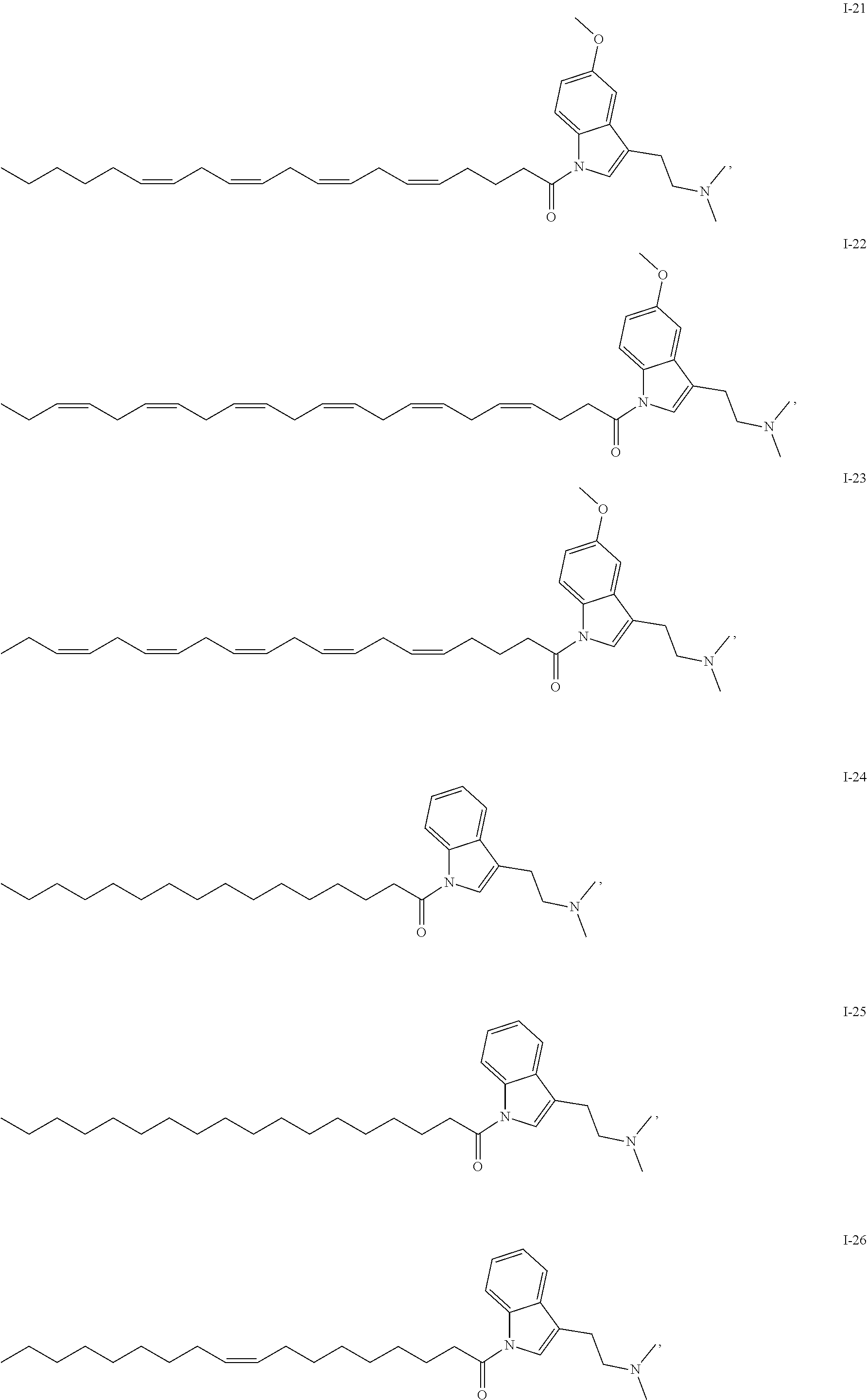

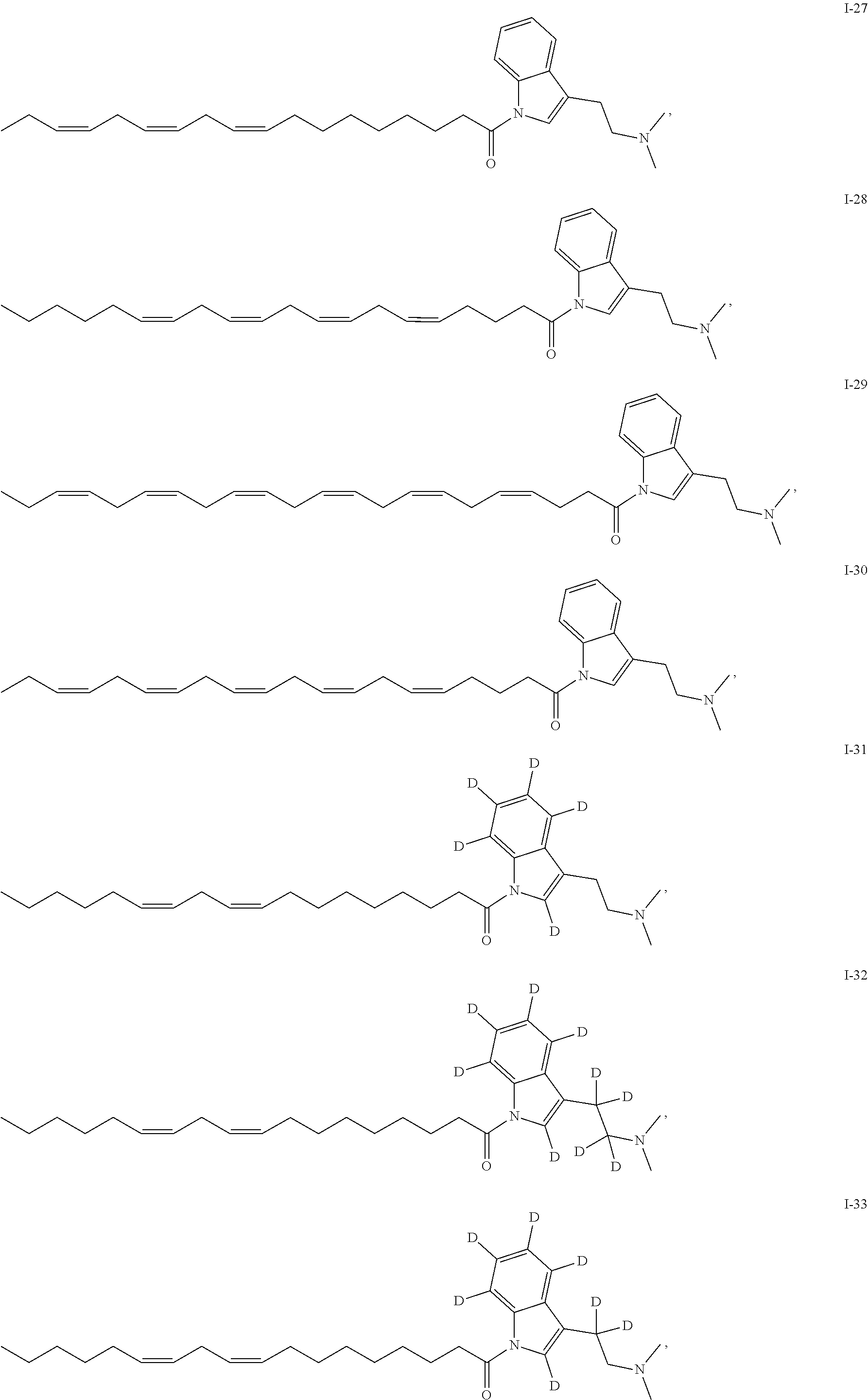

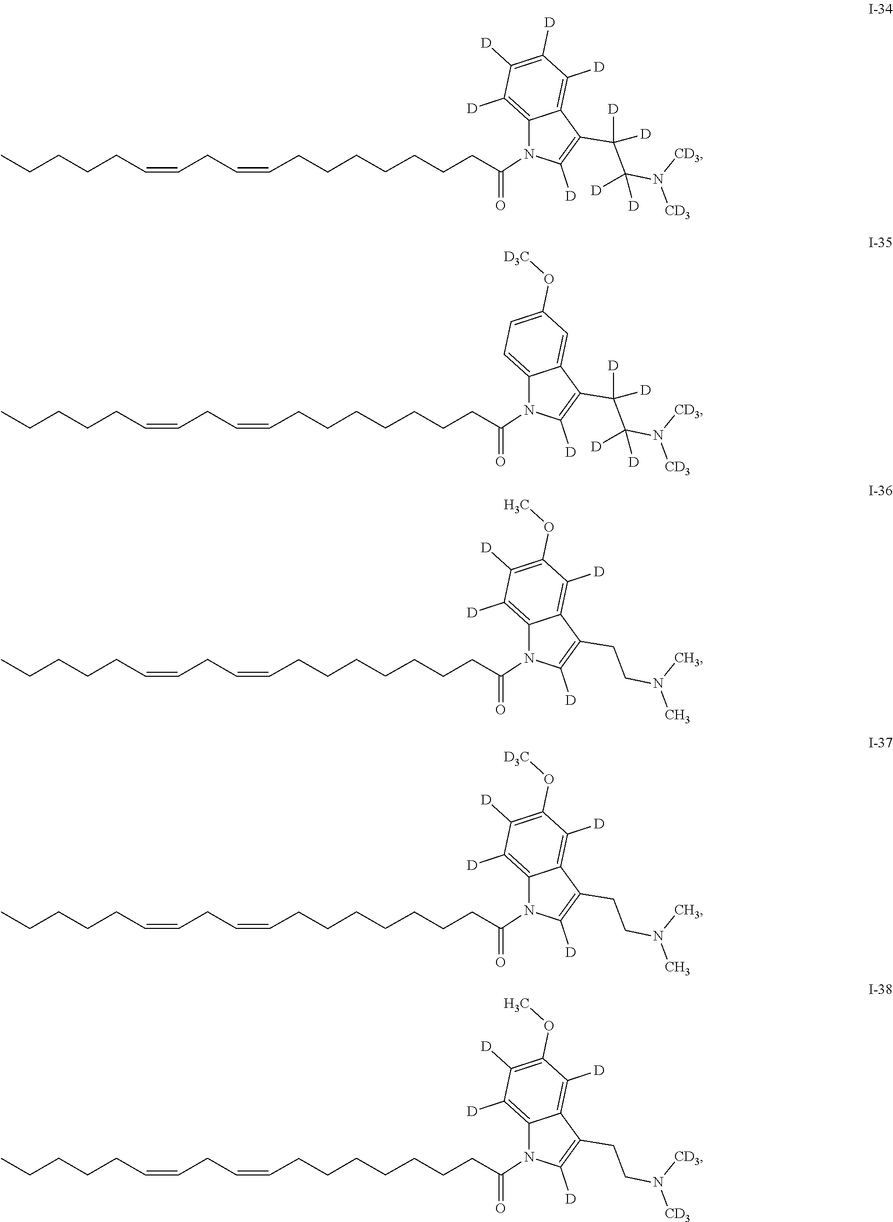

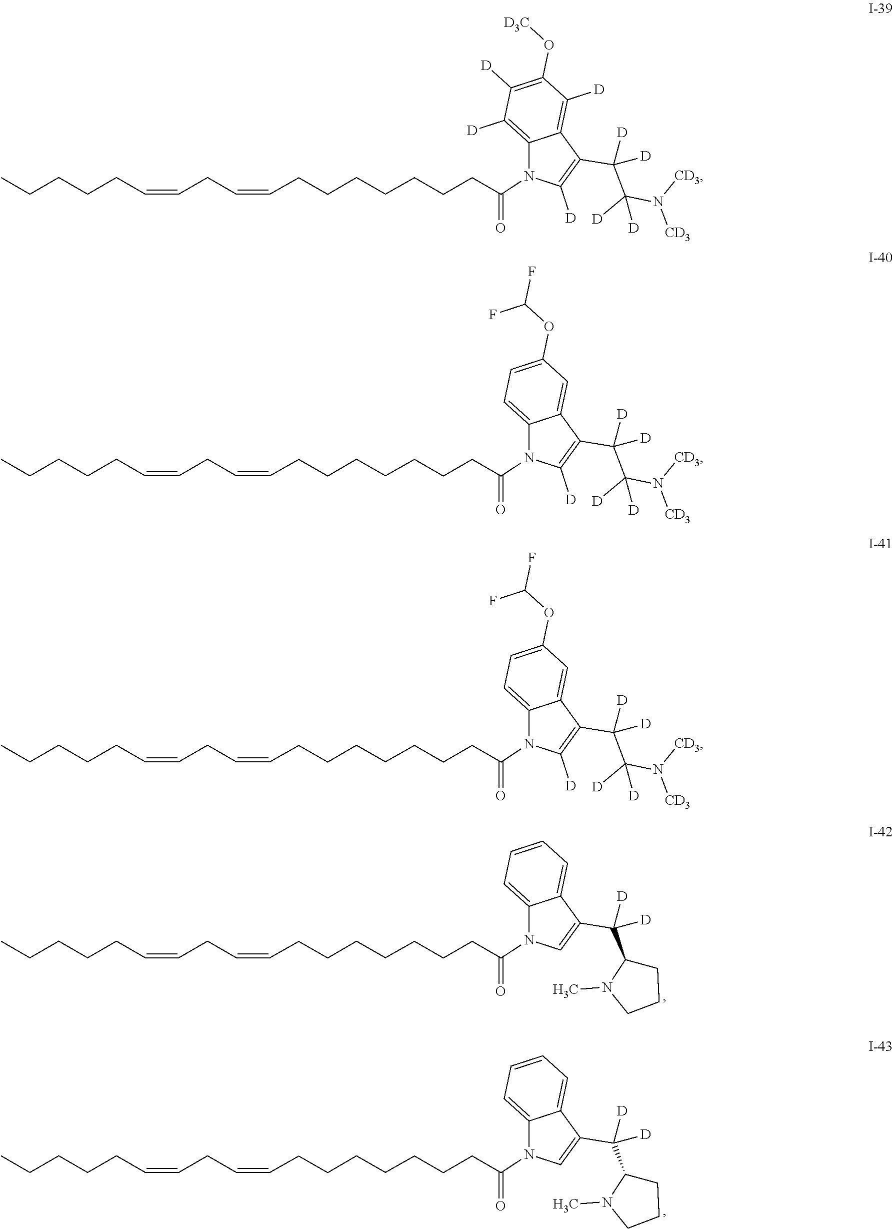

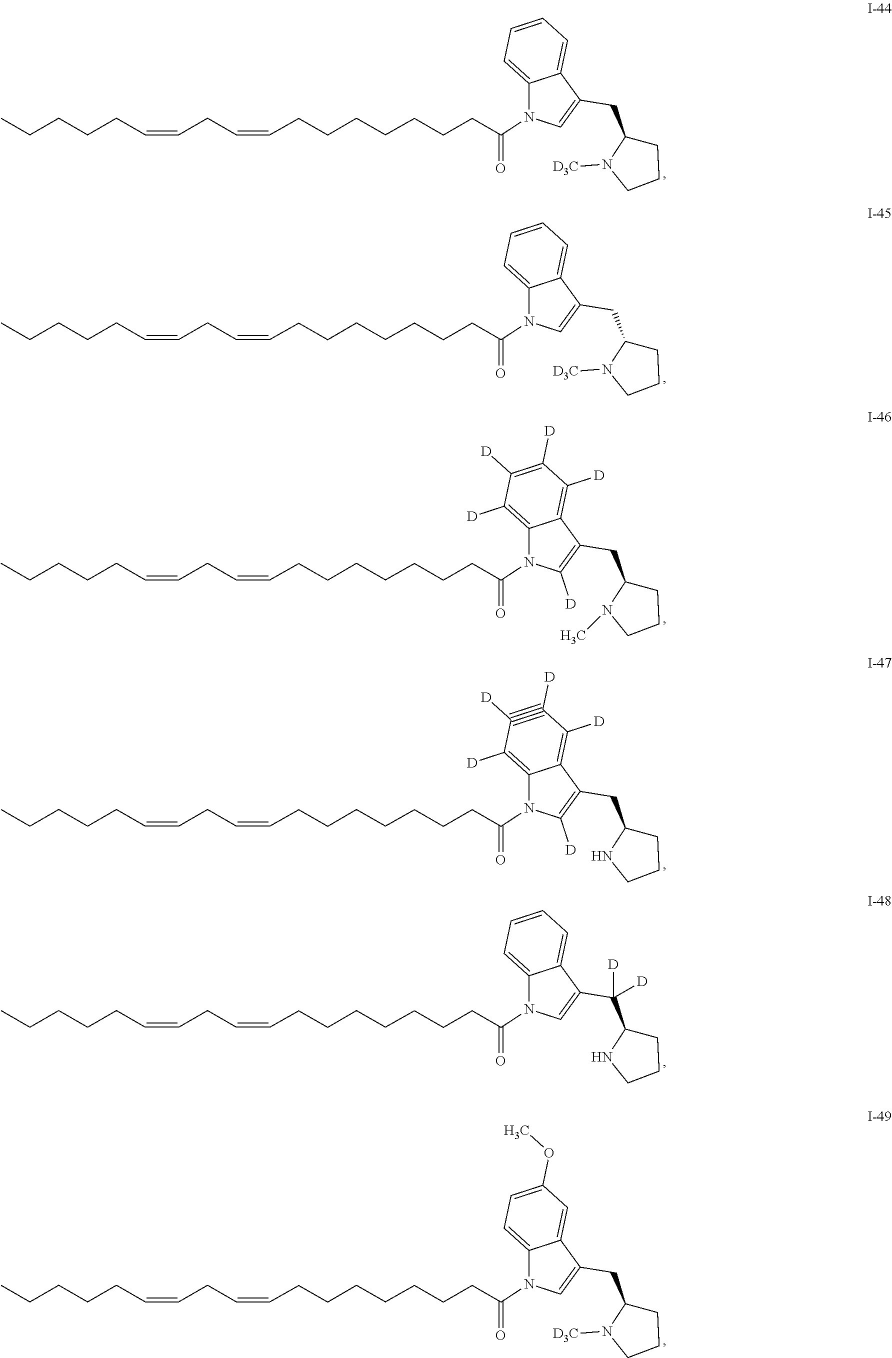 [SMILES: [2H]C([2H])([2H])N1CCC[C@@H]1Cc1cn(C(=O)CCCCCCC/C=C\C/C=C\CCCCC)c2ccccc12 ; [2H]C([2H])([2H])N1CCC[C@H]1Cc1cn(C(=O)CCCCCCC/C=C\C/C=C\CCCCC)c2ccccc12 ; [2H]c1c([2H])c([2H])c2c(c1[2H])c(C[C@H]1CCCN1C)c([2H])n2C(=O)CCCCCCC/C=C\C/C=C\CCCCC ; [2H]C1=c2c(C[C@H]3CCCN3)c([2H])n(C(=O)CCCCCCC/C=C\C/C=C\CCCCC)c2=C([2H])C([2H])C1[2H] ; [2H]C([2H])(c1cn(C(=O)CCCCCCC/C=C\C/C=C\CCCCC)c2ccccc12)[C@H]1CCCN1 ; [2H]C([2H])([2H])N1CCC[C@@H]1Cc1cn(C(=O)CCCCCCC/C=C\C/C=C\CCCCC)c2ccc(OC)cc12]

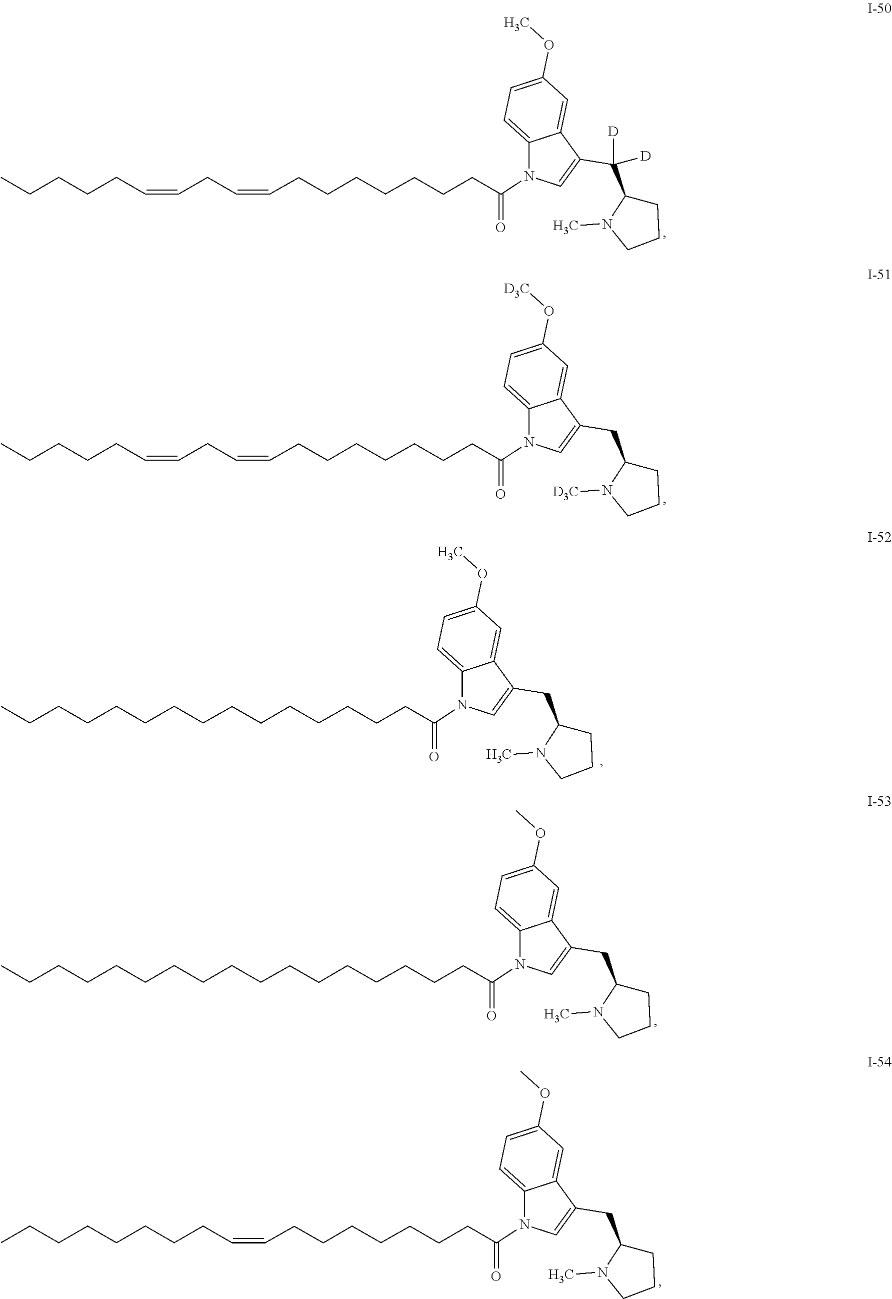 [SMILES: [2H]C([2H])(c1cn(C(=O)CCCCCCC/C=C\C/C=C\CCCCC)c2ccc(OC)cc12)[C@H]1CCCN1C ; [2H]C([2H])([2H])Oc1ccc2c(c1)c(C[C@H]1CCCN1C([2H])([2H])[2H])cn2C(=O)CCCCCCC/C=C\C/C=C\CCCCC ; CCCCCCCCCCCCCCCC(=O)n1cc(C[C@H]2CCCN2C)c2cc(OC)ccc21 ; CCCCCCCCCCCCCCCCCC(=O)n1cc(C[C@H]2CCCN2C)c2cc(OC)ccc21 ; CCCCCCCC/C=C\CCCCCCCC(=O)n1cc(C[C@H]2CCCN2C)c2cc(OC)ccc21]

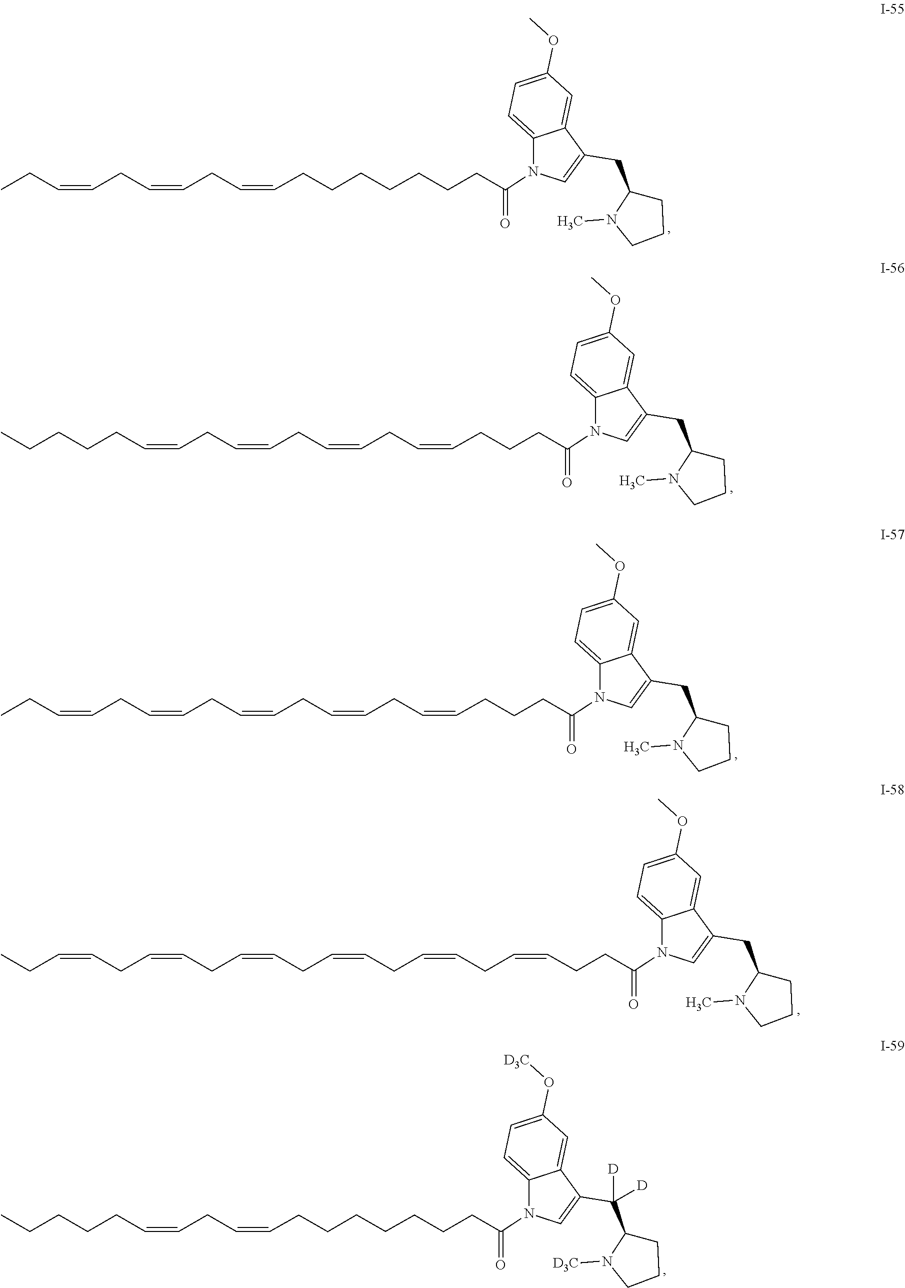 [SMILES: CC/C=C\C/C=C\C/C=C\CCCCCCCC(=O)n1cc(C[C@H]2CCCN2C)c2cc(OC)ccc21 ; CCCCC/C=C\C/C=C\C/C=C\C/C=C\CCCC(=O)n1cc(C[C@H]2CCCN2C)c2cc(OC)ccc21 ; CC/C=C\C/C=C\C/C=C\C/C=C\C/C=C\CCCC(=O)n1cc(C[C@H]2CCCN2C)c2cc(OC)ccc21 ; CC/C=C\C/C=C\C/C=C\C/C=C\C/C=C\C/C=C\CCC(=O)n1cc(C[C@H]2CCCN2C)c2cc(OC)ccc21 ; [2H]C([2H])([2H])Oc1ccc2c(c1)c(C([2H])([2H])[C@H]1CCCN1C([2H])([2H])[2H])cn2C(=O)CCCCCCC/C=C\C/C=C\CCCCC]

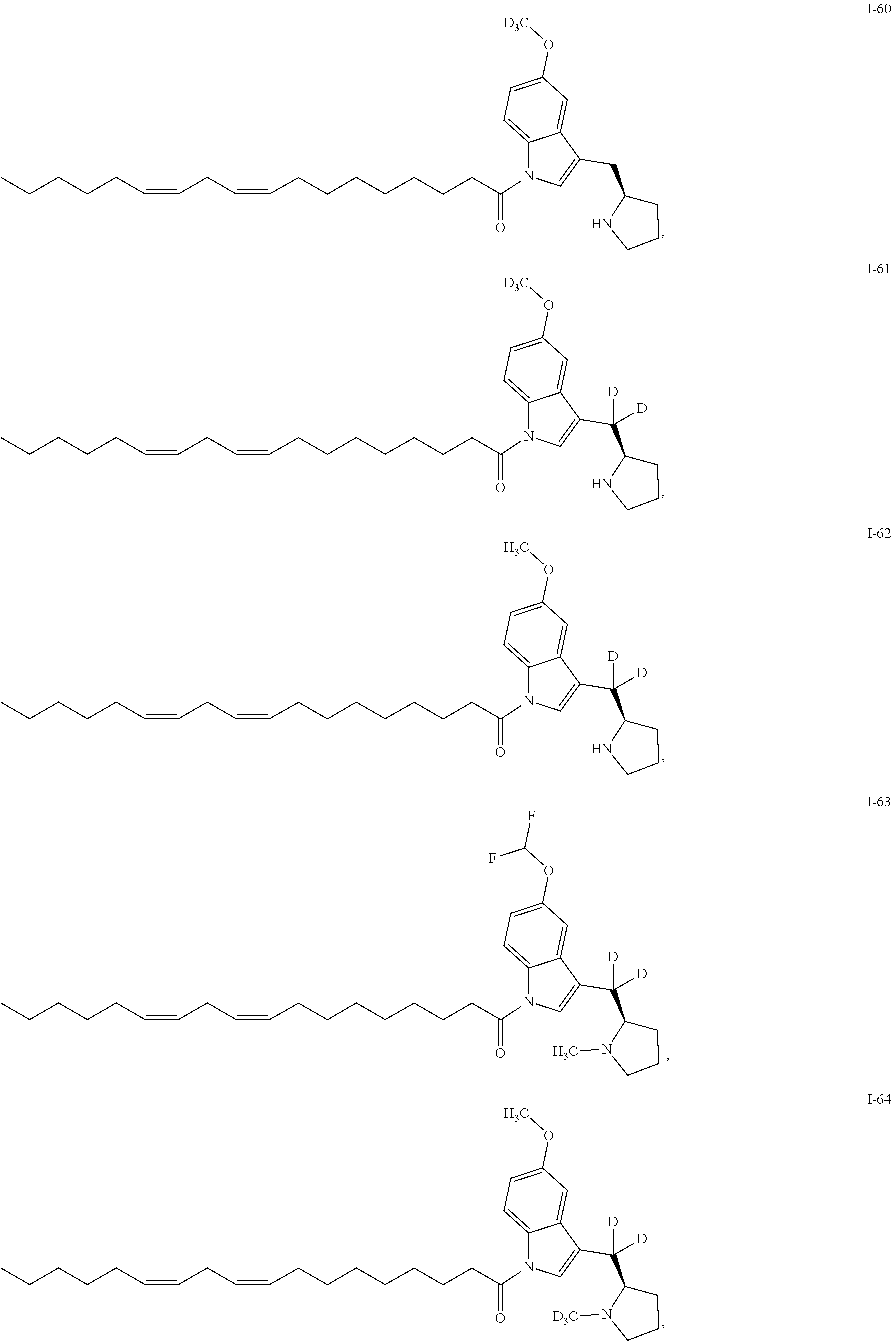 [SMILES: [2H]C([2H])([2H])Oc1ccc2c(c1)c(C[C@H]1CCCN1)cn2C(=O)CCCCCCC/C=C\C/C=C\CCCCC ; [2H]C([2H])([2H])Oc1ccc2c(c1)c(C([2H])([2H])[C@H]1CCCN1)cn2C(=O)CCCCCCC/C=C\C/C=C\CCCCC ; [2H]C([2H])(c1cn(C(=O)CCCCCCC/C=C\C/C=C\CCCCC)c2ccc(OC)cc12)[C@H]1CCCN1 ; [2H]C([2H])(c1cn(C(=O)CCCCCCC/C=C\C/C=C\CCCCC)c2ccc(OC(F)F)cc12)[C@H]1CCCN1C ; [2H]C([2H])(c1cn(C(=O)CCCCCCC/C=C\C/C=C\CCCCC)c2ccc(OC)cc12)[C@H]1CCCN1C([2H])([2H])[2H]]

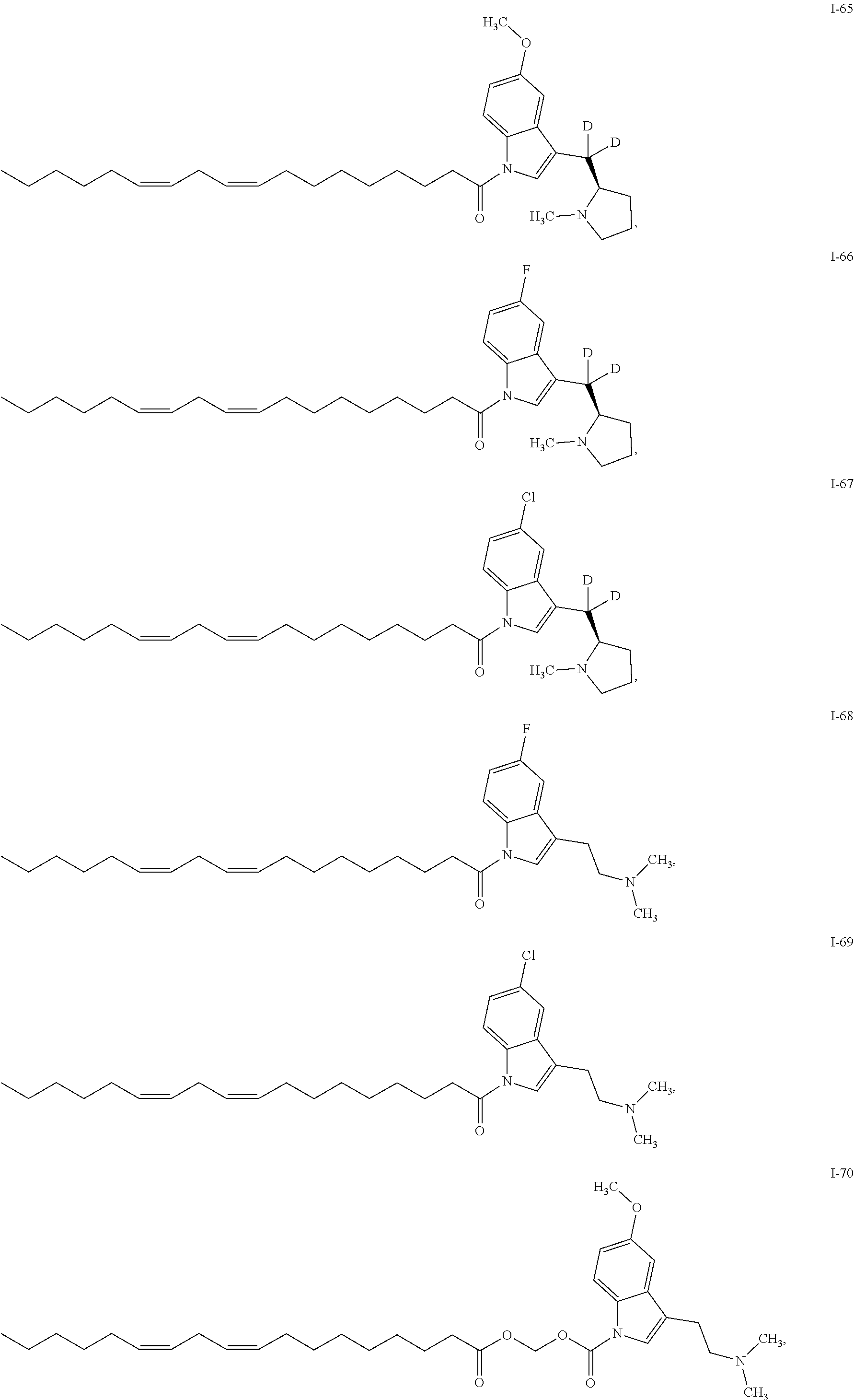 [SMILES: [2H]C([2H])(c1cn(C(=O)CCCCCCC/C=C\C/C=C\CCCCC)c2ccc(OC)cc12)[C@H]1CCCN1C ; [2H]C([2H])(c1cn(C(=O)CCCCCCC/C=C\C/C=C\CCCCC)c2ccc(F)cc12)[C@H]1CCCN1C ; [2H]C([2H])(c1cn(C(=O)CCCCCCC/C=C\C/C=C\CCCCC)c2ccc(Cl)cc12)[C@H]1CCCN1C ; CCCCC/C=C\C/C=C\CCCCCCCC(=O)n1cc(CCN(C)C)c2cc(F)ccc21 ; CCCCC/C=C\C/C=C\CCCCCCCC(=O)n1cc(CCN(C)C)c2cc(Cl)ccc21 ; CCCCC/C=C\C/C=C\CCCCCCCC(=O)OCOC(=O)n1cc(CCN(C)C)c2cc(OC)ccc21]

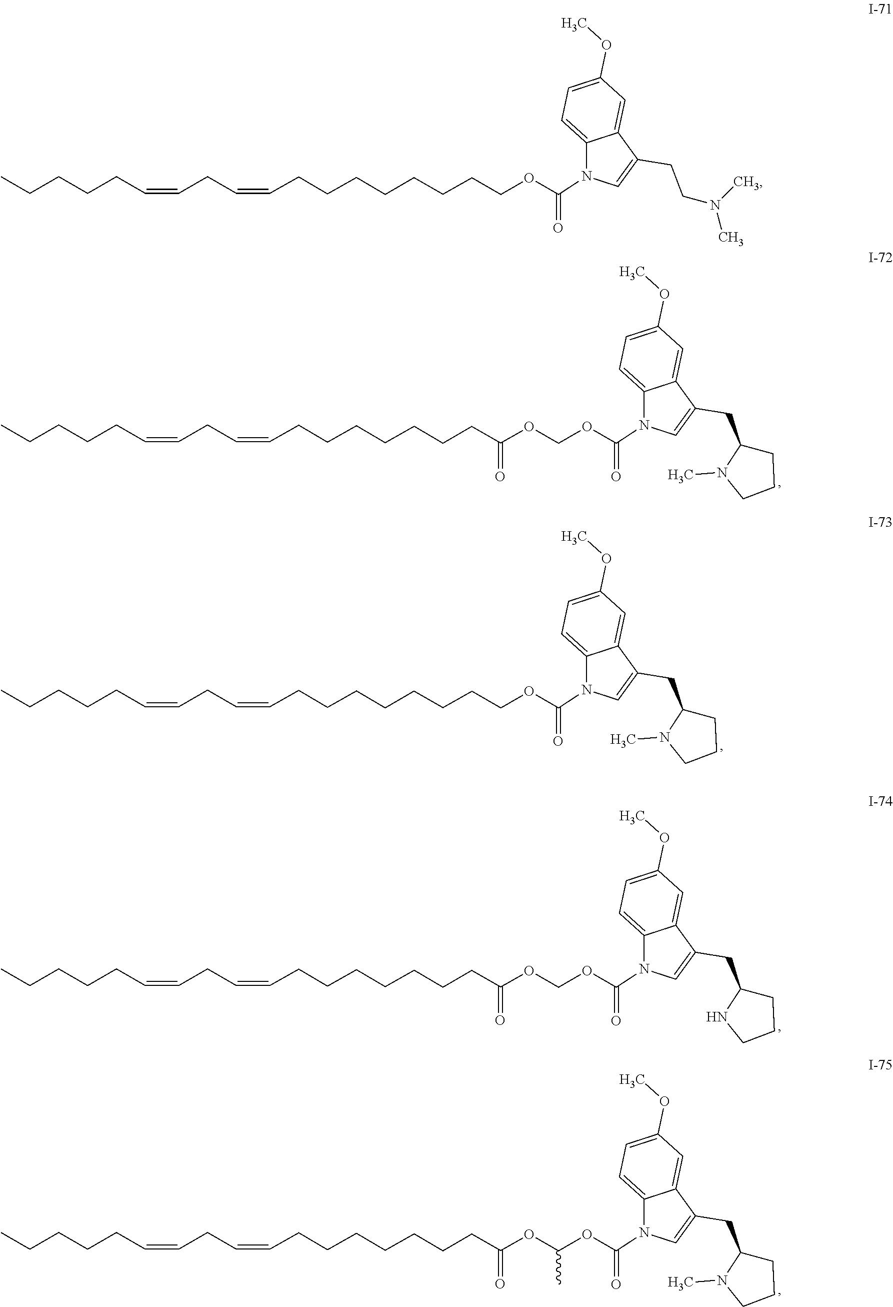 [SMILES: CCCCC/C=C\C/C=C\CCCCCCCCOC(=O)n1cc(CCN(C)C)c2cc(OC)ccc21 ; CCCCC/C=C\C/C=C\CCCCCCCC(=O)OCOC(=O)n1cc(C[C@H]2CCCN2C)c2cc(OC)ccc21 ; CCCCC/C=C\C/C=C\CCCCCCCCOC(=O)n1cc(C[C@H]2CCCN2C)c2cc(OC)ccc21 ; CCCCC/C=C\C/C=C\CCCCCCCC(=O)OCOC(=O)n1cc(C[C@H]2CCCN2)c2cc(OC)ccc21 ; CCCCC/C=C\C/C=C\CCCCCCCC(=O)OC(C)OC(=O)n1cc(C[C@H]2CCCN2C)c2cc(OC)ccc21]

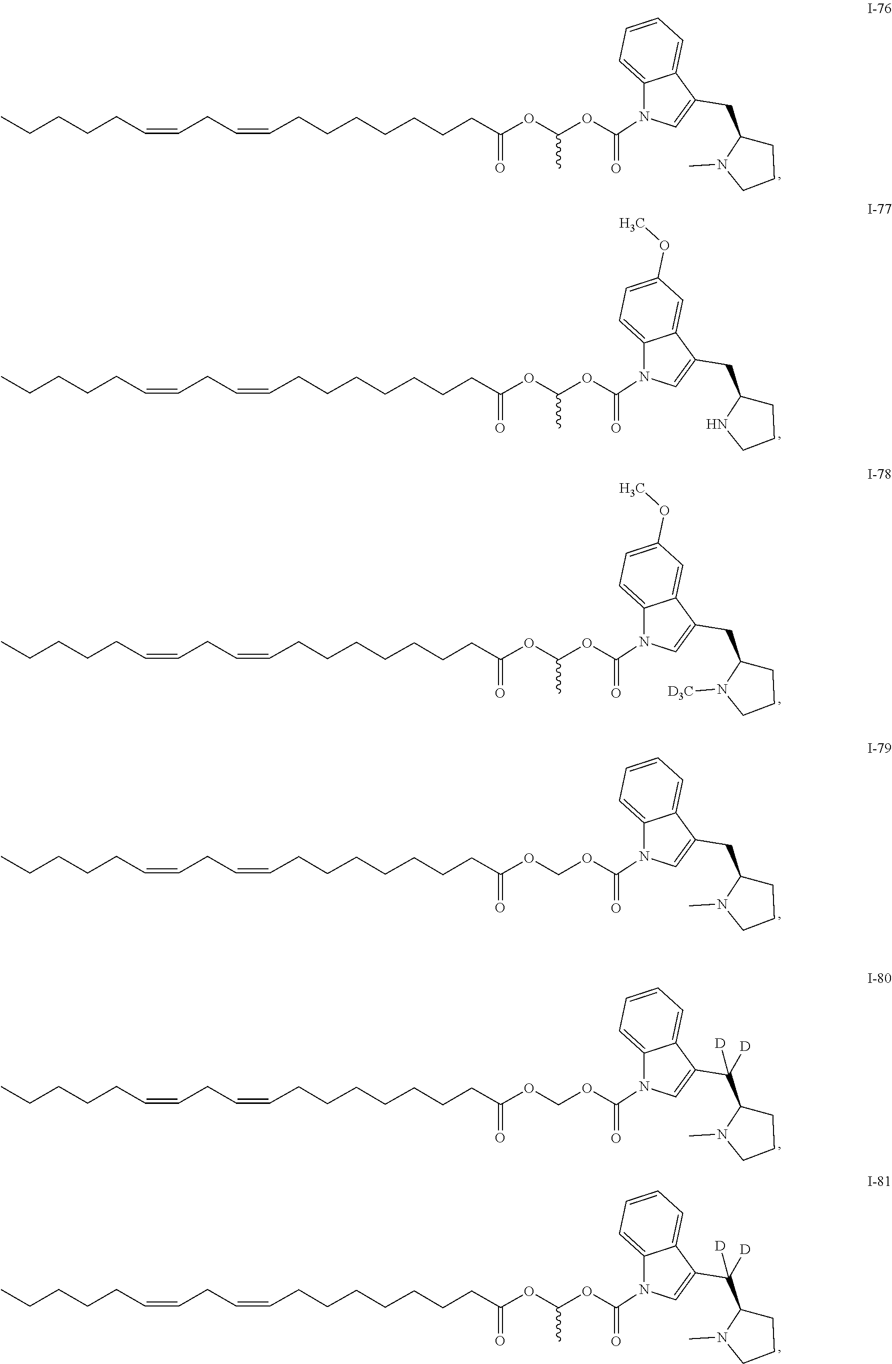

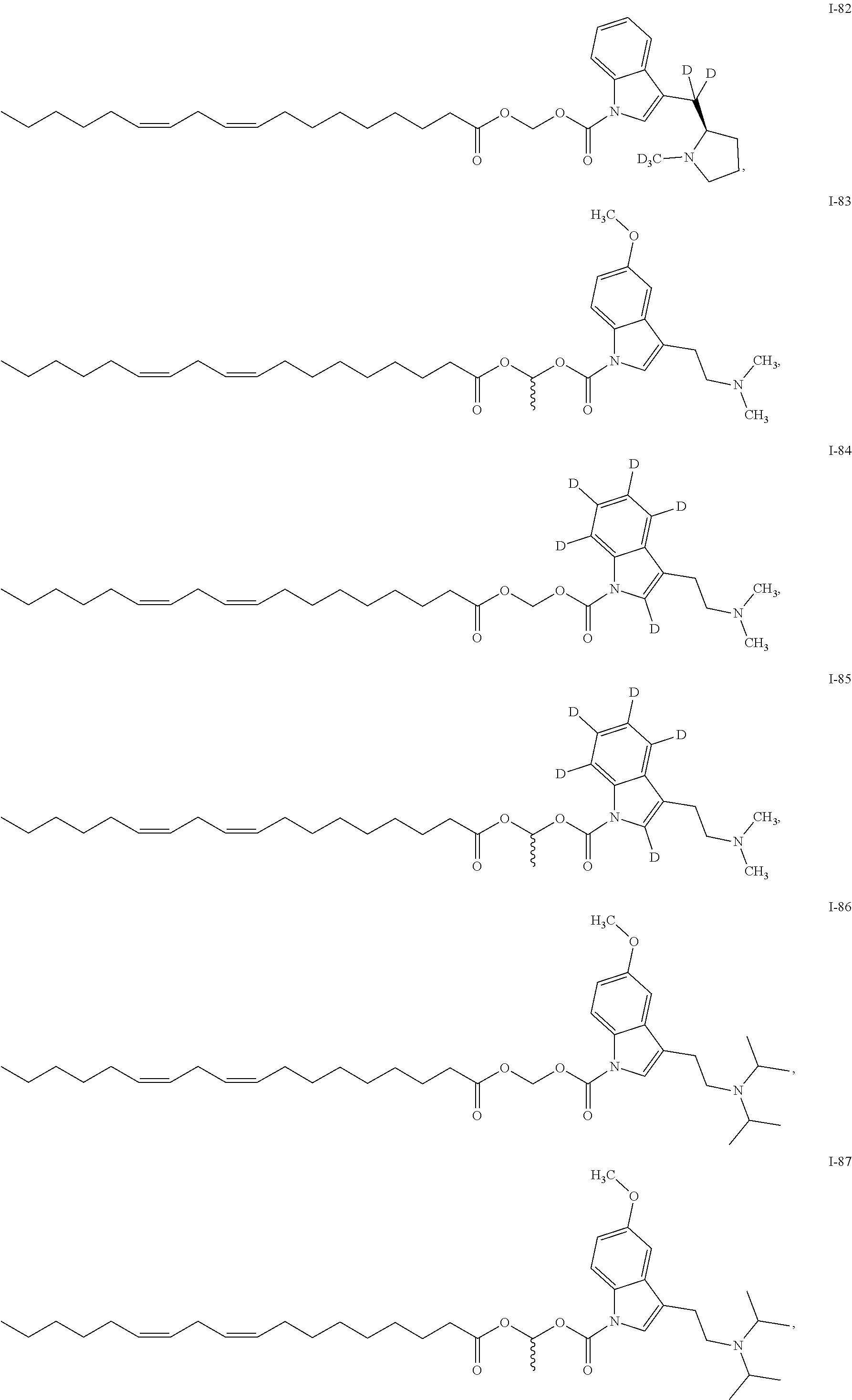

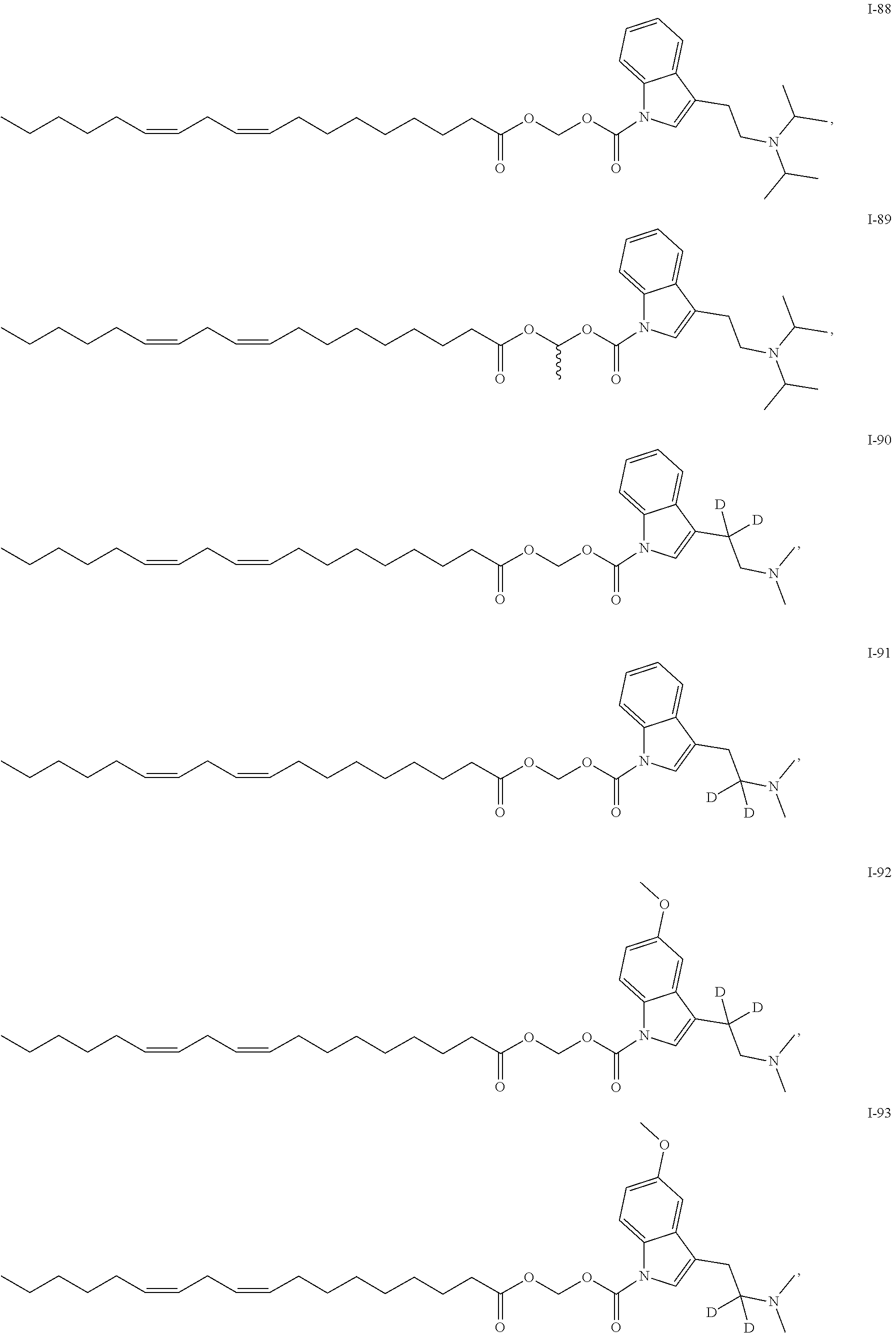

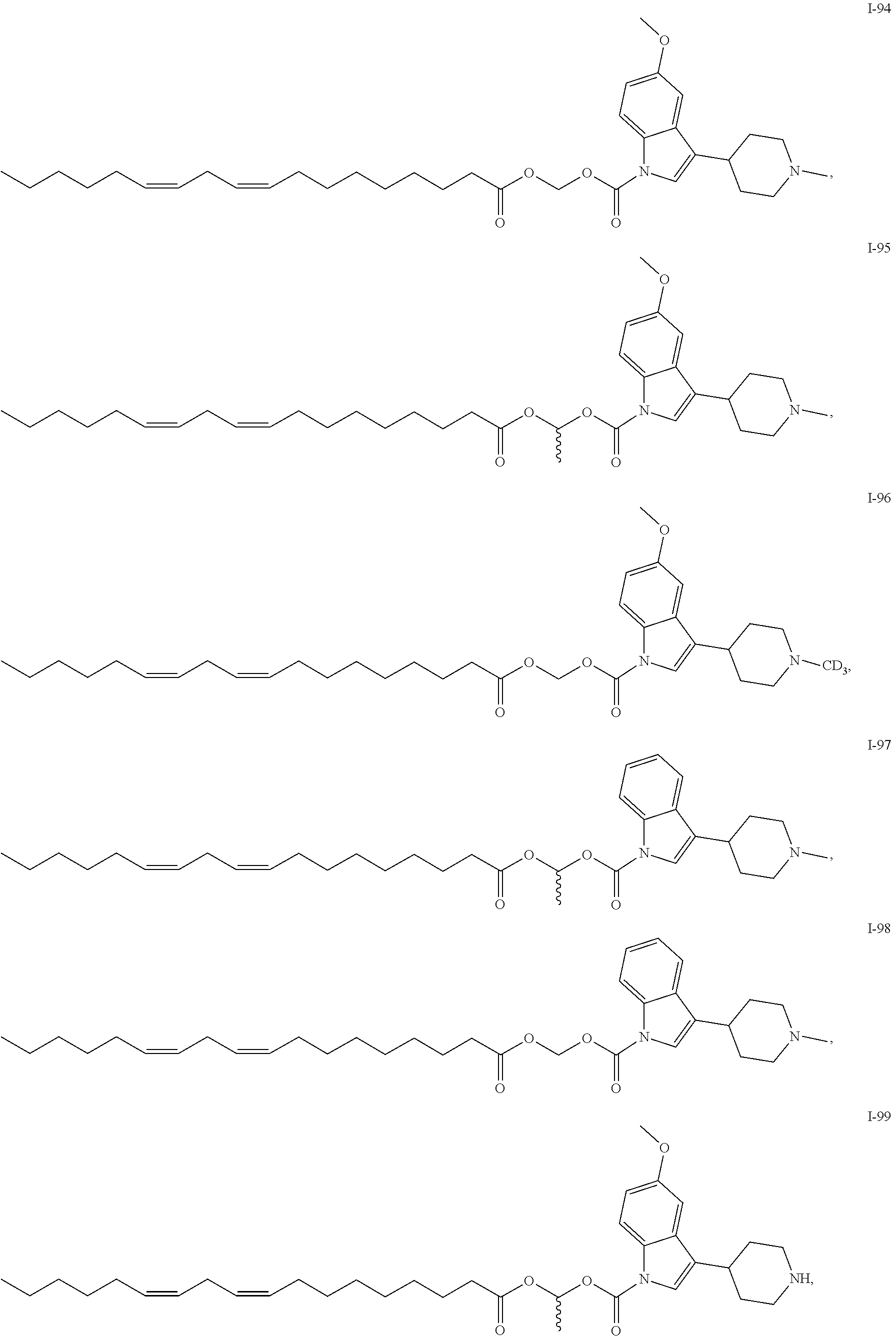

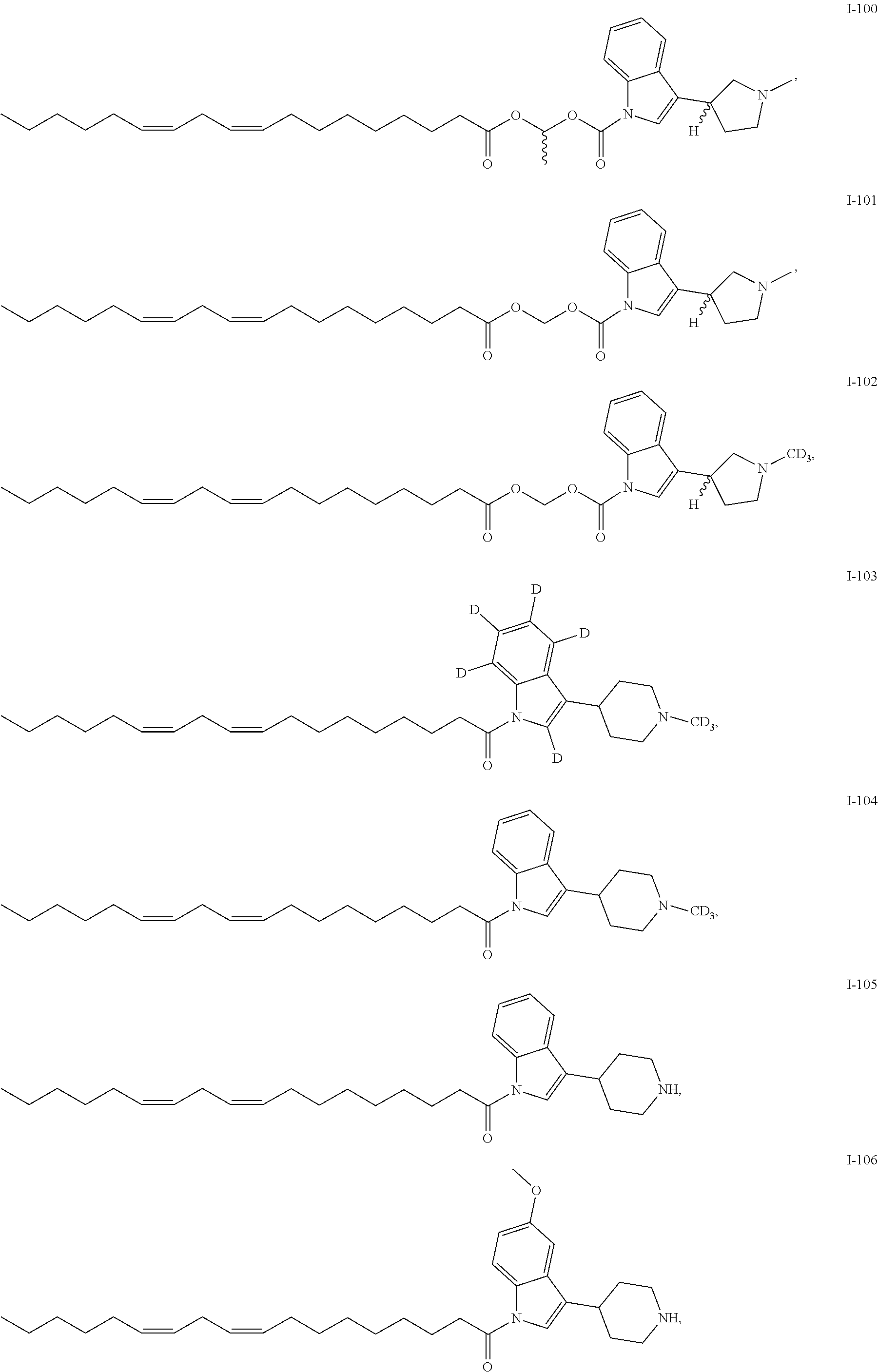

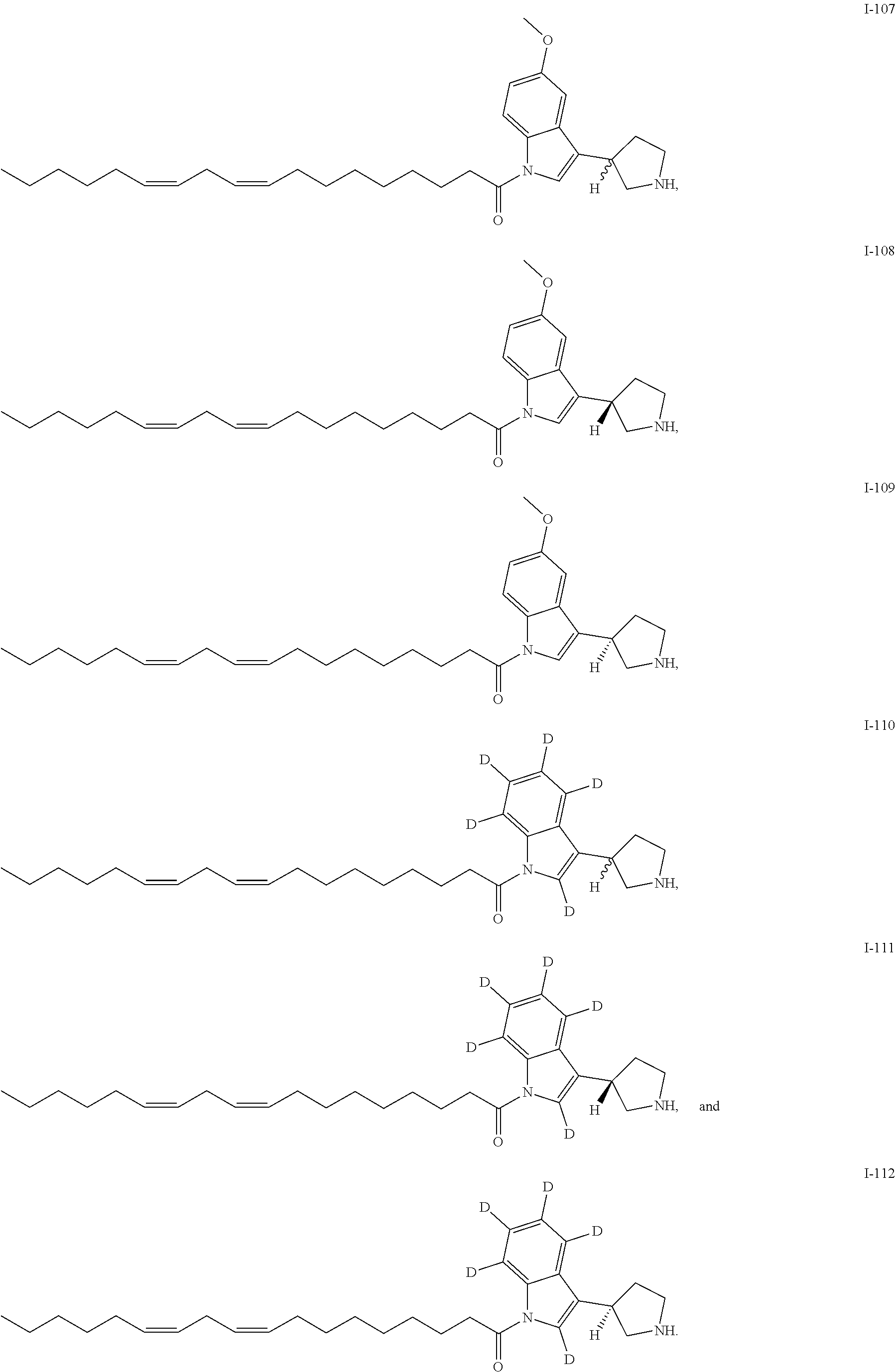

In some embodiments, the pharmaceutically acceptable salt is an acid addition salt or a base addition salt. The selection of a suitable salt may be made by a person skilled in the art. Suitable salts include acid addition salts that may, for example, be formed by mixing a solution of a compound with a solution of a pharmaceutically acceptable acid such as hydrochloric acid, sulfuric acid, acetic acid, trifluoroacetic acid, or benzoic acid. Additionally, acids that are generally considered suitable for the formation of pharmaceutically useful salts from basic pharmaceutical compounds are discussed, for example, by P. Stahl et al, Camille G. (eds.) and Handbook of Pharmaceutical Salts. Properties, Selection and Use. (2002) Zurich: Wiley VCH; S. Berge et al, Journal of Pharmaceutical Sciences 1977 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website).

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. In some embodiments, exemplary acid addition salts also include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates (“mesylates”), naphthalenesulfonates, nitrates, oxalates, phosphates, propionates, salicylates, succinates, sulfates, tartarates, thiocyanates, toluenesulfonates (also known as tosylates) and the like. In some embodiments, the mono- or di-acid salts are formed and such salts exist in either a hydrated, solvated or substantially anhydrous form. In general, acid addition salts are more soluble in water and various hydrophilic organic solvents and generally demonstrate higher melting points in comparison to their free base forms. The selection criteria forthe appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Exemplary organic bases are isopropylamine, diethylamine, ethanolamine, trimethylamine, dicyclohexylamine, choline and caffeine. The selection of the appropriate salt may be useful, for example, so that an ester functionality, if any, elsewhere in a compound is not hydrolyzed. The selection criteria for the appropriate salt will be known to one skilled in the art. In some embodiments, exemplary basic salts also include ammonium salts, alkali metal salts such as sodium, lithium and potassium salts, alkaline earth metal salts such as calcium and magnesium salts, salts with organic bases (for example, organic amines) such as dicyclohexylamine, Abutyl amine, choline and salts with amino acids such as arginine, lysine and the like. Basic nitrogen containing groups may be quarternized with agents such as lower alkyl halides (e.g., methyl, ethyl and butyl chlorides, bromides and iodides), dialkyl sulfates (e.g., dimethyl, diethyl and dibutyl sulfates), long chain halides (e.g., decyl, lauryl and stearyl chlorides, bromides and iodides), aralkyl halides (e.g., benzyl and phenethyl bromides) and others. Compounds carrying an acidic moiety can be mixed with suitable pharmaceutically acceptable salts to provide, for example, alkali metal salts (e.g., sodium or potassium salts), alkaline earth metal salts (e.g., calcium or magnesium salts) and salts formed with suitable organic ligands such as quaternary ammonium salts. Also, in the case of an acid (—COOH) or alcohol group being present, pharmaceutically acceptable esters can be employed to modify the solubility or hydrolysis characteristics of the compound.

All such acid salts and base salts are intended to be pharmaceutically acceptable salts within the scope of the application and all acid and base salts are considered equivalent to the free forms of the corresponding compounds for purposes of the application. In addition, when a compound of the application contains both a basic moiety, such as, but not limited to an aliphatic primary, secondary, tertiary or cyclic amine, an aromatic or heteroaryl amine, pyridine or imidazole and an acidic moiety, such as, but not limited to tetrazole or carboxylic acid, zwitterions (“inner salts”) may be formed and are included within the terms “salt(s)” as used herein. It is understood that certain compounds of the application may exist in zwitterionic form, having both anionic and cationic centers within the same compound and a net neutral charge. Such zwitterions are included within the application.

Solvates of compounds of the application include, for example, those made with solvents that are pharmaceutically acceptable. Examples of such solvents include water (resulting solvate is called a hydrate) and ethanol and the like. Suitable solvents are physiologically tolerable at the dosage administered.

It is understood and appreciated that in some embodiments, compounds of the present application may have at least one chiral center and therefore can exist as enantiomers and/or diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

In some embodiments, the compounds of the present application can also include tautomeric forms, such as keto-enol tautomers and the like. Tautomeric forms can be in equilibrium or sterically locked into one form by appropriate substitution. It is intended that any tautomeric forms which the compounds form, as well as mixtures thereof, are included within the scope of the present application.

The compounds of the present application may further exist in varying amorphous and polymorphic forms and it is contemplated that any amorphous forms, polymorphs, or mixtures thereof, which form are included within the scope of the present application.

The compounds of the present application may further be radiolabeled and accordingly all radiolabeled versions of the compounds of the application are included within the scope of the present application. There the compounds of the application also include those in which one or more radioactive atoms are incorporated within their structure.

### III. Compositions

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier. In embodiments of the application the pharmaceutical compositions are used in the treatment of any of the diseases, disorders or conditions described herein.

The compounds of the application are administered to a subject in a variety of forms depending on the selected route of administration, as will be understood by those skilled in the art. For example, a compound of the application is administered by oral, inhalation, parenteral, buccal, sublingual, insufflation, epidurally, nasal, rectal, vaginal, patch, pump, minipump, topical ortransdermal administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000-20th edition) and in The United States Pharmacopeia: The National Formulary (USP 24 NF19) published in 1999.

Parenteral administration includes systemic delivery routes other than the gastrointestinal (GI) tract and includes, for example intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time.

In some embodiments, a compound of the application is orally administered, for example, with an inert diluent or with an assimilable edible carrier, or it is enclosed in hard or soft shell gelatin capsules, or it is compressed into tablets, or it is incorporated directly with the food of the diet. In some embodiments, the compound is incorporated with excipient and used in the form of ingestible tablets, buccal tablets, troches, capsules, caplets, pellets, granules, lozenges, chewing gum, powders, syrups, elixirs, wafers, aqueous solutions and suspensions and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate), or solvents (e.g. medium chain triglycerides, ethanol, water). In embodiments, the tablets are coated by methods well known in the art. In the case of tablets, capsules, caplets, pellets or granules for oral administration, pH sensitive enteric coatings, such as Eudragits™ designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions are formulated, for example as liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. In some embodiments, liposomes are formed from a variety of phospholipids, such as cholesterol, stearylamine or phosphatidylcholines. For oral administration in a capsule form, useful carriers, solvents or diluents include lactose, medium chain triglycerides, ethanol and dried corn starch.

In some embodiments, liquid preparations for oral administration take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents are added. Such liquid preparations for oral administration are prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., medium chain triglycerides, almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

In some embodiments, a compound of the application is administered parenterally. For example, solutions of a compound of the application are prepared in water suitably mixed with a surfactant such as hydroxypropylcellulose. In some embodiments, dispersions are prepared in glycerol, liquid polyethylene glycols, DMSO and mixtures thereof with or without alcohol and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared and the pH's of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiments, such compositions include mucomimetics such as hyaluronic acid, chondroitin sulfate, hydroxypropyl methylcellulose or polyvinyl alcohol, preservatives such as sorbic acid, EDTA or benzyl chromium chloride and the usual quantities of diluents or carriers. For pulmonary administration, diluents or carriers will be selected to be appropriate to allow the formation of an aerosol.

In some embodiments, a compound of the application is formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles and contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

In some embodiments, compositions for nasal administration are conveniently formulated as aerosols, drops, gels and powders. For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which, for example, take the form of a cartridge or refill for use with an atomising device. Alternatively, the sealed container is a unitary dispensing device such as a single dose nasal inhaler or an aerosol dispenser fitted with a metering valve which is intended for disposal after use. Where the dosage form comprises an aerosol dispenser, it will contain a propellant which is, for example, a compressed gas such as compressed air or an organic propellant such as fluorochlorohydrocarbon. Suitable propellants include but are not limited to dichlorodifluoromethane, trichlorofluoromethane, dichlorotetrafluoroethane, heptafluoroalkanes, carbon dioxide or another suitable gas. In the case of a pressurized aerosol, the dosage unit is suitably determined by providing a valve to deliver a metered amount. In some embodiments, the pressurized container or nebulizer contains a solution or suspension of the active compound. Capsules and cartridges (made, for example, from gelatin) for use in an inhaler or insufflator are, for example, formulated containing a powder mix of a compound of the application and a suitable powder base such as lactose or starch. The aerosol dosage forms can also take the form of a pump-atomizer.

Compositions suitable for buccal or sublingual administration include tablets, lozenges and pastilles, wherein a compound of the application is formulated with a carrier such as sugar, acacia, tragacanth, or gelatin and glycerine. Compositions for rectal administration are conveniently in the form of suppositories containing a conventional suppository base such as cocoa butter.

Suppository forms of the compounds of the application are useful for vaginal, urethral and rectal administrations. Such suppositories will generally be constructed of a mixture of substances that is solid at room temperature but melts at body temperature. The substances commonly used to create such vehicles include but are not limited to theobroma oil (also known as cocoa butter), glycerinated gelatin, other glycerides, hydrogenated vegetable oils, mixtures of polyethylene glycols of various molecular weights and fatty acid esters of polyethylene glycol. See, for example: Remington's Pharmaceutical Sciences, 16th Ed., Mack Publishing, Easton, PA, 1980, pp. 1530-1533 for further discussion of suppository dosage forms.

In some embodiments a compound of the application is coupled with soluble polymers as targetable drug carriers. Such polymers include, for example, polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, in some embodiments, a compound of the application is coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

The compounds of the application are particularly amenable to administration with the air of nano-carrier systems, such as liposomes, micelles, nanoparticles, nano-emulsions, lipidic nano-systems and the like (see for example, Bhat, M. et al. Chem. and Phys. of Lipids, 2021, 236, 105053). Accordingly the present application includes a composition comprising one or more compounds of the application and one or more components of a nano-carrier system.

A compound of the application including pharmaceutically acceptable salts and/or solvates thereof is suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient and from about 1 wt % to about 99.95 wt % or about 30 wt % to about 99.90 wt % of a pharmaceutically acceptable carrier, all percentages by weight being based on the total composition.

In some embodiments, the compounds of the application including pharmaceutically acceptable salts and/or solvates thereof are used are administered in a composition comprising an additional therapeutic agent. Therefore the present application also includes a pharmaceutical composition comprising one of more compounds of the application, or pharmaceutically acceptable salts and/or solvates thereof and an additional therapeutic agent, and optionally one or more pharmaceutically acceptable excipients. In some embodiments, the additional therapeutic agent is another known agent useful for treatment of a disease, disorder or condition by activation of a serotonin receptor, for example those listed in the Methods and Uses section below. In some embodiments, the additional therapeutic agent is a psychoactive drug. In some embodiments, the additional therapeutic agent is another known agent useful for treatment of a disease, disorder or condition by modulation of a serotonin receptor, including activating, inhibiting, or antagonizing. In some embodiments, the additional therapeutic agent is a psychoactive drug that modifies release of serotonin or activates serotonin receptors.

To be clear, in the above, the term “a compound” also includes embodiments wherein one or more compounds are referenced.

### IV. Methods and Uses of the Application

Exemplary compounds of the application elicited psychedelic-like effects in relevant assays, such as the mouse head twitch assay, but surprisingly, in marked contrast to the corresponding compound where the indole nitrogen is unsubstituted, compounds of the application did not induce any signs of serotonin (5-HT) syndrome, including whole body twitches, fore paw treating and Straub tail. Pretreatment of mice with a selective 5-HT_{2A}antagonist M100907 (also known as volinanserin) prior to the administration of the compounds of the application completely blocked the head twitch effect of the exemplary compounds, confirming the involvement of the 5-HT_{2A} receptors on the head twitch response (HTR) induced by the exemplary compounds of the application.

Compounds of the application are useful for treating diseases, disorders or conditions by activating a serotonin receptor. Therefore, the compounds of the present application are useful as medicaments. Accordingly, the application also includes a compound of the application for use as a medicament.

The present application also includes a method of treating a disease, disorder or condition by activation of a serotonin receptor comprising administering a therapeutically effective amount of one or more compounds of the application to a subject in need thereof.

The present application also includes a use of one or more compounds of the application for treatment of a disease, disorder or condition by activation of a serotonin receptor as well as a use of one or more compounds of the application for the preparation of a medicament for treatment of a disease, disorder or condition by activation of a serotonin receptor. The application further includes one or more compounds of the application for use in treating a disease, disorder or condition by activation of a serotonin receptor.

In some embodiments, the serotonin receptor is 5-HT_{2A}. Accordingly, the present application includes a method for activating 5-HT_{2A} in a cell, either in a biological sample or in a patient, comprising administering an effective amount of one or more compounds of the application to the cell. The application also includes a use of one or more compounds of the application for activating 5-HT_{2A} in a cell as well as a use of one or more compounds of the application for the preparation of a medicament for activating 5-HT_{2A} in a cell. The application further includes one or more compounds of the application for use in activating 5-HT_{2A} in a cell.

In some embodiments, the serotonin receptor is 5-HT_{1A}. Accordingly, the present application includes a method for activating 5-HT_{1A} receptors in a cell, either in a biological sample or in a patient, comprising administering an effective amount of one or more compounds of the application to the cell. The application also includes a use of one or more compounds of the application for activating 5-HT_{1A} receptors in a cell as well as a use of one or more compounds of the application for the preparation of a medicament for activating 5-HT_{1A} receptors in a cell. The application further includes one or more compounds of the application for use in activating 5-HT_{1A} receptors in a cell.

The present application also includes a method of treating a disease, disorder or condition by activation of 5-HT_{2A} comprising administering a therapeutically effective amount of one or more compounds of the application to a subject in need thereof. The present application also includes a use of one or more compounds of the application for treatment of a disease, disorder or condition by activation of 5-HT_{2A} as well as a use of one or more compounds of the application for the preparation of a medicament for treatment of a disease, disorder or condition by activation of 5-HT_{2A}. The application further includes one or more compounds of the application for use in treating a disease, disorder or condition by activation of 5-HT_{2A}.

The present application also includes a method of treating a disease, disorder or condition by activation of 5-HT_{1A} comprising administering a therapeutically effective amount of one or more compounds of the application to a subject in need thereof. The present application also includes a use of one or more compounds of the application for treatment of a disease, disorder or condition by activation of 5-HT_{1A} as well as a use of one or more compounds of the application for the preparation of a medicament for treatment of a disease, disorder or condition by activation of 5-HT_{1A}. The application further includes one or more compounds of the application for use in treating a disease, disorder or condition by activation of 5-HT_{1A}.

The disease, disorder or condition may also be treated or treatable via alternative mechanisms, for example by modulation, deactivation, antagonism or reverse agonism of a serotonin receptor, including 5-HT_{2A} and/or 5-HT_{1A}.

In some embodiments, the compounds of the application are useful for preventing, treating and/or reducing the severity of a mental illness disorder and/or condition in a subject. Therefore, in some embodiments, the disease, disorder or condition that is treated by activation of a serotonin receptor is a mental illness. Accordingly, the present application also includes a method of treating a mental illness comprising administering a therapeutically effective amount of one or more compounds of the application to a subject in need thereof. The present application also includes a use of one or more compounds of the application for treatment a mental illness, as well as a use of one or more compounds of the application for the preparation of a medicament for treatment of a mental illness. The application further includes one or more compounds of the application for use in treating a mental illness.

In some embodiments, the mental illness is selected from anxiety disorders such as generalized anxiety disorder, panic disorder, social anxiety disorder and specific phobias; depression such as, hopelessness, loss of pleasure, fatigue and suicidal thoughts; mood disorders, such as depression, bipolar disorder, cancer-related depression, anxiety and cyclothymic disorder; psychotic disorders, such as hallucinations, delusions, schizophrenia; impulse control and addiction disorders, such as pyromania (starting fires), kleptomania (stealing) and compulsive gambling; alcohol addiction; drug addiction, such as opioid addiction; personality disorders, such as antisocial personality disorder, obsessive-compulsive personality disorder and paranoid personality disorder; obsessive-compulsive disorder (OCD), such as thoughts or fears that cause a subject to perform certain rituals or routines; post-traumatic stress disorder (PTSD); stress response syndromes (formerly called adjustment disorders); dissociative disorders, formerly called multiple personality disorder, or “split personality,” and depersonalization disorder; factitious disorders; sexual and gender disorders, such as sexual dysfunction, gender identity disorder and the paraphilia's; somatic symptom disorders, formerly known as a psychosomatic disorder or somatoform disorder; and combinations thereof.

In some embodiments, the disease, disorder or condition that is treated by activation of a serotonin receptor comprises cognitive impairment; ischemia including stroke; neurodegeneration; refractory substance use disorders; sleep disorders; pain, such as social pain, acute pain, cancer pain, chronic pain, breakthrough pain, bone pain, soft tissue pain, nerve pain, referred pain, phantom pain, neuropathic pain, cluster headaches and migraine; obesity and eating disorders; epilepsies and seizure disorders; neuronal cell death; excitotoxic cell death; or a combination thereof.

In some embodiments, the mental illness is selected from hallucinations and delusions and a combination thereof.

In some embodiments, the hallucinations are selected from visual hallucinations, auditory hallucinations, olfactory hallucinations, gustatory hallucinations, tactile hallucinations, proprioceptive hallucinations, equilibrioceptive hallucinations, nociceptive hallucinations, thermoceptive hallucinations and chronoceptive hallucinations, and a combination thereof.

In some embodiments, the disease, disorder or condition that is treated by activation of a serotonin receptor is psychosis or psychotic symptoms. Accordingly, the present application also includes a method of treating psychosis or psychotic symptoms comprising administering a therapeutically effective amount of one or more compounds of the application to a subject in need thereof.

The present application also includes a use of one or more compounds of the application for treatment of psychosis or psychotic symptoms, as well as a use of one or more compounds of the application for the preparation of a medicament for treatment of psychosis or psychotic symptoms. The application further includes one or more compounds of the application for use in treating psychosis or psychotic symptoms.

In some embodiments, administering to said subject in need thereof a therapeutically effective amount of the compounds of the application does not result in a worsening of psychosis or psychotic symptoms such as, but not limited to, hallucinations and delusions. In some embodiments, administering to said subject in need thereof a therapeutically effective amount of the compounds of the application results in an improvement of psychosis or psychotic symptoms such as, but not limited to, hallucinations and delusions. In some embodiments, administering to said subject in need thereof a therapeutically effective amount of the compounds of the application results in an improvement of psychosis or psychotic symptoms.

In some embodiments, the compounds of the application are useful for treating a central nervous system (CNS) disorder in a subject in need of therapy, comprising administering a therapeutically effective amount of a compound of general formula I, or a pharmaceutically acceptable salt thereof to the subject.

Therefore, in some embodiments, the disease, disorder or condition that is treated by activation of a serotonin receptor is a central nervous system (CNS) disease, disorder or condition and/or a neurological disease, disorder or condition. Accordingly, the present application also includes a method of treating a CNS disease, disorder or condition and/or a neurological disease, disorder or condition comprising administering a therapeutically effective amount of one or more compounds of the application to a subject in need thereof. The present application also includes a use of one or more compounds of the application for treatment a CNS disease, disorder or condition and/or a neurological disease, disorder or condition, as well as a use of one or more compounds of the application for the preparation of a medicament for treatment of a CNS disease, disorder or condition and/or a neurological disease, disorder or condition. The application further includes one or more compounds of the application for use in treating a CNS disease, disorder or condition and/or a neurological disease, disorder or condition. In some embodiments the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

In some embodiments, the subject is a mammal. In another embodiment, the subject is human. In some embodiments, the subject is a non-human animal. In some embodiments, the subject is canine. In some embodiments, the subject is feline. Accordingly, the compounds, methods and uses of the present application are directed to both human and veterinary diseases, disorders and conditions.

In some embodiments, the compounds of the application are useful for treating behavioral problems in subjects that are felines or canines.

Therefore, in some embodiments, the disease, disorder or condition that is treated by activation of a serotonin receptor is behavioral problems in subjects that are felines or canines. Accordingly, the present application also includes a method of treating a behavioral problem comprising administering a therapeutically effective amount of one or more compounds of the application to a non-human subject in need thereof. The present application also includes a use of one or more compounds of the application for treatment a behavioral problem in a non-human subject, as well as a use of one or more compounds of the application for the preparation of a medicament for treatment of a behavioral problem in a non-human subject. The application further includes one or more compounds of the application for use in treating a behavioral problem in a non-human subject.

In some embodiments, the behavioral problems are selected from, but are not limited to, anxiety, fear, stress, sleep disturbances, cognitive dysfunction, aggression, excessive noise making, scratching, biting and a combination thereof.

In some embodiments, the non-human subject is canine. In some embodiments, the non-human subject is feline.

The present application also includes a method of treating a disease, disorder or condition by activation of a serotonin receptor comprising administering a therapeutically effective amount of one or more compounds of the application in combination with another known agent useful for treatment of a disease, disorder or condition by activation of a serotonin receptor to a subject in need thereof. The present application also includes a use of one or more compounds of the application in combination with another known agent useful for treatment of a disease, disorder or condition by activation of a serotonin receptor for treatment of a disease, disorder or condition by activation of a serotonin receptor, as well as a use of one or more compounds of the application in combination with another known agent useful for treatment of a disease, disorder or condition by activation of a serotonin receptor for the preparation of a medicament for treatment of a disease, disorder or condition by activation of a serotonin receptor. The application further includes one or more compounds of the application in combination with another known agent useful for treatment of a disease, disorder or condition by activation of a serotonin receptor for use in treating a disease, disorder or condition by activation of a serotonin receptor.

In some embodiments, the disease, disorder or condition that is treated by activation of a serotonin receptor is a mental illness. In some embodiments, the mental illness is selected from hallucinations and delusions and a combination thereof. In some embodiments, the disease, disorder or condition that is treated by activation of a serotonin receptor is a central nervous system (CNS) disorder. In some embodiments, the disease, disorder or condition that is treated by activation of a serotonin receptor is psychosis or psychotic symptoms. In some embodiments, the disease, disorder or condition that is treated by activation of a serotonin receptor is behavioral problems in a non-human subject.

In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected from antipsychotics, including typical antipsychotics and atypical antipsychotics; antidepressants including selective serotonin reuptake inhibitors (SSRIs) and selective norepinephrine reuptake inhibitors (SNRIs), tricyclic antidepressants and monoamine oxidase inhibitors (MAOIs) (e.g. bupropion); anti-anxiety medication including benzodiazepines such as alprazolam; mood stabilizers such as lithium and anticonvulsants such carbamazepine, divalproex (valproic acid), lamotrigine, gabapentin and topiramate.

In some embodiments, the disease, disorder or condition that is treated by activation of a serotonin receptor is selected from attention deficit hyperactivity disorder and attention deficit disorder and a combination thereof. In some embodiments, the disease, disorder or condition that is treated by activation of a serotonin receptor is attention deficit hyperactivity disorder and/or attention deficit disorder and a combination thereof and the one or more compounds of the application are administered in combination with one or more additional treatments for attention deficit hyperactivity disorder and/or attention deficit disorder and a combination thereof. In some embodiments, the additional treatments for attention deficit hyperactivity disorder and/or attention deficit disorder and a combination thereof are selected from methylphenidate, atomoxetine and amphetamine and a combination thereof.

In some embodiments, the disease, disorder or condition that is treated by activation of a serotonin receptor is dementia or Alzheimer's disease and the one or more compounds of the application are administered in combination with one or more additional treatments for dementia or Alzheimer's disease. In some embodiments, the additional treatments for dementia and Alzheimer's disease are selected acetylcholinesterase inhibitors, NMDA antagonists and muscarinic agonists and antagonists, and nicotinic agonists.

In some embodiments, the acetylcholinesterase inhibitors are selected from donepezil, galantamine, rivastigmine, and phenserine, and combinations thereof.

In some embodiments, the NMDA antagonists are selected from MK-801, ketamine, phencyclidine, and memantine, and combinations thereof.

In some embodiments, the nicotinic agonists is nicotine, nicotinic acid, nicotinic alpha7 agonists or alpha2 beta4 agonists or combinations thereof.

In some embodiments, the muscarinic agonists is a muscarinic M1 agonist or a muscarinic M4 agonist, or combinations thereof.

In some embodiments, the muscarinic antagonist is a muscarinic M2 antagonist.

In some embodiments, the disease, disorder or condition that is treated by activation of a serotonin receptor is psychosis or psychotic symptoms and the one or more compounds of the application are administered in combination with one or more additional treatments for psychosis or psychotic symptoms. In some embodiments, the additional treatments for psychosis or psychotic symptom are selected typical antipsychotics and atypical antipsychotics.

In some embodiments, the typical antipsychotics are selected from acepromazine, acetophenazine, benperidol, bromperidol, butaperazine, carfenazine, chlorproethazine, chlorpromazine, chlorprothixene, clopenthixol, cyamemazine, dixyrazine, droperidol, fluanisone, flupentixol, fluphenazine, fluspirilene, haloperidol, levomepromazine, lenperone, Ioxapine, mesoridazine, metitepine, molindone, moperone, oxypertine, oxyprotepine, penfluridol, perazine, periciazine, perphenazine, pimozide, pipamperone, piperacetazine, pipotiazine, prochlorperazine, promazine, prothipendyl, spiperone, sulforidazine, thiopropazate, thioproperazine, thioridazine, thiothixene, timiperone, trifluoperazine, trifluperidol, triflupromazine and zuclopenthixol and combinations thereof.

In some embodiments, the atypical antipsychotics are selected from amoxapine, amisulpride, aripiprazole, asenapine, blonanserin, brexpiprazole, cariprazine, carpipramine, clocapramine, clorotepine, clotiapine, clozapine, iloperidone, levosulpiride, lurasidone, melperone, mosapramine, nemonapride, olanzapine, paliperidone, perospirone, quetiapine, remoxipride, reserpine, risperidone, sertindole, sulpiride, sultopride, tiapride, veralipride, ziprasidone and zotepine, and combinations thereof.

In some embodiments, the disease, disorder or condition that is treated by activation of a serotonin receptor is a mental illness and the one or more compounds of the application are administered in combination with one or more additional treatments for a mental illness. In some embodiments, the additional treatments for a mental illness is selected typical antipsychotics and atypical antipsychotics.

In some embodiments, the treatment methods and uses of the present application comprise a decreased or lower risk of the subject experiencing or having serotonin syndrome. In some embodiments, the decreased or lower risk is in comparison to a compound corresponding to a compound of the application except that the indole nitrogen is unsubstituted. In some embodiments, the treatment methods and uses comprise any detectable decrease or reduction in the incidences of serotonin syndrome, for example, compared to the incidences of serotonin syndrome after administration or use of a compound corresponding to a compound of the application except that the indole nitrogen is unsubstituted, such as 5-methoxytryptamine or tryptamine.

In some embodiments, effective amounts vary according to factors such as the disease state, age, sex and/or weight of the subject or species. In some embodiments, the amount of a given compound or compounds that will correspond to an effective amount will vary depending upon factors, such as the given drug(s) or compound(s), the pharmaceutical formulation, the route of administration, the type of condition, disease or disorder, the identity of the subject being treated and the like, but can nevertheless be routinely determined by one skilled in the art.

In some embodiment, the compounds of the application are administered one, two, three or four times a year. In some embodiments, the compounds of the application are administered at least once a week. However, in another embodiment, the compounds are administered to the subject from about one time per two weeks, three weeks or one month. In another embodiment, the compounds are administered about one time per week to about once daily. In another embodiment, the compounds are administered 1, 2, 3, 4, 5 or 6 times daily. The length of the treatment period depends on a variety of factors, such as the severity of the disease, disorder or condition, the age of the subject, the concentration and/or the activity of the compounds of the application and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration is required. For example, the compounds are administered to the subject in an amount and for duration sufficient to treat the subject.

In some embodiments, the compounds of the application are administered at doses that are hallucinogenic or psychotomimetic and taken in conjunction with psychotherapy or therapy and may occur once, twice, three, or four times a year. However, in some embodiments, the compounds are administered to the subject once daily, once every two days, once every 3 days, once a week, once every two weeks, once a month, once every two months, or once every three months at doses that are not hallucinogenic or psychotomimetic.

A compound of the application is either used alone or in combination with other known agents useful for treating diseases, disorders or conditions by activation of a serotonin receptor, such as the compounds of the application. When used in combination with other known agents useful in treating diseases, disorders by activation of a serotonin receptor, it is an embodiment that a compound of the application is administered contemporaneously with those agents. As used herein, “contemporaneous administration” of two substances to a subject means providing each of the two substances so that they are both active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In some embodiments, a compound of the present application is administered with another therapeutic agent simultaneously or sequentially in separate unit dosage forms or together in a single unit dosage form. Accordingly, the present application provides a single unit dosage form comprising one or more compounds of the application, an additional therapeutic agent and a pharmaceutically acceptable carrier.

The dosage of a compound of the application varies depending on many factors such as the pharmacodynamic properties of the compound, the mode of administration, the age, health and weight of the recipient, the nature and extent of the symptoms, the frequency of the treatment and the type of concurrent treatment, if any and the clearance rate of the compound in the subject to be treated. One of skill in the art can determine the appropriate dosage based on the above factors. In some embodiments, one or more compounds of the application are administered initially in a suitable dosage that is adjusted as required, depending on the clinical response. Dosages will generally be selected to maintain a serum level of the one or more compounds of the application from about 0.01 μg/cc to about 1000 μg/cc, or about 0.1 μg/cc to about 100 μg/cc. As a representative example, oral dosages of one or more compounds of the application will range between about 1 μg per day to about 1000 mg per day for an adult, suitably about 10 μg per day to about 500 mg per day, more suitably about 10 μg per day to about 200 mg per day. For parenteral administration, a representative amount is from about 0.0001 mg/kg to about 10 mg/kg, about 0.0001 mg/kg to about 1 mg/kg, about 0.01 mg/kg to about 0.1 mg/kg or about 0.0001 mg/kg to about 0.01 mg/kg will be administered. For oral administration, a representative amount is from about 0.001 μg/kg to about 10 mg/kg, about 0.1 μg/kg to about 10 mg/kg, about 0.01 μg/kg to about 1 mg/kg or about 0.1 μg/kg to about 1 mg/kg. For administration in suppository form, a representative amount is from about 0.1 mg/kg to about 10 mg/kg or about 0.1 mg/kg to about 1 mg/kg. In some embodiments of the application, compositions are formulated for oral administration and the one or more compounds are suitably in the form of tablets containing 0.1, 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient (one or more compounds of the application) per tablet. In some embodiments of the application the one or more compounds of the application are administered in a single daily, weekly or monthly dose or the total daily dose is divided into two, three or four daily doses.

In some embodiments, the compounds of the application are used or administered in an effective amount which comprises administration of doses or dosage regimens that are devoid of clinically meaningful psychedelic/psychotomimetic actions. In some embodiments, the compounds of the application are used or administered in an effective amount which comprises administration of doses or dosage regimens that provide clinical effects similar to those exhibited by a human plasma psilocin Cmax of 4 ng/mL or less and/or human 5-HT_{2A} human CNS receptor occupancy of 40% or less or those exhibited by a human plasma psilocin Cmax of 1 ng/mL or less and/or human 5-HT_{2A} human CNS receptor occupancy of 30% or less. In some embodiments, the compounds of the application are used or administered in an effective amount which comprises administration of doses or dosage regimens that provide clinical effects similar to those exhibited by a human plasma psilocin Tmax in excess of 60 minutes, in excess of 120 minutes or in excess of 180 minutes.

To be clear, in the above, the term “a compound” also includes embodiments wherein one or more compounds are referenced. Likewise, the term “compounds of the application” also includes embodiments wherein only one compound is referenced.

### V. Preparation of Compounds

Compounds of the present application can be prepared by various synthetic processes. The choice of particular structural features and/or substituents may influence the selection of one process over another. The selection of a particular process to prepare a given compound of the application is within the purview of the person of skill in the art. Some starting materials for preparing compounds of the present application are available from commercial chemical sources or may be extracted from cells, plants, animals or fungi. Other starting materials, for example as described below, are readily prepared from available precursors using straightforward transformations that are well known in the art. In the Schemes below showing some embodiments of methods of preparation of compounds of the application, all variables are as defined in Formula I, unless otherwise stated.

In some embodiments, the compounds of Formula I are prepared as shown in Scheme I:

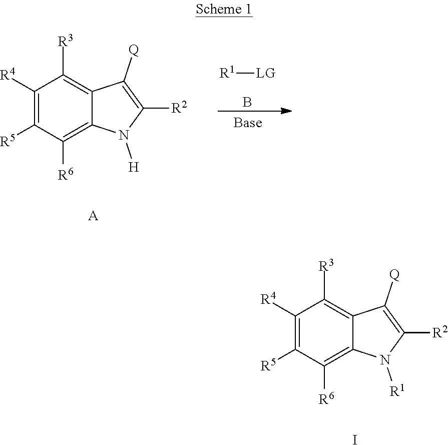

Therefore, in some embodiments, compounds of Formula A, wherein R²—R⁶ and Q are as defined in Formula I are reacted with compounds of Formula B, wherein R¹ is as defined in Formula I and LG is a suitable leaving group in the presence of a base to provide compounds of Formula 1. In some embodiments, LG is halo, such as chloro and the base is a strong base, such as NaH, NaOtBu or lithium bis(trimethylsilyl)amide (LiHMDS).

In some embodiments, compounds of Formula I are also available as shown in Scheme 2:

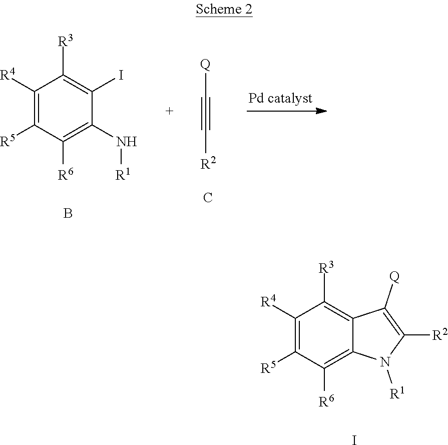

Therefore, in some embodiments, ortho-iodoaniline compounds of Formula B, wherein R¹ and R³—R⁶ are as defined in Formula I, are reacted with alkynes of Formula C, wherein R², and Q are as defined in Formula I, in the presence of a palladium catalyst, for example using the conditions described in Fricke et al., Chem. Eur. J., 2019, 25(4):897-903], to provide the compounds of Formula I. A person skilled in the art would appreciate that the reaction shown in Scheme 2 can also be used to provide compounds of Formula A by using a compound of Formula B wherein R¹ is a suitable protecting group and removing the protecting group to provide the compound of Formula A.

In some embodiments, when Q is

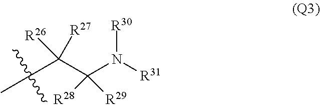

and R²⁶—R³¹ are either H or D, the compounds of Formula A are prepared as shown in Scheme 3:

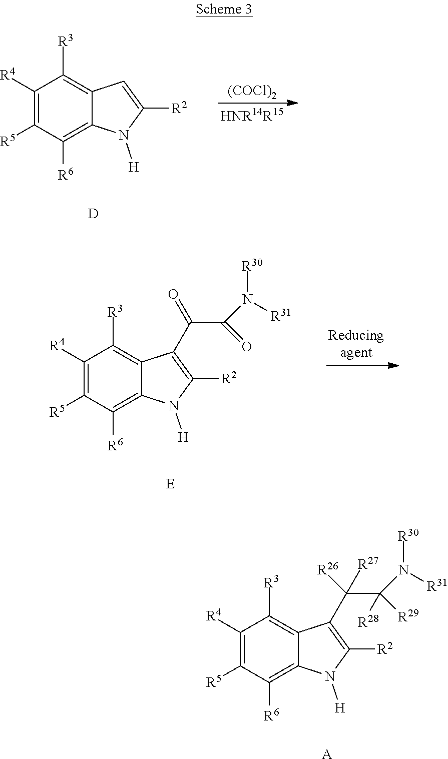

Therefore, in some embodiments, compounds of Formula D, wherein R² and R³—R⁶ are as defined in Formula I, are reacted with oxalyl chloride followed by an amine NHR³⁰R³¹ wherein R³⁰ and R³¹ are as defined in Formula I, to provide compounds of Formula E. Subsequent Al-based reduction of the compounds of Formula E, for example in the presence of lithium borohydride, lithium aluminum hydride or lithium aluminum deuteride, provides compounds of Formula A, wherein Q is

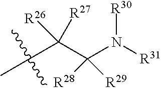

and R²⁶—R²⁷ are either H or D, R² and R³—R⁶ and R³⁰—R³¹ are as defined in Formula I.

In some embodiments, when Q is

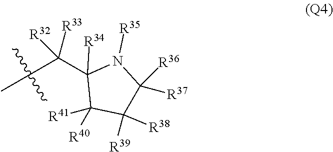

and R³² and R³³ are either H or D, the compounds of Formula A are prepared as shown in Scheme 4:

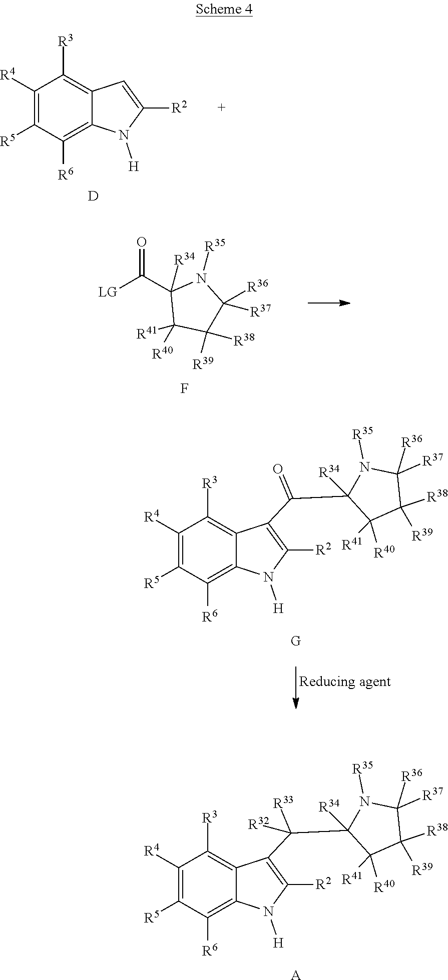

Therefore, in some embodiments, compounds of Formula D, wherein R² and R³—R⁶ are as defined in Formula I, are reacted under basic conditions using, for example, ethyl magnesium bromide with compounds of Formula F, wherein R³⁴ and R³⁶—R⁴¹ are as defined in Formula I, R³⁵ is as defined in Formula I or is a suitable protecting group and LG is a suitable leaving group, such as chloro, to provide compounds of Formula G. Reduction of keto group in the compounds of Formula G, for example using Al-based reducing agents such as lithium borohydride, lithium aluminum hydride or lithium aluminum deuteride, provides the compounds of Formula A, wherein R³² and R³³ are either H or D. If R³⁵ is a protecting group, it is either removed in a separate step or, wherein the protecting group is removed in the presence of Al-based reducing agents is removed during the reduction of the compounds of Formula G. A person skilled in the art would appreciate that a similar reaction sequence can be used to prepare compounds of Formula I, wherein Q is

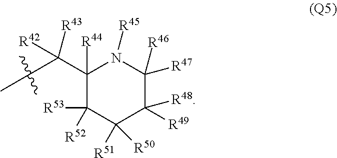

In some embodiments, when Q is

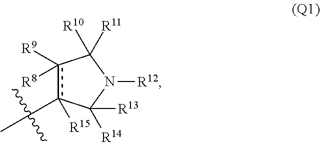

[symbol]

is a single bond, R¹⁵ and R¹² are as defined in Formula I, R⁸ and R⁹ are both H, and R¹⁰, R¹¹, R¹³ and R¹⁴ are either H or D, the compounds of Formula A are prepared as shown in Scheme 5:

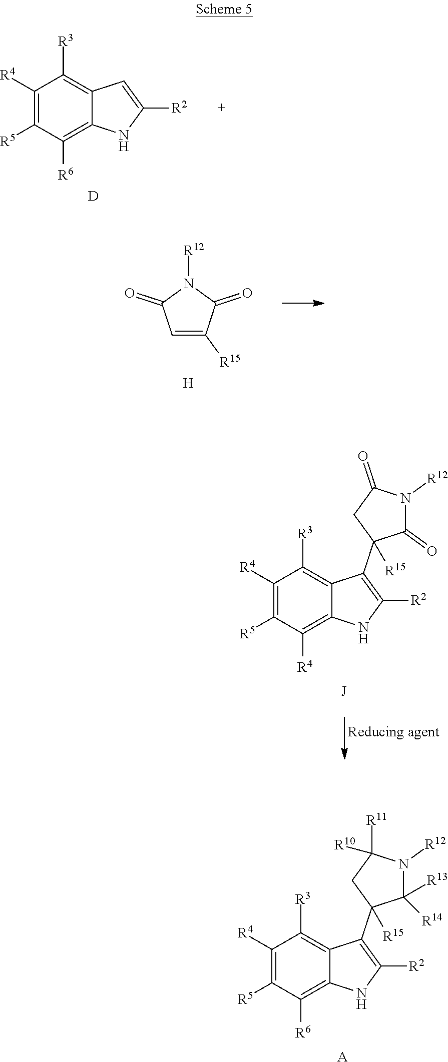

Therefore, in some embodiments, compounds of Formula D, wherein R² and R³—R⁶ are as defined in Formula I, are coupled with compounds of Formula H, wherein R¹² is as defined in Formula I or is a suitable protecting group and R¹⁵ is as defined in Formula I, in a suitable solvent such as acetic acid, to provide compounds of Formula J. Reduction of the keto groups in the compounds of Formula J, for example using Al-based reducing agents such as lithium borohydride, lithium aluminum hydride or lithium aluminum deuteride, provides the compounds of Formula A, wherein Q is

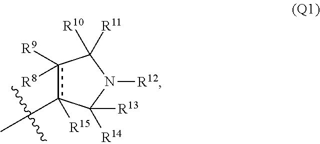

[symbol]

is a single bond, R¹⁵ is as defined in Formula I, R⁸ and R⁹ are both H, and R¹⁰, R¹¹, R¹³ and R¹⁴ are either H or D. If R¹² is a protecting group, it is either removed in a separate step or, wherein the protecting group is removed in the presence of Al-based reducing agents is removed during the reduction of the compounds of Formula J. A person skilled in the art would appreciate that a similar reaction sequence can be used to prepare compounds of Formula I, wherein Q is

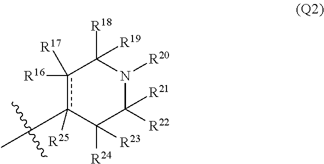

and [symbol] is a single bond.

In some embodiments, when Q is

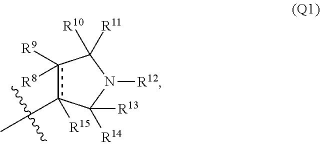

[symbol]

is a single or a double bond, R⁸ and R¹⁵ are H (when [symbol] is a single bond) or are not present (when [symbol] is a double bond), R¹⁰, R¹¹, R¹³ and R¹⁴ all H and R¹² is as defined in Formula I, the compounds of Formula A are prepared as shown in Scheme 6:

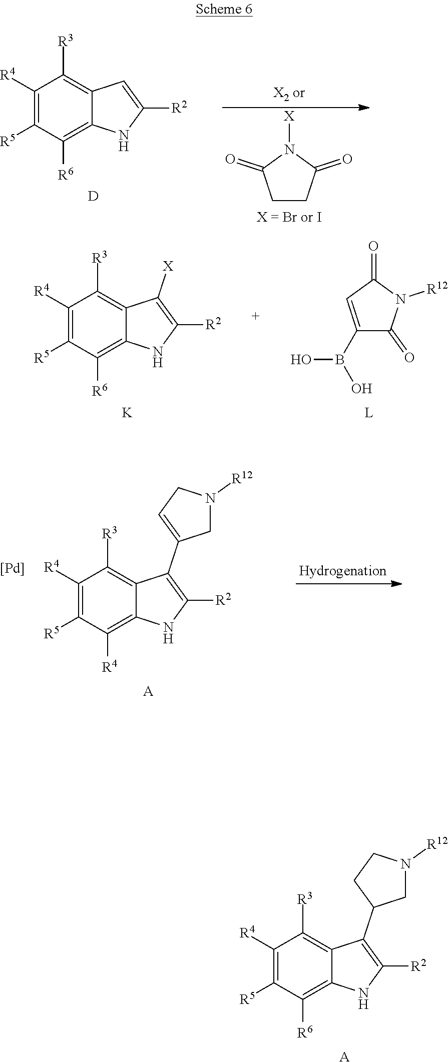

Therefore, in some embodiments, compounds of Formula D, wherein R² and R³—R⁶ are as defined in Formula I, are reacted with a dihalide or N-halosuccinimide compound wherein X is a halide such as Br or I, to provide the intermediate compounds of Formula K which are coupled to the borono maleimido compound of Formula L, wherein R¹² is as defined in Formula I or is a suitable protecting group, in the presence of a palladium catalyst to provide the compound of Formula A, wherein Q is

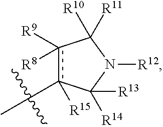

[symbol]

is double bond and R⁸ and R¹⁵ are not present, R¹⁰, R¹¹, R¹³ and R¹⁴ all H and R¹² is as defined in Formula I, which is then hydrogenated by methods known in the art for example, in the presence of palladium on carbon (“Pd/C”), to provide the compound of Formula A wherein Q is

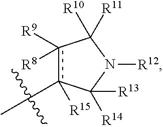

[symbol]

is a single bond, R⁸, R⁹, R¹⁰, R¹¹, R¹³ and R¹⁴ all H and R¹² is as defined in Formula 1. A person skilled in the art would appreciate that a similar reaction sequence can be used to prepare compounds of Formula I, wherein Q is

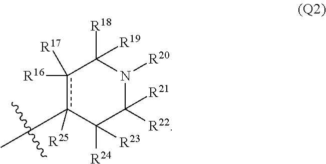

In some embodiments, when Q is

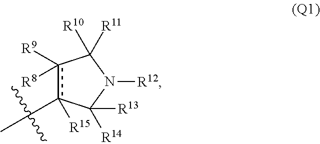

[symbol]

is a single or a double bond, R⁸ and R¹⁵ are H (when [symbol] is a single bond) or are not present (when [symbol] is a double bond) and R⁹, R¹⁰, R¹¹, R¹², R¹³ and R¹⁴ are as defined in Formula I, the compounds of Formula A are prepared as shown in Scheme 7:

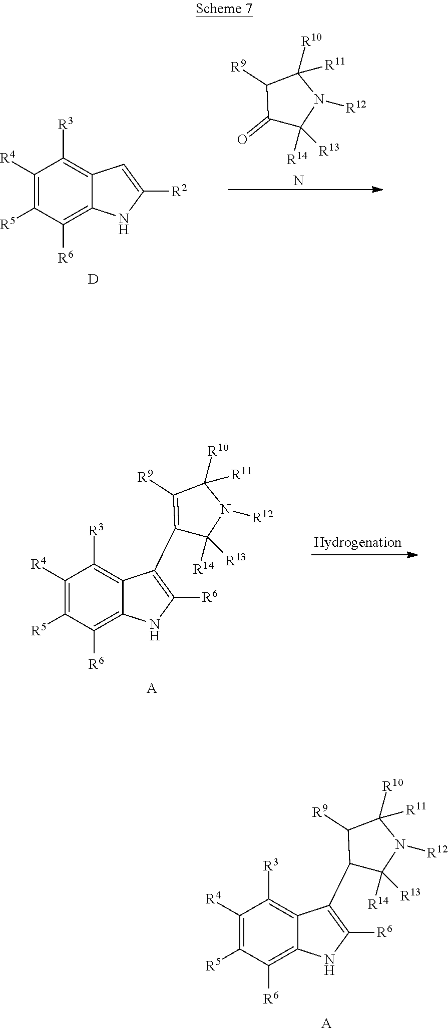

Therefore, in some embodiments, compounds of Formula D, wherein R² and R¹—R⁴ are as defined in Formula I, are reacted with oxo-pyrrolidine Formula N, wherein R⁹, R¹⁰, R¹¹, R¹², R¹³ and R¹⁴ are as defined in Formula I, for example in a suitable solvent such as ethanol (EtOH) at a suitable temperature such as the reflux temperature of the reaction mixture, to provide the compounds of Formula A, wherein Q is

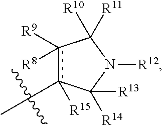

[symbol]

is a double bond, R⁸ and R¹⁵ are not present and R⁹, R¹⁰, R¹¹, R¹², R¹³ and R¹⁴ are as defined in Formula I, which are then reduced by methods known in the art, for example in the presence of palladium on carbon (“Pd/C”), to provide the compounds of Formula A wherein Q is

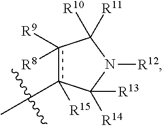

[symbol]

is a single bond, R⁸ and R¹⁵ are H and R⁹, R¹⁰, R¹¹, R¹², R¹³ and R¹⁴ are as defined in Formula I. A person skilled in the art would appreciate that a similar reaction sequence can be used to prepare compounds of Formula I, wherein Q is

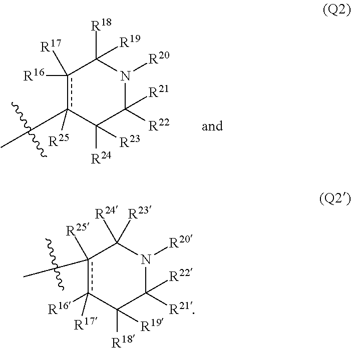

In some embodiments, when Q is

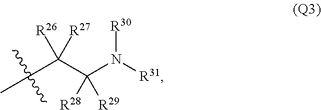

the compounds of Formula I are prepared as shown in Scheme 8:

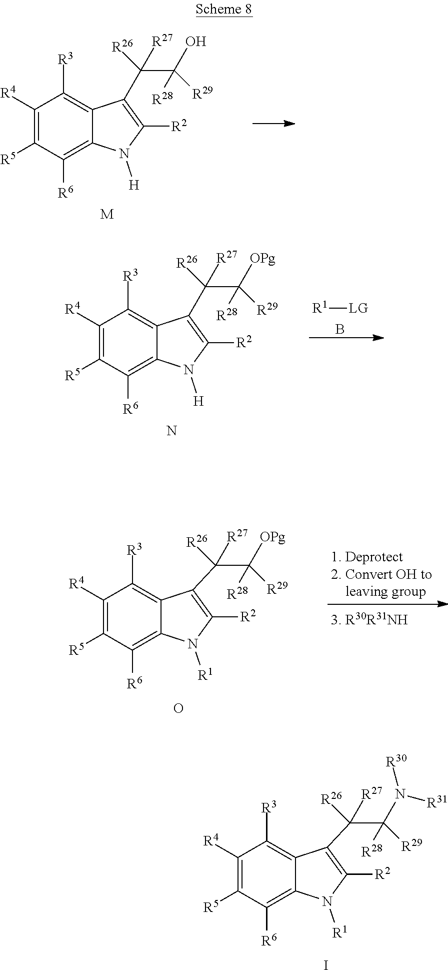

Therefore, in some embodiments, the OH group of the compounds of Formula M, wherein R²—R⁶ and R²⁶—R²⁹ are as defined in Formula I, are protected with a suitable protecting group and the R¹ group installed on the indole nitrogen as described in Scheme 1. The protecting group is then removed and the OH group is converted to a leaving group, for example a mesylate, and the resulting compound is reacted with an amine NHR³⁰R³¹, wherein R³⁰ and R³¹ are as defined in Formula I, to provide compounds of Formula I, wherein Q is

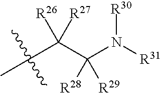

and R²—R⁶, R²⁶—R²⁹ and R³⁰—R³¹ are as defined in Formula I.

In some embodiments, the compounds of Formula I wherein R¹ is C₀₂C₁₋₄alkyleneOC(O)R⁷ or CO₂C₁₋₄alkyleneC(O)R⁷ are prepared as shown in Scheme 9:

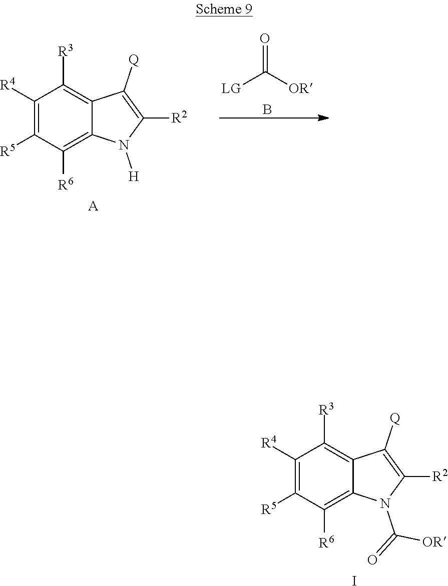

Therefore, in some embodiments, compounds of Formula A, wherein R²—R⁶ and Q are as defined in Formula I are reacted with compounds of Formula B, wherein R′ is C₁₋₄alkyleneOC(O)R⁷ or C₁₋₄alkyleneC(O)R⁷ and LG is a suitable leaving group such as a halide or 1H-imidazole, and R⁷ is as defined in Formula I, in the presence of a base to provide the compounds of Formula I. In some embodiments, and the base is, for example, 1,8-diazabicyclo-[5.4.0]undec-7-ene (DBU), NaH, NaOtBu or lithium bis(trimethylsilyl)amide (LiHMDS).

In some embodiments, the compounds of Formula I wherein R¹ is CO₂C₁₋₄alkyleneOC(O)R⁷ or CO₂C₁₋₄alkyleneC(O)R⁷ are prepared as shown in Scheme 10:

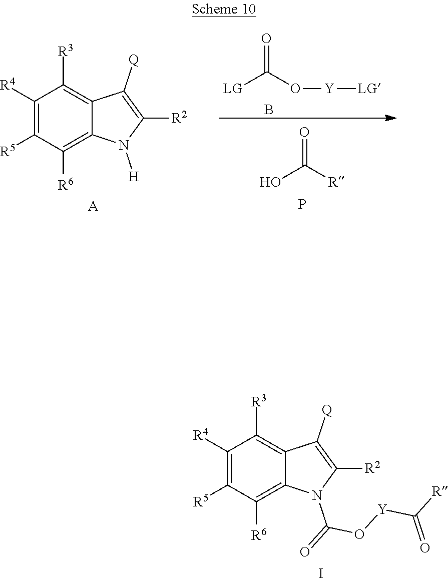

Therefore, in some embodiments, compounds of Formula A, wherein R²—R⁶ and Q are as defined in Formula I are reacted with compounds of Formula B, wherein Y is C₁₋₆alkylene and LG and LG′ are suitable leaving groups (which may be the same or different), such as a halide or 1H-imidazole, wherein LG′ is located on any of the carbons of the C₁₋₆alkylene, and a carboxylic acid B2, wherein R″ is OR⁷ or R⁷ wherein R⁷ is as defined in Formula I, in the presence of a base to provide the compounds of Formula I. In some embodiments, the base is a base, is for example, N-methylmoropholine, triethyamine or sodium hydroxide.

Compounds of Formula I, D or A, wherein one or more of R², R³—R⁶ are deuterium are available, for example, using a hydrogen-deuterium exchange reaction on a suitable starting substrate, wherein this exchange reaction is catalyzed by Pd/C in D₂O as described in Esaki, H. et al., Tetrahedron, 2006, 62:10954-10961, and modifications thereof known to a person skilled in the art.

Compounds of Formula I wherein R⁴ is OCD₃ are available, for example, using methods as described in Xu, Y—Z and Chen, C. J. Label Compd. Radiopharm. (2006), 49:897-902, and modifications thereof and modifications thereof known to a person skilled in the art.

Compounds of Formula B, C, D, F, H, L, M, N and P, as well as other reagents used in the above Schemes, are available from commercial sources or can be readily prepared using methods known in the art.

A person skilled in the art would appreciate that further manipulation of the substituent groups using known chemistry can be performed on the intermediates and final compounds in the Schemes above to provide alternative compounds of the application.

Salts of compounds of the application may be formed by methods known to those of ordinary skill in the art, for example, by reacting a compound of the application with an amount of acid or base, such as an equivalent amount, in a medium such as one in which the salt precipitates or in aqueous medium followed by lyophilization.

The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

Isotopically-enriched compounds of the application and pharmaceutically acceptable salts and/or solvates thereof, can be prepared without undue experimentation by conventional techniques well known to those skilled in the art or by processes analogous to those described in the Schemes and Examples herein using suitable isotopically-enriched reagents and/or intermediates.

Throughout the processes described herein it is to be understood that, where appropriate, suitable protecting groups will be added to and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities and ways to circumvent them by carrying out appropriate transformations and synthetic steps in a suitable order, will be readily understood to one skilled in the art. Examples of transformations are given herein and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

### EXAMPLES

The following non-limiting examples are illustrative of the present application.

### General Methods

All starting materials used herein were commercially available or earlier described in the literature. The ¹H and ¹³C NMR spectra were recorded either on Bruker 300, Bruker DPX400 or Varian+400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals as appearing in the recordings is generally indicated, for example as s: singlet, br s: broad singlet, d: doublet, t: triplet, q: quartet, m: multiplet. Unless otherwise indicated, in the tables below, ¹H NMR data was obtained at 400 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### A. Synthesis of Compounds

### Example 1: 2-(1H-Indol-3-yl-2,4,5,6,7-d₅)—N,N-bis(methyl-d₃)ethan-1-amine (3)

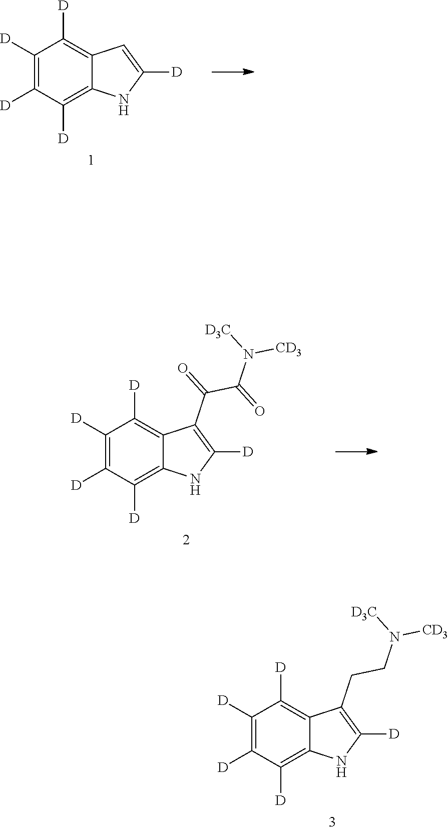

Synthesis of 2-(1H-indol-3-yl-2,4,5,6,7-d₅)—N,N-bis(methyl-d₃)-2-oxoacetamide (2): A solution of 1H-indole-2,4,5,6,7-d₅ (1.4 g, 11.368 mmol) in dry ether (30 mL) was treated with oxalyl chloride (0.75 mL, 11.368 mmol) at 0° C. The reaction was brought to room temperature and stirred for additional 16 h. The reaction was cooled to 0° C., treated with bis(methyl-d₃)amine hydrochloride (3.48 g, 39.788 mmol, free based with Et₃N in THF (80 mL)) over a period of 5 min. The reaction was brought to room temperature and stirred for 4 h. The reaction was quenched with water (100 mL) and product was extracted into ethyl acetate (2×75 mL). Combined ethyl acetate layer was washed with brine (25 mL) and dried (Na₂SO₄). Solvent was evaporated and crude was purified by flash column chromatography (MeOH:CH₂Cl₂, 5:95) on silica gel to obtain the title compound 2 (1.65 g, 63.9%) as light-yellow solid. ESI-MS (m/z, %): 249 (M+Na, 100)

Synthesis of 2-(1H-indol-3-yl-2,4,5,6,7-d₅)—N,N-bis(methyl-d₃)ethan-1-amine (3): A suspension of Lithium aluminum hydride (0.82 g, 21.820 mmol) in dry THF (10 mL) was treated with 2-(1H-indol-3-yl-2,4,5,6,7-d₅)—N,N-bis(methyl-d₃)-2-oxoacetamide (0.62 g, 2.727 mmol) in dry THF (20 mL) at 0° C. over a period of 10 min. The reaction was brought to room temperature, then refluxed for additional 16 hours. The reaction was cooled to 0° C., then quenched with sequential addition of water (0.82 mL), 2 N NaOH solution (0.82 mL) and water (0.82 mL) over a period of 15 min. The reaction was brought to room temperature, stirred for additional 30 min. Solid was filtered off and washed with THF (2×50 mL). Combined THF layer was evaporated and crude was purified by column chromatography (2 M NH₃ in MeOH:CH₂Cl₂, 5:95) on silica gel to obtain the title compound 3 (0.48 g, 88.8%) as pale-yellow solid. ¹H NMR (DMSO-d₆): δ 10.76 (s, 1H), 7.52-7.50 (m, 0.09H), 7.34-7.32 (m, 0.04H), 7.14 (d, 0.05H, J=1.5 Hz), 7.07-7.05 (m, 0.16H), 6.98-6.96 (m, 0.22H), 2.83-2.79 (m, 2H), 2.53-2.49 (m, 2H); ESI-MS (m/z, %): 200 (MH⁺, 100).

### Example 2: 2-(5-(Methoxy-d₃)-1H-indol-3-yl)-N,N-bis(methyl-d₃)ethan-1-amine-1,1,2,2-d₄ (6)

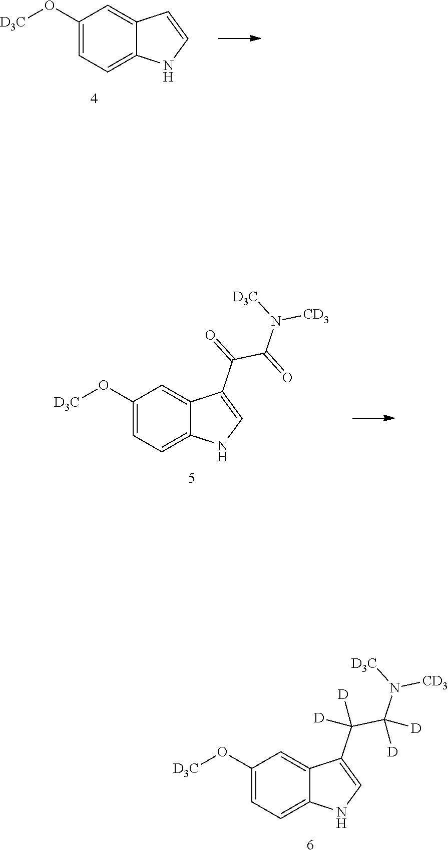

Synthesis of 2-(5-(methoxy-d₃)-1H-indol-3-yl)-N,N-bis(methyl-d₃)-2-oxoacetamide (5): A solution of 5-(methoxy-d₃)-1H-indole (1.04 g, 6.924 mmol) in dry ether (20 mL) was treated with oxalyl chloride (0.58 mL, 6.924 mmol) at 0° C. The reaction was brought to room temperature and stirred for additional 16 h. The reaction was cooled to 0° C., treated with bis(methyl-d₃)amine hydrochloride (2.1 g, 24.235 mmol, free based with Et₃N in THF (50 mL)) over a period of 5 min. The reaction was brought to room temperature and stirred for 4 h. The reaction was quenched with water (100 mL), worked-up and purified as described for compound 2 to obtain the title compound 5 (1.16 g, 66%) as a pale-yellow solid. ¹H NMR (DMSO-d₆): δ 12.19 (s, 1H), 8.03 (d, 1H, J=3.0 Hz), 7.61 (d, 1H, J=3.0 Hz), 7.43 (d, 1H, J=6.0 Hz), 6.91 (dd, 1H, J=3.0, 6.0 Hz); ESI-MS (m/z, %): 278 (M+Na, 100), 256 (MH⁺).

Synthesis of 2-(5-(methoxy-d₃)-1H-indol-3-yl)-N,N-bis(methyl-d₃)ethan-1-amine-1,1,2,2-d₄ (6): A suspension of Lithium aluminum deuteride (1.0 g, 23.812 mmol) in dry THF (10 mL) was treated with 2-(5-(methoxy-d₃)-1H-indol-3-yl)-N,N-bis(methyl-d₃)-2-oxoacetamide (0.76 g, 2.976 mmol) in dry THF (20 mL) at 0° C. over a period of 10 min. The reaction was brought to room temperature, then refluxed for additional 16 hours. The reaction was worked-up and purified as described for compound 3 to obtain the title compound 6 (0.59 g, 85.7%) as pale-yellow solid. ¹H NMR (DMSO-d₆): δ 10.59 (s, 1H), 7.22 (d, 1H, J=6.0 Hz), 7.09 (d, 1H, J=3.0 Hz), 6.97 (d, 1H, J=3.0 Hz), 6.71 (dd, 1H, J=3.0, 6.0 Hz); ESI-MS (m/z, %): 232 (MH⁺, 100).

### Example 3: (R)-3-((1-(Methyl-d₃)pyrrolidin-2-yl)methyl-d₂)-1H-indole (9)

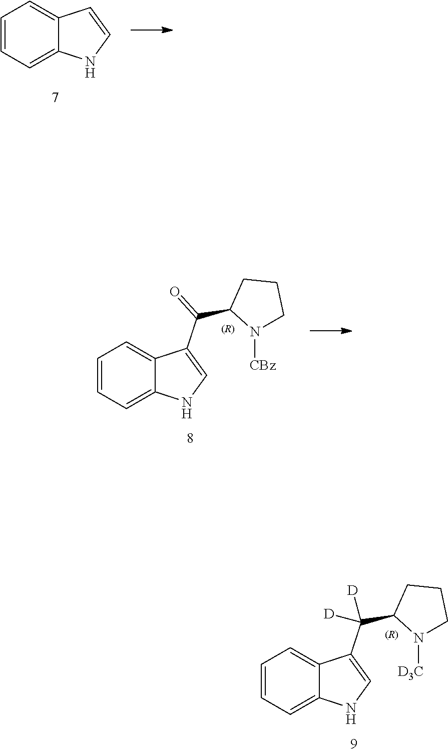

Synthesis of (R)-2-(2-(1H-indole-3-carbonyl)pyrrolidin-1-yl)-1-phenyl-2λ²-ethan-1-one (8): A solution of (2-oxo-2-phenyl-1λ²-ethyl)-D-proline (15.4 g, 61.782 mmol) in dry THF (70 mL) was treated with thionyl chloride (9.0 mL, 123.56 mmol) at 0° C. The reaction was brought to room temperature, then refluxed for 2 h. The reaction was brought to room temperature, solvent was evaporated and crude was dried under vacuum to obtain the corresponding acid chloride.

A solution of 1H-indole (7.23 g, 61.782 mmol) in dry CH₂Cl₂ (50 mL) at 5-10° C. was treated with above crude acid chloride in dry CH₂Cl₂ (50 mL) and ethyl magnesium bromide (41.2 mL, 123.56 mmol, 3 M in THF) simultaneously over a period of 15 min. and stirred at same temperature for further 15 min. The reaction was quenched with con. HCl (10 mL) followed by water (150 mL) and product was extracted into CH₂Cl₂ (2×150 mL). CH₂Cl₂ layer was washed with sat. NaHCO₃ solution (50 mL), brine (25 mL) and dried (Na₂SO₄). Solvent was evaporated and crude was purified by flash column chromatography (CH₂Cl₂ to EtOAc:CH₂Cl₂, 1:4) on silica gel to obtain the title compound 8 (13.0 g, 60.4%) as a light brown solid. ¹H NMR (CDCl₃): δ 9.54, 9.24 (2s, 1H), 8.43, 8.20 (2d, 1H, J=3.0 Hz), 7.70-7.64 (m, 1H), 7.44-7.00 (m, 8H), 5.29-5.01 (m, 3H), 3.81-3.63 (m, 2H), 2.28-2.25 (m, 1H), 2.09-1.94 (m, 3H); ESI-MS (m/z, %): 371 (M+Na, 100), 349 (MH⁺).

Synthesis of (R)-3-((1-(methyl-d₃)pyrrolidin-2-yl)methyl-d₂)-1H-indole (9): A suspension of lithium aluminum deuteride (0.50 g, 11.911 mmol) in dry THF (10 mL) was treated with (R)-2-(2-(1H-indole-3-carbonyl)pyrrolidin-1-yl)-1-phenyl-2λ²-ethan-1-one (0.83 g, 2.382 mmol) in dry THF (15 mL) at 0° C. over a period of 10 min. The reaction was brought to room temperature, then refluxed for additional 16 hours. The reaction was worked-up and purified as described for compound 3 to obtain the title compound 9 (0.5 g, 77%) as a beige solid. ¹H NMR (DMSO-d₆): δ 10.77 (s, 1H), 7.51 (d, 1H, J=6.0 Hz), 7.33 (d, 1H, J=6.0 Hz), 7.14 (d, 1H, J=3.0 Hz), 7.07 (dd, 1H, J=3.0 Hz), 7.05-6.95 (m, 1H), 3.00-2.95 (m, 1H), 2.36-2.32 (m, 1H), 2.14-2.07 (m, 1H), 1.71-1.43 (m, 4H); ESI-MS (m/z, %): 219 (MH⁺, 100).

### Example 4: (R)-3-(Pyrrolidin-2-ylmethyl-d₂)-1H-indole (11)

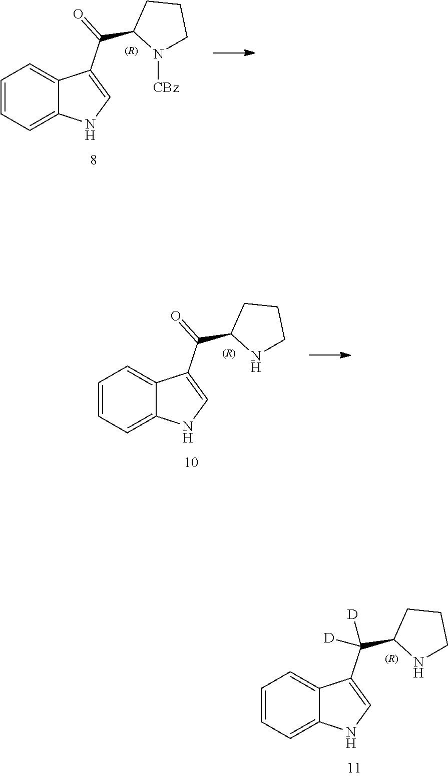

Synthesis of (R)-3-prolyl-1H-indole (10): A suspension of (R)-2-(2-(1H-indole-3-carbonyl)pyrrolidin-1-yl)-1-phenyl-2λ²-ethan-1-one (1.2 g, 3.444 mmol) in methanol (25 mL) was treated with 10% palladium on carbon (1.2 g) and hydrogenated using Parr apparatus at 33 PSI for 50 min. The reaction was filtered through a pad celite and washed with methanol (3×25 mL). Combined methanol layer was evaporated and crude was purified by crystallization from a mixture of CH₂Cl₂:hexanes (1:1) to obtain the title compound 10 (0.48 g, 65%) as an off-white solid. ¹H NMR (DMSO-d₆): δ 8.41 (d, 1H, J=3.0 Hz), 8.21 (dd, 1H, J=3.0 Hz), 7.50-7.46 (m, 1H), 7.25-7.18 (m, 2H), 4.42-4.39 (m, 1H), 3.10-3.04 (m, 1H), 2.79-2.75 (m, 1H), 2.20-2.18 (m, 1H), 1.76-1.62 (m, 3); ESI-MS (m/z, %): 215 (MH⁺, 100).

Synthesis of (R)-3-(pyrrolidin-2-ylmethyl-d₂)-1H-indole (11): A suspension of (R)-3-prolyl-1H-indole (0.45 g, 2.100 mmol) in dry THF (20 mL) was treated with lithium aluminum deuteride (0.26 g, 6.300 mmol) at 0° C. The reaction was brought to room temperature, then refluxed for additional 16 hours. The reaction was worked-up and purified as described for compound 3 to obtain the title compound 11 (0.13 g, 31%) as a pale-yellow solid. ¹H NMR (DMSO-d₆): δ 10.79 (s, 1H), 7.53 (d, 1H, J=6.0 Hz), 7.34 (d, 1H, J=6.0 Hz), 7.08 (d, 1H, J=3.0 Hz), 7.05 (dd, 1H, J=3.0, 6.0 Hz), 6.98 (dd, 1H, J=3.0, 6.0 Hz), 3.29-3.22 (m, 1H), 2.95-2.88 (m, 1H), 2.75-2.69 (m, 1H), 1.76-1.57 (m, 3H), 1.36-1.29 (m, 1H); ESI-MS (m/z, %): 203 (MH⁺, 100).

### Example 5: (R)-5-Methoxy-3-((1-(methyl-d₃)pyrrolidin-2-yl)methyl)-1H-indole (15)

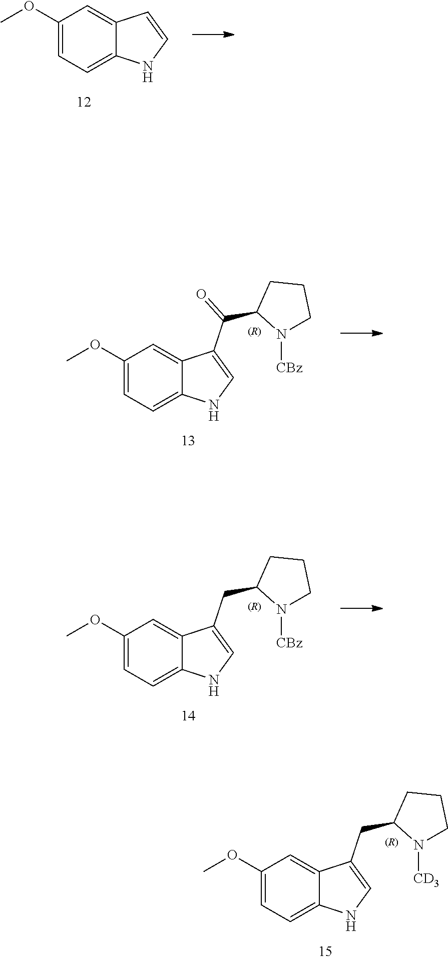

Synthesis of (R)-2-(2-(5-methoxy-1H-indole-3-carbonyl)pyrrolidin-1-yl)-1-phenyl-2λ²-ethan-1-one (13): A solution of (2-oxo-2-phenyl-1λ²-ethyl)-D-proline (17.4 g, 69.806 mmol) in dry THF (100 mL) was treated with thionyl chloride (10.2 mL, 139.613 mmol) at 0° C. The reaction was brought to room temperature, then refluxed for 2 h. The reaction was brought to room temperature, solvent was evaporated and crude was dried under vacuum to obtain the corresponding acid chloride.

A solution of 5-methoxy-1H-indole (10.2 g, 69.806 mmol) in dry CH₂Cl₂ (100 mL) at 5-10° C. was treated with above crude acid chloride in dry CH₂Cl₂ (40 mL) and ethyl magnesium bromide (46.5 mL, 139.61 mmol, 3 M in THF) simultaneously over a period of 15 min. and stirred at same temperature for further 15 min. The reaction was quenched, worked-up and purified as described for compound 8 to obtain the title compound 13 (18.9 g, 71.5%) as a yellow solid.; ESI-MS (m/z, %): 401 (M+Na, 100), 379 (MH⁺).

Synthesis of (R)-2-(2-((5-methoxy-1H-indol-3-yl)methyl)pyrrolidin-1-yl)-1-phenyl-2λ²-ethan-1-one (14): A solution of (R)-2-(2-(5-methoxy-1H-indole-3-carbonyl)pyrrolidin-1-yl)-1-phenyl-2λ²-ethan-1-one (1.6 g, 4.227 mmol) in dry THF (25 mL) was treated with a solution of lithium borohydride (8.45 mL, 16.911 mmol, 2 M solution in THF) at room temperature over a period of 5 min. and the reaction was refluxed for additional 4 hours. The reaction was cooled to 0° C., quenched with the careful addition of methanol (10 mL) over a period of 15 min. The reaction was brought to room temperature and stirred for additional 1 h. The reaction was treated with sat. NaHCO₃ solution (30 mL) and product was extracted into ethyl acetate (3×50 mL). Combined ethyl acetate layer was washed with brine (20 mL) and dried (Na₂SO₄). Solvent was evaporated and crude was purified by flash column chromatography (MeOH; CH₂Cl₂, 2:98) on silica gel to obtain the title compound 14 (1.17 g, 76%) as a pale yellow semi-solid. ¹H NMR (CDCl₃): δ 7.91 (s, 1H), 7.46-7.18 (m, 6H), 7.05-6.85 (m, 3H), 5.27-5.20 (m, 2H), 3.70 (S, 3H), 3.51-3.44 (m, 2H), 3.48-3.44 (m, 1H), 3.35-3.30 (m, 1H), 2.82-2.71 (m, 1H), 1.70-1.60 (m, 4H); ESI-MS (m/z, %): 387 (M+Na, 100).

Synthesis of (R)-5-methoxy-3-((1-(methyl-d₃)pyrrolidin-2-yl)methyl)-1H-indole (15): A suspension of (R)-2-(2-((5-methoxy-1H-indol-3-yl)methyl)pyrrolidin-1-yl)-1-phenyl-2λ²-ethan-1-one (0.95 g, 2.606 mmol) in dry THF (30 mL) was treated with lithium aluminum deuteride (0.27 g, 6.516 mmol) at 0° C. The reaction was brought to room temperature, then refluxed for additional 16 hours. The reaction was worked-up and purified as described for compound 3 to obtain the title compound 15 (0.5 g, 78%) as light brown glue. ¹H NMR (DMSO-d₆): δ 10.61 (s, 1H), 7.22 (d, 1H, J=6.0 Hz), 7.10 (d, 1H, J=3.0 Hz), 6.98 (d, 1H, J=3.0 Hz), 6.71 (d, 1H, J=3.0 Hz), 3.76 (s, 3H), 3.02-2.96 (m, 2H), 2.50-2.30 (m, 2H), 2.15-2.09 (m, 1H), 1.75-1.43 (m, 4H); ESI-MS (m/z, %): 248 (MH⁺, 100).

### Example 6: (R)-5-(Methoxy-d₃)-3-((1-(methyl-d₃)pyrrolidin-2-yl)methyl)-1H-indole (18)

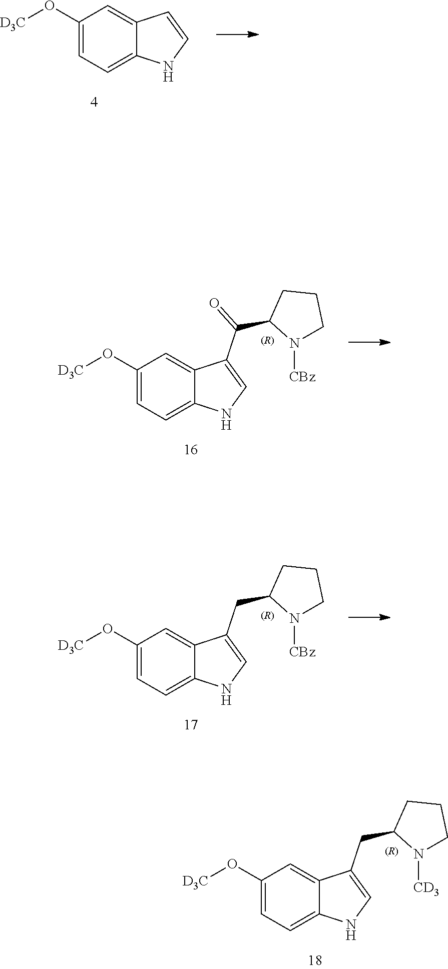

Synthesis of (R)-2-(2-(5-(methoxy-d₃)-1H-indole-3-carbonyl)pyrrolidin-1-yl)-1-phenyl-2λ²-ethan-1-one (16): A solution of (2-oxo-2-phenyl-1λ²-ethyl)-D-proline (11.74 g, 47.099 mmol) in dry THF (70 mL) was treated with thionyl chloride (6.87 mL, 94.198 mmol) at 0° C. The reaction was brought to room temperature, then refluxed for 2 h. The reaction was brought to room temperature, solvent was evaporated and crude was dried under vacuum to obtain the corresponding acid chloride.

A solution of 5-(methoxy-d₃)-1H-indole (7.0 g, 47.099 mmol) in dry CH₂Cl₂ (50 mL) at 5-10° C. was treated with above crude acid chloride in dry CH₂Cl₂ (50 mL) and ethyl magnesium bromide (31.4 mL, 94.198 mmol, 3 M in THF) simultaneously over a period of 15 min. and stirred at same temperature for further 15 min. The reaction was quenched, worked-up and purified as described for compound 8 to obtain the title compound 16 (12.0 g, 66.8%) as a pale-yellow solid. ¹H NMR (CDCl₃): δ 9.63, 8.68 (2s, 1H), 7.67-7.31 (m, 5H), 7.12-6.76 (m, 3H), 5.30-5.04 (m, 3H), 3.83-3.63 (m, 2H), 2.30-2.25 (m, 1H), 2.12-1.93 (m, 3H); ESI-MS (m/z, %): 404 (M+Na, 100), 382 (MH⁺).

Synthesis of (R)-2-(2-((5-methoxy-1H-indol-3-yl)methyl)pyrrolidin-1-yl)-1-phenyl-2λ²-ethan-1-one (17): A solution of (R)-2-(2-(5-(methoxy-d₃)-1H-indole-3-carbonyl)pyrrolidin-1-yl)-1-phenyl-2λ²-ethan-1-one (1.25 g, 3.227 mmol) in dry THF (25 mL) was treated with a solution of lithium borohydride (6.55 mL, 16.911 mmol, 2 M solution in THF) at room temperature over a period of 5 min. and the reaction was refluxed for additional 4 hours. The reaction was worked-up and purified as described for compound 14 to obtain the title compound 17 (1.16 g, 66%) as a pale-yellow solid. ¹H NMR (CDCl₃): δ 7.92 (s, 1H), 7.45-7.27 (m, 6H), 7.01-6.84 (m, 3H), 5.32-5.19 (m, 2H), 3.49-3.30 (m, 2H), 3.23-3.22 (m, 1H), 3.19-3.12 (m, 1H), 2.81-2.71 (m, 1H), 2.80-2.65 (m, 4H); ESI-MS (m/z, %): 390 (M+Na, 100), 368 (MH⁺).

Synthesis of (R)-5-(methoxy-d₃)-3-((1-(methyl-d₃)pyrrolidin-2-yl)methyl)-1H-indole (18): A suspension of (R)-2-(2-((5-methoxy-1H-indol-3-yl)methyl)pyrrolidin-1-yl)-1-phenyl-2λ²-ethan-1-one (0.81 g, 2.204 mmol) in dry THF (30 mL) was treated with lithium aluminum deuteride (0.23 g, 5.510 mmol) at 0° C. The reaction was brought to room temperature, then refluxed for additional 16 hours. The reaction was worked-up and purified as described for compound 3 to obtain the title compound 18 (0.45 g, 81.8%) as light brown glue. ¹H NMR (DMSO-d₆): δ 10.60 (s, 1H), 7.21 (d, 1H, J=6.0 Hz), 7.09 (d, 1H, J=3.0 Hz), 6.97 (d, 1H, J=3.0 Hz), 6.71 (dd, 1H, J=3.0, 6.0 Hz), 3.18 (d, 1H, J=6.0 Hz), 3.02-2.95 (m, 2H), 2.43-2.33 (m, 1H), 2.14-2.08 (m, 1H), 1.74-1.45 (m, 4H); ESI-MS (m/z, %): 251 (MH⁺, 100).

### Example 7: (R)-5-(Methoxy-d₃)-3-((1-(methyl-d₃)pyrrolidin-2-yl)methyl-d₂)-1H-indole (19)

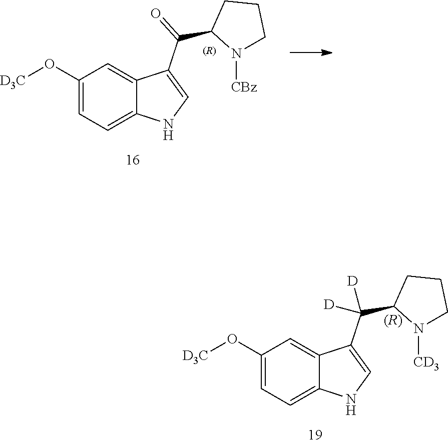

Synthesis of (R)-5-(methoxy-d₃)-3-((1-(methyl-d₃)pyrrolidin-2-yl)methyl-d₂)-1H-indole (19): A suspension of (R)-2-(2-(5-(methoxy-d₃)-1H-indole-3-carbonyl)pyrrolidin-1-yl)-1-phenyl-2λ²-ethan-1-one (0.85 g, 2.228 mmol) in dry THF (30 mL) was treated with lithium aluminum deuteride (0.46 g, 11.141 mmol) at 0° C. The reaction was brought to room temperature, then refluxed for additional 16 hours. The reaction was worked-up and purified as described for compound 3 to obtain the title compound 19 (0.5 g, 89%) as light brown glue. ¹H NMR (DMSO-d₆): δ 10.60 (s, 1H), 7.21 (d, 1H, J=6.0 Hz), 7.09 (d, 1H, J=3.0 Hz), 6.97 (d, 1H, J=3.0 Hz), 6.71 (dd, 1H, J=3.0 Hz), 2.99-2.95 (m, 1H), 2.35-2.31 (m, 1H), 2.14-2.07 (m, 1H), 1.75-1.41 (m, 4H); ESI-MS (m/z, %): 253 (MH⁺, 100).

### Example 8: (R)-5-Methoxy-3-((1-(methyl-d₃)pyrrolidin-2-yl)methyl-d₂)-1H-indole (20)

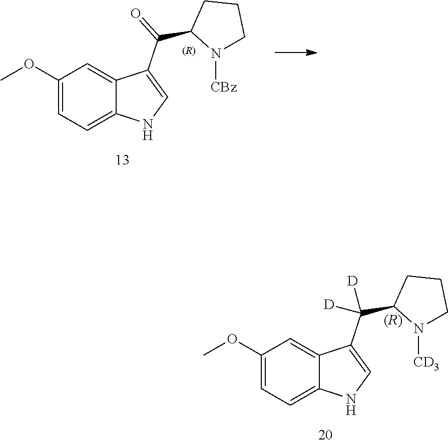

Synthesis of (R)-5-methoxy-3-((1-(methyl-d₃)pyrrolidin-2-yl)methyl-d₂)-1H-indole (20): A suspension of (R)-2-(2-(5-methoxy-1H-indole-3-carbonyl)pyrrolidin-1-yl)-1-phenyl-2λ²-ethan-1-one (0.91 g, 2.404 mmol) in dry THF (30 mL) was treated with lithium aluminum deuteride (0.5 g, 12.023 mmol) at 0° C. The reaction was brought to room temperature, then refluxed for additional 16 hours. The reaction was worked-up and purified as described for compound 3 to obtain the title compound 20 (0.46 g, 76.6%) as pale-yellow glue. ¹H NMR (DMSO-d₆): δ 10.61 (s, 1H), 7.22 (d, 1H, J=6.0 Hz), 7.09 (d, 1H, J=1.5 Hz), 6.98 (d, 1H, J=3.0 Hz), 6.72 (dd, 1H, J=3.0, 6.0 Hz), 3.76 (s, 3H), 3.00-2.95 (m, 1H), 2.36-2.32 (m, 1H), 2.14-2.08 (m, 1H), 1.76-1.42 (m, 4H); ESI-MS (m/z, %): 250 (MH⁺, 100).

### Example 9: (R)-5-(Methoxy-d₃)-3-(pyrrolidin-2-ylmethyl)-1H-indole (22)

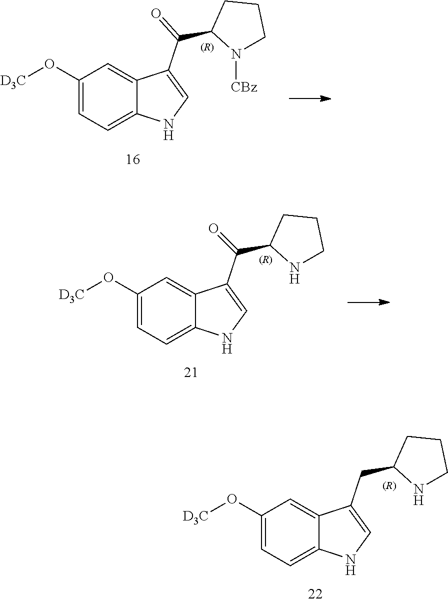

Synthesis of (R)-5-(methoxy-d₃)-3-prolyl-1H-indole (21): A suspension of (R)-2-(2-(5-(methoxy-d₃)-1H-indole-3-carbonyl)pyrrolidin-1-yl)-1-phenyl-2λ²-ethan-1-one (3.0 g, 7.864 mmol) in methanol (100 mL) was treated with 10% palladium on carbon (3.0 g) and hydrogenated using Parr apparatus at 33 PSI for 24 h. The reaction was filtered through a pad celite and washed with methanol (3×25 mL). Combined methanol layer was evaporated and crude was purified by crystallization from a mixture of CH₂Cl₂:hexanes (1:1) to obtain the title compound 21 (0.475 g, 24.4%) as light-yellow solid. ¹H NMR (DMSO-d₆): δ11.89 (brs, 1H), 8.32 (s, 1H), 7.72 (d, 1H, J=3.0 Hz), 7.38 (d, 1H, J=9.0 Hz), 6.85 (dd, 1H, J=6.0 Hz), 4.38-4.35 (m, 1H), 3.07-3.03 (m, 1H), 2.78-2.72 (m, 1H), 2.20-2.14 (m, 1H), 1.73-1.61 (m, 3H); ESI-MS (m/z, %): 248 (MH⁺, 100).

Synthesis of (R)-5-(methoxy-d₃)-3-(pyrrolidin-2-ylmethyl)-1H-indole (22): A suspension of (R)-5-(methoxy-d₃)-3-prolyl-1H-indole (0.23 g, 0.930 mmol) in dry THF (20 mL) was treated with lithium aluminum hydride (0.105 g, 2.790 mmol) at 0° C. The reaction was brought to room temperature, then refluxed for additional 16 hours. The reaction was worked-up and purified as described for compound 3 to obtain the title compound 22 (0.075 g, 35.7%) as a pale yellow semi-solid. ¹H NMR (DMSO-d₆): δ 10.63 (s, 1H), 7.23 (dd, 1H, J=3.0 Hz), 7.12 (d, 1H, J=3.0 Hz), 7.00 (s, 1H), 6.72 (dd, 1H, J=6.0 Hz), 3.31-3.24 (m, 2H), 2.96-2.91 (m, 1H), 2.82-2.67 (m, 2H), 1.77-1.60 (m, 3H), 1.39-1.32 (m, 1H); ESI-MS (m/z, %): 234 (MH⁺, 100).

### Example 10: (R)-5-(Methoxy-d₃)-3-(pyrrolidin-2-ylmethyl-d₂)-1H-indole (23)

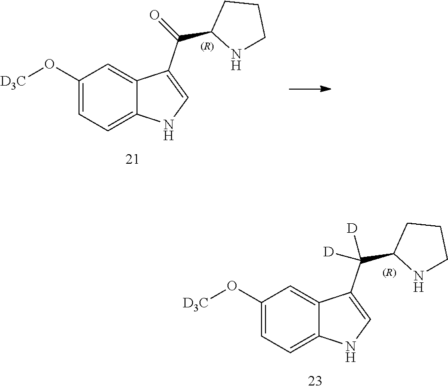

Synthesis of (R)-5-(methoxy-d₃)-3-(pyrrolidin-2-ylmethyl-d₂)-1H-indole (23): A suspension of (R)-5-(methoxy-d₃)-3-prolyl-1H-indole (0.24 g, 0.970 mmol) in dry THF (25 mL) was treated with lithium aluminum deuteride (0.122 g, 2.911 mmol) at 0° C. The reaction was brought to room temperature, then refluxed for additional 16 hours. The reaction was worked-up and purified as described for compound 3 to obtain the title compound 23 (0.05 g, 21.7%) as a light brown semi-solid. ¹H NMR (DMSO-d₆): δ 10.63 (s, 1H), 7.23 (dd, 1H, J=3.0 Hz), 7.11 (d, 1H, J=1.5 Hz), 7.00 (d, 1H, J=1.5 Hz), 6.70 (dd, 1H, J=3.0 Hz), 3.26-3.17 (m, 1H), 2.96-2.91 (m, 1H), 2.77-2.71 (m, 1H), 1.77-1.59 (m, 3H), 1.44-1.30 (m, 1H); ESI-MS (m/z, %): 235 (MH⁺, 100).

### Example 11: (R)-5-Methoxy-3-(pyrrolidin-2-ylmethyl-d₂)-1H-indole (25)

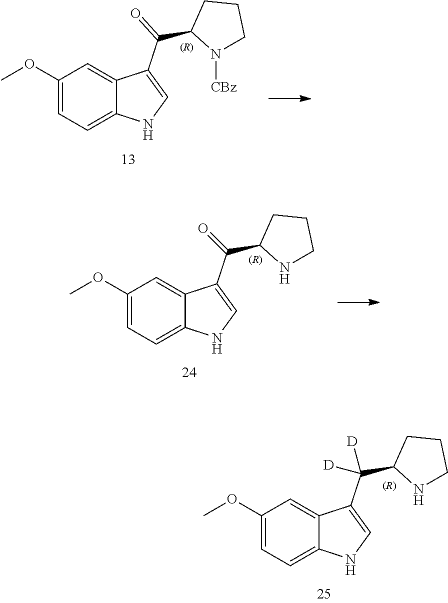

Synthesis of (R)-5-methoxy-3-prolyl-1H-indole (24): A suspension of (R)-2-(2-(5-methoxy-1H-indole-3-carbonyl)pyrrolidin-1-yl)-1-phenyl-2λ²-ethan-1-one (4.65 g, 12.287 mmol) in methanol (100 mL) was treated with 10% palladium on carbon (4.5 g) and hydrogenated using Parr apparatus at 33 PSI for 2 h. The reaction was filtered through a pad of celite and washed with methanol (3×50 mL). Combined methanol layer was evaporated and crude was purified by crystallization from a mixture of CH₂Cl₂:hexanes (1:1) to obtain the title compound 24 (1.1 g, 36.6%) as a light-yellow solid. ¹H NMR (DMSO-d₆): δ 8.32 (s, 1H), 7.72 (d, 1H, J=1.5 Hz), 7.38 (d, 1H, J=6.0 Hz), 6.86 (dd, 1H, J=3.0, 6.0 Hz), 4.39-4.35 (m, 1H), 3.79 (s, 3H), 3.09-3.03 (m, 1H), 2.78-2.72 (m, 1H), 2.21-2.14 (m, 1H), 1.76-1.58 (m, 3H); ESI-MS (m/z, %): 245 (MH⁺, 100).

Synthesis of (R)-5-methoxy-3-(pyrrolidin-2-ylmethyl-d₂)-1H-indole (25): A suspension of (R)-5-methoxy-3-prolyl-1H-indole (0.44 g, 1.801 mmol) in dry THF (25 mL) was treated with lithium aluminum deuteride (0.226 g, 5.403 mmol) at 0° C. The reaction was brought to room temperature, then refluxed for additional 16 hours. The reaction was worked-up and purified as described for compound 3 to obtain the title compound 25 (0.2 g, 47.8%) as a pale-yellow solid. ¹H NMR (DMSO-d₆): δ 10.63 (s, 1H), 7.23 (d, 1H, J=3.0 Hz), 7.11 (d, 1H, J=1.5 Hz), 7.00 (d, 1H, J=1.5 Hz), 6.71 (dd, 1H, J=3.0, 6.0 Hz), 3.76 (s, 3H), 3.27-3.23 (m, 1H), 2.95-2.90 (m, 1H), 2.76-2.70 (m, 1H), 1.78-1.56 (m, 3H), 1.38-1.29 (m, 1H); ESI-MS (m/z, %): 233 (MH⁺, 100).

### Example 12: 2-(1H-Indol-3-yl-2,4,5,6,7-d₅)—N,N-dimethylethan-1-amine (27)

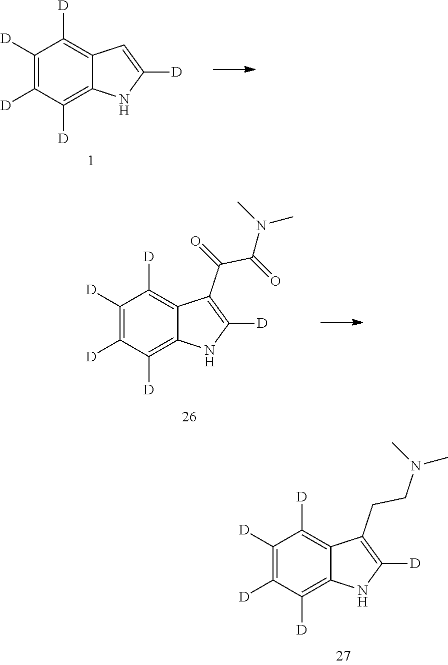 [SMILES: [2H]c1cc2c([2H])c([2H])c([2H])c([2H])c2[nH]1 ; [2H]c1[nH]c2c([2H])c([2H])c([2H])c([2H])c2c1C(=O)C(=O)N(C)C ; [2H]c1[nH]c2c([2H])c([2H])c([2H])c([2H])c2c1CCN(C)C]

Synthesis of 2-(1H-indol-3-yl-2,4,5,6,7-d₅)—N,N-dimethyl-2-oxoacetamide (26): A solution of 1H-indole-2,4,5,6,7-d₅ (0.8 g, 6.49 mmol) in dry ether (30 mL) was treated with oxalyl chloride (0.55 mL, 6.49 mmol) at 0° C. The reaction was brought to room temperature and stirred for additional 16 h. The reaction was cooled to 0° C., treated with dimethylamine solution (16.22 mL, 32.45 mmol, 2 M in THF) over a period of 5 min. The reaction was brought to room temperature and stirred for 4 h. The reaction was worked-up and purified as described for compound 2 to obtain the title compound 26 (1.1 g, 76.5%) as a light brown solid. ¹H NMR (CDCl₃) 10.09 (s, 1H), 8.35-8.33 (m, 0.17H), 7.76 (d, 0.05H, J=1.5 Hz), 7.38-7.26 (m, 0.85H), 3.11 (s, 3H), 3.06 (s, 3H): 5; ESI-MS (m/z, %): 244 (M+Na), 243 (100).

Synthesis of 2-(1H-indol-3-yl-2,4,5,6,7-d₅)—N,N-dimethylethan-1-amine (27): A suspension of lithium aluminum hydride (1.34 g, 35.431 mmol) in dry THF (20 mL) was treated with 2-(1H-indol-3-yl-2,4,5,6,7-d₅)—N,N-dimethyl-2-oxoacetamide (0.98 g, 4.428 mmol) in dry THF (30 mL) at 0° C. over a period of 10 min. The reaction was brought to room temperature, then refluxed for additional 16 hours. The reaction was worked-up and purified as described for compound 3 to obtain the title compound 27 (0.75 g, 87.6%) as a pale-yellow solid. ¹H NMR (DMSO-d₆): δ 10.76 (s, 1H), 7.51-7.49 (m, 0.18H), 7.34-7.32 (m, 0.04H), 7.14 (d, 0.05H, J=1.5 Hz), 7.07-7.05 (m, 0.32H), 6.99-6.96 (m, 0.41H), 2.84-2.80 (m, 2H), 2.54-2.50 (m, 2H), 2.22 (s, 6H); ESI-MS (m/z, %): 194 (MH⁺), 193 (100).

### Example 13: (R)-3-((1-Methylpyrrolidin-2-yl)methyl-d₂)-1H-indole (29)

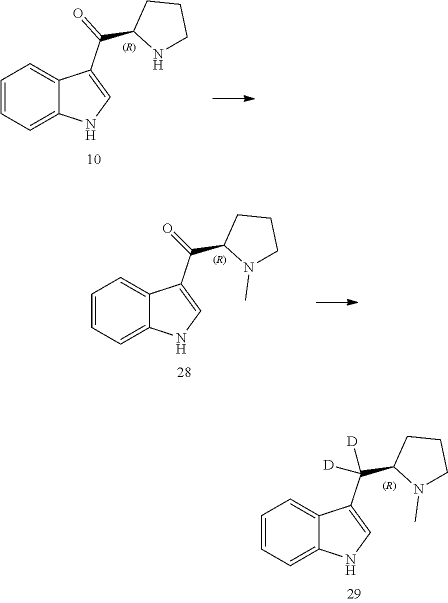

Synthesis of 3-(methyl-D-prolyl)-1H-indole (28): A suspension of (R)-3-prolyl-1H-indole (1.02 g, 4.760 mmol) in dichloroethane (50 mL) was treated with acetic acid (0.81 mL, 14.282 mmol) followed by formaldehyde solution (1.06 mL, 14.282 mmol) at 0° C. The reaction was brought to room temperature and stirred for additional 6 h. The reaction was treated with sodium triacetoxy borohydride (3.02 g, 14.282 mmol) at 0° C. The reaction was brought to room temperature and stirred for over night (18 h). The reaction was quenched with 4 N NaOH solution (50 mL) and product was extracted into chloroform (4×50 mL). Combined chloroform layer was washed with brine (25 mL) and dried (Na₂SO₄). Solvent was evaporated and crude was purified by column chromatography (2 M NH₃ in MeOH:CH₂Cl₂, 5:95) on silica gel to obtain the title compound 28 (0.47 g, 43.5%) as a light brown solid. ¹H NMR (DMSO-d₆): δ 11.90 (s, 1H), 8.54 (d, 1H, J=3.0 Hz), 8.28-8.20 (m, 1H), 7.49-7.46 (m, 1H), 7.25-7.13 (m, 2H), 3.46 (dd, 1H, J=3.0, 6.0 Hz), 3.13-3.08 (m, 1H), 2.14 (s, 3H), 2-12-2.10 (m, 1H), 1.93-1.74 (m, 4H); ESI-MS (m/z, %): 228 (MH⁺, 100).

Synthesis of (R)-3-((1-methylpyrrolidin-2-yl)methyl-d₂)-1H-indole (29): A suspension of lithium aluminum deuteride (0.25 g, 6.044 mmol) in dry THF (10 mL) was treated with 3-(methyl-D-prolyl)-1H-indole (0.46 g, 2.014 mmol) in dry THF (20 mL) at 0° C. over a period of 10 min. The reaction was brought to room temperature, then refluxed for additional 16 hours. The reaction was worked-up and purified as described for compound 3 to obtain the title compound 29 (0.31 g, 71.0%) as a pale-yellow solid. ¹H NMR (DMSO-d₆): δ 10.77 (s, 1H), 7.50 (dd, 1H, J=3.0 Hz), 7.34-7.31 (m, 1H), 7.13 (d, 1H, J=1.5 Hz). 7.07-7.03 (m, 1H), 6.99-6.95 (m, 1H), 3.39-3.34 (m, 1H), 3.19-2.95 (m, 1H), 2.37 (s, 3H), 2.12-2.07 (m, 1H), 1.69-1.42 (m, 4H); ESI-MS (m/z, %): 217 (MH⁺, 100).

### Example 14: (R)-5-Methoxy-3-((1-methylpyrrolidin-2-yl)methyl-d₂)-1H-indole (31)

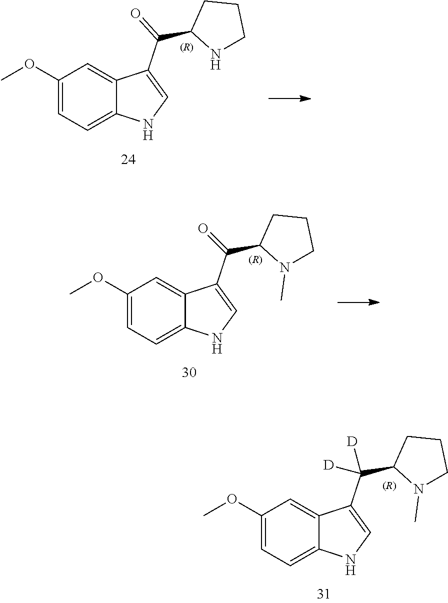

Synthesis of 5-methoxy-3-(methyl-D-prolyl)-1H-indole (30): A suspension of (R)-5-methoxy-3-prolyl-1H-indole (0.56 g, 2.304 mmol) in dichloroethane (30 mL) was treated with acetic acid (0.39 mL, 6.913 mmol) followed by formaldehyde solution (0.51 mL, 6.913 mmol, 37% in water) at 0° C. The reaction was brought to room temperature and stirred for additional 6 h. The reaction was treated with sodium triacetoxy borohydride (1.46 g, 6.913 mmol) at 0° C. The reaction was brought to room temperature and stirred for over night (18 h). The reaction was worked-up and purified as described for compound 28 to obtain the title compound 30 (0.19 g, 32%) as a light brown solid. ¹H NMR (CDCl₃): δ 9.08 (brs, 1H), 8.33 (d, 1H, J=3.0 Hz), 8.02 (dd, 1H, J=3.0, 4.5 Hz), 7.34 (dd, 1H, J=3.0, 4.5 Hz), 6.96-6.93 (m, 1H), 3.89 (s, 3H), 3.48-3.44 (m, 1H), 3.29-3.25 (m, 1H), 2.29-2.26 (m, 1H), 2.07-1.86 (m, 4H); ESI-MS (m/z, %): 259 (MH⁺, 100).

Synthesis of (R)-5-methoxy-3-((1-methylpyrrolidin-2-yl)methyl-d₂)-1H-indole (31): A suspension of lithium aluminum deuteride (0.112 g, 2.670 mmol) in dry THF (10 mL) was treated with 5-methoxy-3-(methyl-D-prolyl)-1H-indole (0.23 g, 0.890 mmol) in dry THF (20 mL) at 0° C. over a period of 10 min. The reaction was brought to room temperature, then refluxed for additional 16 hours. The reaction was worked-up and purified as described for compound 3 to obtain the title compound 31 (0.14 g, 63.6%) as a pale-yellow glue. ¹H NMR (DMSO-d₆): δ 10.61 (s, 1H), 7.22 (d, 1H, J=6.0 Hz), 7.09 (d, 1H, J=3.0 Hz), 6.97 (d, 1H, J=3.0 Hz), 6.71 (dd, 1H, J=3.0, 6.0 Hz), 3.77 (s, 3H), 3.42-3.34 (m, 1H), 3.19-2.96 (m, 1H), 2.36 (s, 3H), 2.15-2.08 (m, 1H), 1.76-1.43 (m, 4H); ESI-MS (m/z, %): 247 (MH⁺, 100).

### Example 15: (9Z,12Z)-1-(3-(2-(Dimethylamino)ethyl)-5-methoxy-1H-indol-1-yl)octadeca-9,12-dien-1-one (I-1)

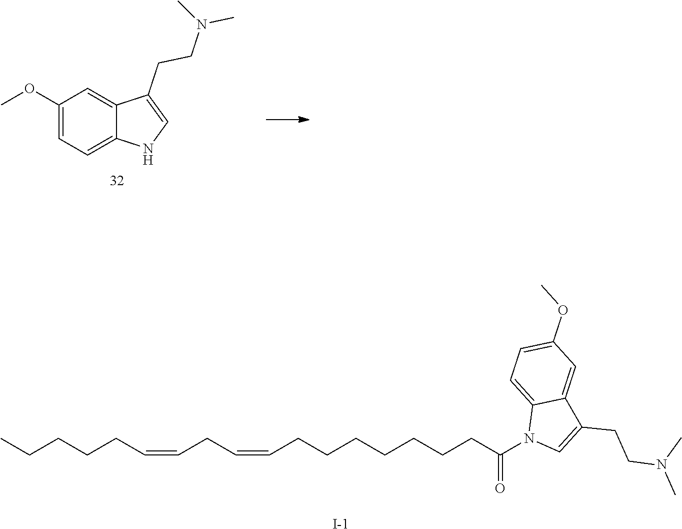

Synthesis of (9Z,12Z)-1-(3-(2-(dimethylamino)ethyl)-5-methoxy-1H-indol-1-yl)octadeca-9,12-dien-1-one (I-1): A solution of linoleic acid (3.85 g, 13.744 mmol) in dry CH₂Cl₂ (50 mL) was treated with oxalyl chloride (1.74 mL, 20.615 mmol) followed by 1 drop of dry DMF at room temperature and stirred for additional 2 h. Solvent was evaporated and crude product was dried on high vacuum to obtain the corresponding acid chloride.

A solution of 2-(5-methoxy-1H-indol-3-yl)-N,N-dimethylethan-1-amine (1.5 g, 6.872 mmol) in dry DMF (20 mL) was cooled to 0° C. and treated with sodium hydride (0.82 g, 20.615 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was quenched with water (250 mL), and product was extracted into ethyl acetate (2×100 mL). Combined organic layer was washed with brine (50 mL) and dried (Na₂SO₄). Solvent was evaporated and crude was purified by column chromatography (2 M NH₃ in MeOH:CH₂Cl₂, 5:95) on silica gel to obtain the title compound 1-1 (2.4 g, 72.7%) as a pale-yellow oil. ¹H NMR (CDCl₃): δ 8.37 (d, 1H, J=6.0 Hz), 7.09 (d, 1H, J=3.0 Hz), 7.01-6.96 (m, 2H), 5.44-5.32 (m, 4H), 3.90 (s, 3H), 2.90-2.78 (m, 4H), 2.69-2.65 (m, 2H), 2.38 (s, 6H), 2.09-2.06 (m, 4H), 1.86-1.82 (m, 4H), 1.41-1.29 (m, 14H), 0.91 (t, 3H, J=6.0 Hz); ESI-MS (m/z, %): 481 (MH⁺, 100).

### Example 16: (9Z,12Z)-1-(3-(((R)-1-(Methyl-d₃)pyrrolidin-2-yl)methyl-d₂)-1H-indol-1-yl)octadeca-9,12-dien-1-one (I-2)

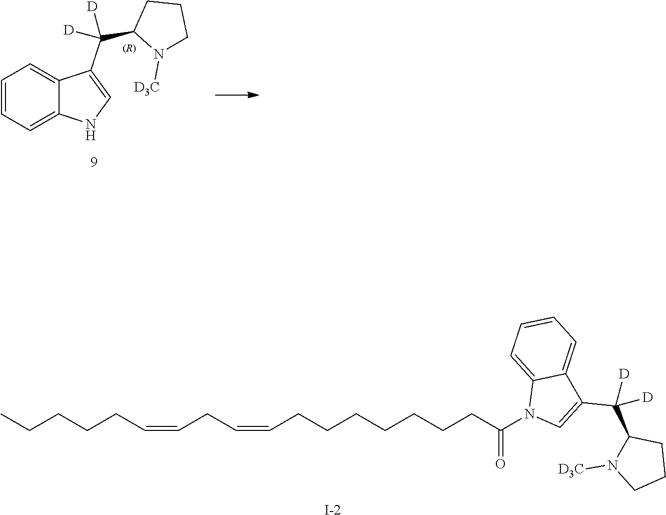

Synthesis of (9Z,12Z)-1-(3-(((R)-1-(methyl-d₃)pyrrolidin-2-yl)methyl-d₂)-1H-indol-1-yl)octadeca-9,12-dien-1-one (I-2): A solution of linoleic acid (0.77 g, 2.735 mmol) in dry CH₂Cl₂ (20 mL) was treated with oxalyl chloride (0.3 mL, 3.647 mmol) followed by 1 drop of dry DMF at room temperature and stirred for additional 2 h. Solvent was evaporated and crude product was dried on high vacuum to obtain the corresponding acid chloride.

A solution of (R)-3-((1-(methyl-d₃)pyrrolidin-2-yl)methyl-d₂)-1H-indole (0.2 g, 0.911 mmol) in dry DMF (10 mL) cooled to 0° C. and treated with sodium hydride (0.145 g, 3.647 mmol, 60% in mineral oil), brought to room temperature and stirred for 30 min. The reaction was cooled to 0° C., treated with above crude acid chloride in dry DMF (10 mL). The reaction was brought to room temperature and stirred for over night (18 h). The reaction was worked-up and purified as described for compound 1-1 to obtain the title compound 1-2 (0.271 g, 61.7%) as a light brown oil. ¹H NMR (CDCl₃): δ 8.49 (d, 1H, J=6.0 Hz), 7.55 (d, 1H, J=6.0 Hz), 7.40-7.31 (m, 3H), 5.42-5.33 (m, 4H), 2.92 (t, 2H, J=6.0 Hz), 2.80 (t, 2H, J=6.0 Hz), 2.35-2.25 (m, 1H), 2.10-2.05 (m, 4H), 1.91-1.66 (m, 6H), 1.48-1.29 (m, 16H), 0.90 (t, 3H, J=3.0 Hz); ESI-MS (m/z, %): 482 (MH⁺, 100).

### Example 17: (9Z,12Z)-1-(5-Methoxy-3-(((R)-1-(methyl-d₃)pyrrolidin-2-yl)methyl-d₂)-1H-indol-1-yl)octadeca-9,12-dien-1-one (I-3)

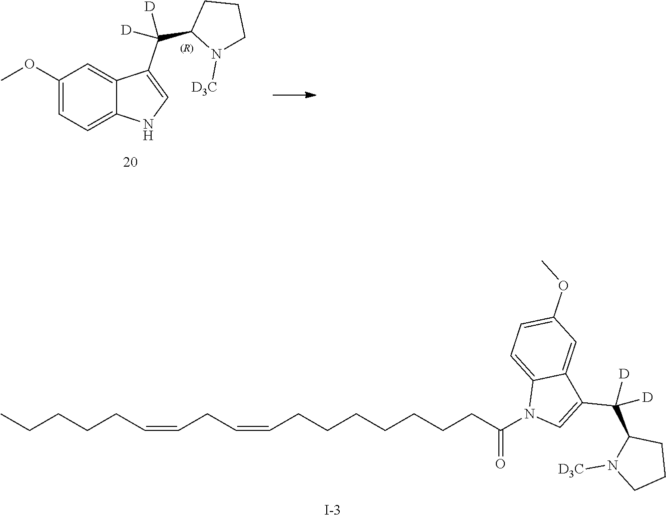

Synthesis of (9Z,12Z)-1-(5-methoxy-3-(((R)-1-(methyl-d₃)pyrrolidin-2-yl)methyl-d₂)-1H-indol-1-yl)octadeca-9,12-dien-1-one (I-3): A solution of linoleic acid (0.67 g, 2.406 mmol) in dry CH₂Cl₂ (20 mL) was treated with oxalyl chloride (0.27 mL, 3.208 mmol) followed by 1 drop of dry DMF at room temperature and stirred for additional 2 h. Solvent was evaporated and crude product was dried on high vacuum to obtain the corresponding acid chloride.

A solution of (R)-5-methoxy-3-((1-(methyl-d₃)pyrrolidin-2-yl)methyl-d₂)-1H-indole (0.2 g, 0.802 mmol) in dry DMF (10 mL) cooled to 0° C. and treated with sodium hydride (0.128 g, 3.208 mmol, 60% in mineral oil), brought to room temperature and stirred for 30 min. The reaction was cooled to 0° C., treated with above crude acid chloride in dry DMF (10 mL). The reaction was brought to room temperature and stirred for over night (18 h). The reaction was worked-up and purified as described for compound 1-1 to obtain the title compound 1-3 (0.16 g, 39%) as a brown oil. ¹H NMR (CDCl₃): δ 8.38 (d, 1H, J=6.0 Hz), 7.36 (s, 1H), 7.01-6.96 (m, 2H), 5.42-5.34 (m, 4H), 3.90 (s, 3H), 2.89 (t, 2H, J=6.0 Hz), 2,80 (t, 2H, J=6.0 Hz), 2.10-1.55 (m, 11H), 1.47-1.29 (m, 16H), 0.90 (t, 3H, J=3.0 Hz); ESI-MS (m/z, %): 512 (MH⁺, 100).

### Example 18: (9Z,12Z)-1-(3-(2-(Bis(methyl-d₃)amino)ethyl-1,1,2,2-d₄)-5-(methoxy-d₃)-1H-indol-1-yl)octadeca-9,12-dien-1-one (I-4)

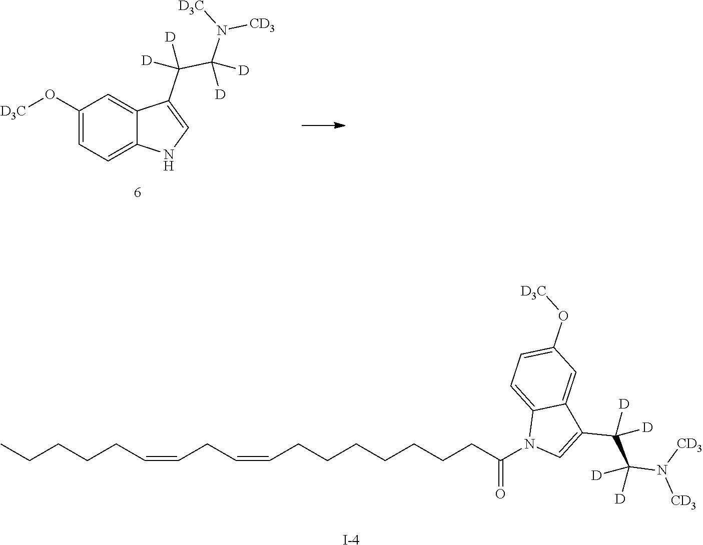

Synthesis of (9Z,12Z)-1-(3-(2-(bis(methyl-d₃)amino)ethyl-1,1,2,2-d₄)-5-(methoxy-d₃)-1H-indol-1-yl)octadeca-9,12-dien-1-one (I-4): A solution of linoleic acid (0.72 g, 2.592 mmol) in dry CH₂Cl₂ (20 mL) was treated with oxalyl chloride (0.3 mL, 3.457 mmol) followed by 1 drop of dry DMF at room temperature and stirred for additional 2 h. Solvent was evaporated and crude product was dried on high vacuum to obtain the corresponding acid chloride.

A solution of 2-(5-(methoxy-d₃)-1H-indol-3-yl)-N,N-bis(methyl-d₃)ethan-1-amine-1,1,2,2-d₄ (0.2 g, 0.864 mmol) in dry DMF (10 mL) cooled to 0° C. and treated with sodium hydride (0.138 g, 3.457 mmol, 60% in mineral oil), brought to room temperature and stirred for 30 min. The reaction was cooled to 0° C., treated with above crude acid chloride in dry DMF (10 mL). The reaction was brought to room temperature and stirred for over night (18 h). The reaction was worked-up and purified as described for compound 1-1 to obtain the title compound 1-4 (0.295 g, 69.2%) as a brown oil. ¹H NMR (CDCl₃): δ 8.37 (s, 1H), 7.29 (d, 1H, J=3.0 Hz), 7.01-6.96 (m, 2H), 5.44-5.32 (m, 4H), 2.90-2.78 (m, 4H), 2.10-1.80 (m, 6H), 1.41-1.29 (m, 14H), 0.91 (t, 3H, J=6.0 Hz); ESI-MS (m/z, %): 494 (MH⁺, 100).

### Example 19: (R)-3-((1-Methylpyrrolidin-2-yl)methyl)-1H-indole (37)

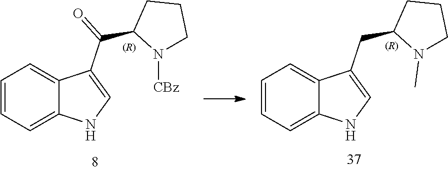

Synthesis of (R)-3-((1-methylpyrrolidin-2-yl)methyl)-1H-indole (37): A suspension of lithium aluminum hydride (2.83 g, 74.626 mmol) in dry THF (40 mL) was treated with (R)-2-(2-(1H-indole-3-carbonyl)pyrrolidin-1-yl)-1-phenyl-2λ²-ethan-1-one (5.2 g, 14.925 mmol) in dry THF (60 mL) at 0° C. over a period of 10 min. The reaction was brought to room temperature, then refluxed for additional 16 hours. The reaction was worked-up and purified as described for compound 3 to obtain the title compound 37 (2.78 g, 87%) as an off-white solid. ¹H NMR (CDCl₃): δ 8.10 (brs, 1H), 7.65 (d, 1H, J=6.0 Hz), 7.39 (d, 1H, J=6.0 Hz), 7.24-7.13 (m, 2H), 7.05 (d, 1H, J=1.5 Hz), 3.26-3.14 (m, 2H), 2.68-2.62 (m, 1H), 2.54-2.47 (m, 4H), 2.29-2.22 (m, 1H), 1.88-1.60 (m, 4H); ESI-MS (m/z, %): 215 (MH⁺, 100).

### Example 20: (R)-5-Methoxy-3-((1-methylpyrrolidin-2-yl)methyl)-1H-indole (38)

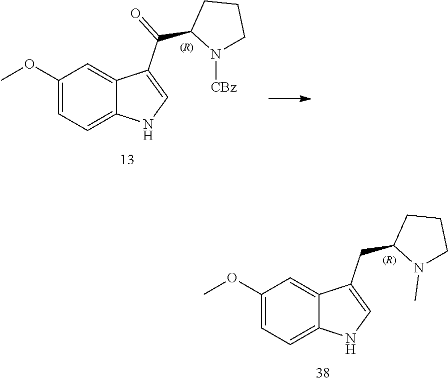

Synthesis of (R)-5-methoxy-3-((1-methylpyrrolidin-2-yl)methyl)-1H-indole (38): A suspension of lithium aluminum hydride (2.53 g, 66.723 mmol) in dry THF (40 mL) was treated with (R)-2-(2-(5-methoxy-1H-indole-3-carbonyl)pyrrolidin-1-yl)-1-phenyl-2λ²-ethan-1-one (5.05 g, 13.344 mmol) in dry THF (60 mL) at 0° C. over a period of 10 min. The reaction was brought to room temperature, then refluxed for additional 16 hours. The reaction was worked-up and purified as described for compound 3 to obtain the title compound 38 (2.5 g, 76.7%) as light brown glue. ¹H NMR (CDCl₃): δ 7.98 (brs, 1H), 7.27 (d, 1H, J=6.0 Hz), 7.09 (d, 1H, J=3.0 Hz), 7.03 (d, 1H, J=1.5 Hz), 6.89 (dd, 1H, J=3.0, 6.0 Hz), 3.90 (s, 3H), 3.20-3.14 (m, 2H), 2.65-2.59 (m, 1H), 2.51 (s, 3H), 2.27-2.22 (m, 1H), 1.91-1.56 (m, 5H); ESI-MS (m/z, %): 245 (MH⁺, 100).

### Example 21: (9Z,12Z)-1-(3-(((R)-1-Methylpyrrolidin-2-yl)methyl)-1H-indol-1-yl)octadeca-9,12-dien-1-one (I-5)

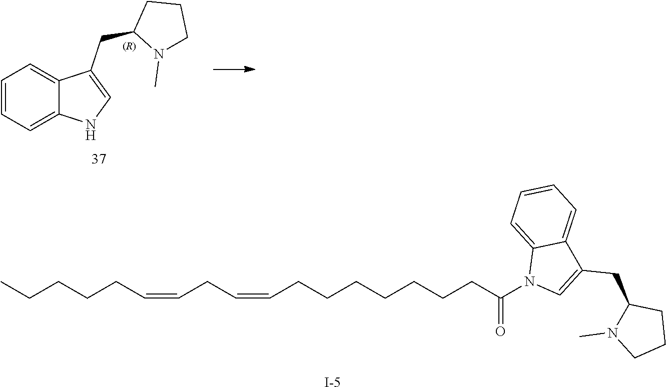

Synthesis of (9Z,12Z)-1-(3-(((R)-1-methylpyrrolidin-2-yl)methyl)-1H-indol-1-yl)octadeca-9,12-dien-1-one (I-5): A solution of linoleic acid (2.78 g, 9.938 mmol) in dry CH₂Cl₂ (40 mL) was treated with oxalyl chloride (1.12 mL, 13.25 mmol) followed by 1 drop of dry DMF at room temperature and stirred for additional 2 h. Solvent was evaporated and crude product was dried on high vacuum to obtain the corresponding acid chloride.

A solution of (R)-3-((1-methylpyrrolidin-2-yl)methyl)-1H-indole (0.71 g, 3.312 mmol) in dry DMF (20 mL) cooled to 0° C. and treated with sodium hydride (0.53 g, 13.25 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound 1-1 to obtain the title compound 1-5 (0.9 g, 57%) as a brown oil. ¹H NMR (CDCl₃): δ 8.49 (d, 1H, J=6.0 Hz), 7.57 (d, 1H, J=6.0 Hz), 7.40-7.31 (m, 3H), 5.43-5.35 (m, 4H), 3.18-3.12 (m, 2H), 2.92 (t, 2H, J=6.0 Hz), 2.81 (t, 2H, J=6.0 Hz), 2.64-2.58 (m, 1H), 2.50 (s, 3H), 2.30-2.25 (m, 1H), 2.11-2.05 (m, 4H), 1.88-1.58 (m, 6H), 1.48-1.29 (m, 16H), 0.92 (t, 3H, J=3.0 Hz); ESI-MS (m/z, %): 477 (MH⁺, 100).

### Example 22: (9Z,12Z)-1-(5-Methoxy-3-(((R)-1-methylpyrrolidin-2-yl)methyl)-1H-indol-1-yl)octadeca-9,12-dien-1-one (I-6)

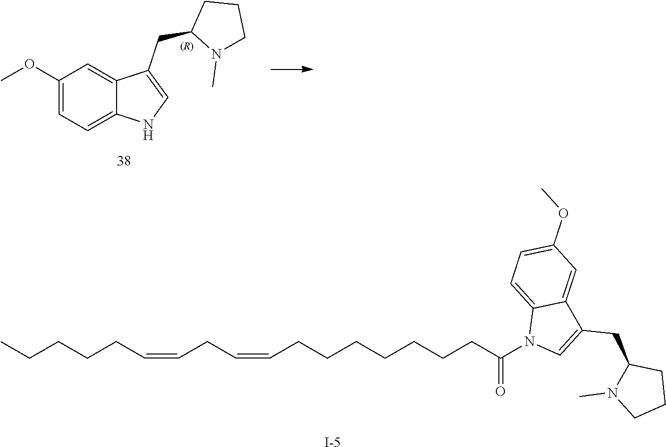

Synthesis of (9Z,12Z)-1-(5-methoxy-3-(((R)-1-methylpyrrolidin-2-yl)methyl)-1H-indol-1-yl)octadeca-9,12-dien-1-one (I-6): A solution of linoleic acid (2.78 g, 9.945 mmol) in dry CH₂Cl₂ (40 mL) was treated with oxalyl chloride (1.12 mL, 13.26 mmol) followed by 1 drop of dry DMF at room temperature and stirred for additional 2 h. Solvent was evaporated and crude product was dried on high vacuum to obtain the corresponding acid chloride.

A solution of (R)-5-methoxy-3-((1-methylpyrrolidin-2-yl)methyl)-1H-indole (0.81 g, 3.315 mmol) in dry DMF (20 mL) cooled to 0° C. and treated with sodium hydride (0.53 g, 13.26 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound I-I to obtain the title compound 1-6 (0.94 g, 56%) as a brown oil. ¹H NMR (CDCl₃): δ 8.38 (d, 1H, J=6.0 Hz), 7.29 (s, 1H), 7.02-6.97 (m, 2H), 5.43-5.36 (m, 4H), 3.90 (s, 3H), 2.89 (t, 2H, J=6.0 Hz), 2.80 (t, 2H, J=6.0 Hz), 2.61-2.56 (m, 2H), 2.50 (s, 3H), 2.33-2.29 (m, 1H), 2.11-2.05 (m, 4H), 1.91-1.61 (m, 6H), 1.49-1.29 (m, 16H), 0.92 (t, 3H, J=3.0 Hz); ESI-MS (m/z, %): 507 (MH+, 100).

### Example 23: (9Z,12Z)-1-(3-(2-(Bis(methyl-d₃)amino)ethyl)-1H-indol-1-yl-2,4,5,6,7-d₅)octadeca-9,12-dien-1-one (I-7)

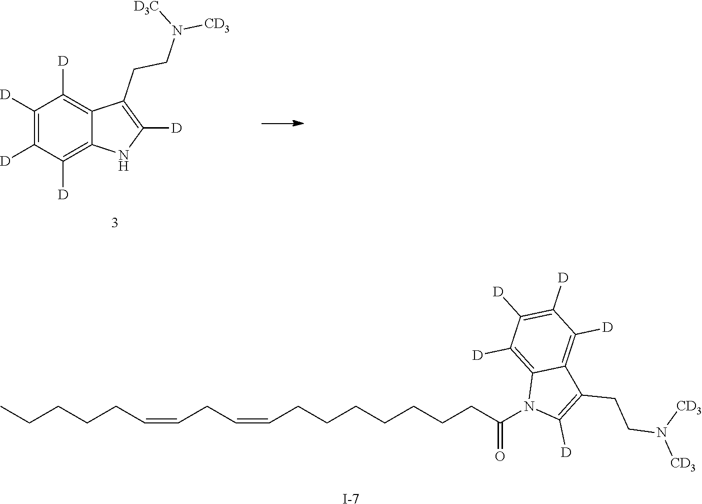

Synthesis of (9Z,12Z)-1-(3-(2-(bis(methyl-d₃)amino)ethyl)-1H-indol-1-yl-2,4,5,6,7-d₅)octadeca-9,12-dien-1-one (I-7): A solution of linoleic acid (1.05 g, 9.945 mmol) in dry CH₂Cl₂ (20 mL) was treated with oxalyl chloride (0.42 mL, 5.016 mmol) followed by 1 drop of dry DMF at room temperature and stirred for additional 2 h. Solvent was evaporated and crude product was dried on high vacuum to obtain the corresponding acid chloride.

A solution of 2-(1H-indol-3-yl-2,4,5,6,7-d₅)—N,N-bis(methyl-d₃)ethan-1-amine (0.25 g, 1.254 mmol) in dry DMF (10 mL) cooled to 0° C. and treated with sodium hydride (0.2 g, 5.016 mmol, 60% in mineral oil), brought to room temperature and stirred for 30 min. The reaction was cooled to 0° C., treated with above crude acid chloride in dry DMF (10 mL). The reaction was brought to room temperature and stirred for over night (18 h). The reaction was worked-up and purified as described for compound 1-1 to obtain the title compound 1-7 (0.44 g, 75.8%) as a pale-yellow oil. ¹H NMR (CDCl₃): δ 7.57-7.55 (m, 0.1H), 7.38-7.37 (m, 0.16H), 7.32-7.29 (m, 0.31H), 5.44-5.32 (m, 4H), 2.93-2.89 (m, 4H), 2.80 (t, 2H, J=6.0 Hz), 2.69-2.65 (m, 2H), 2.11-2.03 (m, 4H), 1.89-1.82 (m, 2H), 1.49-1.28 (m, 14H), 0.93-0.88 (m, 3H); ESI-MS (m/z, %): 462 (MH⁺, 100).

### Example 24: (9Z,12Z)-1-(3-(2-(Dimethylamino)ethyl)-1H-indol-1-yl)octadeca-9,12-dien-1-one (I-8)

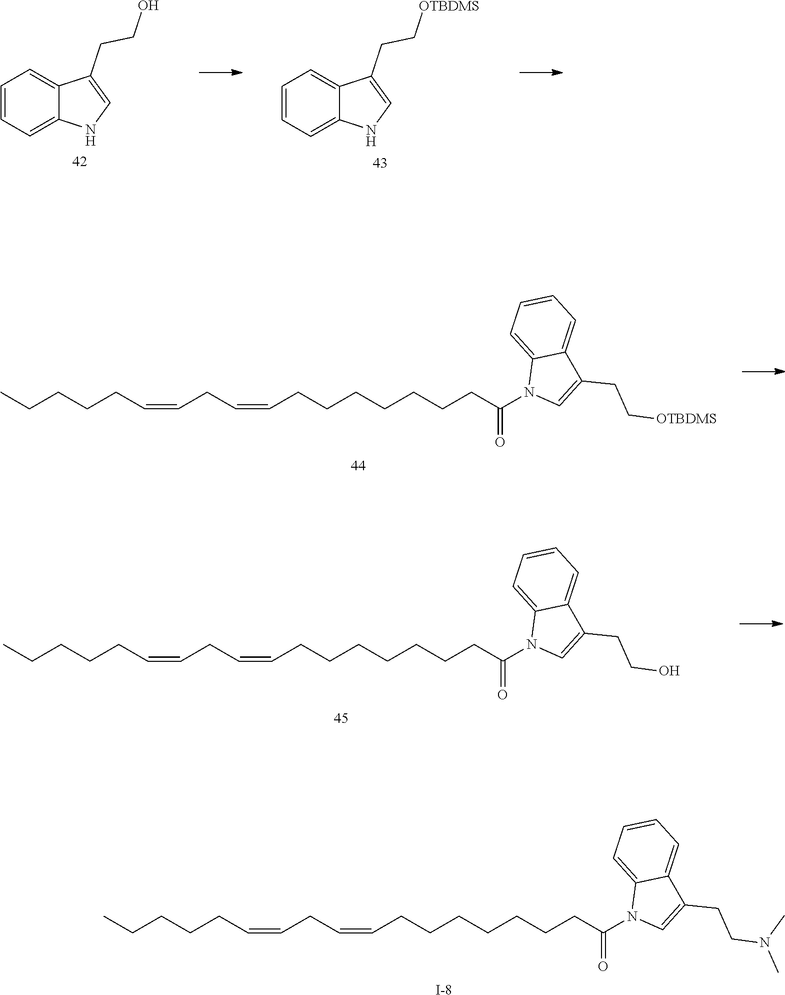

Synthesis of 3-(2-((tert-butyldimethylsilyl)oxy)ethyl)-1H-indole (43): A solution of 2-(1H-indol-3-yl)ethan-1-ol (5.0 g, 31.017 mmol) and imidazole (4.22 g, 62.035 mmol) in dry DMF (70 mL) was treated with TBDMSCl (7.0 g, 46.525 mmol) in DMF (40 mL) at 0° C. over a period of 10 min. The reaction was brought to room temperature and stirred for additional 1 h. The reaction was quenched with water (300 mL) and product was extracted into ethyl acetate (2×100 mL). Combined ethyl acetate layer was washed with water (50 mL), brine (50 mL) and dried (Na₂SO₄). Solvent was evaporated and crude was purified by flash column chromatography (hexanes to CH₂Cl₂:hexanes, 1:4) on silica gel to obtain the title compound 43 (8.5 g, quantitative) as a yellow to orange oil. ¹H NMR (CDCl₃): δ 7.99 (brs, 1H), 7.67-7.64 (m, 1H), 7.40-7.38 (m, 1H), 7.25-7.14 (m, 2H), 7.08 (d, 1H, J=3.0 Hz), 3.95-3.91 (m, 2H), 3.06-3.02 (m, 2H), 0.96 (s, 9H), 0.08 (s, 6H).

Synthesis of (9Z,12Z)-1-(3-(2-((tert-butyldimethylsilyl)oxy)ethyl)-1H-indol-1-yl)octadeca-9,12-dien-1-one (44): A solution of linoleic acid (4.0 g, 14.52 mmol) in dry CH₂Cl₂ (50 mL) was treated with oxalyl chloride (1.84 mL, 21.78 mmol) followed by 1 drop of dry DMF at room temperature and stirred for additional 2 h. Solvent was evaporated and crude product was dried on high vacuum to obtain the corresponding acid chloride.

A solution of 3-(2-((tert-butyldimethylsilyl)oxy)ethyl)-1H-indole (2.0 g, 7.26 mmol) in dry DMF (20 mL) cooled to 0° C. and treated with sodium hydride (0.87 g, 21.78 mmol, 60% in mineral oil), brought to room temperature and stirred for 30 min. The reaction was cooled to 0° C., treated with above crude acid chloride in dry DMF (30 mL). The reaction was brought to room temperature and stirred for over night (18 h). The reaction was worked-up and purified as described for compound 33 to obtain the title compound 44 (2.5 g, 64.1%) as a light-yellow oil. ¹H NMR (CDCl₃): δ 8.48 (d, 1H, J=6.0 Hz), 7.55 (d, 1H, J=3.0, 6.0 Hz), 7.39-7.28 (m, 3H), 5.43-5.35 (m, 4H), 3.93 (t, 2H, J=6.0 Hz), 2.96-2.88 (m, 4H), 2.80 (t, 2H, J=6.0 Hz), 2.10-2.05 (m, 4H), 1.88-1.84 (m, 2H), 1.47-1.29 (m, 14H), 0.93-0.86 (m, 12H), 0.05 (s, 6H).

Synthesis of (9Z,12Z)-1-(3-(2-hydroxyethyl)-1H-indol-1-yl)octadeca-9,12-dien-1-one (45): A solution of (9Z,12Z)-1-(3-(2-((tert-butyldimethylsilyl)oxy)ethyl)-1H-indol-1-yl)octadeca-9,12-dien-1-one (2.4 g, 4.462 mmol) in dry THF (20 mL) was treated with TBAF (8.9 mL, 8.924 mmol) at 0° C. over a period of 10 min. The reaction was brought to room temperature and stirred for additional 1 h. The reaction was quenched with sat. NH₄Cl solution (50 mL) and product was extracted into ethyl acetate (2×50 mL). Combined ethyl acetate layer was washed with brine (50 mL) and dried (Na₂SO₄). Solvent was evaporated and crude was purified by flash column chromatography (EtOAc:Hexanes, 5:95) to obtain the title compound 45 (0.9 g, 47.6%) as a pale-yellow oil. ¹H NMR (CDCl₃): δ 8.49 (d, 1H, J=6.0 Hz), 7.57 (dd, 1H, J=1.5, 6.0 Hz), 7.40-7.38 (m, 2H), 7.33 (dd, 1H, J=3.0, 6.0 Hz), 5.42-5.35 (m, 4H), 3.98 (t, 2H, J=6.0 Hz), 3.03-2.99 (m, 2H), 2.91 (t, 2H, J=6.0 Hz), 2.80 (t, 2H, J=6.0 Hz), 2.10-2.05 (m, 4H), 1.87-1.84 (m, 2H), 1.49-1.29 (m, 11H), 0.93-0.88 (6H).

Synthesis of (9Z,12Z)-1-(3-(2-(dimethylamino)ethyl)-1H-indol-1-yl)octadeca-9,12-dien-1-one (I-8): A solution of (9Z,12Z)-1-(3-(2-hydroxyethyl)-1H-indol-1-yl)octadeca-9,12-dien-1-one (0.9 g, 2.125 mmol) and triethylamine (0.44 mL, 3.187 mmol) in dry CH₂Cl₂ (15 mL) was treated with MsCl (0.18 mL, 2.337 mmol) at 0° C. The reaction was brought to room temperature and stirred for additional 1.5 h. The reaction was quenched with the addition of brine (50 mL) and product was extracted into CH₂Cl₂ (2×50 mL). Combined CH₂Cl₂ layer was dried (Na₂SO₄) and solvent was evaporated to obtain the crude mesylated intermediate.

A solution of above crude mesylated intermediate in dry THF (10 mL) was treated with N,N-dimethylamine solution (20 mL, 19.930 mmol) and stirred at 100° C. in a sealed tube for additional 4.5 h. The reaction was brought to room temperature, solvent was evaporated and crude was purified by column chromatography (2 M NH₃ in MeOH:CH₂Cl₂, 5:95) on silica gel to obtain the title compound 1-8 (0.56 g, 58.5%) as a light brown oil. ¹H NMR (CDCl₃): δ 8.48 (d, 1H, J=6.0 Hz), 7.58-7.56 (m, 1H), 7.40-7.30 (m, 3H), 5.43-5.33 (m, 4H), 2.95-2.79 (m, 4H), 2.71 (t, 2H, J=3.0 Hz), 2.69 (t, 2H, J=3.0 Hz), 2.39 (s, 6H), 2.12-2.06 (m, 4H), 1.88-1.84 (m, 2H), 1.50-1.29 (m, 14H), 0.92 (t, 3H, 6.0 Hz); ESI-MS (m/z, %): 451 (MH⁺, 100).

### Example 25: (9Z,12Z)-1-(3-(2-(Bis(methyl-d₃)amino)ethyl-1, 1,2,2-d₄)-4-fluoro-1H-indol-1-yl)octadeca-9,12-dien-1-one (I-9)

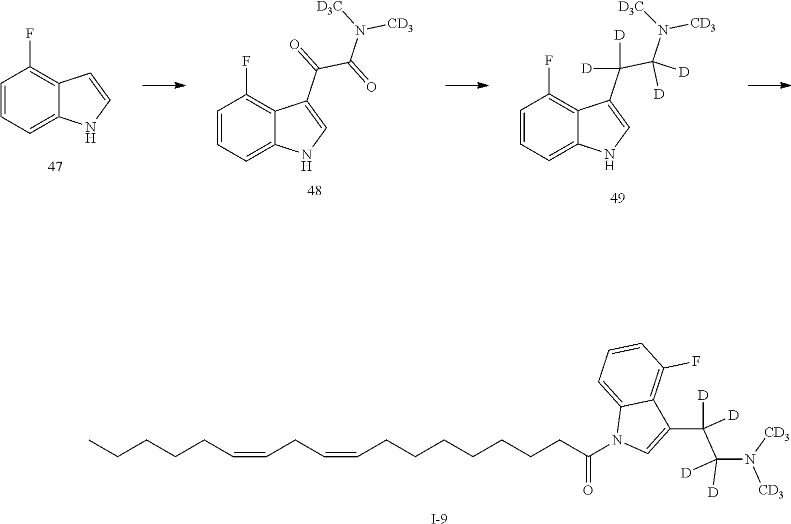

Synthesis of 2-(4-fluoro-1H-indol-3-yl)-N,N-bis(methyl-d₃)-2-oxoacetamide (48): A solution of 4-fluoro-1H-indole (1.0 g, 7.39 mmol) in dry ether (20 mL) was treated with oxalyl chloride (0.63 mL, 7.39 mmol) at 0° C. The reaction was brought to room temperature and stirred for additional 16 h. The reaction was cooled to 0° C., treated with bis(methyl-d₃)amine hydrochloride (1.62 g, 18.49 mmol, free based with K₂CO₃ in THF) over a period of 5 min. The reaction was brought to room temperature and stirred for 4 h. The reaction was worked-up and purified as described for compound 2 to obtain the title compound 48 (0.49 g, 27.6%) as an off-white solid. ¹H NMR (CDCl₃): δ 10.71 (s, 1H), 7.72 (d, 1H, J=3.0 Hz), 7.15-7.10 (m, 2H), 6.94-6.87 (m, 1H); ESI-MS (m/z, %): 263 (M+Na, 100).

Synthesis of 2-(4-fluoro-1H-indol-3-yl)-N,N-bis(methyl-d₃)ethan-1-amine-1,1,2,2-d₄ (49): A suspension of Lithium aluminum deuteride (0.19 g, 1.12 mmol) in dry THF (5 mL) was treated with 2-(4-fluoro-1H-indol-3-yl)-N,N-bis(methyl-d₃)-2-oxoacetamide (0.27 g, 1.33 mmol) in dry THF (10 mL) at 0° C. over a period of 10 min. The reaction was brought to room temperature, then refluxed for additional 16 hours, worked-up and purified as described for compound 3 to obtain the title compound 49 (0.1 g, 42%) as a pale yellow semi-solid. ¹H NMR (DMSO-d₆): δ 11.08 (s, 1H), 7.17-7.14 (m, 2H), 7.05-6.97 (m, 1H), 6.75-6.67 (m, 1H); ESI-MS (m/z, %): 217 (MH⁺, 100).

Synthesis of (9Z,12Z)-1-(3-(2-(bis(methyl-d₃)amino)ethyl-1,1,2,2-d₄)-4-fluoro-1H-indol-1-yl)octadeca-9,12-dien-1-one (I-9): A solution of linoleic acid (2.33 g, 8.320 mmol) in dry CH₂Cl₂ (30 mL) was treated with oxalyl chloride (0.94 mL, 11.094 mmol) followed by 1 drop of dry DMF at room temperature and stirred for additional 2 h. Solvent was evaporated and crude product was dried on high vacuum to obtain the corresponding acid chloride.

A solution of 2-(4-fluoro-1H-indol-3-yl)-N,N-bis(methyl-d₃)ethan-1-amine-1,1,2,2-d₄ (0.6 g, 2.773 mmol) in dry DMF (20 mL) cooled to 0° C. and treated with sodium hydride (0.44 g, 11.094 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound 1-1 to obtain the title compound 1-9 (0.37 g, 28%) as a pale-yellow oil. ¹H NMR (CDCl₃): δ 8.28 (d, 1H, J=6.0 Hz), 7.31-7.26 (m, 2H), 6.96 (dd, 1H, J=6.0 Hz), 5.45-5.33 (m, 4H), 2.90 (t, 2H, J=6.0 Hz), 2.81 (t, 2H, J=6.0 Hz), 2.10-2.07 (m, 4H), 1.87-1.84 (m, 2H), 1.45-1.29 (m, 14H), 0.92 (t, 3H, J=3.0 Hz); ESI-MS (m/z, %): 479 (MH⁺, 100).

### Example 26: 3-(Piperidin-4-yl)-1H-indole-2,4,5,6,7-d₅ (52)

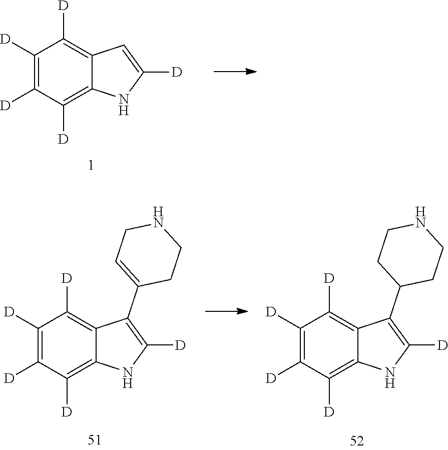 [SMILES: [2H]c1cc2c([2H])c([2H])c([2H])c([2H])c2[nH]1 ; [2H]c1[nH]c2c([2H])c([2H])c([2H])c([2H])c2c1C1=CCNCC1 ; [2H]c1[nH]c2c([2H])c([2H])c([2H])c([2H])c2c1C1CCNCC1]

Synthesis of 3-(1,2,3,6-tetrahydropyridin-4-yl)-1H-indole-2,4,5,6,7-d₅ (51): A solution of 1H-indole-2,4,5,6,7-d₅ (2.15 g, 17.454 mmol) in methanol (50 mL) was treated with potassium hydroxide (4.89 g, 43.635 mmol), followed by piperidin-4-one hydrochloride (6.70 g, 43.635 mmol) at room temperature and the resulting mixture was refluxed for additional 16 h. The reaction was brought to room temperature, methanol was evaporated, diluted with water (250 mL). Solid was filtered off, washed with water (3×25 mL) and dried on high vacuum to obtain the title compound 51 (3.33 g, 94%) as a yellow solid. ¹H NMR (DMSO-d₆): δ 11.07 (s, 1H), 7.81 (s, 0.08H), 7.38-7.35 (m, 0.1H), 7.11-7.09 (m, 0.1H), 7.03-7.01 (m, 0.14H), 6.18-6.16 (m, 1H), 3.43-3.40 (m, 2H), 2.94 (t, 2H, J=3.0 Hz), 2.40-2.36 (m, 2H); ESI-MS (m/z, %): 204 (MH⁺, 100).

Synthesis of 3-(piperidin-4-yl)-1H-indole-2,4,5,6,7-d₅ (52): A solution of 3-(1,2,3,6-tetrahydropyridin-4-yl)-1H-indole-2,4,5,6,7-d₅ (1.45 g, 7.132 mmol) in methanol (40 mL) was treated with Pd—C(0.25 g) and hydrogenated under hydrogen atm. (balloon pressure) for 2 h. The reaction was filtered and washed with methanol (3×25 mL). Combined methanol layer was evaporated and crude was crystallized from CH₂Cl₂:Hexanes (1:3) to obtain the title compound 52 (1.17 g, 80%) as light-yellow solid. ¹H NMR (DMSO-d₆): δ 10.75 (s, 1H), 7.56 (s, 0.07H), 7.33 (s, 0.05H), 7.06-7.05 (m, 0.14H), 6.96-6.94 (m, 0.14H), 3.05-3.00 (m, 2H), 2.87-2.2.79 (m, 1H), 2.67-2.61 (m, 2H), 1.89-1.85 (m, 2H), 1.61-1.51 (m, 2H); ESI-MS (m/z, %): 206 (MH⁺, 100).

### Example 27: 3-(1-Methylpiperidin-4-yl)-1H-indole-2,4,5,6,7-d₅ (54)

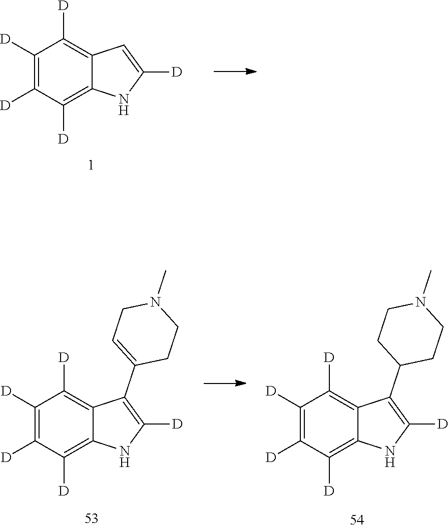 [SMILES: [2H]c1cc2c([2H])c([2H])c([2H])c([2H])c2[nH]1 ; [2H]c1[nH]c2c([2H])c([2H])c([2H])c([2H])c2c1C1=CCN(C)CC1 ; [2H]c1[nH]c2c([2H])c([2H])c([2H])c([2H])c2c1C1CCN(C)CC1]

Synthesis of 3-(1-methyl-1,2,3,6-tetrahydropyridin-4-yl)-1H-indole-2,4,5,6,7-d₅ (53): A solution of 1H-indole-2,4,5,6,7-d₅ (3.1 g, 25.166 mmol), 1-methylpiperidin-4-one (5.81 mL, 50.332 mmol), pyrrolidine (6.3 mL, 75.499 mmol) in ethanol (50 mL) was refluxed for additional 24 h. The reaction was brought to room temperature, then cooled to 0° C., stirred for 30 min. Solid was filtered, washed with cold ethanol (2×15 mL) and dried under high vacuum to obtain the title compound 53 (4.25 g, 77.7%) as a white solid. ¹H NMR (DMSO-d₆): δ 11.11 (s, 1H), 7.82 (s, 0.08H), 7.39 (t, 0.09H, J=3.0 Hz), 7.12 (t, 0.09H, J=3.0 Hz), 7.06-7.04 (m, 0.14H), 6.14 (t, 1H, J=3.0 Hz), 3.08-3.06 (m, 2H), 2.61-2.52 (m, 4H), 2.31 (s, 3H); ESI-MS (m/z, %): 218 (MH⁺, 100).

Synthesis of 3-(1-methylpiperidin-4-yl)-1H-indole-2,4,5,6,7-d₅ (54): A suspension of 3-(1-methyl-1,2,3,6-tetrahydropyridin-4-yl)-1H-indole-2,4,5,6,7-d₅ (1.43 g, 6.580 mmol) in methanol (40 mL) was treated with Pd—C(0.25 g) and hydrogenated under hydrogen atm. (balloon pressure) for 2 h. The reaction was filtered and washed with methanol (3×25 mL). Combined methanol layer was evaporated and crude was purified by flash column chromatography (2 M NH₃ in MeOH:CH₂Cl₂, 5:95) on silica gel to obtain the title compound 54 (1.34 g, 93%) as white solid. ¹H NMR (CDCl₃): δ 8.08 (bra, 1H), 7.70 (d, 0.08H, J=3.0 Hz), 7.39 (s, 0.06H), 7.23 (d, 0.11H, J=3.0 Hz), 7.14 (d, 0.16H, J=1.5 Hz), 7.01 (d, 0.06H, J=1.5 Hz), 3.05-3.00 (m, 2H), 2.89-2.82 (m, 2H), 2.38 (s, 3H), 2.20-2.07 (m, 4H), 1.94-1.80 (m, 2H); ESI-MS (m/z, %): 220 (MH⁺, 100).

### Example 28: 3-(1-(Methyl-d₃)piperidin-4-yl)-1H-indole (57)

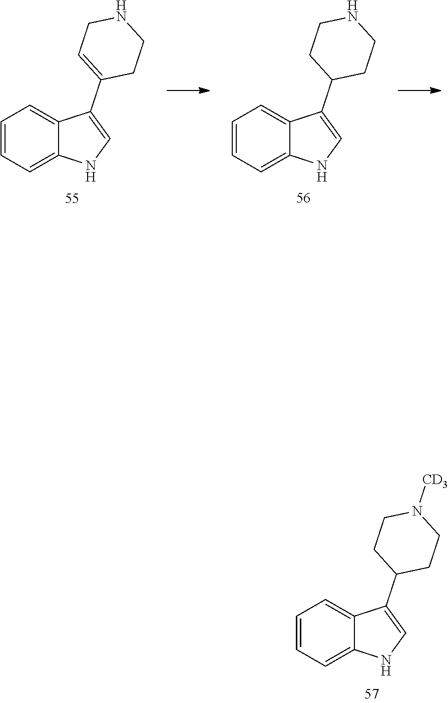 [SMILES: C1=C(c2c[nH]c3ccccc23)CCNC1 ; c1ccc2c(C3CCNCC3)c[nH]c2c1 ; [2H]C([2H])([2H])N1CCC(c2c[nH]c3ccccc23)CC1]

Synthesis of 3-(piperidin-4-yl)-1H-indole (56): A suspension of 3-(1,2,3,6-tetrahydropyridin-4-yl)-1H-indole (4.0 g, 20.175 mmol) in methanol (60 mL) was treated with Pd—C(0.4 g) and hydrogenated under hydrogen atm. (balloon pressure) for 18 h. The reaction was filtered and washed with methanol (3×25 mL). Combined methanol layer was evaporated and crude was crystallized from CH₂Cl₂:Hexanes (1:4) to obtain the title compound 56 (3.65 g, 90.3%) as a light-yellow solid. ¹H NMR (DMSO-d₆): δ 10.76 (s, 1H), 7.56 (d, 1H, J=6.0 Hz), 7.33 (d, 1H, J=6.0 Hz), 7.07-7.03 (m, 2H), 6.96 (dd, 1H, J=6.0, 9.0 Hz), 3.05-3.00 (m, 2H), 2.85-2.79 (m, 1H), 2.68-2.61 (m, 2H), 1.89-1.85 (m, 2H), 1.61-1.51 (m, 2H); ESI-MS (m/z, %): 201 (MH⁺, 100).

Synthesis of 3-(1-(methyl-d₃)piperidin-4-yl)-1H-indole (57): A solution of 3-(piperidin-4-yl)-1H-indole (0.13 g, 0.649 mmol) in dry DMF (5 mL) was treated with triethyl amine (0.27 mL, 1.947 mmol), followed by iodo-methane-d₃ (0.06 mL, 0.973 mmol) at room temperature and stirred for additional 24 h. The reaction was diluted with water (50 mL) and product was extracted into ethyl acetate (2×25 mL). Combined ethyl acetate layer was washed with brine (25 mL) and dried (Na₂SO₄). Solvent was evaporated and crude was purified by flash column chromatography (2 M NH₃ in MeOH:CH₂Cl₂, 5:95) on silica gel to obtain the title compound 57 (0.053 g, 37.8%) as an off-white solid. ¹H NMR (CDCl₃): δ 8.04 (brs, 1H), 7.69 (d, 1H, J=6.0 Hz), 7.39 (d, 1H, J=6.0 Hz), 7.23-7.7.19 (m, 1H), 7.15-7.11 (m, 1H), 7.01 (d, 1H, J=1.5 Hz), 3.04-2.99 (m, 2H), 2.89-2.81 (m, 1H), 2.20-2.07 (m, 4H), 1.93-1.83 (m, 2H); ESI-MS (m/z, %): 218 (MH⁺, 100).

### Example 29: (9Z,12Z)-1-(3-(1-Methylpiperidin-4-yl)-1H-indol-1-yl)octadeca-9,12-dien-1-one (I-10)

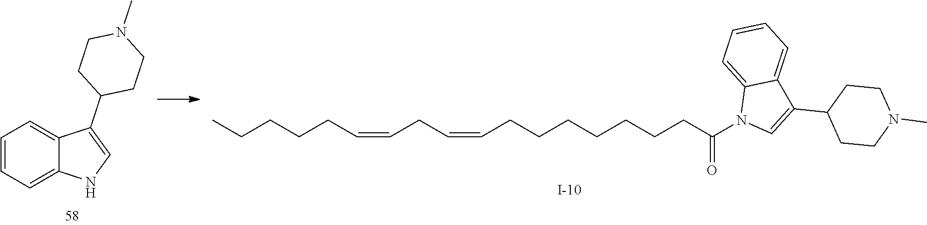

Synthesis of (9Z,12Z)-1-(3-(1-methylpiperidin-4-yl)-1H-indol-1-yl)octadeca-9,12-dien-1-one (I-10): A solution of linoleic acid (4.71 g, 16.797 mmol) in dry CH₂Cl₂ (50 mL) was treated with oxalyl chloride (1.89 mL, 22.397 mmol) followed by 1 drop of dry DMF at room temperature and stirred for additional 2 h. Solvent was evaporated and crude product was dried on high vacuum to obtain the corresponding acid chloride.

A solution of 3-(1-methylpiperidin-4-yl)-1H-indole (1.2 g, 5.599 mmol) in dry DMF (20 mL) cooled to 0° C. and treated with sodium hydride (0.89 g, 22.397 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound 1-1 to obtain the title compound 1-10 (2.16 g, 80.8%) as a light brown oil. ¹H NMR (CDCl₃): δ 8.49 (d, 1H, J=6.0 Hz), 7.60 (d, 1H, J=6.0 Hz), 7.39-7.22 (m, 2H), 7.22 (s, 1H), 5.44-5.34 (m, 4H), 3.06-3.02 (m, 2H), 2.89 (t, 2H, J=6.0 Hz), 2.80 (t, 2H, J=6.0 Hz), 2.39 (s, 3H), 2.21-2.05 (m, 8H), 1.91-1.81 (m, 3H), 1.49-1.28 (m, 16H), 0.90 (t, 3H, J=3.0 Hz); ESI-MS (m/z, %): 477 (MH⁺, 100).

### Example 30: (9Z,12Z)-1-(5-Methoxy-3-(1-methylpiperidin-4-yl)-1H-indol-1-yl)octadeca-9,12-dien-1-one (I-11)

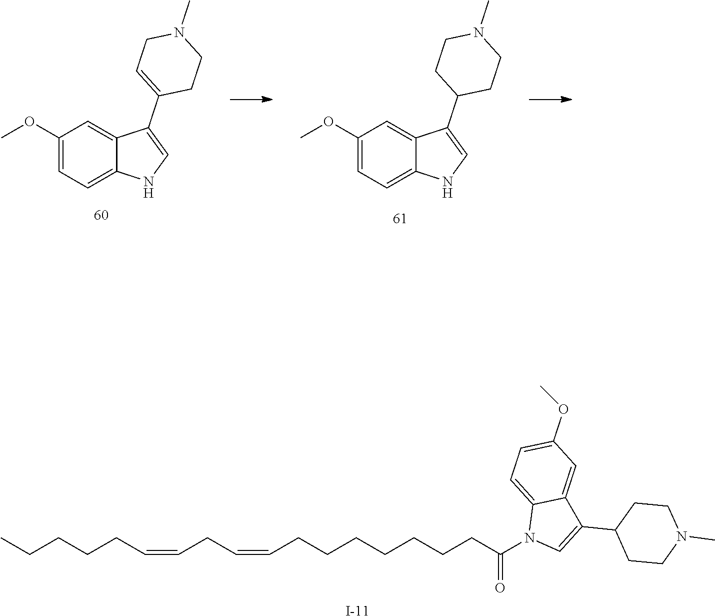

Synthesis of 5-methoxy-3-(1-methylpiperidin-4-yl)-1H-indole (61): A suspension of 5-methoxy-3-(1-methyl-1,2,3,6-tetrahydropyridin-4-yl)-1H-indole (4.05 g, 16.713 mmol) in ethanol (50 mL) was treated with Pd—C(0.4 g) and hydrogenated under hydrogen atm. (balloon pressure) for 18 h. The reaction was filtered through a pad of celite and washed with methanol (3×25 mL). Combined methanol layer was evaporated and dried on high vacuum to obtain the title compound 61 (3.95 g, 97%) as an off-white solid. ¹H NMR (DMSO-d₆): δ 10.62 (s, 1H), 7.24 (d, 1H, J=9.0 Hz), 7.06 (d, 1H, J=3.0 Hz), 7.01 (d, 1H, J=3.0 Hz), 6.72 (dd, 1H, J=1.5, 9.0 Hz), 3.77 (s, 3H), 2.89-2.85 (m, 2H), 2.72-2.65 (m, 1H), 2.22 (s, 3H), 2.08-2.01 (m, 2H), 1.95-1.90 (m, 2H), 1.74-1.64 (m, 2H).

Synthesis of (9Z,12Z)-1-(5-methoxy-3-(1-methylpiperidin-4-yl)-1H-indol-1-yl)octadeca-9,12-dien-1-one (I-11): A solution of linoleic acid (4.64 g, 16.575 mmol) in dry CH₂Cl₂ (50 mL) was treated with oxalyl chloride (1.87 mL, 22.101 mmol) followed by 1 drop of dry DMF at room temperature and stirred for additional 2 h. Solvent was evaporated and crude product was dried on high vacuum to obtain the corresponding acid chloride.

A solution of 5-methoxy-3-(1-methylpiperidin-4-yl)-1H-indole (1.35 g, 5.525 mmol) in dry DMF (20 mL) cooled to 0° C. and treated with sodium hydride (0.88 g, 22.101 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound 1-1 to obtain the title compound 1-11 (1.62 g, 57.8%) as a brown oil. ¹H NMR (CDCl₃): δ 8.39 (d, 1H, J=6.0 Hz), 7.19 (s, 1H), 7.02 (d, 1H, J=1.5 Hz), 6.97 (dd, 1H, J=3.0, 6.0 Hz), 5.42-5.32 (m, 4H), 3.89 (s, 3H), 3.06-3.01 (m, 2H), 2.86 (t, 2H, J=6.0 Hz), 2.80 (t, 2H, J=6.0 Hz), 2.39 (s, 3H), 2.20-2.05 (m, 7H), 1.87-1.80 (m, 4H), 1.48-1.28 (m, 16H), 0.90 (t, 3H, J=3.0 Hz); ESI-MS (m/z, %): 507 (MH⁺, 100).

### Example 31: 3-(Pyrrolidin-3-yl)-1H-indole-2,4,5,6,7-d₅ (64)

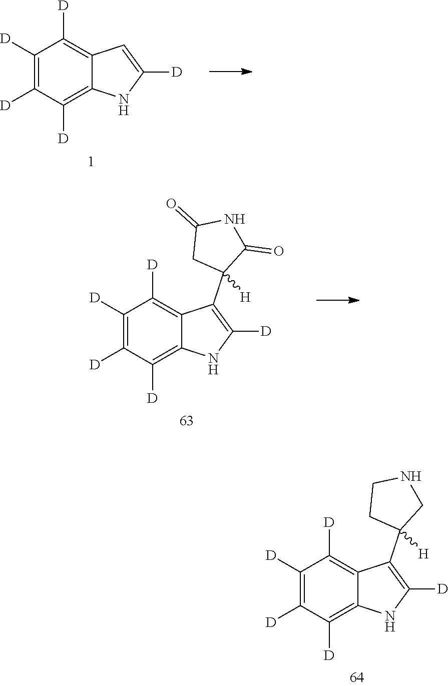 [SMILES: [2H]c1cc2c([2H])c([2H])c([2H])c([2H])c2[nH]1 ; [2H]c1[nH]c2c([2H])c([2H])c([2H])c([2H])c2c1C1CC(=O)NC1=O ; [2H]c1[nH]c2c([2H])c([2H])c([2H])c([2H])c2c1C1CCNC1]

Synthesis of 3-(1H-indol-3-yl-2,4,5,6,7-d₅)pyrrolidine-2,5-dione (63): A solution of 1H-indole-2,4,5,6,7-d₅ (2.3 g, 18.671 mmol), maleimide (1.99 g, 20.538 mmol) in acetic acid was refluxed for 3 days. Solvent was evaporated and crude was crystallized from a mixture of IPA:hexanes:CH₂Cl₂, (7:2.5:0.5) to obtain the title compound 63 (2.72 g, 66.5%) as a yellow solid.

Synthesis of 3-(pyrrolidin-3-yl)-1H-indole-2,4,5,6,7-d₅ (64): A suspension of Lithium aluminum hydride (1.38 g, 36.488 mmol) in dry THF (20 mL) was treated with 3-(1H-indol-3-yl-2,4,5,6,7-d₅)pyrrolidine-2,5-dione (1.0 g, 4.561 mmol) in dry THF (30 mL) at 0° C. over a period of 10 min. The reaction was brought to room temperature, then refluxed for additional 16 hours, worked-up and purified as described for compound 3 to obtain the title compound 64 (0.4 g, 45.9%) as a dark brown semi-solid. ¹H NMR (DMSO-d₆): δ 10.80 (s, 1H), 7.57-7.55 (m, 0.13H), 7.35 (s, 0.09H), 7.17 (s, 1H), 7.08-7.07 (m, 0.18H), 6.98-6.97 (m, 0.19H), 3.42-3.20 (m, 2H), 3.06-2.94 (m, 2H), 2.80-2.76 (m, 1H), 2.22-2.14 (m, 1H), 1.88-1.79 (m, 1H); ESI-MS (m/z, %): 192 (MH⁺), 191 (M⁺, 100).

### Example 32: 3-(1-Methylpyrrolidin-3-yl)-1H-indole-2,4,5,6,7-d₅ (66)

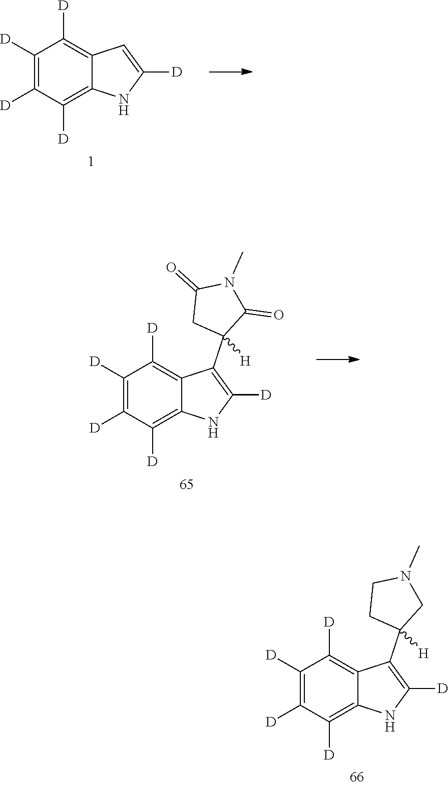 [SMILES: [2H]c1cc2c([2H])c([2H])c([2H])c([2H])c2[nH]1 ; [2H]c1[nH]c2c([2H])c([2H])c([2H])c([2H])c2c1C1CC(=O)N(C)C1=O ; [2H]c1[nH]c2c([2H])c([2H])c([2H])c([2H])c2c1C1CCN(C)C1]

Synthesis of 3-(1H-indol-3-yl-2,4,5,6,7-d₅)-1-methylpyrrolidine-2,5-dione (65): A solution of 1H-indole-2,4,5,6,7-d₅ (5.1 g, 41.402 mmol), N-methyl maleimide (5.06 g, 45.543 mmol) in acetic acid was refluxed for 3 days. Solvent was evaporated and crude was crystallized from a mixture of IPA:hexanes, (9:1) to obtain the title compound 65 (4.33 g, 44.8%) as a yellow solid. ¹H NMR (DMSO-d₆): δ 11.04 (s, 1H), 4.39-4.34 (m, 1H), 3.31-3.19 (m, 1H), 2.93 (s, 3H), 2.85-2.76 (m, 1H).

Synthesis of 3-(1-methylpyrrolidin-3-yl)-1H-indole-2,4,5,6,7-d₅ (66): A suspension of Lithium aluminum hydride (1.3 g, 34.292 mmol) in dry THF (20 mL) was treated with 3-(1H-indol-3-yl-2,4,5,6,7-d₅)-1-methylpyrrolidine-2,5-dione (1.0 g, 4.286 mmol) in dry THF (30 mL) at 0° C. over a period of 10 min. The reaction was brought to room temperature, then refluxed for additional 16 hours, worked-up and purified as described for compound 3 to obtain the title compound 66 (0.4 g, 45.4%) as a pale-yellow solid. ¹H NMR (CDCl₃): δ 8.05 (s, 1H), 7.69 (s, 0.1H), 7.38 (s, 0.1H), 7.22 (s, 0.13H), 7.14 (s, 0.22H), 7.07-7.04 (m, 0.72H), 3.77-3.68 (m, 1H), 3.17-3.13 (m, 1H), 2.93-2.88 (m, 1H), 2.69-2.60 (m, 2H), 2.47 (s, 3H), 2.46-2.42 (m, 1H), 2.09-2.05 (m, 1H); ESI-MS (m/z, %): 206 (MH⁺), 205 (M⁺, 100).

### Example 33: (9Z,12Z)-1-(3-(1-Methylpyrrolidin-3-yl)-1H-indol-1-yl)octadeca-9,12-dien-1-one (I-12)

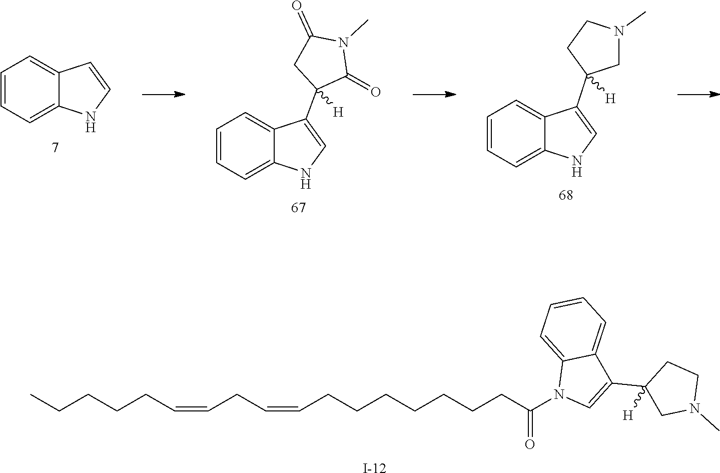

Synthesis of 3-(1H-indol-3-yl)-1-methylpyrrolidine-2,5-dione (67): A solution of 1H-indole (12.5 g, 106.70 mmol), N-methyl maleimide (13.0 g, 117.37 mmol) in acetic acid (75 mL) was refluxed for 3 days. Solvent was evaporated and crude was crystallized from ethyl acetate to obtain the title compound 67 (12.0 g, 49.3%) as a light-yellow solid. ¹H NMR (DMSO-d₆): δ 11.06 (s, 1H), 7.41-7.35 (m, 3H), 7.14-7.09 (m, 1H), 7.03-6.99 (m, 1H), 4.40-4.36 (m, 1H), 3.34 (s, 3H), 3.28-3.21 (m, 1H), 2.84-2.78 (m, 1H).

Synthesis of 3-(1-methylpyrrolidin-3-yl)-1H-indole (68): A suspension of Lithium aluminum hydride (2.3 g, 60.635 mmol) in dry THF (25 mL) was treated with 3-(1H-indol-3-yl)-1-methylpyrrolidine-2,5-dione (1.73 g, 7.579 mmol) in dry THF (35 mL) at 0° C. over a period of 10 min. The reaction was brought to room temperature, then refluxed for additional 16 hours, worked-up and purified as described for compound 3 to obtain the title compound 68 (1.26 g, 83.4%) as a beige solid. ¹H NMR (CDCl₃): δ 8.23 (brs, 1H), 7.69 (dd, 1H, J=1.5, 6.0 Hz), 7.38 (dd, 1H, J=1.5, 6.0 Hz), 7.24-7.12 (m, 2H), 7.03 (d, 1H, J=1.5 Hz), 3.77-3.69 (m, 1H), 3.18-3.14 (m, 1H), 2.94-2.88 (m, 1H), 2.70-2.62 (m, 2H), 2.48 (s, 3H), 2.46-2.43 (m, 1H), 2.09-2.02 (m, 1H); ESI-MS (m/z, %): 201 (MH⁺).

Synthesis of (9Z,12Z)-1-(3-(1-methylpyrrolidin-3-yl)-1H-indol-1-yl)octadeca-9,12-dien-1-one (I-12): A solution of linoleic acid (3.5 g, 12.598 mmol) in dry CH₂Cl₂ (50 mL) was treated with oxalyl chloride (1.42 mL, 16.798 mmol) followed by 1 drop of dry DMF at room temperature and stirred for additional 2 h. Solvent was evaporated and crude product was dried on high vacuum to obtain the corresponding acid chloride.

A solution of 3-(1-methylpyrrolidin-3-yl)-1H-indole (0.9 g, 4.199 mmol) in dry DMF (20 mL) cooled to 0° C. and treated with sodium hydride (0.67 g, 16.798 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound 1-1 to obtain the title compound 1-12 (1.65 g, 85%) as a light brown oil. ¹H NMR (CDCl₃): δ 8.49 (d, 1H, J=6.0 Hz), 7.60 (d, 1H, J=6.0 Hz), 7.40-7.29 (m, 3H), 5.43-5.35 (m, 4H), 3.71-3.60 (m, 1H), 3.25-3.21 (m, 1H), 2.97-2.78 (m, 6H), 2.55 (s, 3H), 2.50-2.45 (m, 2H), 2.12-2.05 (m, 5H), 1.88-1.84 (m, 2H), 1.50-1.29 (m, 14H), 0.92 (t, 3H, J=6.0 Hz); ESI-MS (m/z, %): 463 (MH⁺, 100).

### Example 34: 3-(1-Methylpyrrolidin-3-yl-2,2,5,5-d₄)-1H-indole-2,4,5,6,7-d₅ (70)

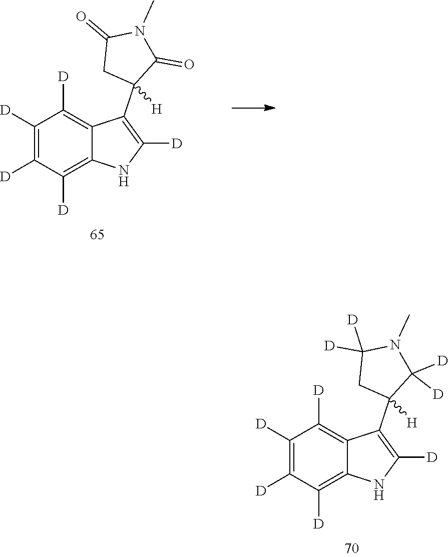 [SMILES: [2H]c1[nH]c2c([2H])c([2H])c([2H])c([2H])c2c1C1CC(=O)N(C)C1=O ; [2H]c1[nH]c2c([2H])c([2H])c([2H])c([2H])c2c1C1CC([2H])([2H])N(C)C1([2H])[2H]]

Synthesis of 3-(1-methylpyrrolidin-3-yl-2,2,5,5-d₄)-1H-indole-2,4,5,6,7-d₅ (70): A suspension of Lithium aluminum deuteride (1.44 g, 34.292 mmol) in dry THF (20 mL) was treated with 3-(1H-indol-3-yl-2,4,5,6,7-d5)-1-methylpyrrolidine-2,5-dione (1.0 g, 4.286 mmol) in dry THF (30 mL) at 0° C. over a period of 10 min. The reaction was brought to room temperature, then refluxed for additional 16 hours, worked-up and purified as described for compound 3 to obtain the title compound 70 (0.425 g, 47.4%) as a pale-yellow solid. ¹H NMR (CDCl₃): δ 8.29 (s, 1H), 7.70-7.68 (m, 0.08H), 7.38-7.36 (m, 0.09H), 7.23-7.21 (m, 0.12H), 7.15-7.12 (m, 0.23H), 7.03-7.02 (m, 0.82H), 3.74-3.70 (m, 1H), 2.48 (s, 3H), 2.45-2.39 (m, 1H), 2.08-2.03 (m, 1H); ESI-MS (m/z, %): 210 (MH⁺), 209 (M⁺, 100).

### Example 35: (9Z,12Z)-1-(5-Methoxy-3-(1-methylpyrrolidin-3-yl)-1H-indol-1-yl)octadeca-9,12-dien-1-one (I-13)

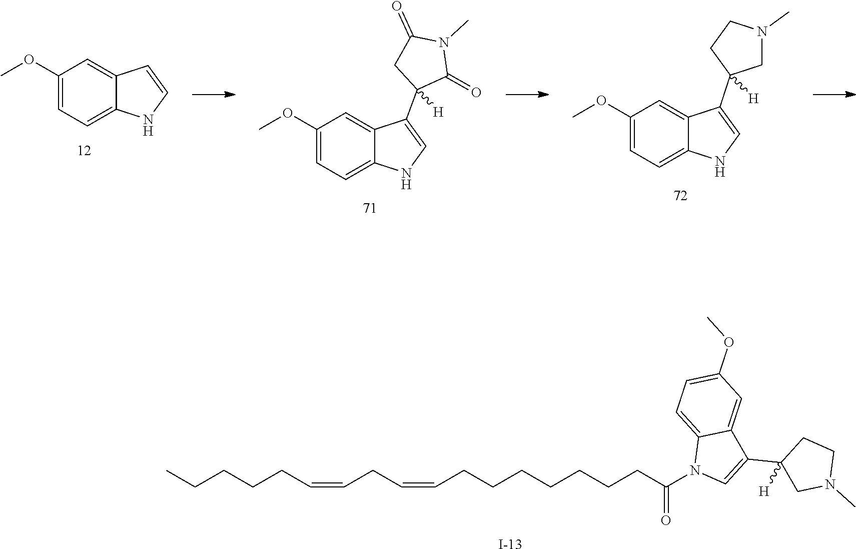

Synthesis of 3-(5-methoxy-1H-indol-3-yl)-1-methylpyrrolidine-2,5-dione (71): A solution of 5-methoxy-1H-indole (12.3 g, 83.559 mmol), N-methyl maleimide (10.2 g, 91.915 mmol) in acetic acid (80 mL) was refluxed for 3 days. Solvent was evaporated and crude was crystallized from a mixture of IPA:hexanes:CH₂Cl₂, (7:2.5:0.5) to obtain the title compound 71 (17.2 g, 79.7%) as a brown solid. ¹H NMR (CDCl₃): δ 10.89 (s, 1H), 7.29-7.26 (m, 2H), 6.89 (d, 1H, J=3.0 Hz), 6.77 (dd, 1H, J=3.0, 6.0 Hz), 4.36-4.32 (m, 1H), 3.74 (s, 3H), 3.34-3.26 (m, 1H), 2.93 (s, 3H), 2.84-2.75 (m, 1H).

Synthesis of 5-methoxy-3-(1-methylpyrrolidin-3-yl)-1H-indole (72): A suspension of Lithium aluminum hydride (2.35 g, 61.950 mmol) in dry THF (25 mL) was treated with 3-(5-methoxy-1H-indol-3-yl)-1-methylpyrrolidine-2,5-dione (2.0 g, 7.743 mmol) in dry THF (40 mL) at 0° C. over a period of 10 min. The reaction was brought to room temperature, then refluxed for additional 16 hours, worked-up and purified as described for compound 3 to obtain the title compound 72 (1.3 g, 73%) as a light brown glue. ¹H NMR (CDCl₃): δ 8.18 (brs, 1H), 7.27 (d, 1H, J=6.0 Hz), 7.14 (d, 1H, J=3.0 Hz), 7.02 (d, 1H, J=1.5 Hz), 6.89 (dd, 1H, J=3.0, 6.0 Hz), 3.90 (s, 3H), 3.73-3.65 (m, 1H), 3.18-3.14 (m, 1H), 2.94-2.89 (m, 1H), 2.71-2.60 (m, 2H), 2.48 (s, 3H), 2.45-2.42 (m, 1H), 2.08-2.02 (m, 1H); ESI-MS (m/z, %): 231 (MH⁺, 100).

Synthesis of (9Z,12Z)-1-(5-methoxy-3-(1-methylpyrrolidin-3-yl)-1H-indol-1-yl)octadeca-9,12-dien-1-one (I-13): A solution of linoleic acid (3.28 g, 11.723 mmol) in dry CH₂Cl₂ (40 mL) was treated with oxalyl chloride (1.32 mL, 15.630 mmol) followed by 1 drop of dry DMF at room temperature and stirred for additional 2 h. Solvent was evaporated and crude product was dried on high vacuum to obtain the corresponding acid chloride.

A solution of 5-methoxy-3-(1-methylpyrrolidin-3-yl)-1H-indole (0.9 g, 3.907 mmol) in dry DMF (20 mL) cooled to 0° C. and treated with sodium hydride (0.62 g, 15.630 mmol, 60% in mineral oil), brought to room temperature and stirred for 30 min. The reaction was cooled to 0° C., treated with above crude acid chloride in dry DMF (20 mL). The reaction was brought to room temperature and stirred for over night (18 h). The reaction was worked-up and purified as described for compound I-1 to obtain the title compound 1-13 (1.65 g, 85.7%) as a brown oil. ¹H NMR (CDCl₃): δ 8.39 (d, 1H, J=6.0 Hz), 7.30 (d, 1H, J=3.0 Hz), 7.09 (d, 1H, J=3.0 Hz), 6.98 (dd, 1H, J=3.0, 6.0 Hz), 5.43-5.34 (m, 4H), 3.90 (s, 3H), 3.65-3.61 (m, 1H), 3.17 (t, 1H, J=6.0 Hz), 2.92-2.72 (m, 7H), 2.52 (s, 3H), 2.45-2.43 (m, 1H), 2.12-2.03 (m, 5H), 1.87-1.81 (m, 2H), 1.49-1.29 (m, 14H), 0.92 (t, 3H, J=6.0 Hz); ESI-MS (m/z, %): 493 (MH⁺, 100).

### Example 36: 5-Methoxy-3-(1-methylpyrrolidin-3-yl-2,2,5,5-d₄)-1H-indole (74)

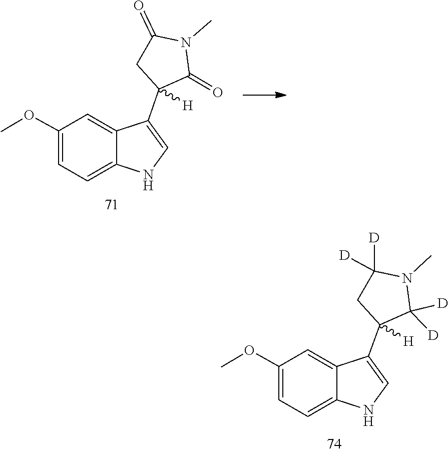 [SMILES: COc1ccc2[nH]cc(C3CC(=O)N(C)C3=O)c2c1 ; [2H]C1([2H])CC(c2c[nH]c3ccc(OC)cc23)C([2H])([2H])N1C]

Synthesis of 5-methoxy-3-(1-methylpyrrolidin-3-yl-2,2,5,5-d₄)-1H-indole (74): A suspension of Lithium aluminum deuteride (2.21 g, 52.657 mmol) in dry THF (25 mL) was treated with 3-(5-methoxy-1H-indol-3-yl)-1-methylpyrrolidine-2,5-dione (1.7 g, 6.582 mmol) in dry THF (40 mL) at 0° C. over a period of 10 min. The reaction was brought to room temperature, then refluxed for additional 16 hours, worked-up and purified as described for compound 3 to obtain the title compound 74 (1.0 g, 65%) as a light brown glue. ¹H NMR (CDCl₃): δ 8.03 (brs, 1H), 7.29 (d, 1H, J=3.0 Hz), 7.13 (d, 1H, J=1.5 Hz), 7.02 (d, 1H, J=1.5 Hz), 6.89 (dd, 1H, J=3.0, 6.0 Hz), 3.89 (s, 3H), 3.69-3.64 (m, 1H), 2.47 (s, 3H); 2.43-2.38 (m, 1H), 2.05-2.00 (m, 1H); ESI-MS (m/z, %): 235 (MH⁺, 100).

### Example 37: 5-Methoxy-3-(1-(methyl-d₃)piperidin-4-yl)-1H-indole (77)

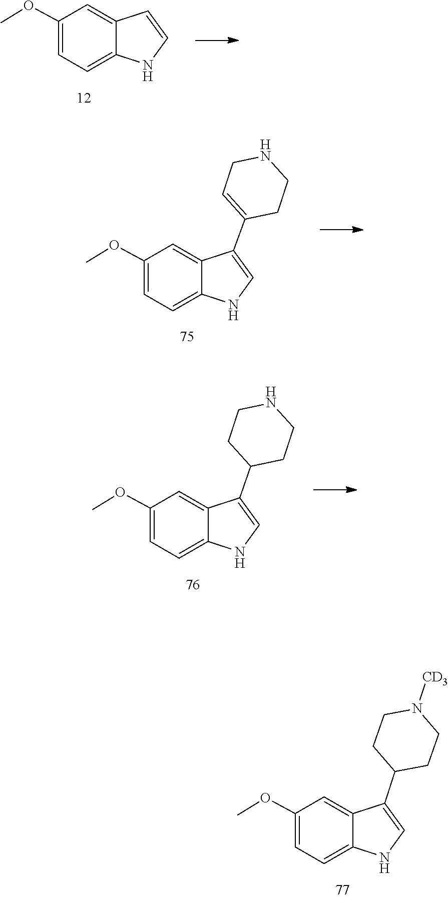 [SMILES: COc1ccc2[nH]ccc2c1 ; COc1ccc2[nH]cc(C3=CCNCC3)c2c1 ; COc1ccc2[nH]cc(C3CCNCC3)c2c1 ; [2H]C([2H])([2H])N1CCC(c2c[nH]c3ccc(OC)cc23)CC1]

Synthesis of 5-methoxy-3-(1,2,3,6-tetrahydropyridin-4-yl)-1H-indole (75): A solution of 5-methoxy-1H-indole (10.5 g, 71.331 mmol) in methanol (200 mL) was treated with potassium hydroxide (20.0 g, 356.657 mmol), followed by piperidin-4-one hydrochloride (27.4 g, 178.328 mmol) at room temperature and the resulting mixture was refluxed for additional 18 h. The reaction was brought to room temperature, methanol was evaporated, diluted with water (250 mL). Solid was filtered off, washed with water (3×25 mL) and dried on high vacuum to obtain the title compound 75 (14.35 g, 88%) as a yellow solid. ¹H NMR (DMSO-d₆): δ 10.96 (s, 1H), 7.34-7.27 (m, 3H), 6.79 (dd, 1H, J=3.0, 6.0 Hz), 6.15-6.13 (m, 1H), 3.78 (s, 3H), 3.45-3.43 (m, 2H), 2.97-2.94 (m, 2H), 2.40-2.37 (m, 2H); ESI-MS (m/z, %): 229 (MH⁺, 100).

Synthesis of 5-methoxy-3-(piperidin-4-yl)-1H-indole (76): A suspension of 5-methoxy-3-(1,2,3,6-tetrahydropyridin-4-yl)-1H-indole (10.2 g, 44.689 mmol) in methanol (200 mL) was treated with Pd—C(1.0 g) and hydrogenated under hydrogen atm. (balloon pressure) for 18 h. The reaction was filtered through a pad of celite and washed with methanol (3×25 mL). Combined methanol layer was evaporated, crude was crystallized from CH₂Cl₂:hexanes (1:2) to obtain the title compound 76 (9.05 g, 88%) as a yellow solid. ¹H NMR (DMSO-d₆): δ 10.61 (s, 1H), 7.24 (d, 1H, J=6.0 Hz), 7.04-7.03 (m, 2H), 6.73 (dd, 1H, J=3.0, 6.0 Hz), 3.78 (s, 3H), 3.06-3.02 (m, 2H), 2.85-2.81 (m, 1H), 2.79-2.64 (m, 2H), 1.91-1.86 (m, 2H), 1.61-1.51 (m, 2H); ESI-MS (m/z, %): 231 (MH⁺, 100).

Synthesis of 5-methoxy-3-(1-(methyl-d₃)piperidin-4-yl)-1H-indole (77): A solution of 5-methoxy-3-(piperidin-4-yl)-1H-indole (0.31 g, 1.346 mmol) in dry DMF (5 mL) was treated with triethyl amine (0.75 mL, 5.384 mmol), followed by iodo-methane-d₃ (0.16 mL, 2.692 mmol) at room temperature and stirred for additional 24 h. The reaction was worked-up and purified as described for compound 57 to obtain the title compound 77 (0.05 g, 15%) as an off-white solid. ¹H NMR (CDCl₃): δ 7.90 (brs, 1H), 7.27 (s, 1H), 7.11 (d, 1H, J=3.0 Hz), 7.00 (d, 1H, J=1.5 Hz), 6.89 (dd, 1H, J=3.0, 6.0 Hz), 3.90 (s, 3H), 3.04-3.01 (m, 2H), 2.83-2.76 (m, 1H), 2.21-2.07 (m, 4H), 1.92-1.82 (m, 2H); ESI-MS (m/z, %): 248 (MH⁺, 100).

### Example 38: (9Z,12Z)-1-(5-Methoxy-3-(1-(methyl-d₃)piperidin-4-yl)-1H-indol-1-yl)octadeca-9,12-dien-1-one (I-14)

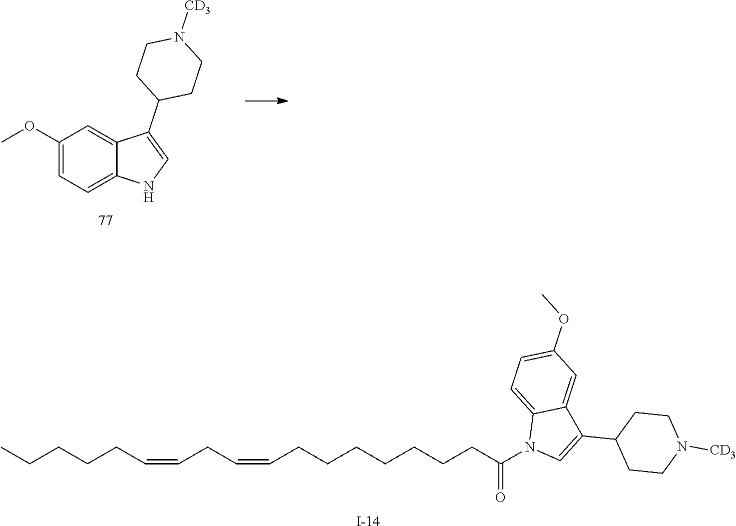

Synthesis of (9Z,12Z)-1-(5-methoxy-3-(1-(methyl-d₃)piperidin-4-yl)-1H-indol-1-yl)octadeca-9,12-dien-1-one (I-14): A solution of linoleic acid (2.58 g, 9.218 mmol) in dry CH₂Cl₂ (30 mL) was treated with oxalyl chloride (1.03 mL, 12.288 mmol) followed by 1 drop of dry DMF at room temperature and stirred for additional 2 h. Solvent was evaporated and crude product was dried on high vacuum to obtain the corresponding acid chloride.

A solution of 5-methoxy-3-(1-(methyl-d₃)piperidin-4-yl)-1H-indole (0.76 g, 3.072 mmol) in dry DMF (10 mL) was cooled to 0° C. and treated with sodium hydride (0.49 g, 12.288 mmol, 60% in mineral oil), brought to room temperature and stirred for 30 min. The reaction was cooled to 0° C., treated with above crude acid chloride in dry DMF (10 mL). The reaction was brought to room temperature and stirred for over night (18 h). The reaction was worked-up and purified as described for compound I-1 to obtain the title compound 1-14 (0.83 g, 53%) as a pale-yellow oil. ¹H NMR (CDCl₃): δ 8.38 (d, 1H, J=6.0 Hz), 7.28 (s, 1H), 7.19 (s, 1H), 7.01 (d, 1H, J=3.0 Hz), 6.96 (dd, 1H, J=3.0, 6.0 Hz), 5.42-5.31 (m, 4H), 3.89 (s, 3H), 3.11-3.08 (m, 2H), 2.87-2.72 (m, 4H), 2.30-2.22 (m, 2H), 2.09-2.04 (m, 6H), 1.94-1.80 (m, 4H), 1.47-1.28 (m, 14H), 0.90 (t, 3H, J=3.0 Hz); ESI-MS (mz, %): 510 (MH⁺, 100).

### B. Biology Testing

### Example B-1: FLIPR Assay: Human 5-HT_{2A}

1. Assessment of the activated effect of exemplary compounds of Formula I targeting on human 5-HT_{2A} (h5-HT_{2A}) receptor under agonist mode:

### Compound Preparation and Assay Controls

I.a. Reagent and Materials:

| Regents | Vendor | Cat# |
|---|---|---|
| DMEM | Gibco | 10569010 |
| FBS | Hyclone | SH30406 |
| Penicillin- | Invitrogen | 15140 |
| Streptomycin | | |
| Hygromycin B | Invivogen | Ant-hg-5 |
| G418 | Invitrogen | 11811031 |
| Tetracycline | Abcam | ab141223 |
| hydrochloride | | |
| DPBS | Gibco | 14190250 |
| DMSO | Millipore | 1029312500 |
| Probenecid | Sigma | P8761 |
| FLIPR Calcium 6 | Molecular | R8191 |
| Assay Kit | Device | |
| HEPES | Invitrogen | 15630 |
| Hank's Buffered | Invitrogen | 14025 |
| Saline Solution | | |
| Serotonin HCl | Selleck | S4244 |

I.b. Instrumentation and Consumables:

| Item | Supplier | Cat# |
|---|---|---|
| Fluorometric Imaging | Molecular | Tetra |
| Plate Reader (FLIPR) | Device | |
| Countess Automated | Invitrogen | Countess |
| Cell Counter | | |
| Cell Counting | Invitrogen | C10312 |
| Chamber Slides | | |
| STERI-CYCLE CO₂ | Thermo | 371 |
| Incubator | | |
| 1300 Series Class II | Thermo | 1389 |
| Biological Safety Cabinet | | |
| Table-type Large | Cence | L550 |
| Capacity Low Speed | | |
| Centrifuge | | |
| Centrifuge | Eppendorf | 5702 |
| Echo | Labcyte | 550 |
| Echo | Labcyte | 655 |
| Electro-thermal incubator | Shanghai Yiheng | DHP-9031 |
| plate shaker | IKA | MS3 digital |
| Water Purification System | ULUPURE | UPH-III-20T |
| Versatile and Universal | Mettler | S220 |
| pH and Conductivity Meters | Toledo | |
| 384-Well plate | Corning | 356663 |
| 384-Well LDV | LABCYTE | LP-0200 |
| Clear microplate | | |
| 384-Well | LABCYTE | PP-0200 |
| Polypropylene microplate | | |
| 384-well compound plate | Corning | 3657 |
| T25 cell culture flask | Corning | 430639 |
| 50 mL Polypropylene | JET | CFT011500 |
| Centrifuge Tube | | |
| 15 mL Polypropylene | JET | CFT011150 |
| Centrifuge Tube | | |

I.c. Experimental Methods and Procedures:

1. Cells were cultured in cell culture medium (DMEM containing 10% FBS, 1× penicillin-streptomycin 300 μg/ml G418 and 100 μg/ml hygromycin B) at 37° C., 5% (v/v) CO2.

2. One day before the assays, the cells were detached using TrypLE™ Express and cells were counted using cell counter. Only cells with >85% viability were used for the assay.

3. 20000 cells/well were seeded in 30 μl/well culture medium to a 384-well cell plate and cells were incubated overnight at 37° C., 5% (v/v) CO2.

4. On the assay day, 2×dye solution was prepared following the manual of the FLIPR® Calcium 6 Assay Kit: i. The dye was diluted with assay buffer (20 mM HEPES in 1×HBSS, PH7.4); ii. Probenecid was added to the final concentration of 5 mM; iii. Vortexed vigorously for 1-2 minutes.

5. Medium was removed from cell plate by flicking the cell plate on towel papers.

6. 10 μl of assay buffer and 10 μl of 2×dye solution was added to each well of the cell plate.

7. The cell plate was placed on plate shaker, the plate was agitated at 600 rpm for 2 minutes. The plate was incubated at 37° C. for 2 hours followed by additional 15-minute incubation at 25° C.

8. 3×compound in assay buffer was prepared: a. Reference compounds were diluted to required concentration with DMSO. The compounds were added to a 384-well compound plate; b. Serial dilutions were performed; c. 10 mM test compounds were added to the compound plate, and 3-fold serial dilutions were performed. d. Transferred 60 nl/well of compounds from source plate to a 384-well compound plate (Corning, 3657) by using an Echo; e. Add 20 μl/well assay buffer to the compound plate; f. Mixed the plate on plate shaker for 2 mins;

9. The cell plate, compound plate and tips were put into FLIPR, 10 μl of 3× compound was transferred to the cell plate per well with FLIPR.

### Data Analysis

i. The normalized fluorescence reading (RFU) was calculated as shown follow, while Fmax and Fmin stand for maximum and minimum of calcium signal during defined time window: RFU=Fmax−Fmin

ii. Calculated the percentage activation by using following equation:

$$\% Activation = \frac{( RFUcompound - RFU low control )}{( RFU top concentration of reference agonist - RFU low control )} \times 100 \%$$

lii. Calculated EC₅₀ by fitting % activation against log of compound concentrations with Hill equation using XLfit.

The compounds of the application were found to be 5-HT_{2A} agonists. The results of representative compounds are presented as EC50 provided in Table 1. The letter “A” indicates an EC50<1,000 nM; “B” indicates and EC50>1,000 nM but <10,000 nM; and “C” indicates and EC50>10,000 nM.

TABLE 1
Effect of exemplary compounds of Formula I targeting
on human 5-HT_{2A} (h5-HT_{2A}) receptor under agonist mode

| | h5-HT_{2A} |
|---|---|
| Example ID# | EC₅₀ [nM] |
| 5-MeO-DMT | A |
| DMT | A |
| I-10 | C |
| I-11 | C |
| I-14 | C |

### Results & Discussion

Exemplary compounds of Formula I were evaluated functionally using FLIPR assay for their effect on h5-HT_{2A} receptor under agonist mode. EC₅₀ (nM) concentrations are illustrated in Table 1. This assay confirmed that compounds of the application are effective agonists of the target human 5-HT_{2A} receptors.

### II. Human 5-HT_{2A}: Radioligand Binding Assay

### II.1. Materials and Instruments

| Materials | Vendor | Cat# |
|---|---|---|
| Ketanserin Hydrochloride, | PerkinElmer | NET791250UC |
| [Ethylene-3H]- | | |
| Ketanserin | MedChemExpress | HY-10562 |
| Bovine Serum | Sigma | A1933 |
| Albumin (BSA) | | |
| Calcium chloride (CaCl₂) | Sigma | C5670 |
| Tris(hydroxymethyl)amino- | Alfa Aesar | A18494 |
| methane (Tris) | | |
| Polyethylenimine, | Sigma | 408727 |
| branched (PEI) | | |

### II.2. Instrumentation and Consumables

| Item | Supplier | Cat# |
|---|---|---|
| Microbeta² Microplate | PerkinElmer | 2450-0060 |
| Counter | | |
| UniFilter-96 GF/B | PerkinElmer | 6005177 |
| TopSeal | Biotss | SF-800 |
| MicroBeta | PerkinElmer | D961962 |
| Filtermate-96 | | |
| Seven Compact | Mettler | S220 |
| pH meter | Toledo | |
| Ultrapure | Sichuan | UPH-III-20T |
| Water Meter | Ulupure | |
| Benchtop | Hunan | L550 |
| Centrifuge | Xiangyi | |
| Microplate Shaker | Allsheng | MX100-4A |
| 384-Well Polypropylene | Labcyte | PP-0200 |
| Microplate | | |
| 96 Round | Corning | 3799 |
| Well Plate | | |
| 96 Round Deep | Axygen | P-DW-11-C |
| Well Plate | | |
| Echo | LABCYTE | 550 |

### II.3 Experiment Procedure

i. Prepared the assay buffer following the table below;

| Reagent | Concentration |
|---|---|
| Tris | 50 mM |
| CaCl₂ | 4 mM |
| BSA | 0.1% (w/v) |

Adjust pH to 7.4 followed by 0.2 μM sterile filtration

ii. Preparation of 8 doses of reference and test compounds starting from 10 mM stock solution as requested by 5-fold serial dilutions with 100%;

iii. Prepared (v/v) DMSO: a. 50 μl/well of 0.5% (v/v) PEI was added to UniFilter-96 GF/B plates. The plates were sealed and incubates at 4° C. for 3 hrs; b. After incubation, the plates were washed 3 times with ice-cold wash buffer (50 mM Tris, pH7.4);

iv. Preparation of assay plates: a. Cell membrane were diluted with assay buffer and 330 μl/well was added to 96 round deep well plates to reach a concentration of 20 μg/well; b. 8 concentrations of reference or test compounds were prepared and 110 μl/well wa added to 96 round deep well plates; c. [3H]-ketanserin was diluted with assay buffer to 5 nM (5× final concentration) and 110 μl/well was added to 96 round deep well plates.

v. The plate was centrifuged at 1000 rpm for 30 secs and then agitated at 600 rpm, R.T. for 5 min.

vi. The plates were sealed and incubates at 27° C. for 90 min.

vii. The incubation was stopped by vacuum filtration onto GF/B filter plates followed by 4 times washing with ice-cold wash buffer (50 mM Tris, pH7.4).

viii. The plates were dried at 37° C. for 45 min.

ix. The filter plates were sealed and 40 μl/well of scintillation cocktail was added.

x. The plate was read by using a Microbeta2 microplate counter.

### Data Analysis:

For reference and test compounds, the results are expressed as % Inhibition, using the normalization equation: N=100−100×(U−C2)/(C1−C2), where U is the unknown value, C1 is the average of high controls, and C2 is the average of low controls. The IC50 is determined by fitting percentage of inhibition as a function of compound concentrations with Hill equation using XLfit.

### Results and Discussion

The results of potential competition binding properties of the representative compounds targeting on human 5-hydroxytryptamine receptors 2A (5-HT_{2A}) are summarized in Table 2. The results of representative compounds are presented as IC₅₀ provided in Table 2. The symbol “#” indicates an IC₅₀<2,000 nM; “##” indicates and IC50>2,000 nM but <5,000 nM; and “##” indicates IC₅₀>5,000 nM.

Table 2: Effect of exemplary compounds of Formula I using Radioligand binding assay on human 5-HT_{2A} receptor

| | h5-HT_{2A} |
|---|---|
| Example ID# | IC₅₀ [nM] |
| 5-MeO-DMT | # |
| DMT | # |
| I-1 | # |
| I-2 | # |
| I-3 | # |
| I-4 | # |
| I-5 | # |
| I-6 | # |
| I-7 | ## |
| I-8 | # |
| I-9 | ## |
| I-10 | # |
| I-12 | ## |
| I-13 | ## |
| I-14 | ## |

### Results & Discussion

Exemplary compounds of Formula I were evaluated using radioligand binding assay on human 5-HT_{2A} receptor. 10₅₀ (nM) concentrations are illustrated in Table 2. This assay confirms that compounds of the application are effective ligands of the target human 5-HT_{2A} receptors.

### Example B-2: Human, Rat and Mouse Liver Microsomes Stability

### Objective

The objective of this study was to estimate in vitro metabolic stability of representative compounds of the application in pooled human and male mouse liver microsomes. The concentrations of parent compounds in reaction systems were evaluated by LC-MS/MS for estimating the stability in pooled human and male mouse liver microsomes. The in vitro intrinsic clearances of test compounds were determined as well.

### Protocol

A master solution in the “Incubation Plate” containing phosphate buffer, ultra-pure H₂O, MgCl₂ solution and liver microsomes was made according to Table 3. The mixture was pre-warmed at 37° C. water bath for 5 minutes.

### Preparation of Master Solution

| | Stock | | | Final | |
|---|---|---|---|---|---|
| Reagent | Concentration | | Volume | Concentration | |
| Phosphate buffer | 200 | mM | 200 μL | 100 | mM |
| Ultra-pure H₂O | — | | 106 μL | — | |
| MgCl₂ solution | 50 | mM | 40 μL | 5 | mM |
| Microsomes | 20 | mg/mL | 10 μL | 0.5 | mg/mL |

40 μL of 10 mM NADPH solution was added to each well. The final concentration of NADPH was 1 mM. The negative control samples were prepared by replacing NADPH with 40 μL of ultra-pure H₂O. Samples were prepared in duplicate. Negative controls were prepared in singlet.

The reaction was started with the addition of 4 μL of 200 μM test compounds or control compounds to each master solution to get the final concentration of 2 μM. This study was performed in duplicate.

Aliquots of 50 μL were taken from the reaction solution at 0, 15, 30, 45 and 60 minutes. The reaction solutions were stopped by the addition of 4 volumes of cold methanol with IS (100 nM alprazolam, 200 nM imipramine, 200 nM labetalol and 2 μM ketoprofen). Samples were centrifuged at 3,220 g for 40 minutes. Aliquot of 90 μL of the supernatant was mixed with 90 μL of ultra-pure H2O and then was used for LC-MS/MS analysis.

LC/MS analysis was performed for all samples from this study using a Shimadzu liquid chromatograph separation system equipped with degasser DGU-20A_{5R}; solvent delivery unit LC-30AD; system controller SIL-30AC; column oven CTO-30A; CTC Analytics HTC PAL System. Mass spectrometric analysis was performed using an Triple Quad™ 5500 instrument.

All calculations were carried out using Microsoft Excel. Peak area ratios of test compound to internal standard (listed in the below table) were determined from extracted ion chromatograms.

All calculations were carried out using Microsoft Excel. Peak areas were determined from extracted ion chromatograms. The slope value, k, was determined by linear regression of the natural logarithm of the remaining percentage of the parent drug vs. incubation time curve.

The in vitro half-life (in vitro t_{1/2}) was determined from the slope value:

$$in vitro {t}_{1 / 2} = - ( 0.693 / k )$$

Conversion of the in vitro t_{1/2}(min) into the in vitro intrinsic clearance (in vitro CLᵢₙₜ, in μL/min/mg proteins) was done using the following equation (mean of duplicate determinations):

$$in vitro {CL}_{int} = ( \frac{0.693}{( {t}_{1 / 2} )} ) * ( \frac{volume of incubation ( µL )}{amount of proteins ( mg )} )$$

For the compound or control compound that showed an initial fast disappearance followed by a slow disappearance, only the time points that were within the initial rate were included in the calculation.

### Results & Discussion

Human, rat and mouse liver microsomes contain a wide variety of drug metabolizing enzymes and are commonly used to support in vitro ADME (absorption, distribution, metabolism and excretion) studies. These microsomes are used to examine the potential first-pass metabolism by-products of orally administered drugs. Representative compounds of the application were evaluated for their stability in human, rat and mouse liver microsomes. A majority of the compounds of the application in three species, human, rat and mouse liver microsomes were recovered within a 60 minute time period indicating that the compounds were not rapidly cleared (see Table 3 for representative compounds of Formula I).

TABLE 3
Metabolic stability in Liver Microsomes of representative
compounds of Formula I, control compound verapamil
in human, rat, mouse and dog

| | | | | Scaled- | Predicted |
|---|---|---|---|---|---|
| Com- | | | CLᵢₙₜ | up | hepatic |
| pound | | T_{1/2} | (μL/min/mg | CLᵢₙₜ | CL |
| ID | Species | (min) | protein) | (mL/min/Kg) | (mL/min/kg) |
| Verapamil | Human | 12.56 | 110.39 | 138.45 | 18.01 |
| | Rat | 5.51 | 251.33 | 450.38 | 49.17 |
| | Mouse | 4.54 | 305.25 | 1335.45 | 84.32 |
| | Dog | 11.21 | 123.60 | 308.12 | 28.08 |
| I-2 | Human | 216.45 | 6.40 | 8.03 | 5.79 |
| | Rat | 45.83 | 30.24 | 54.19 | 27.34 |
| | Mouse | 51.58 | 26.87 | 117.56 | 50.97 |
| | Dog | 44.26 | 31.32 | 78.07 | 22.14 |
| I-5 | Human | 186.87 | 7.42 | 9.30 | 6.42 |
| | Rat | 55.05 | 25.18 | 45.12 | 24.83 |
| | Mouse | 78.39 | 17.68 | 77.35 | 41.60 |
| | Dog | 48.81 | 28.40 | 70.79 | 21.51 |
| I-6 | Human | 292.67 | 4.74 | 5.94 | 4.62 |
| | Rat | 129.44 | 10.71 | 19.19 | 14.24 |
| | Mouse | 114.34 | 12.12 | 53.03 | 33.37 |
| | Dog | 40.04 | 34.61 | 86.28 | 22.75 |
| I-8 | Human | 718.98 | 1.93 | 2.42 | 2.16 |
| | Rat | 146.88 | 9.44 | 16.91 | 12.94 |
| | Mouse | 862.34 | 1.61 | 7.03 | 6.52 |
| | Dog | 73.09 | 18.96 | 47.27 | 18.69 |

TABLE 4
Metabolic stability ij Liver Microsomes of representative
compounds of Formula I, control compound verapamil in
human, rat, mouse and dog with and without NADPH

| Compound | | Assay | Remaining Percentage (%) | | |
|---|---|---|---|---|---|
| ID | Species | Format | 0 min | 30 min | 60 min |
| Verapamil | Human | With NADPH | 100.00 | 8.65 | 3.65 |
| | | Without NADPH | 100.00 | 88.32 | 87.96 |
| | Rat | With NADPH | 100.00 | 2.31 | 0.38 |
| | | Without NADPH | 100.00 | 99.32 | 95.61 |
| | Mouse | With NADPH | 100.00 | 1.03 | 0.00 |
| | | Without NADPH | 100.00 | 103.67 | 105.90 |
| | Dog | With NADPH | 100.00 | 10.84 | 2.45 |
| | | Without NADPH | 100.00 | 91.00 | 93.77 |
| I-2 | Human | With NADPH | 100.00 | 87.61 | 82.52 |
| | | Without NADPH | 100.00 | 114.91 | 83.75 |
| | Rat | With NADPH | 100.00 | 56.08 | 40.37 |
| | | Without NADPH | 100.00 | 94.64 | 84.47 |
| | Mouse | With NADPH | 100.00 | 71.53 | 44.66 |
| | | Without NADPH | 100.00 | 95.81 | 80.93 |
| | Dog | With NADPH | 100.00 | 66.91 | 39.08 |
| | | Without NADPH | 100.00 | 94.42 | 91.63 |
| I-5 | Human | With NADPH | 100.00 | 86.52 | 80.05 |
| | | Without NADPH | 100.00 | 85.69 | 80.25 |
| | Rat | With NADPH | 100.00 | 69.74 | 46.98 |
| | | Without NADPH | 100.00 | 97.35 | 95.87 |
| | Mouse | With NADPH | 100.00 | 74.79 | 58.84 |
| | | Without NADPH | 100.00 | 80.19 | 90.42 |
| | Dog | With NADPH | 100.00 | 65.44 | 42.66 |
| | | Without NADPH | 100.00 | 87.66 | 90.58 |
| I-6 | Human | With NADPH | 100.00 | 91.33 | 86.76 |
| | | Without NADPH | 100.00 | 89.58 | 83.22 |
| | Rat | With NADPH | 100.00 | 83.04 | 72.53 |
| | | Without NADPH | 100.00 | 93.17 | 92.62 |
| | Mouse | With NADPH | 100.00 | 76.15 | 69.51 |
| | | Without NADPH | 100.00 | 81.25 | 91.07 |
| | Dog | With NADPH | 100.00 | 49.51 | 35.40 |
| | | Without NADPH | 100.00 | 84.09 | 91.76 |
| I-8 | Human | With NADPH | 100.00 | 91.39 | 94.38 |
| | | Without NADPH | 100.00 | 91.04 | 100.94 |
| | Rat | With NADPH | 100.00 | 91.29 | 75.35 |
| | | Without NADPH | 100.00 | 107.31 | 109.93 |
| | Mouse | With NADPH | 100.00 | 105.41 | 95.29 |
| | | Without NADPH | 100.00 | 87.39 | 85.47 |
| | Dog | With NADPH | 100.00 | 66.41 | 56.62 |
| | | Without NADPH | 100.00 | 84.36 | 92.44 |

### Example B-3: Psychedelic-Like Effect of Compounds of Formula I

The effect of different doses of representative compounds of Formula I were evaluated on head-twitch response (HTR) and other behavioural responses indicative of serotonin syndrome as behavior-based models of psychedelic activity.

### Protocols

### Mouse Head Twitch

Male, C57BL/6J mice (body weight range 20-30 g) were dosed with the appropriate dose of test article, and following a 1-minute pre-treatment time, placed in individual observation chambers. Animals were visually assessed for the incidence head twitches continuously over a 1 hr period. Head twitches were defined as a rapid jerk of the head which was not elicited by an external tactile stimulus (Come and Pickering, Psychopharmacologia, 1967, 11(1): 65-78). Each head twitch was individually counted by a trained observer, and the data expressed as the mean+SEM of 6-10 mice per group. Mice were used in a single experiment only.

### Rat Behavioural Test

Male, Sprague-Dawley rats (body weight range 250-400 g) were dosed with the appropriate dose of test article and following a 1-minute pre-treatment time, placed in locomotor activity boxes (dimensions 17″ W×17″ L×12″ H) and continuously monitored for a 1 hr period with data collected into 10 minute time bins. Animals were visually assessed for overt behavioural signs, including behaviours characteristic of 5-HT_{2A} receptor activation (wet dog shakes, back muscle contractions), 5-HT_{2A} receptor activation (yawning, penile grooming) and 5-HT_{1A} behaviours (forepaw treading, hindlimb abduction) (Halberzettl et al, Behav Brain Res. 256: 328-345, 2013). Additional behavioural and somatic signs characteristic of 5-HT syndrome (e.g. tremor, salivation, flat body posture, core body temperature change) were also measured. Simultaneously, the spontaneous activity of the rats was measured using an automated tracking system (Med Associates, VT, USA). Activity data collected included total distance traveled, rearing counts and ambulatory episodes. All data were expressed as the mean+SEM of 6-10 rats per group.

### Drug Discrimination in the Rat

Male Sprague-Dawley rats were initially food restricted by presentation of 18-20 g food at day end (single housing). After 7 days acclimatisation to the food restriction procedure, they were trained daily to lever press for food (45 mg Bioserve pellet) in standard 2-lever operant conditioning chambers controlled by Med-PC software over a period of 1 week (Med. Associates Ins., St. Albans, VT). The rats were trained to lever press for food to an FR10 value (i.e 10 lever presses for a single food reward). Once stable food responding was acquired to both response levers, discrimination training began. Over a period of 20-50 training sessions, the rats were trained to associate one lever to a 5-MeO-DMT training dose of 1 mg/kg SC, and the second lever to a neutral stimulus (saline, SC) (Winter et al, Pharmacol Biochem Behav. 87(4): 472-480, 2007). Training sessions lasted 30-min or until the delivery of 50 pellets and continued until the animals attained appropriate stimulus control (defined as six consecutive sessions where animals made no more than 16 lever presses before the delivery of the first reward, and at least 95% total responses on the appropriate lever). The rats continued to receive daily food ration in their home cage at day end.

Once trained, tests of substitution were conducted. On test days, both levers were designated active, i.e., every 10th response on either lever resulted in delivery of a food pellet. Test sessions continued until 50 pellets had been obtained or 30 min had elapsed. During these sessions response rate was also measured.

### Results and Discussion

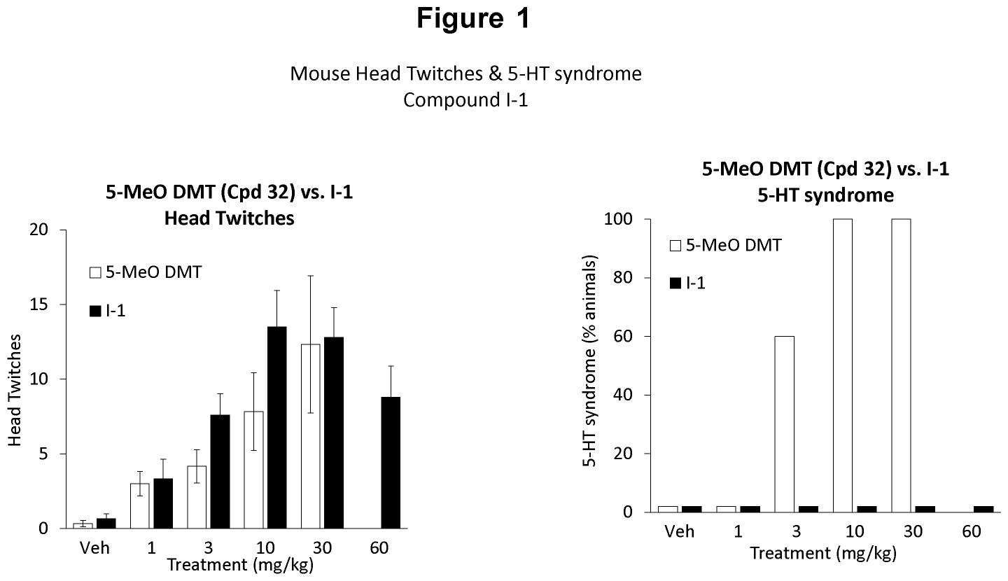

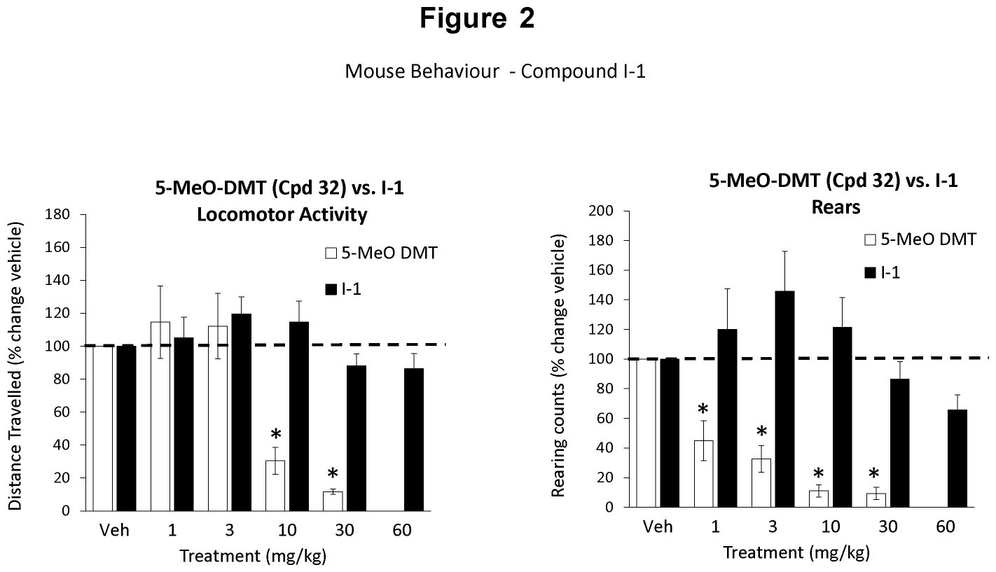

Exemplary compound 1-1 (I-60 mg/kg SC) elicited a robust head twitch response expression that was less variable compared to 5-MeO-DMT (Cpd. 32) (FIG. 1). The ED₅₀ for compound 1-1 was 2.8 mg/kg compared to an ED₅₀ of 8 mg/kg SC for 5-MeO-DMT (Cpd. 32). The duration of HT expression at 10-30 mg/kg was slightly longer for compound 1-1 compared to 5-MeO-DMT (0-30 min vs. 0-20 min). In marked contrast to 5-MeO-DMT (Cpd. 32), 1-1 did not induce signs of 5-HT syndrome—i.e. whole body twitches, FPT, Straub tail—up to 60 mg/kg SC (FIG. 1). Compound I-1 (I-30 mg/kg) treatment did not reduce motor activity or rearing to the same extent as 5-MeO-DMT (FIG. 2).

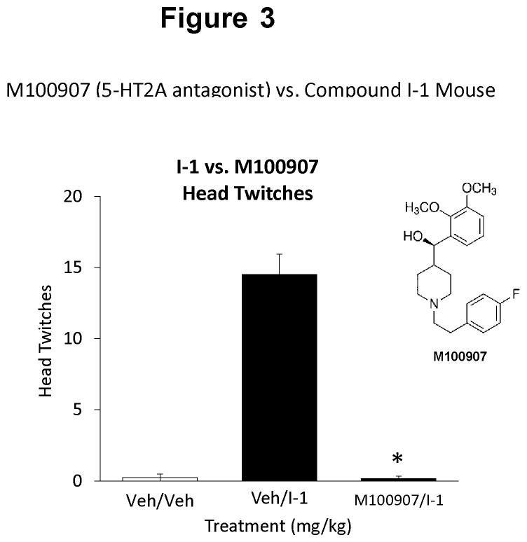

To evaluate the involvement of 5-HT_{2A} receptor on the HTR induced by exemplary compounds of Formula I, mice were pretreated with the selective 5-HT_{2A} antagonist M100907 (also known as volinanserin) prior to the administration of the compound of I-1. As expected, pretreatment with the antagonist completely blocked the effect of exemplary compounds of Formula I on HTR (FIG. 3).

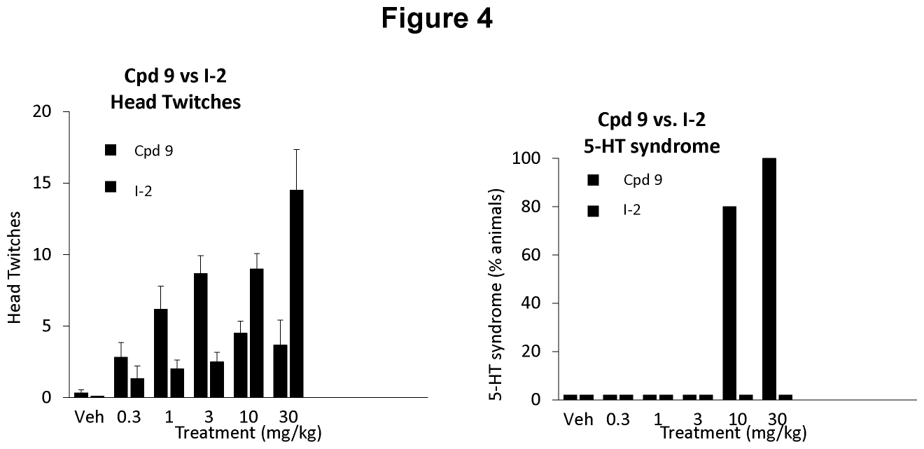

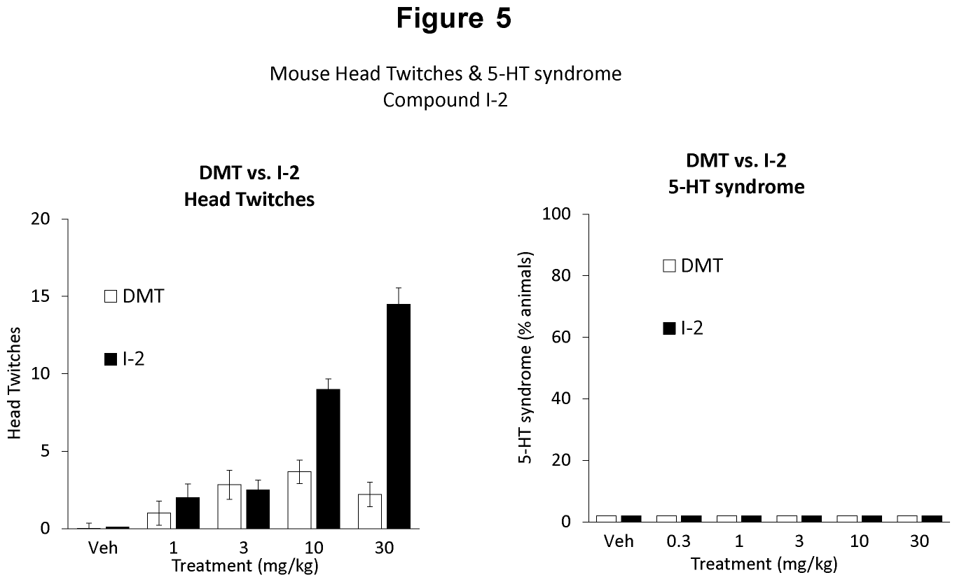

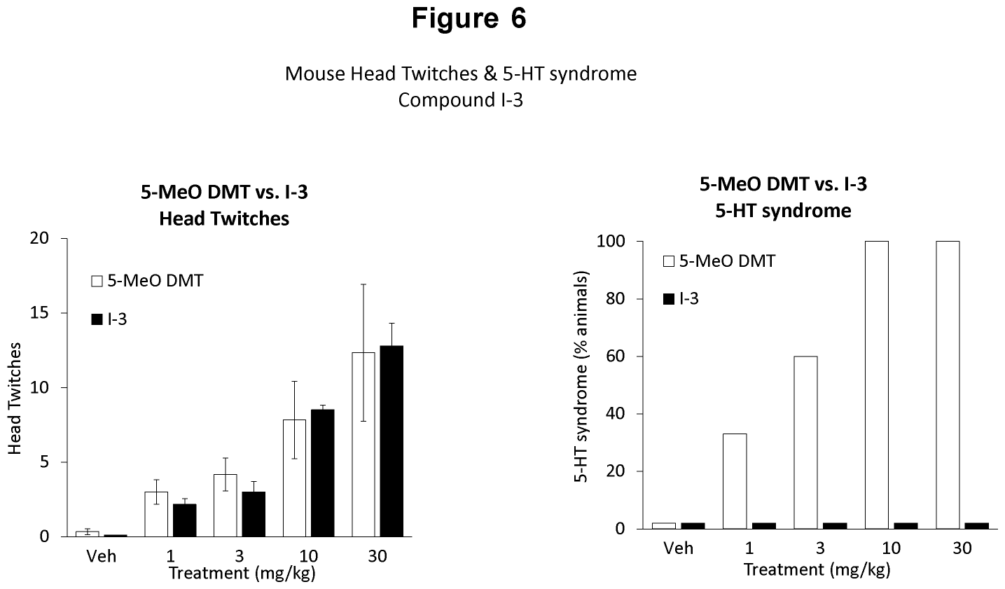

Similar to exemplary compound I-1, exemplary compound 1-2 elicited a robust head twitch response and no signs of 5-HT syndrome as compared to its corresponding parent compound (Cpd. 9, FIG. 4) and compared to DMT (FIG. 5). Compound 1-3 also elicited robust head twitch response and no signs of 5-HT syndrome as compared to 5-MeO-DMT (Cpd 32) (FIG. 6).

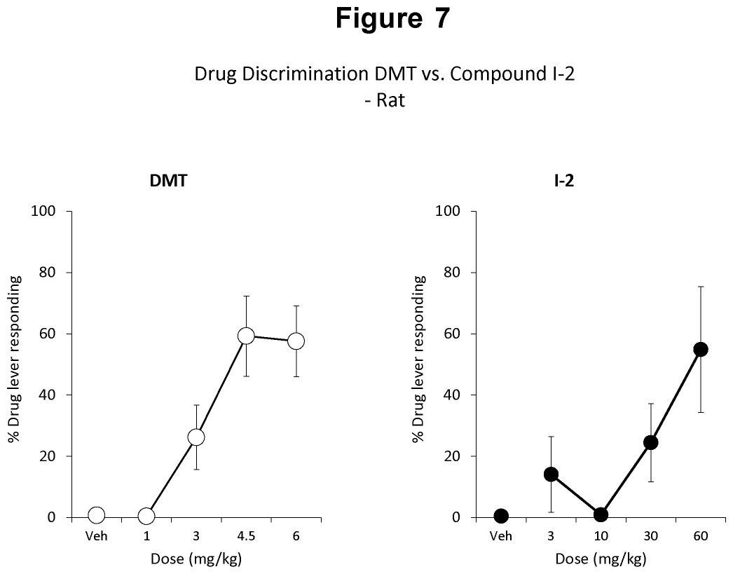

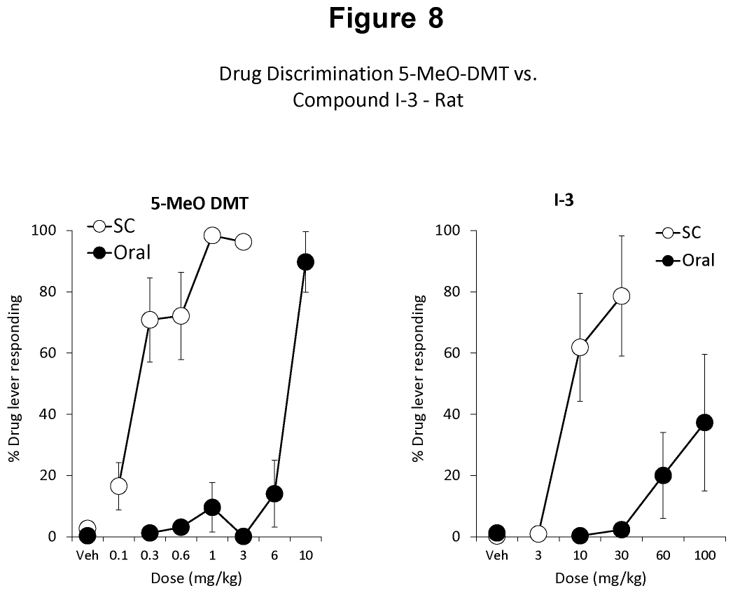

To establish ability of compounds to generalize to the known psychedelics, 5-MeO-DMT (Cpd 32) and DMT, compound 1-2, and compound 1-3 were evaluated in a drug discrimination assay in which rats were trained to discriminate between a 1 mg/kg training dose of 5-MeO-DMT and saline administration. DMT (ED50˜3 mg/kg) and compound 1-2 (ED50˜30 mg/kg) showed a similar level of partial generalization (˜60%) to the 5-MeO-DMT cue (FIG. 7). Both 5-MeO-DMT (ED50˜0.2 mg/kg) and compound 1-3 (ED50˜6 mg/kg) showed generalization to the 5-MeO-DMT cue following SC administration (FIG. 8). Following oral administration, larger doses of both 5-MeO-DMT (10 mg/kg) and 1-3 (100 mg/kg) were required to show generalization to the 5-MeO-DMT cue (FIG. 8).

Further data from rat behavioral studies are summarized in the Table 5, where 0=absent, 0/+=equivocal/marginal, +=present and ++=marked.

TABLE 5

| | Dose | | | | Decrease |
|---|---|---|---|---|---|
| | Range | | | | Core |
| | (mg/kg | | 5-HT | Hypoloco- | Body |
| Compound | SC) | HTR | Syndrome | motion | Temperature |
| 5-MeO-DMT | 1-30 | + | + | ++ | + |
| I-1 | 1-60 | + | 0 | 0 | 0 |
| Cpd. 20 | 0.3-30 | + | + | ++ | ++ |
| I-2 | 0.3-30 | + | 0/+ | 0 | 0 |
| Cpd. 9 | 0.3-30 | + | + | ++ | ++ |
| I-3 | 0.3-30 | + | 0/+ | 0 | 0 |

While the present application has been described with reference to examples, it is to be understood that the scope of the claims should not be limited by the embodiments set forth in the examples, but should be given the broadest interpretation consistent with the description as a whole.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the application described and claimed herein.

1. A compound of Formula I: 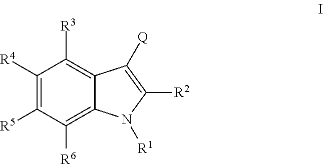
or a pharmaceutically acceptable salt and/or solvate thereof,
wherein:
R¹ is selected from C(O)R⁷, CO₂R⁷, C(O)N(R⁷)(R⁷), S(O)R⁷, SO₂R, C₀₂C₁₋₄alkyleneOC(O)R⁷, C(O)C₁₋₄alkyleneOC(O)R⁷ and CO₂C₁₋₄alkyleneC(O)R⁷;
Q is selected from Q1, Q2, Q2′, Q3, Q4, Q5, and Q6: 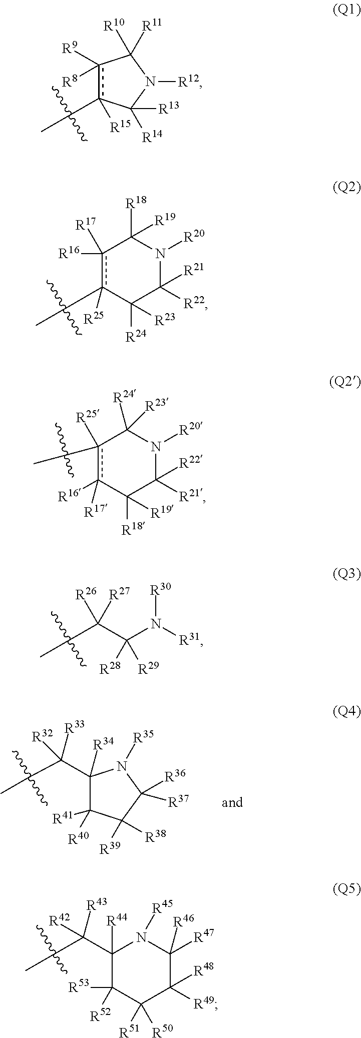 [symbol]
is a single bond or a double bond provided that when [symbol] in Q1 is a double bond then R⁹ and R¹⁵ are not present, when [symbol] in Q2 is a double bond then R¹⁷ and R²⁵ are not present and when [symbol] in Q2′ is a double bond then R^{17′} and R^{25′} are not present;
R², R⁵ and R⁶ are independently selected from H, halo, CN, C₁₋₆alkyl, C₂₋₆alkenyl, C₂₋₆alkynyl and C₁₋₆alkoxy;
one of R³ and R⁴ is selected from H, halo, C₁₋₆alkyl, C₂₋₆alkenyl, C₂₋₆alkynyl and C₁₋₆alkoxy and the other of R³ and R⁴ is selected from A, H, halo, C₁₋₆alkyl, C₂₋₆alkenyl, C₂₋₆alkynyl and C₁₋₆alkoxy;
A is selected from OR⁵⁴, OP(O)(OR⁵⁴)(OR⁵⁵), N(R⁵⁴)(R⁵⁵), SR⁵⁴, S(O)R⁵⁴, SO₂R⁵⁴, C(O)R⁵⁴, CO₂R⁵⁴, C(O)N(R⁵⁴)(R⁵⁵), C(NR⁵⁶)R⁵⁴, C(NR⁵⁶)NR⁵⁴R⁵⁵, C(NR⁵⁶)OR⁵⁴, aryl, C₃₋₁₀cycloalkyl, 3- to 10-membered heterocycloalkyl comprising 1 to 4 heteromoeities independently selected from O, S, S(O), SO₂, N and NR⁵⁴ and 5- to 10-membered heteroaryl comprising 1 to 4 heteromoeities independently selected from O, S, S(O), SO₂, N and NR⁵⁴, wherein the C₁₋₆alkyl, C₂₋₆alkenyl, C₂₋₆alkynyl, C₃₋₇cycloalkyl, aryl, 3- to 10-membered heterocycloalkyl and 5- to 10-membered heteroaryl are optionally substituted with one or more substituents independently selected from halo, CN, OR⁵⁷, CO₂R⁵⁷, N(R⁵⁷)(R⁵⁸) and SR⁵⁷, and wherein the C₃₋₁₀cycloalkyl, aryl, 3- to 10-membered heterocycloalkyl and 5- to 10-membered heteroaryl are each further optionally substituted with a substituent selected from CO₂R⁵⁹, C(O)N(R⁵⁹)(R⁶⁰) S(O)R⁵⁹, SO₂R⁵⁹, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁰ and 5- to 6-membered heteroaryl comprising 1 to 2 ring heteromoieties independently selected from O, S, S(O), SO₂, N, and NR⁶⁰;
R⁷ is selected from C₇₋₃₀alkyl, C₇₋₃₀alkenyl and C₇₋₃₀alkynyl, wherein the C₇₋₃₀alkyl, C₇₋₃₀alkenyl and C₇₋₃₀alkynyl, are optionally substituted with one or more substituents independently selected from halo, OR⁶¹, N(R⁶¹)(R⁶²) and SR⁶¹ and/or are optionally interrupted by one to six heteromoieties independently selected from 0, C(O), CO₂ and NR⁶³;
R^{7′} is selected from H and C₁₋₆alkyl;
R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R^{16′}, R^{17′}, R^{18′}, R^{19′}, R^{21′}, R²², R²³, R²⁴, R^{25′}, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, halo and C₁₋₆alkyl;
R⁵⁴ is selected from H, C₁₋₆alkyl, C₂₋₆alkenyl, C₂₋₆alkynyl, C₃₋₁₀cycloalkyl, aryl, 3- to 10-membered heterocycloalkyl comprising 1 to 4 heteromoeities independently selected from O, S, S(O), SO₂, N and NR⁶⁴ and 5- to 10-membered heteroaryl comprising 1 to 4 heteromoeities independently selected from O, S, S(O), SO₂, N and NR⁶⁴, wherein said C₁₋₆alkyl, C₂₋₆alkenyl, C₂₋₆alkynyl, C₃₋₁₀cycloalkyl, 3- to 10-membered heterocycloalkyl and 5- to 10-membered heteroaryl are optionally substituted by one or more substituents independently selected from CN, OR⁶⁵, CO₂R⁶⁵, N(R⁶⁵)(R⁶⁶) and SR⁶⁵, and wherein the C₃₋₁₀cycloalkyl, aryl, 3- to 10-membered heterocycloalkyl and 5- to 10-membered heteroaryl are each further optionally substituted with a substituent selected from CO₂R⁶⁷, C(O)N(R⁶⁷)(R⁶⁸), S(O)R⁶⁷, SO₂R⁶⁷, C₁₋₆alkyl, C₂₋₆alkenyl, C₂₋₆alkynyl, C₃₋₆cycloalkyl, phenyl, 3- to 6-membered heterocycloalkyl comprising 1 to 2 ring heteromoieties independently selected from O, S, S(O), SO₂, N, and NR⁶⁹ and 5- to 6-membered heteroaryl comprising 1 to 2 ring heteromoieties independently selected from O, S, S(O), SO₂, N, and NR⁶⁹;
R¹², R²⁰, R^{20′}, R³⁵ and R⁴⁵ are independently selected from H, C₁₋₆alkyl and C(O)C₁₋₆alkyl;
R³⁰ and R³¹ are independently selected from H, C₁₋₆alkyl and C(O)C₁₋₆alkyl, or R³⁰ and R³¹, together with the N atom to which they are bound, form a 3- to 8-membered heterocyclic ring which optionally comprises one or two additional heteromoieties independently selected from O, S, S(O), SO₂, N, and NR⁷⁰;
R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H and C₁₋₆alkyl; and
wherein all available hydrogen atoms are optionally and independently substituted with a fluorine atom or chlorine atom and all available atoms are optionally substituted with alternate isotope thereof.

2. The compound of claim 1, wherein Q is selected from: 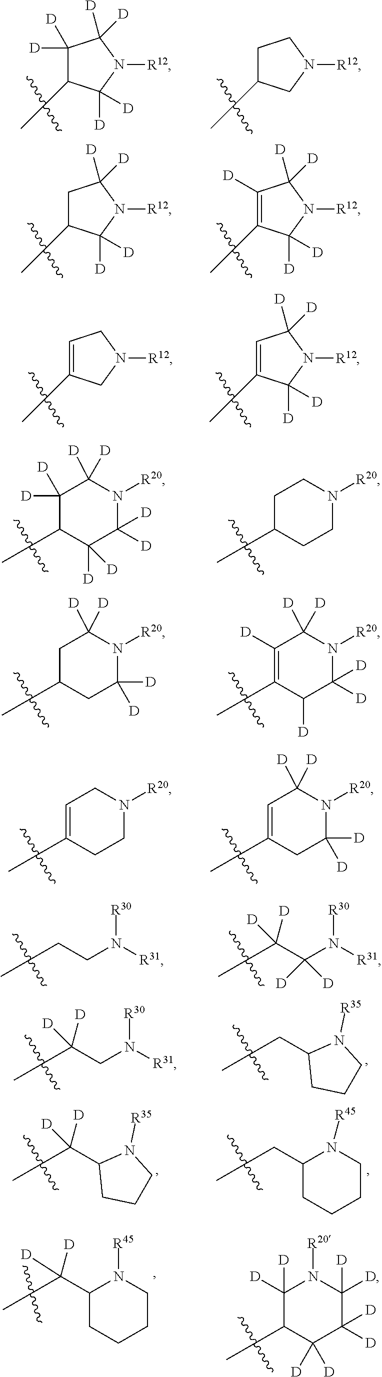 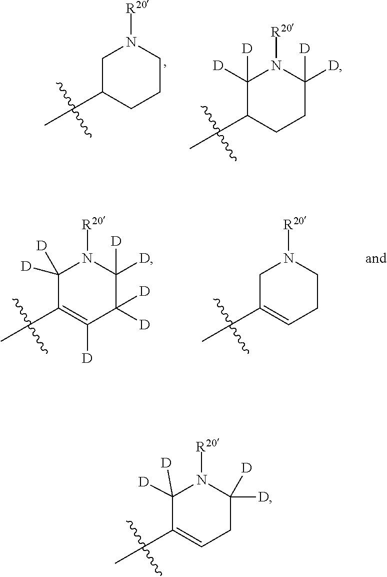
wherein R¹², R²⁰, R²⁰, R³⁰, R³¹, R³⁵ and R⁴⁵ are independently selected from H, D, C₁₋₆alkyl, C₁₋₆fluoroalkyl and C₁₋₆deuteroalkyl, or R³⁰ and R³¹, together with the N atom to which they are bound, form a 3- to 6-membered heterocyclic ring which optionally comprises one or two additional heteromoieties independently selected from O, N and NR⁷⁰.

3. The compound of claim 2, wherein R¹², R²⁰, R²⁰, R³⁰, R³¹, R³⁵ and R⁴⁵ are independently selected from H, CH₃, CD₃, CF₂H and CF₃; or
R³⁰ and R³¹, together with the N atom to which they are bound, form a 5- or 6-membered heterocyclic ring which optionally comprises one additional heteromoeity selected from O and NR⁷⁰.

4. The compound of any one of claims 1 to 3, wherein R², R⁵ and R⁶ are independently selected from H, D, Cl, F, C₁₋₄alkyl and C₁₋₄deuteroalkyl.

5. The compound of claim 5, wherein all of R², R⁵ and R⁶ are 0.

6. The compound of claim 5, wherein all of R², R⁵ and R⁶ are H.

7. The compound of any one of claims 1 to 6, wherein one of R³ and R⁴ is selected from H, D, F, C, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy and the other of R³ and R⁴ is selected from A, H, D, F, C, C₁₋₆alkyl, C₁₋₆fluoroalkyl, C₁₋₆deuteroalkyl, C₁₋₆alkoxy, C₁₋₆fluoroalkoxy and C₁₋₆deuteroalkoxy.

8. The compound of claim 7, wherein both R³ and R⁴ are D.

9. The compound of claim 7, wherein both R³ and R⁴ are H.

10. The compound of any one of claims 1 to 7, wherein R³ is A and R⁴ is H or D.

11. The compound of any one of claims 1 to 7, R³ is H or D and R⁴ is A.

12. The compound of any one of claims 1 to 7, 10 and 11 wherein A is selected from OR⁵⁴, OP(O)(OR⁵⁴)(OR⁵⁵), N(R⁵⁴)(R⁵⁵), C(O)R⁵⁴, CO₂R⁵⁴ and C(O)N(R⁵⁴)(R⁵⁵), wherein R⁵⁴ is selected from H, C₁₋₆alkyl, C₁₋₆fluoroalkyl, C₁₋₆deuteroalkyl, C₃₋₁₀cycloalkyl, aryl, 3- to 6-membered heterocycloalkyl comprising 1 to 3 heteromoeities independently selected from O, N and NR⁶⁴ and 5- to 6-membered heteroaryl comprising 1 to 3 heteromoeities independently selected from O, N and NR⁶⁴, wherein said C₁₋₆alkyl, C₁₋₆fluoroalkyl, C₁₋₆deuteroalkyl, C₃₋₆cycloalkyl, 3- to 6-membered heterocycloalkyl and 5- to 6-membered heteroaryl are optionally substituted by one or two substituents independently selected from CN, OR⁶⁵, CO₂R⁶⁵ and N(R⁶⁵)(R⁶⁶), and wherein the C₃₋₆cycloalkyl, aryl, 3- to 6-membered heterocycloalkyl and 5- to 6-membered heteroaryl are each further optionally substituted with a substituent selected from CO₂R⁶⁷, C(O)N(R⁶⁷)(R⁶⁸), C₁₋₆alkyl, C₁₋₆fluoroalkyl, C₁₋₆deuteroalkyl, C₃₋₆cycloalkyl, phenyl, 3- to 6-membered heterocycloalkyl comprising 1 to 2 ring heteromoieties independently selected from O, N, and NR⁶⁹ and 5- to 6-membered heteroaryl comprising 1 to 2 ring heteromoieties independently selected from O, N and NR⁶⁹; and R⁵⁵ is selected from H, C₁₋₆alkyl, C₁₋₆fluoroalkyl and C₁₋₆deuteroalkyl.

13. The compound of claim 12, wherein A is selected from OR⁵⁴, OP(O)(OR⁵⁴)(OR⁵⁵), N(R⁵⁴)(R⁵⁵), C(O)R⁵⁴, CO₂R⁵⁴ and C(O)N(R⁵⁴)(R⁵⁵), wherein R⁵⁴ and R55 are independently selected from H, C₁₋₆alkyl, C₁₋₆fluoroalkyl and C₁₋₆deuteroalkyl.

14. The compound of claim 12, wherein A is selected from OR⁵⁴ and OP(O)(OR⁵⁴)(OR⁵⁵), wherein R⁵⁴ and R⁵⁵ are independently selected from H, CH₃, CF₃, CF₂H and CD₃.

15. The compound of any one of claims 1 to 14, wherein R¹ is selected from C(O)R⁷, CO₂R⁷, CO₂CH₂OC(O)R⁷ and CO₂CH(CH₃)OC(O)R⁷.

16. The compound of any one of claims 1 to 15, wherein R⁷ is selected from C₇₋₃₀alkyl and C₇₋₃₀alkenyl, wherein the C₇₋₃₀alkyl and C₇₋₃₀alkenyl are optionally substituted with one or more substituents independently selected from D, Cl, F and OR⁶¹, and/or are optionally interrupted by one to four O and/or CO₂.

17. The compound of claim 16, wherein R⁷ is selected from C₇₋₃₀alkyl and C₇₋₃₀alkenyl, wherein the C₇₋₃₀alkyl and C₇₋₃₀alkenyl are optionally substituted with one or more substituents independently selected from F and D, and/or are optionally interrupted by one to four 0 and/or 002.

18. The compound of claim 17, wherein R⁷ is selected from C₇₋₃₀alkyl and C₇₋₃₀alkenyl, wherein the C₇₋₃₀alkyl and C₇₋₃₀alkenyl are optionally substituted with one or more substituents independently selected from F and D, and/or are optionally interrupted by one to two 0 and/or 002.

19. The compound of any one of claims 1 to 15, wherein the alkyl or alkene group of R⁷ is an alkyl or alkenyl group present in a fatty acid, wherein all available H atoms are optionally substituted with deuterium.

20. The compound of claim 19, wherein the fatty acid is selected from:

| | Lipid | Chemical |
|---|---|---|
| Common Name | Number | Name |
| linoleic acid (LA) | 18:2 (n-6) | all-cis-9,12- |
| | | octadecadienoic acid, |
| rumenic acid | 18:2 (n-6) | 9Z,11E- |
| (conjugated | | octadecadienoic acid, |
| linoleic acid) | | |
| conjugated | 18:2 (n-6) | 10E,12Z- |
| linoleic acid | | octadecadienoic acid, |
| conjugated | 18:2 (n-6) | 9Z,12E- |
| linoleic acid | | octadecadienoic acid, |
| gamma-linolenic | 18:3 (n-6) | all-cis-6,9,12- |
| acid (GLA) | | octadecatrienoic acid, |
| calendic acid | 18:3 (n-6) | 8E,10E,12Z- |
| | | octadecatrienoic acid, |
| eicosadienoic | 20:2 (n-6) | all-cis-11,14- |
| acid | | eicosadienoic acid, |
| dihomo-gamma- | 20:3 (n-6) | all-cis-8,11,14- |
| linolenic | | eicosatrienoic acid, |
| acid (DGLA) | | |
| arachidonic | 20:4 (n-6) | all-cis-5,8,11,14- |
| acid (AA, ARA) | | eicosatetraenoic acid |
| docosadienoic | 22:2 (n-6) | all-cis-13,16- |
| acid | | docosadienoic acid, |
| adrenic acid | 22:4 (n-6) | all-cis-7,10,13,16- |
| | | docosatetraenoic acid, |
| osbond acid | 22:5 (n-6) | all-cis-4,7,10,13,16- |
| | | docosapentaenoic acid, |
| tetracosatetraenoic | 24:4 (n-6) | all-cis-9,12,15,18- |
| acid | | tetracosatetraenoic acid, |
| tetracosapentaenoic | 24:5 (n-6) | all-cis-6,9,12,15,18- |
| acid | | tetracosapentaenoic acid, |
| α-linolenic | 18:3 (n-3) | all-cis-9,12,15- |
| acid (ALA) | | octadecatrienoic acid, |
| stearidonic | 18:4 (n-3) | all-cis-6,9,12,15- |
| acid (SDA) | | octadecatetraenoic acid, |
| hexadecatrienoic | 16:3 (n-3) | all-cis-7,10,13- |
| acid (HTA) | | hexadecatrienoic acid, |
| eicosatrienoic | 20:3 (n-3) | all-cis-11,14,17- |
| acid (ETE) | | eicosatrienoic acid, |
| eicosatetraenoic | 20:4 (n-3) | all-cis-8,11,14,17- |
| acid (ETA) | | eicosatetraenoic acid, |
| eicosapentaenoic | 20:5 (n-3) | all-cis-5,8,11,14,17- |
| acid (EPA) | | eicosapentaenoic acid, |
| heneicosapentaenoic | 21:5 (n-3) | all-cis-6,9,12,15,18- |
| acid (HPA) | | heneicosapentaenoic acid, |
| docosapentaenoic | 22:5 (n-3) | all-cis-7,10,13,16,19- |
| acid (DPA) | | docosapentaenoic acid, |
| docosahexaenoic | 22:6 (n-3) | all-cis-4,7,10,13,16,19- |
| acid (DHA) | | docosahexaenoic acid, |
| tetracosapentaenoic | 24:5 (n-3) | all-cis-9,12,15,18,21- |
| acid | | tetracosapentaenoic acid, |
| tetracosahexaenoic | 24:6 (n-3) | all-cis-6,9,12,15,18,21- |
| acid (Nisinic acid) | | tetracosahexaenoic acid, |
| myristoleic acid | 14:1 (n-5) | 9Z-tetradecenoic acid, |
| palmitoleic acid | 16:1 (n-7) | (9Z)-hexadecenoic acid, |
| sapienic acid | 16:1 (n-10) | (6Z)-hexadecenoic acid, |
| oleic acid | 18-1 (n-9) | (9Z)-octadecenoic acid, |
| elaidic acid | 18:1 (n-9) | (E)-octadecenoic acid, |
| vaccenic acid | 18:1 (n-7) | (11E)-octadecenoic acid, |
| eruric acid | 22-1 (n-9) | (13Z)-Docosenoic acid, |
| caprylic acid | 8:0 | octanoic acid, |
| capric acid | 10:0 | decanoic acid, |
| lauric acid | 12:0 | dodecanoic acid, |
| myristic acid | 14:0 | tetradecanoic acid, |
| palmitic acid | 16:0 | hexadecenoic acid, |
| stearic acid | 18:0 | octadecanoic acid, |
| arachidic acid | 20:0 | Icosanoic acid, |
| behenic acid | 22:0 | docosanoic acid, |
| lignoceric acid | 24:0 | tetracosanoic acid, and |
| cerotic acid | 26:0 | hexacosanoic acid, |

wherein all available H atoms are optionally substituted with deuterium.

21. The compound of any one of claims 1 to 20, wherein R^{7′} is selected from H, C₁₋₆alkyl, C₁₋₆fluoroalkyl and C₁₋₆deuteroalkyl.

22. The compound of any one of claims 1 to 21, wherein R⁸, R⁹, R¹⁰, R¹¹, R¹³, R¹⁴, R¹⁵, R¹⁶, R¹⁷, R¹⁸, R¹⁹, R²¹, R²², R²³, R²⁴, R²⁵, R^{16′}, R^{17′}, R^{18′}, R^{19′}, R^{21′}, R^{22′}, R^{23′}, R^{24′}, R^{25′}, R²⁶, R²⁷, R²⁸, R²⁹, R³², R³³, R³⁴, R³⁶, R³⁷, R³⁸, R³⁹, R⁴⁰, R⁴¹, R⁴², R⁴³, R⁴⁴, R⁴⁶, R⁴⁷, R⁴⁸, R⁴⁹, R⁵⁰, R⁵¹, R⁵² and R⁵³ are independently selected from H, O, F, Cl, C₁₋₆alkyl, C₁₋₆fluoroalkyl and C₁₋₆deuteroalkyl.

23. The compound of any one of claims 1 to 22, wherein R⁵⁵, R⁵⁶, R⁵⁷, R⁵⁸, R⁵⁹, R⁶⁰, R⁶¹, R⁶², R⁶³, R⁶⁴, R⁶⁵, R⁶⁶, R⁶⁷, R⁶⁸, R⁶⁹ and R⁷⁰ are independently selected from H, C₁₋₆alkyl, C₁₋₆fluoroalkyl and C₁₋₆deuteroalkyl.

24. The compound of claim 1 selected from 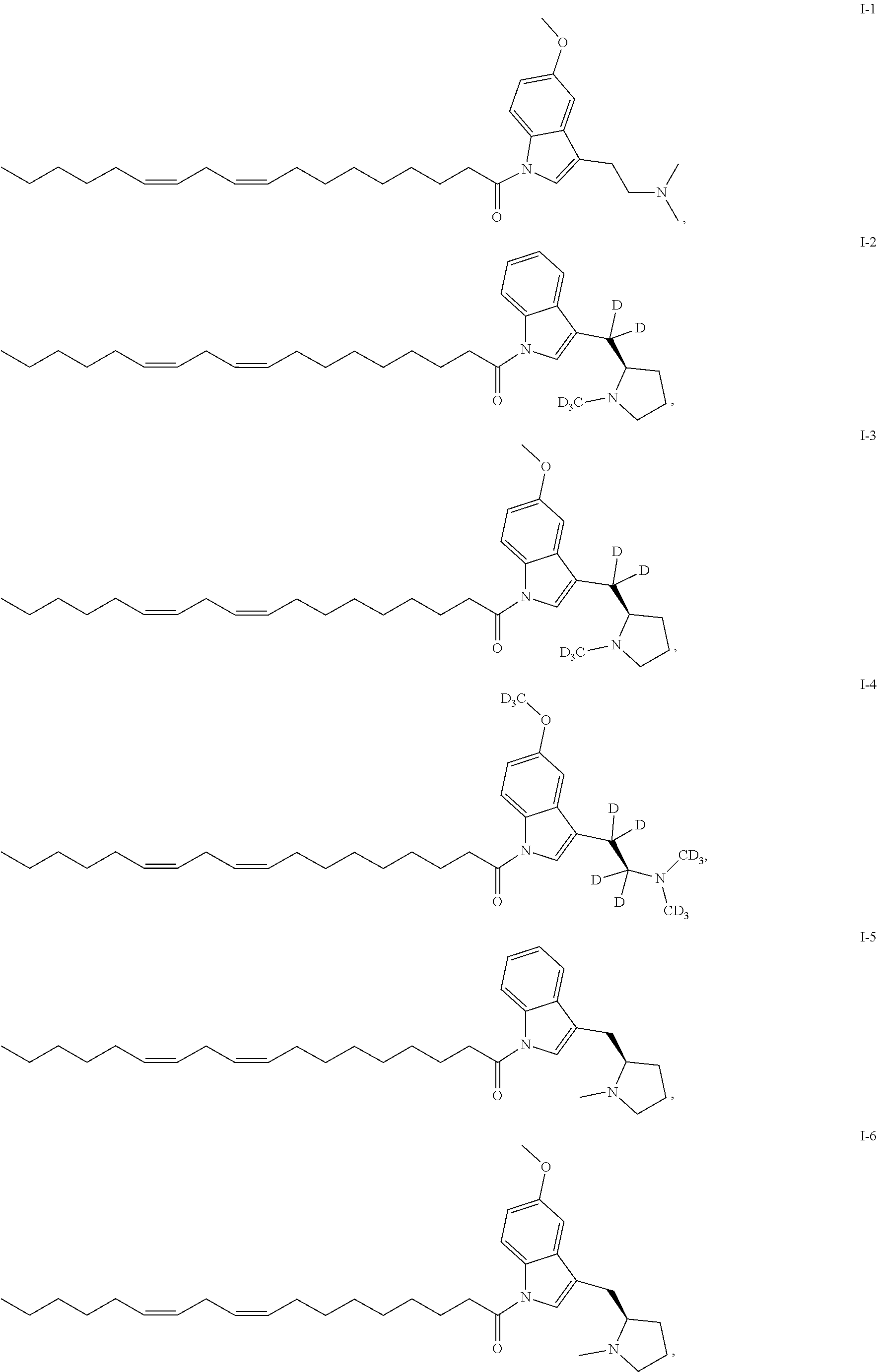 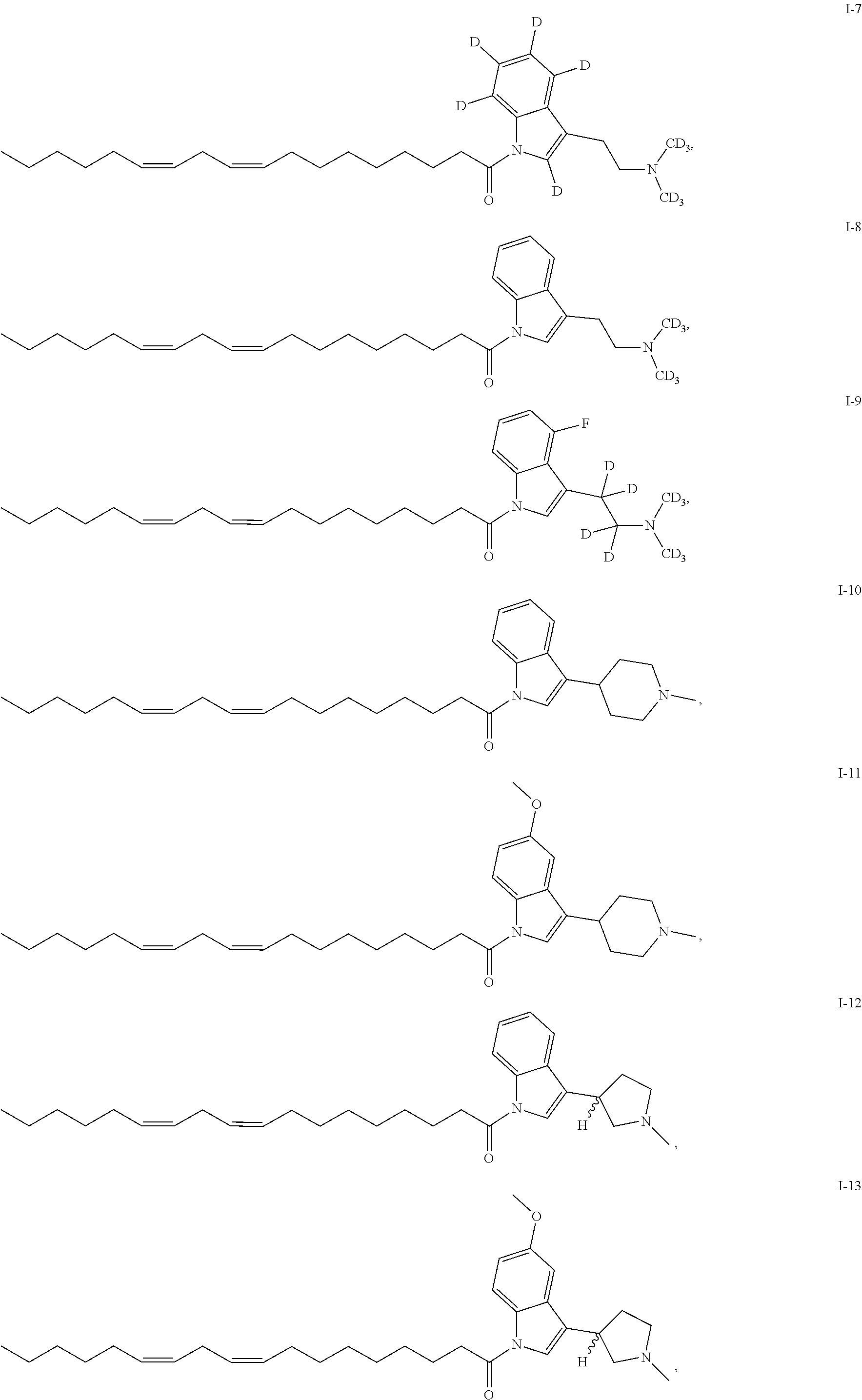 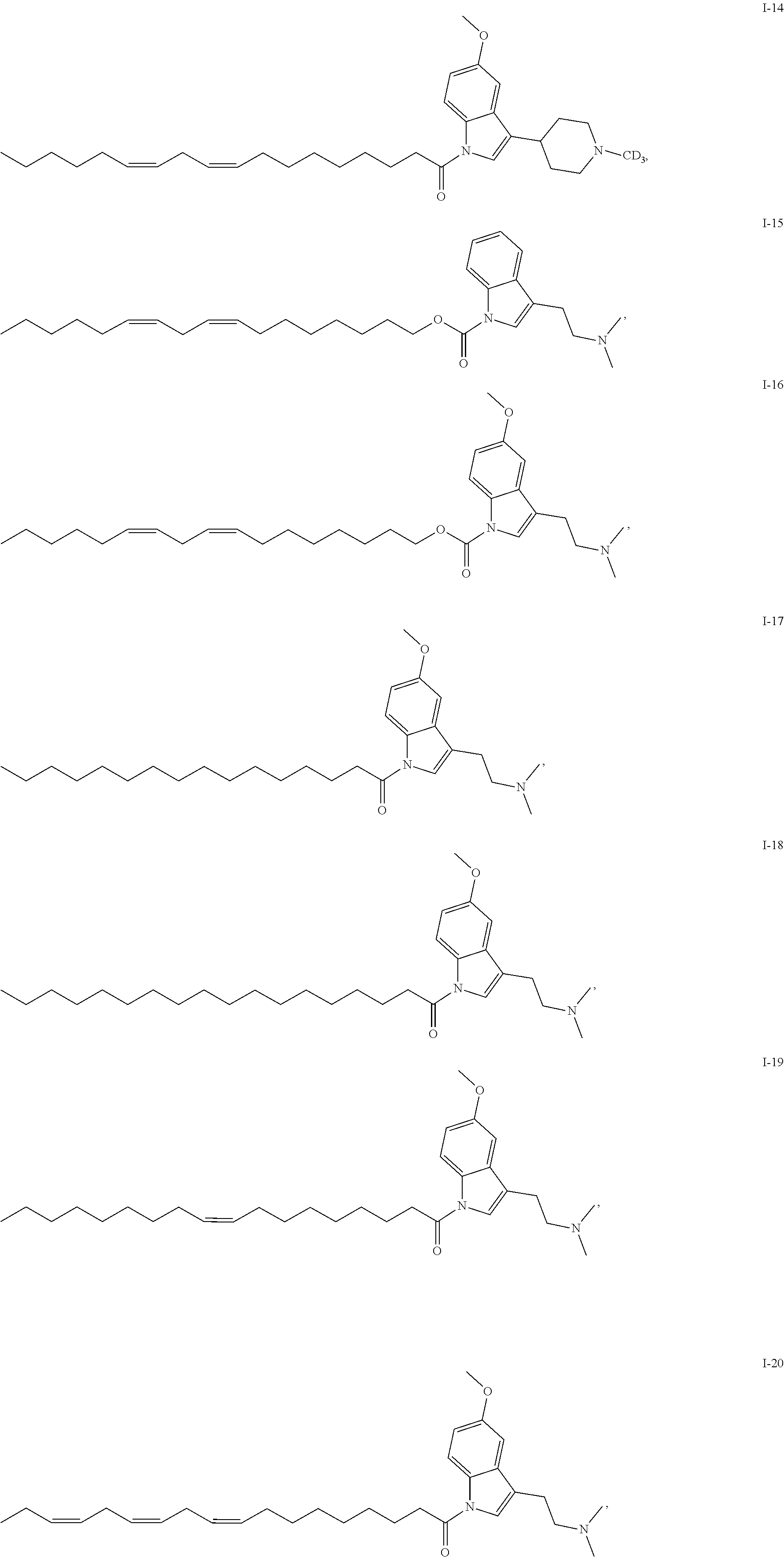 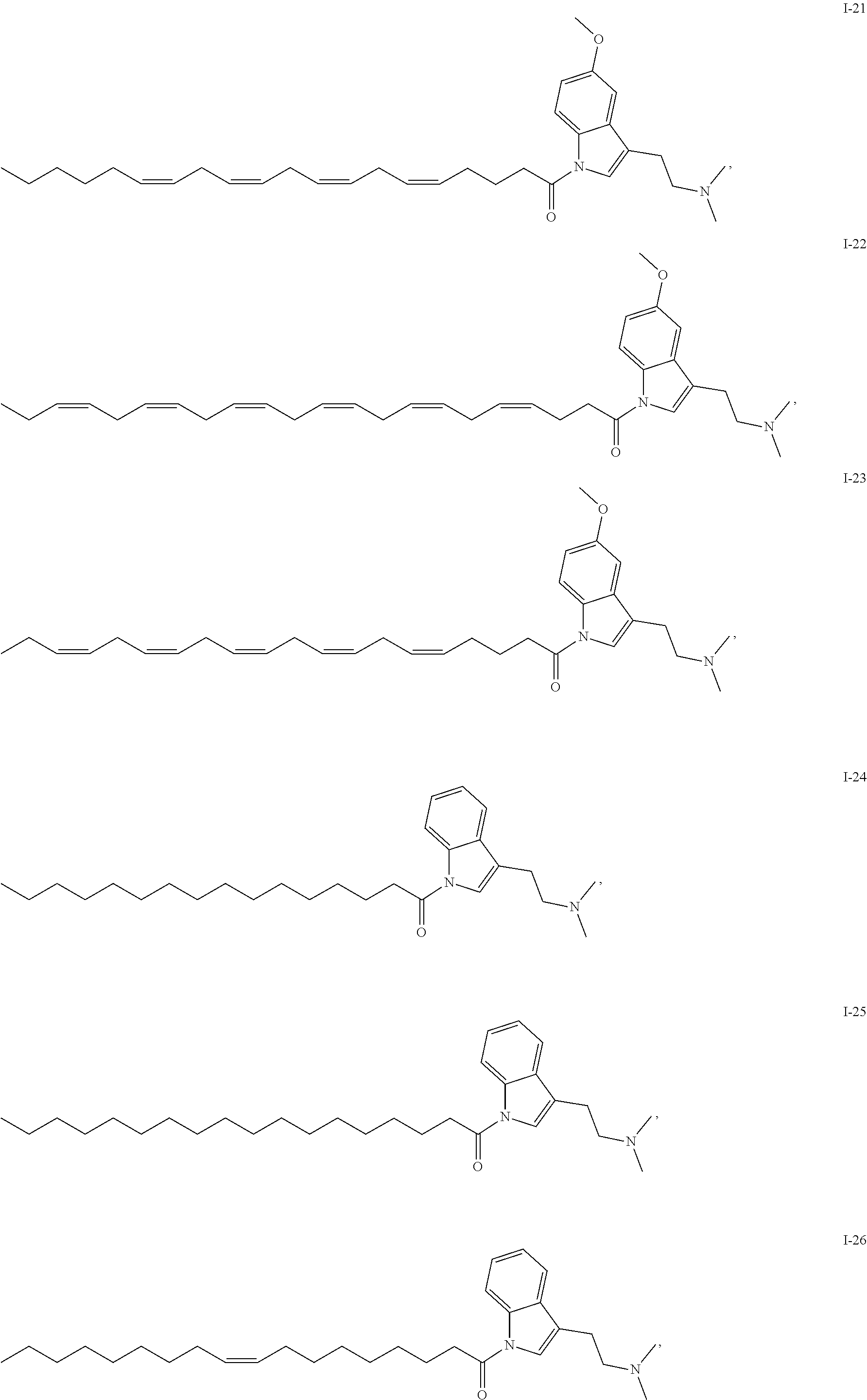 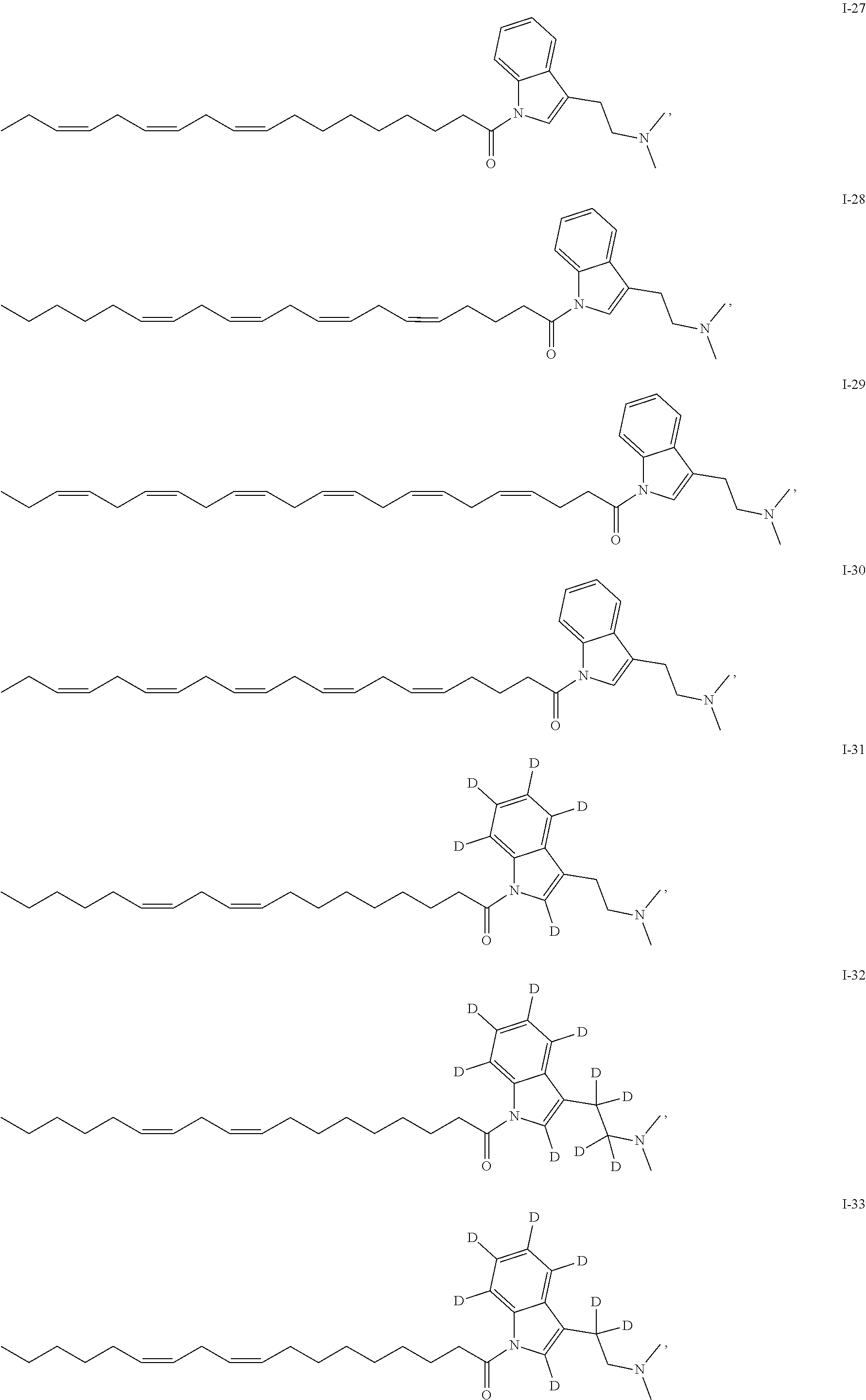 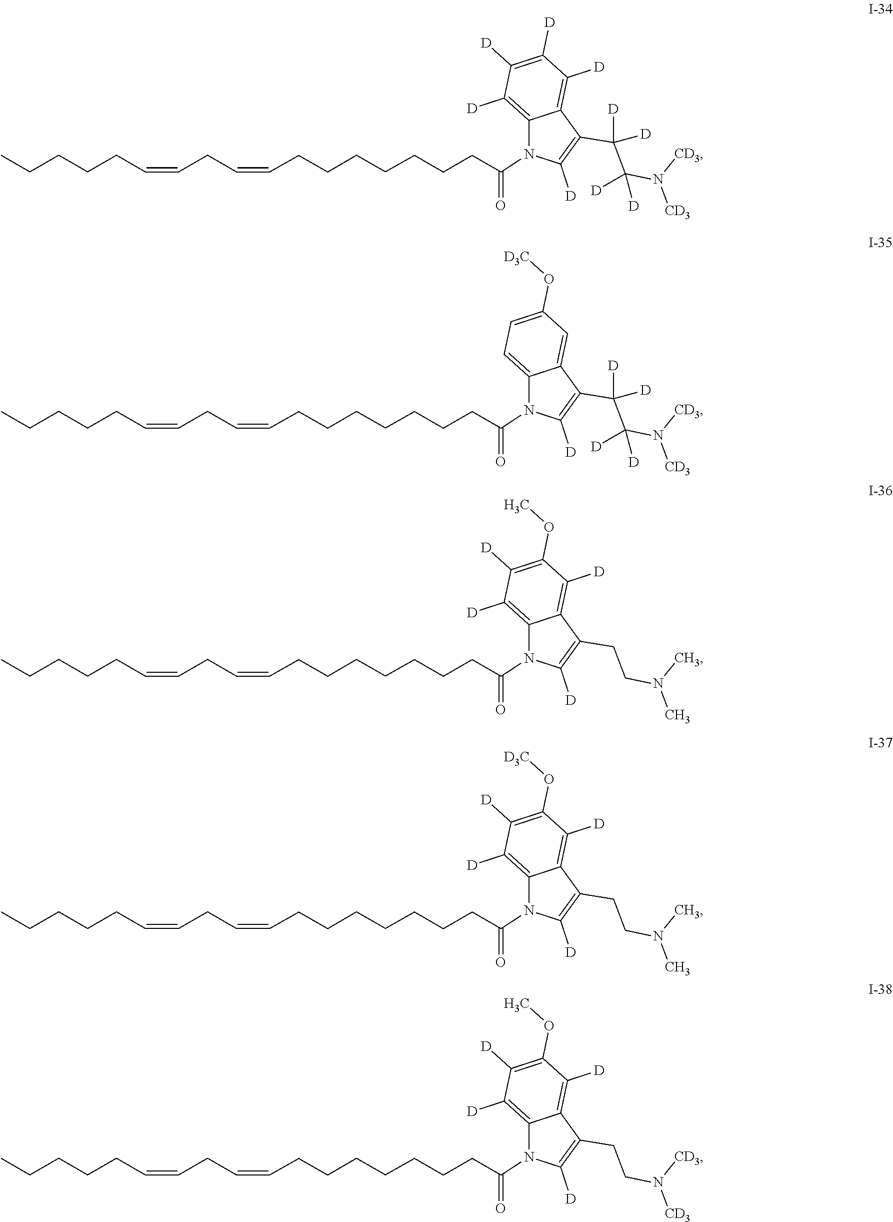 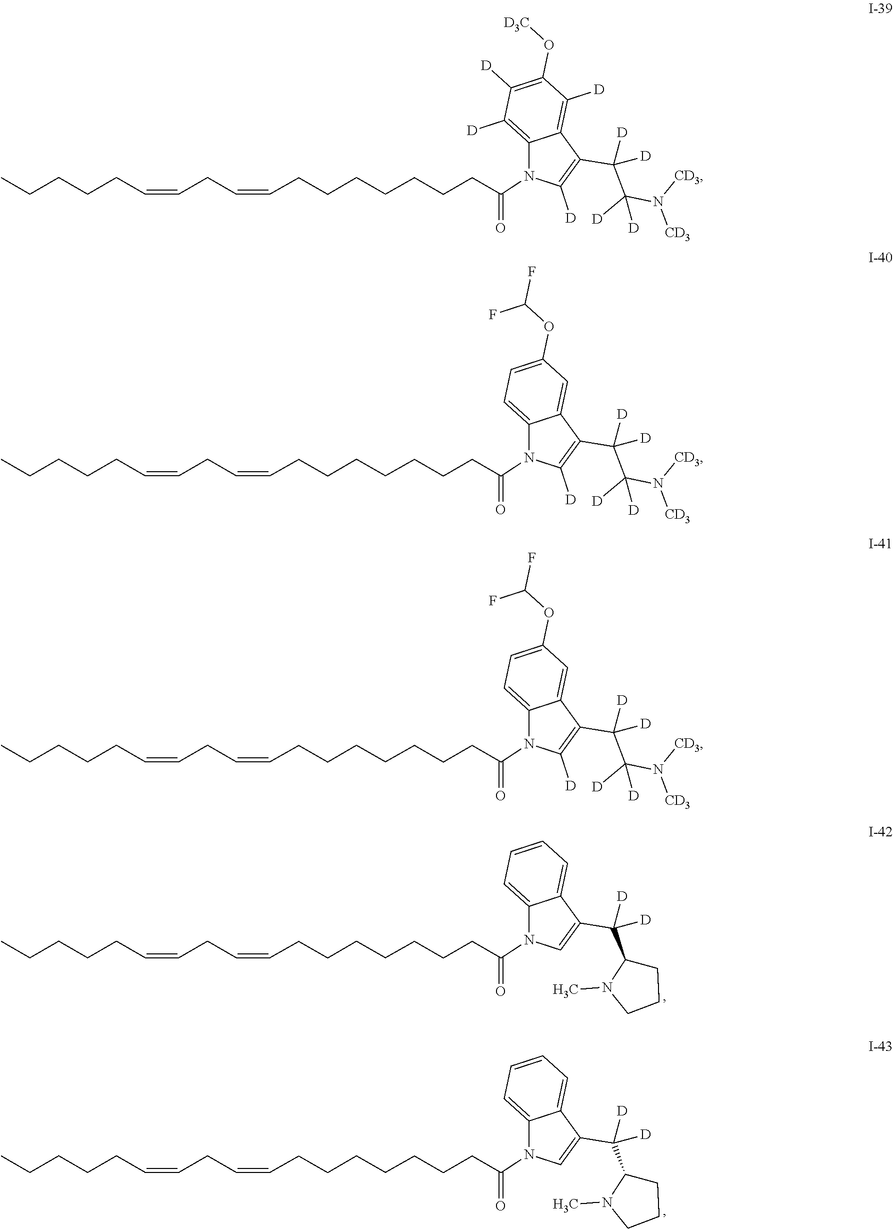 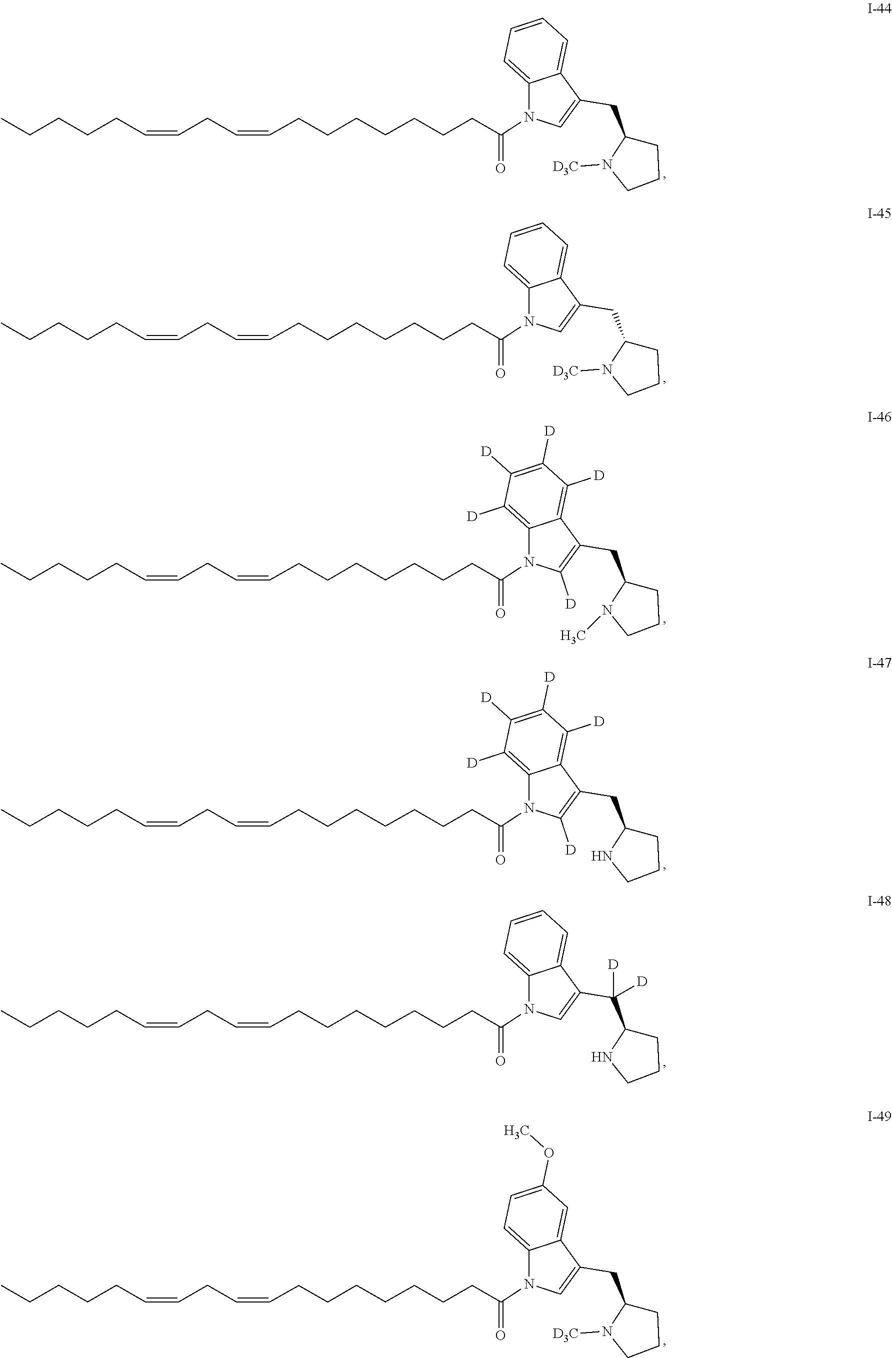 [SMILES: [2H]C([2H])([2H])N1CCC[C@@H]1Cc1cn(C(=O)CCCCCCC/C=C\C/C=C\CCCCC)c2ccccc12 ; [2H]C([2H])([2H])N1CCC[C@H]1Cc1cn(C(=O)CCCCCCC/C=C\C/C=C\CCCCC)c2ccccc12 ; [2H]c1c([2H])c([2H])c2c(c1[2H])c(C[C@H]1CCCN1C)c([2H])n2C(=O)CCCCCCC/C=C\C/C=C\CCCCC ; [2H]c1c([2H])c([2H])c2c(c1[2H])c(C[C@H]1CCCN1)c([2H])n2C(=O)CCCCCCC/C=C\C/C=C\CCCCC ; [2H]C([2H])(c1cn(C(=O)CCCCCCC/C=C\C/C=C\CCCCC)c2ccccc12)[C@H]1CCCN1 ; [2H]C([2H])([2H])N1CCC[C@@H]1Cc1cn(C(=O)CCCCCCC/C=C\C/C=C\CCCCC)c2ccc(OC)cc12] 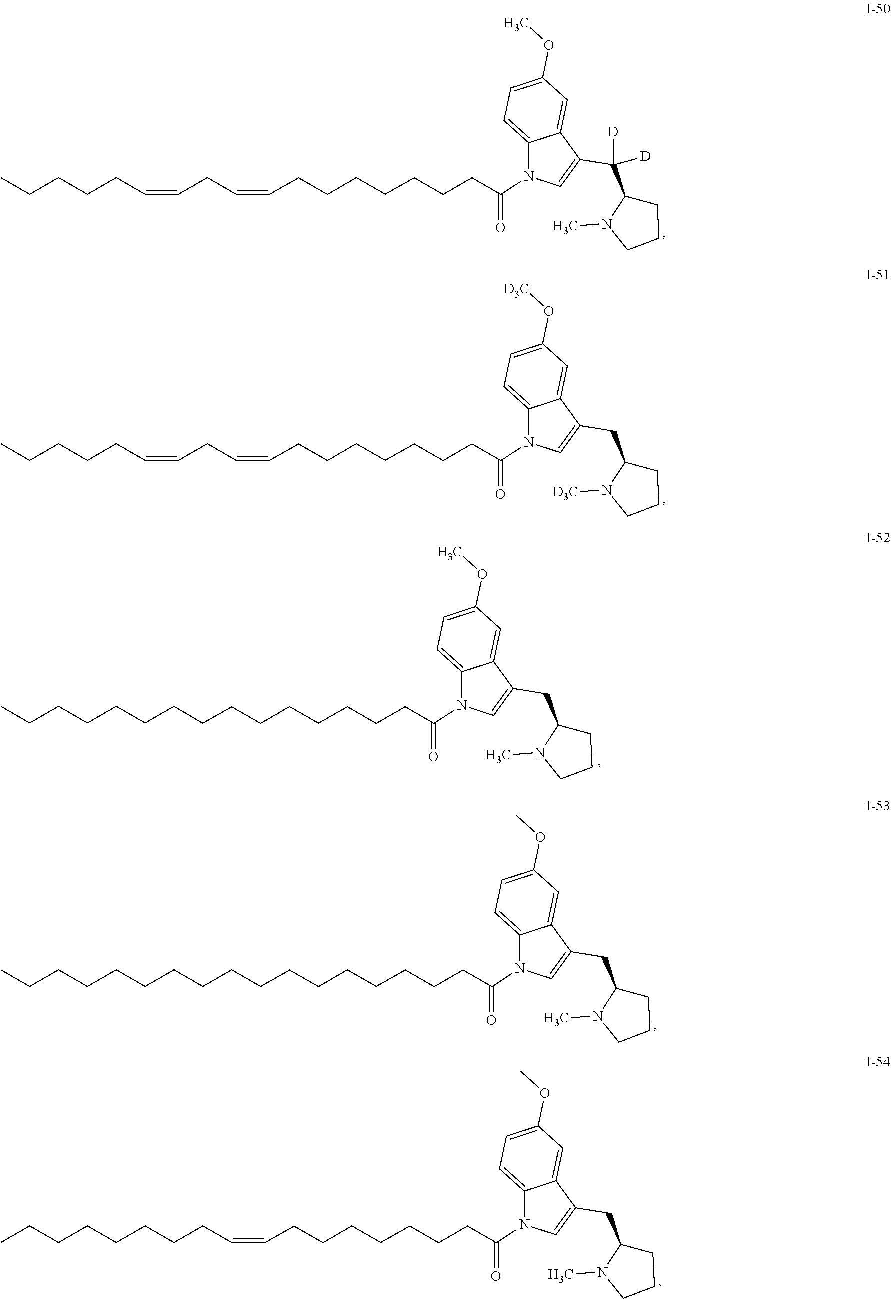 [SMILES: [2H]C([2H])(c1cn(C(=O)CCCCCCC/C=C\C/C=C\CCCCC)c2ccc(OC)cc12)[C@H]1CCCN1C ; [2H]C([2H])([2H])Oc1ccc2c(c1)c(C[C@H]1CCCN1C([2H])([2H])[2H])cn2C(=O)CCCCCCC/C=C\C/C=C\CCCCC ; CCCCCCCCCCCCCCCC(=O)n1cc(C[C@H]2CCCN2C)c2cc(OC)ccc21 ; CCCCCCCCCCCCCCCCCC(=O)n1cc(C[C@H]2CCCN2C)c2cc(OC)ccc21 ; CCCCCCCC/C=C\CCCCCCCC(=O)n1cc(C[C@H]2CCCN2C)c2cc(OC)ccc21] 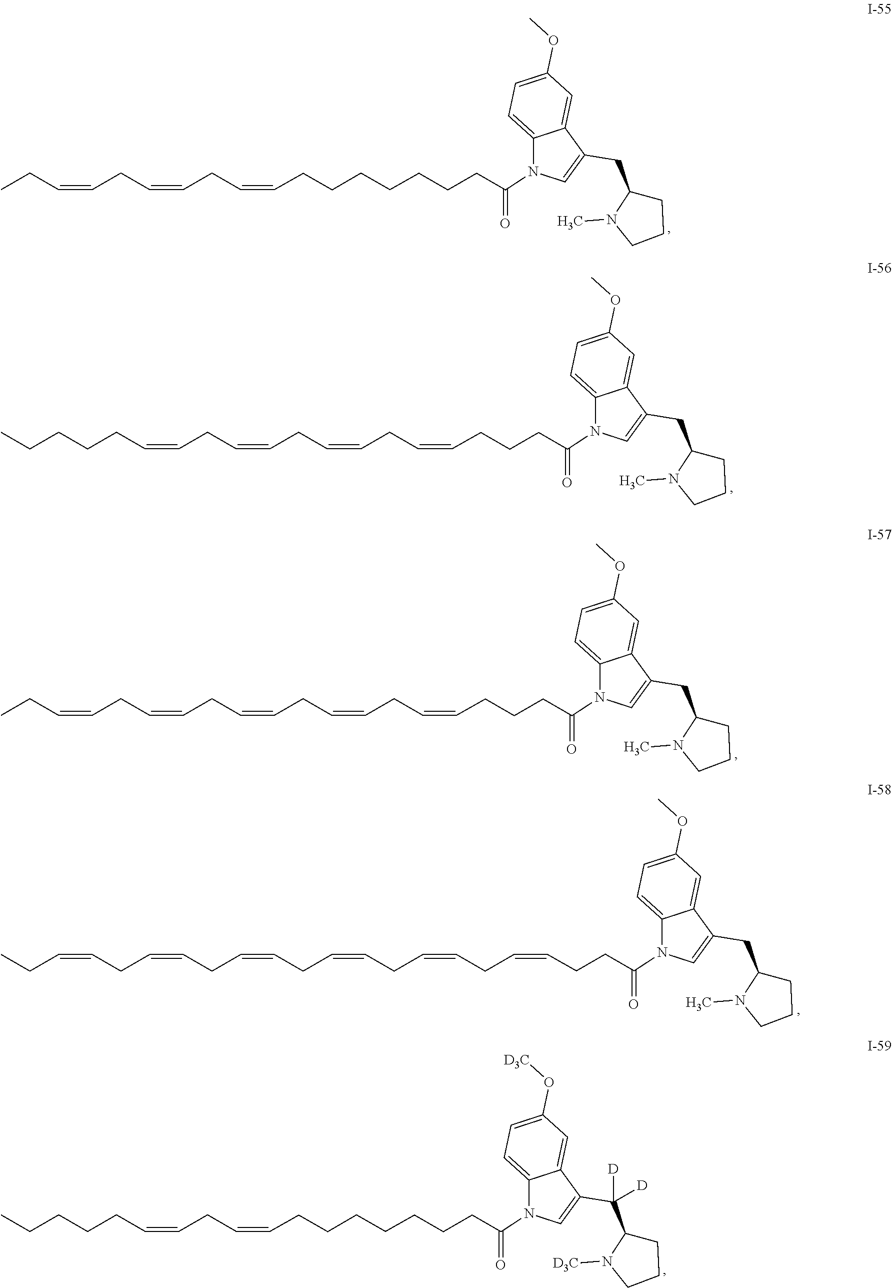 [SMILES: CC/C=C\C/C=C\C/C=C\CCCCCCCC(=O)n1cc(C[C@H]2CCCN2C)c2cc(OC)ccc21 ; CCCCC/C=C\C/C=C\C/C=C\C/C=C\CCCC(=O)n1cc(C[C@H]2CCCN2C)c2cc(OC)ccc21 ; CC/C=C\C/C=C\C/C=C\C/C=C\C/C=C\CCCC(=O)n1cc(C[C@H]2CCCN2C)c2cc(OC)ccc21 ; CC/C=C\C/C=C\C/C=C\C/C=C\C/C=C\C/C=C\CCC(=O)n1cc(C[C@H]2CCCN2C)c2cc(OC)ccc21 ; [2H]C([2H])([2H])Oc1ccc2c(c1)c(C([2H])([2H])[C@H]1CCCN1C([2H])([2H])[2H])cn2C(=O)CCCCCCC/C=C\C/C=C\CCCCC] 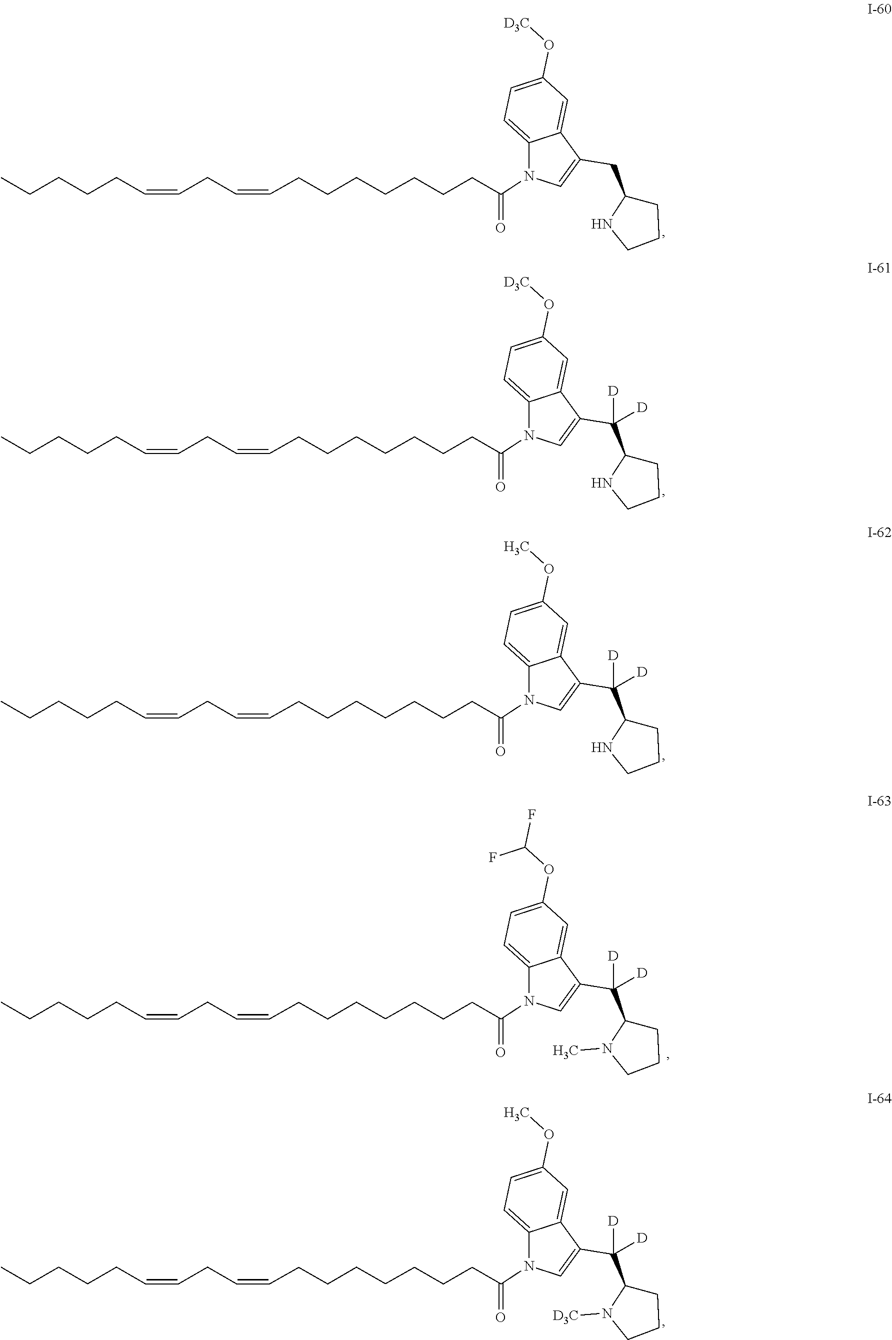 [SMILES: [2H]C([2H])([2H])Oc1ccc2c(c1)c(C[C@H]1CCCN1)cn2C(=O)CCCCCCC/C=C\C/C=C\CCCCC ; [2H]C([2H])([2H])Oc1ccc2c(c1)c(C([2H])([2H])[C@H]1CCCN1)cn2C(=O)CCCCCCC/C=C\C/C=C\CCCCC ; [2H]C([2H])(c1cn(C(=O)CCCCCCC/C=C\C/C=C\CCCCC)c2ccc(OC)cc12)[C@H]1CCCN1 ; [2H]C([2H])(c1cn(C(=O)CCCCCCC/C=C\C/C=C\CCCCC)c2ccc(OC(F)F)cc12)[C@H]1CCCN1C ; [2H]C([2H])(c1cn(C(=O)CCCCCCC/C=C\C/C=C\CCCCC)c2ccc(OC)cc12)[C@H]1CCCN1C([2H])([2H])[2H]] 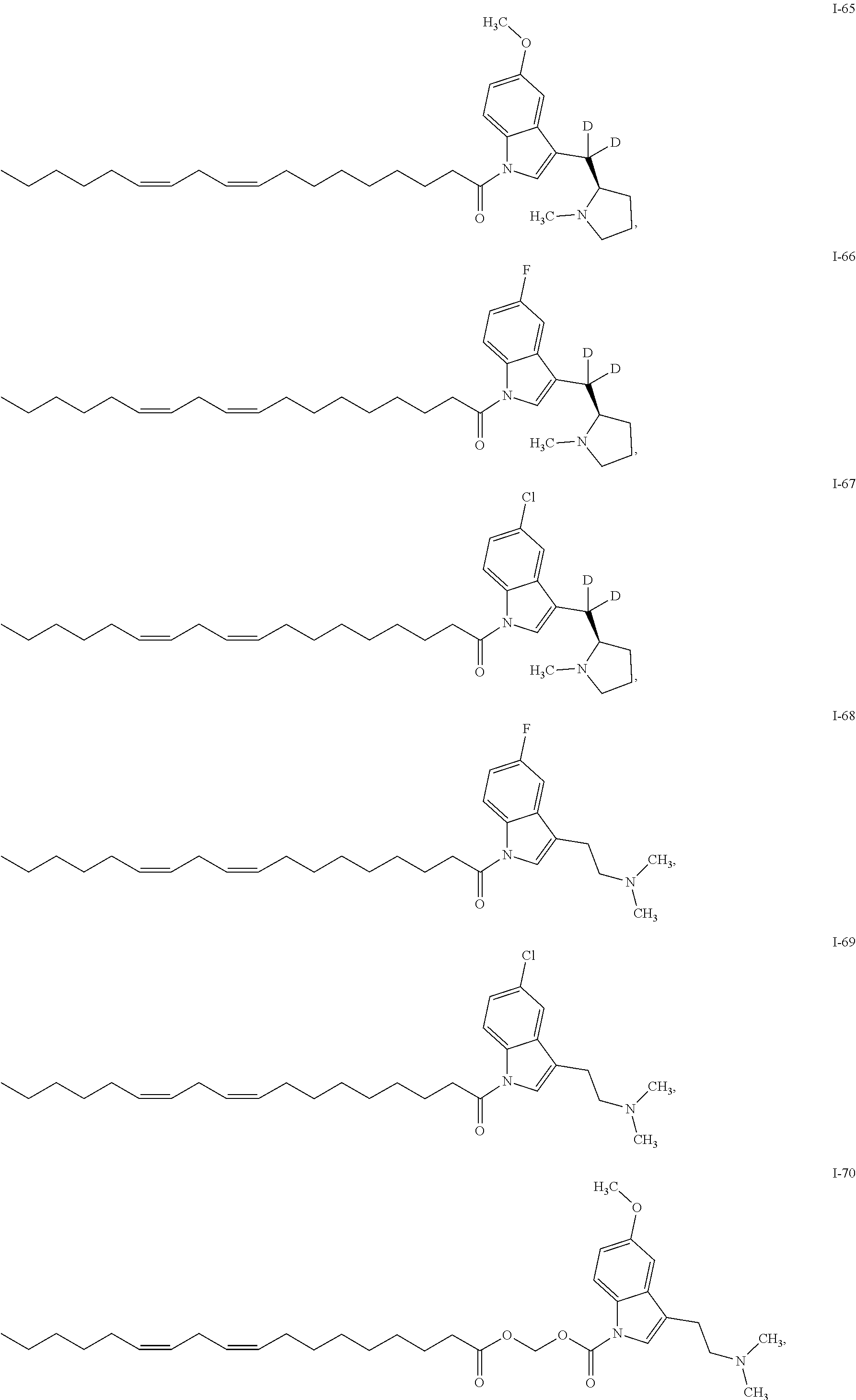 [SMILES: [2H]C([2H])(c1cn(C(=O)CCCCCCC/C=C\C/C=C\CCCCC)c2ccc(OC)cc12)[C@H]1CCCN1C ; [2H]C([2H])(c1cn(C(=O)CCCCCCC/C=C\C/C=C\CCCCC)c2ccc(F)cc12)[C@H]1CCCN1C ; [2H]C([2H])(c1cn(C(=O)CCCCCCC/C=C\C/C=C\CCCCC)c2ccc(Cl)cc12)[C@H]1CCCN1C ; CCCCC/C=C\C/C=C\CCCCCCCC(=O)n1cc(CCN(C)C)c2cc(F)ccc21 ; CCCCC/C=C\C/C=C\CCCCCCCC(=O)n1cc(CCN(C)C)c2cc(Cl)ccc21 ; CCCCC/C=C\C/C=C\CCCCCCCC(=O)OCOC(=O)n1cc(CCN(C)C)c2cc(OC)ccc21] 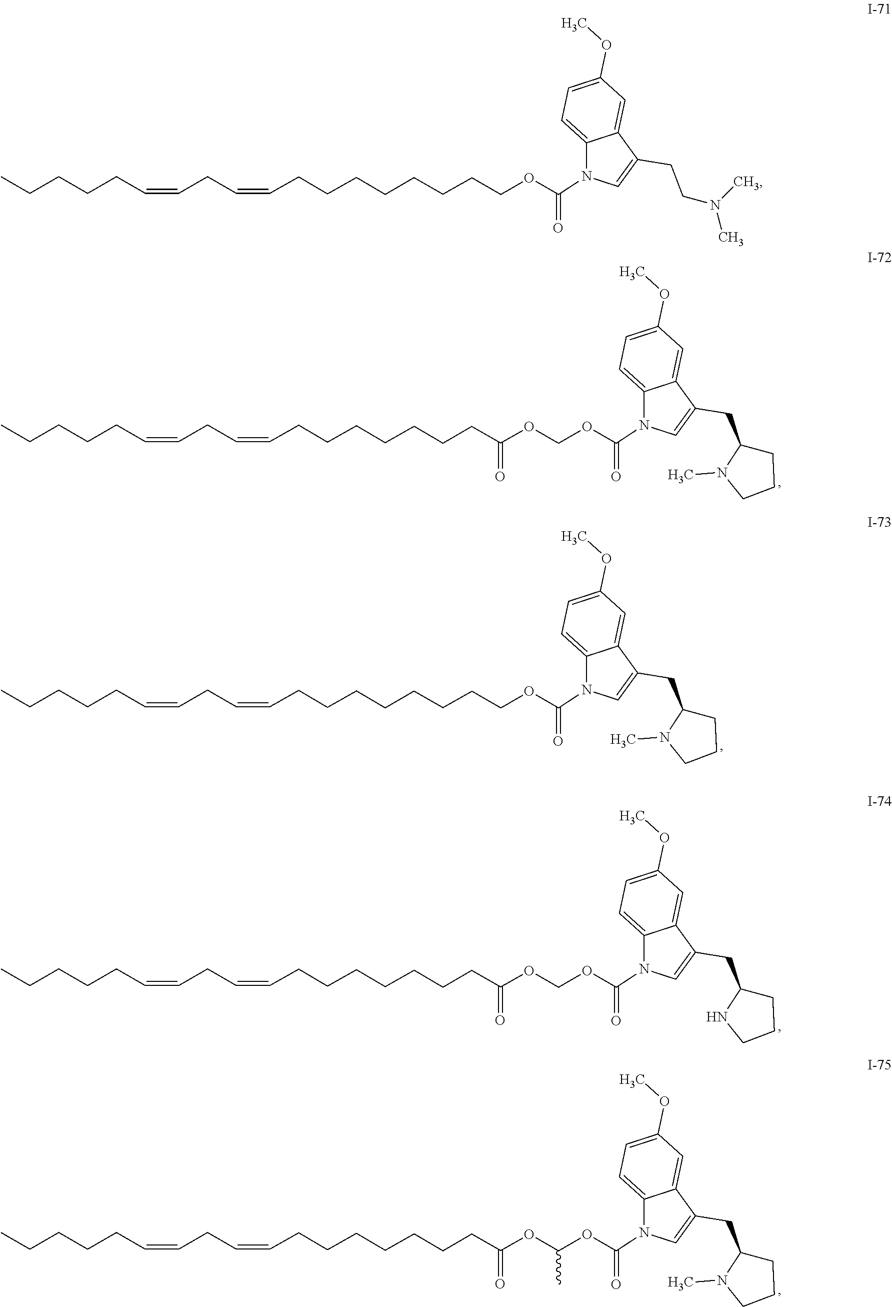 [SMILES: CCCCC/C=C\C/C=C\CCCCCCCCOC(=O)n1cc(CCN(C)C)c2cc(OC)ccc21 ; CCCCC/C=C\C/C=C\CCCCCCCC(=O)OCOC(=O)n1cc(C[C@H]2CCCN2C)c2cc(OC)ccc21 ; CCCCC/C=C\C/C=C\CCCCCCCCOC(=O)n1cc(C[C@H]2CCCN2C)c2cc(OC)ccc21 ; CCCCC/C=C\C/C=C\CCCCCCCC(=O)OCOC(=O)n1cc(C[C@H]2CCCN2)c2cc(OC)ccc21 ; CCCCC/C=C\C/C=C\CCCCCCCC(=O)OC(C)OC(=O)n1cc(C[C@H]2CCCN2C)c2cc(OC)ccc21] 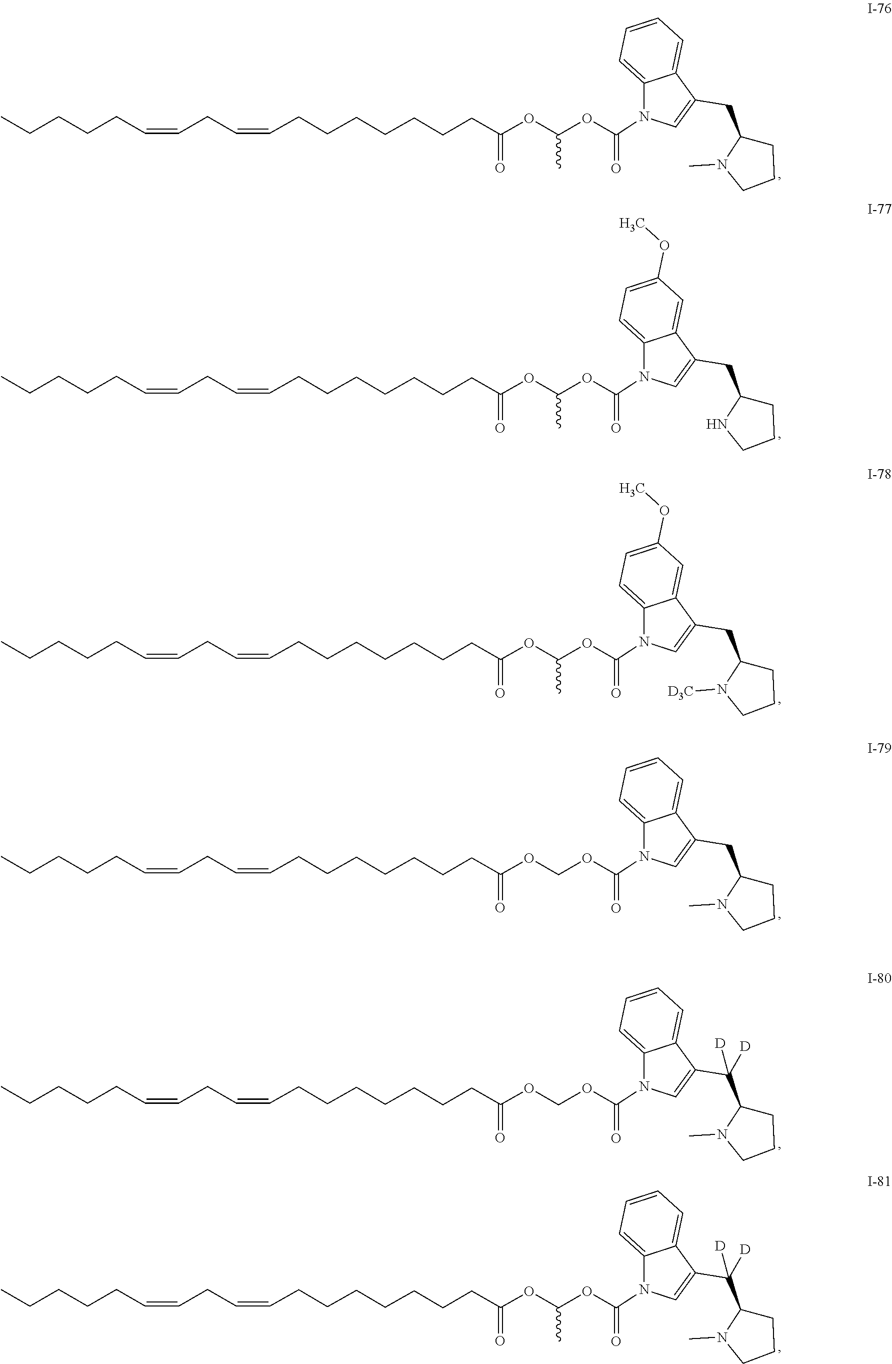 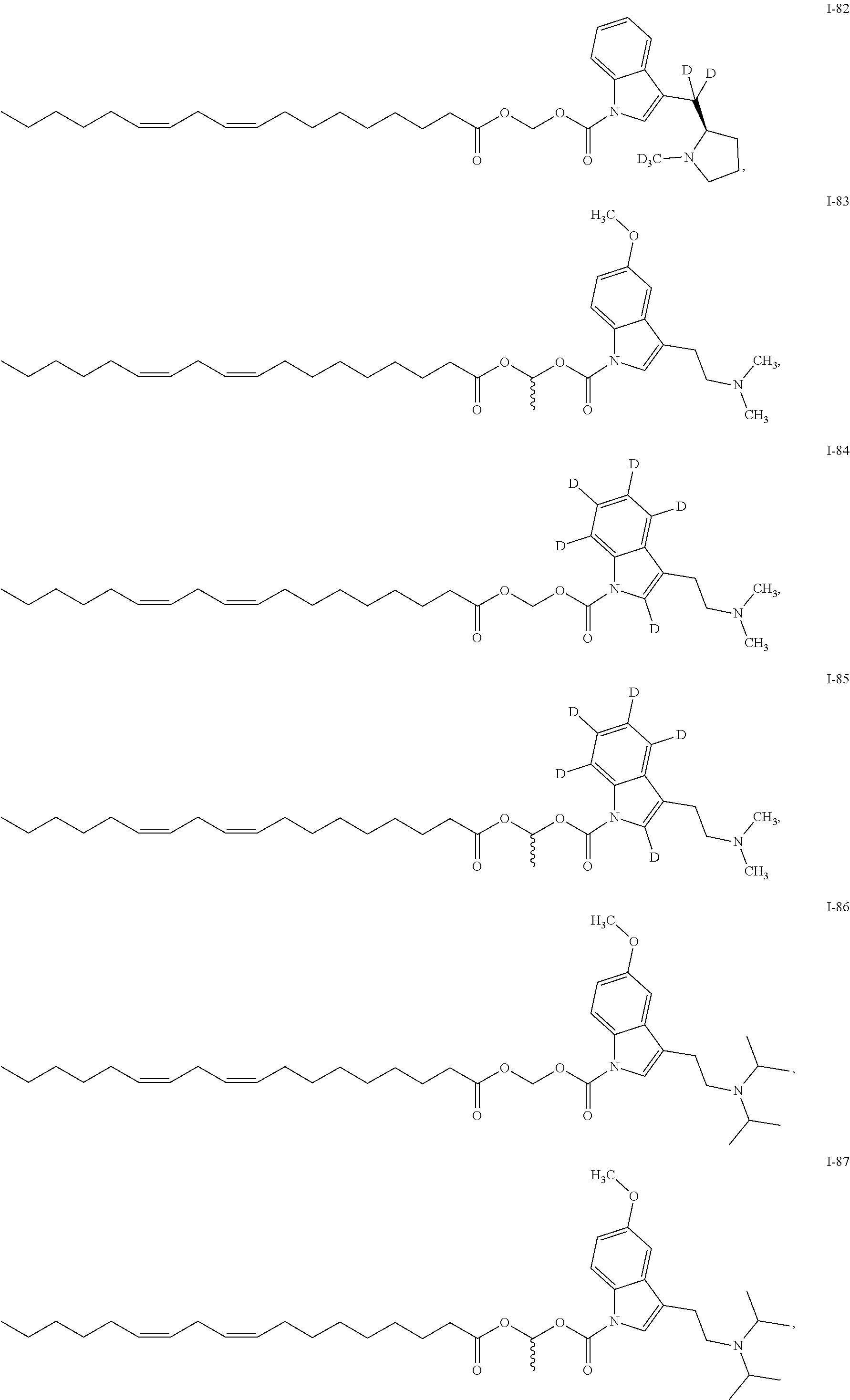 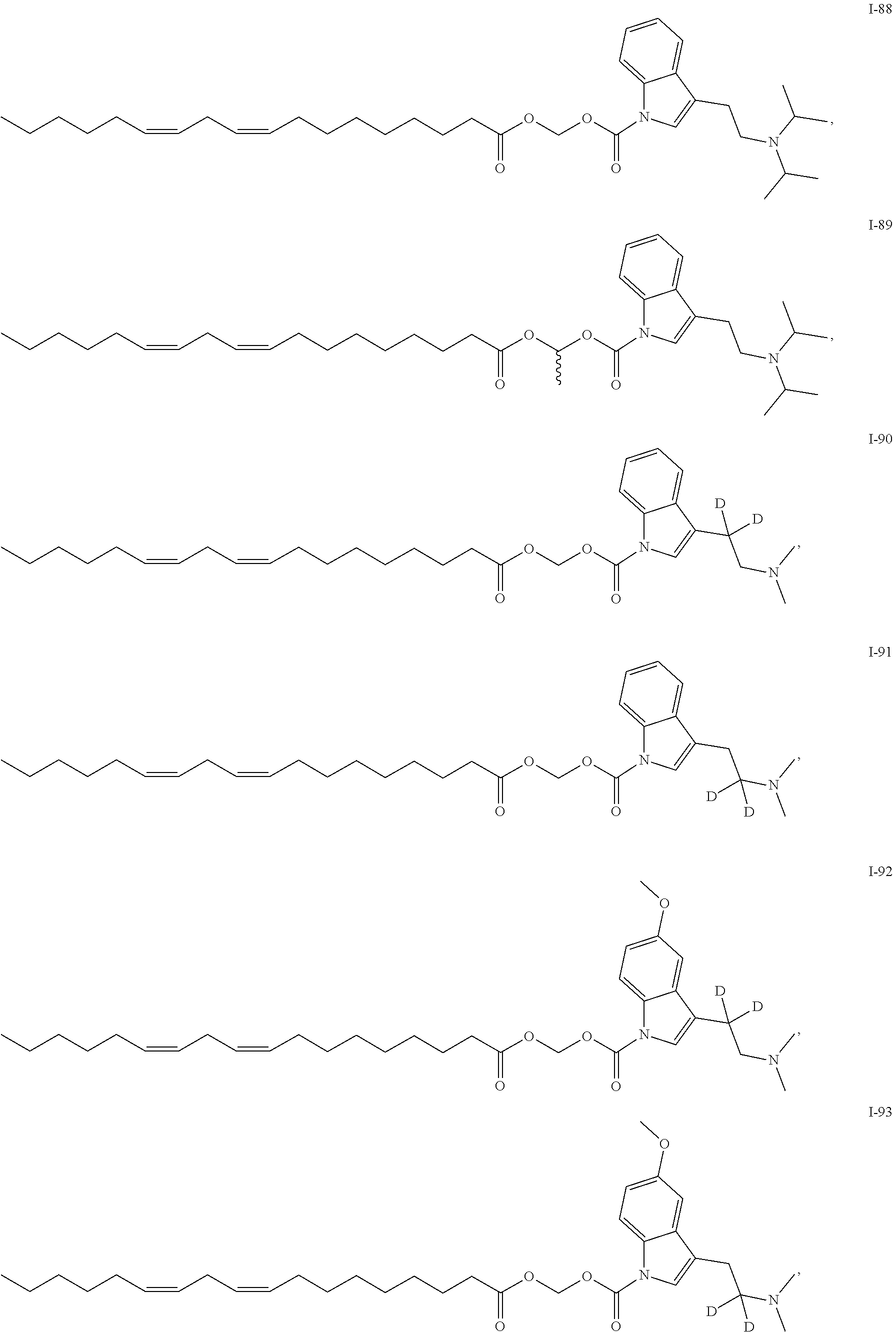 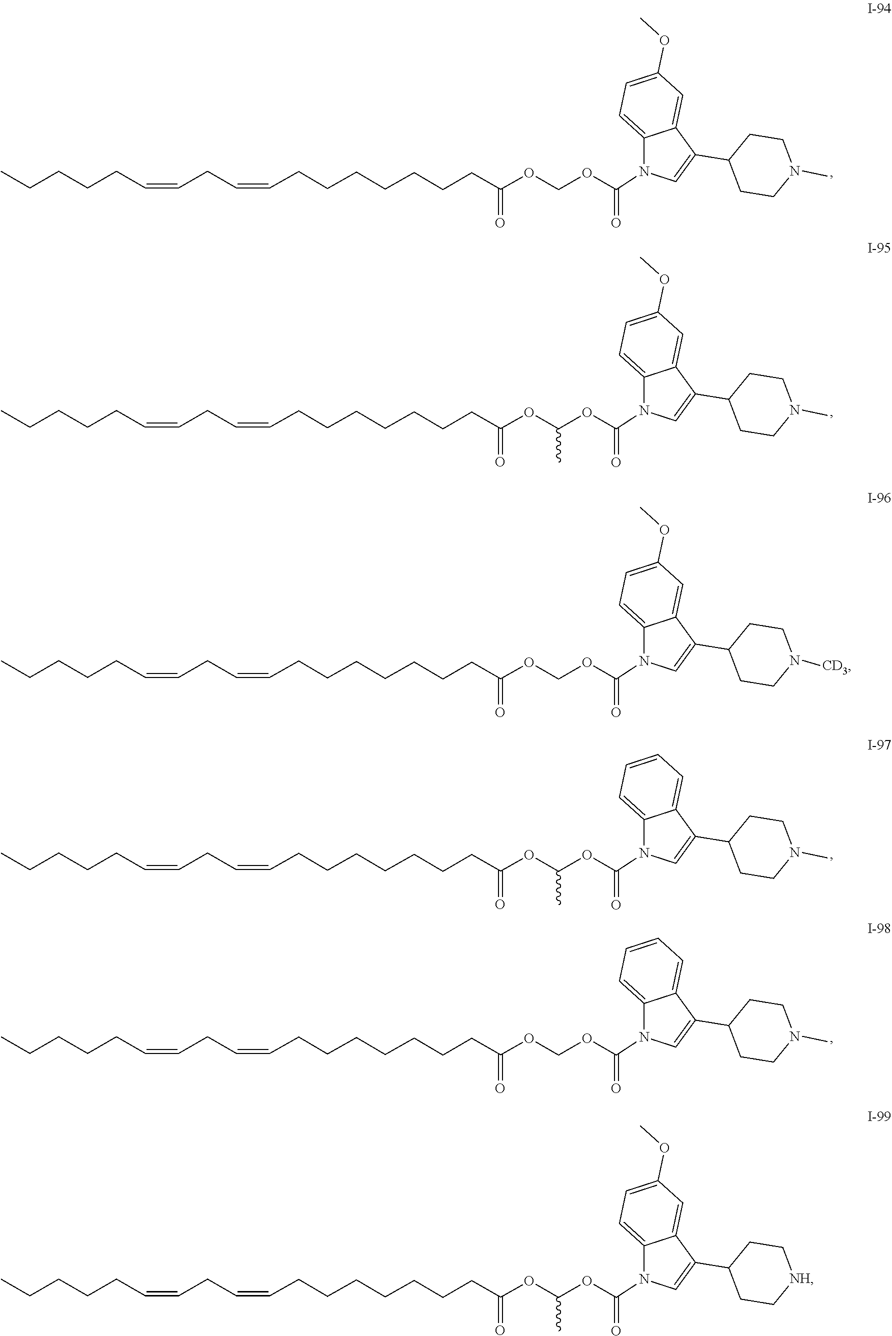 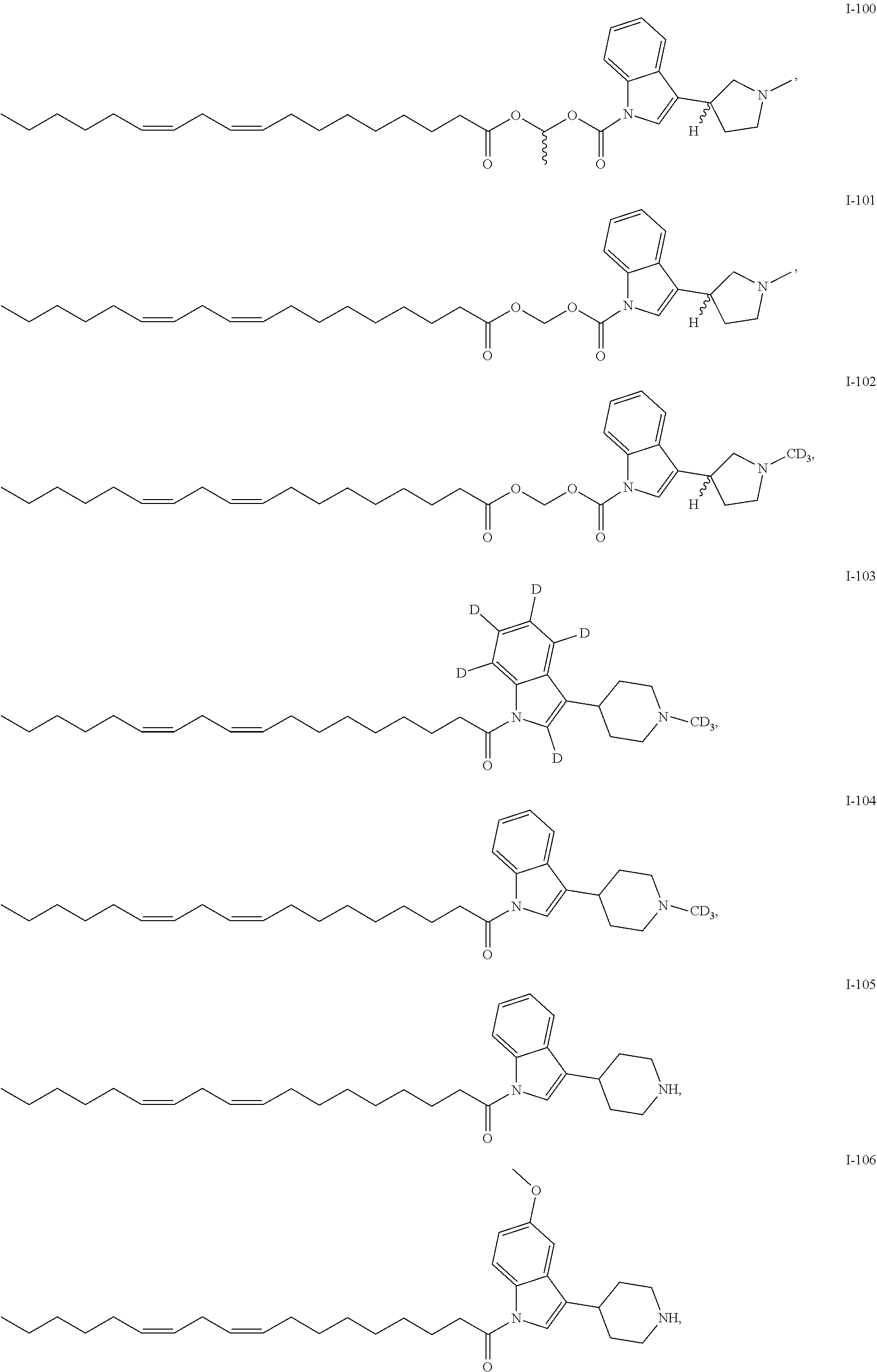 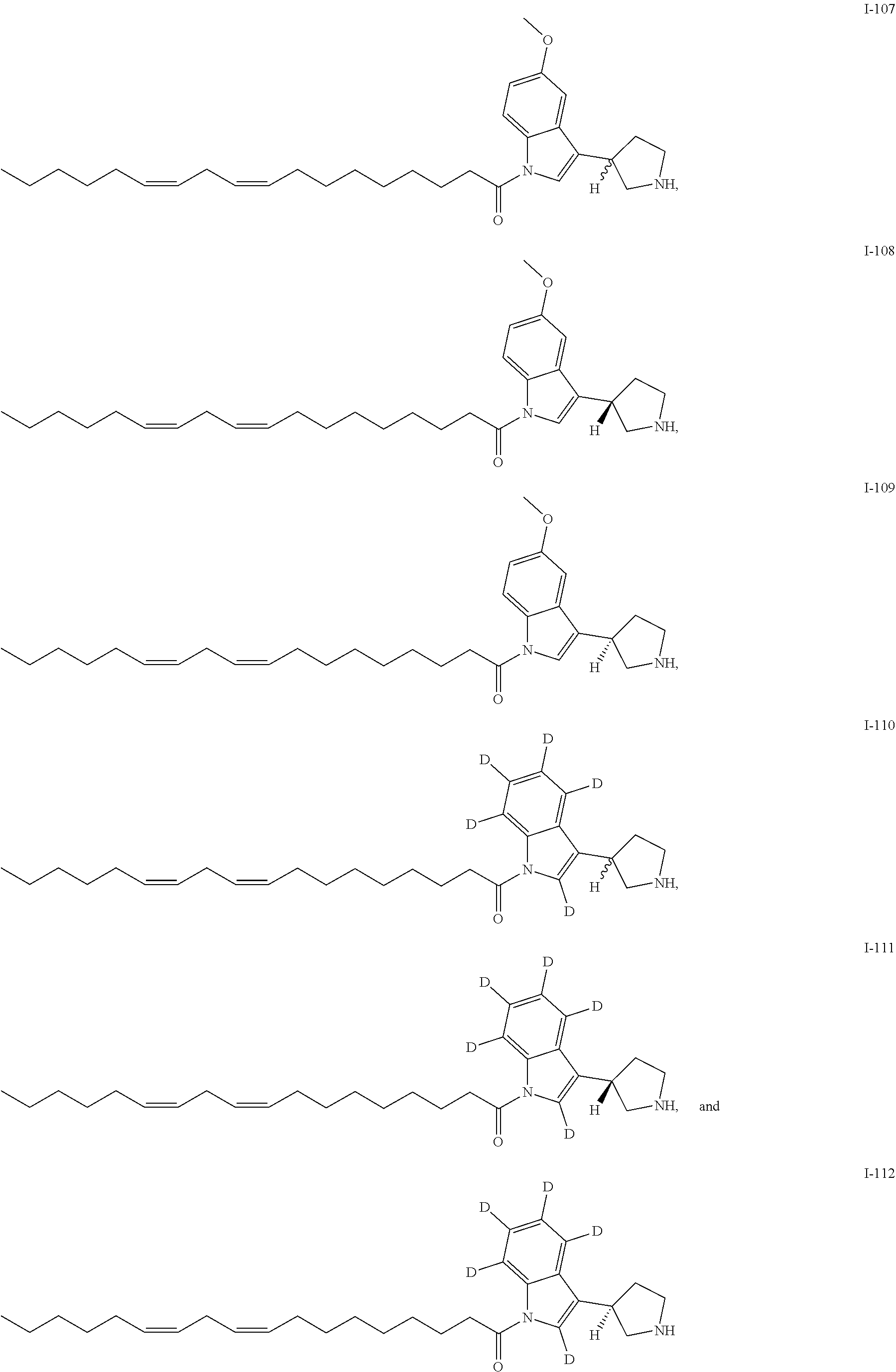
or a pharmaceutically acceptable salt and/or solvate thereof.

25. A composition comprising one or more compounds of any one of claims 1 to 24 and a carrier.

26. A pharmaceutical composition comprising one or more compounds of any one of claims 1 to 24 and pharmaceutically acceptable carrier.

27. A composition comprising one or more compounds of any one of claims 1 to 24 and one or more components of a nano-carrier system.

28. The composition of claim 27, wherein the nano-carrier system is selected from liposomes, micelles, nanoparticles, nano-emulsions and lipidic nano-systems.

29. A method of treating a disease, disorder or condition by activation of a serotonin receptor comprising administering a therapeutically effective amount of one or more compounds of any one of claims 1 to 24 to a subject in need thereof.

30. A method of treating a mental illness comprising administering a therapeutically effective amount of any one of claims 1 to 24 to a subject in need thereof.

31. The method of claim 30, wherein the mental illness is selected from hallucinations and delusions and a combination thereof.

32. The method of claim 30, wherein the mental illness is selected anxiety disorders; depression; mood disorders; psychotic disorders; impulse control and addiction disorders; drug addiction; obsessive-compulsive disorder (OCD); post-traumatic stress disorder (PTSD); stress response syndromes; dissociative disorders; depersonalization disorder; factitious disorders; sexual and gender disorders; and somatic symptom disorders and combinations thereof.

33. A method of treating psychosis or psychotic symptoms comprising administering a therapeutically effective amount of one or more compounds of any one of claims 1 to 24 to a subject in need thereof.

34. A method of treating a central nervous system (CNS) disease, disorder or condition and/or a neurological disease, disorder or condition comprising administering a therapeutically effective amount of one or more compounds of any one of claims 1 to 24 to a subject in need thereof.

35. The method of claim 34, wherein the CNS disease, disorder or condition and/or neurological disease, disorder or condition is selected from neurological diseases including neurodevelopmental diseases and neurodegenerative diseases such as Alzheimer's disease; presenile dementia; senile dementia; vascular dementia; Lewy body dementia; cognitive impairment, Parkinson's disease and Parkinsonian related disorders such as Parkinson dementia, corticobasal degeneration, and supranuclear palsy; epilepsy; CNS trauma; CNS infections; CNS inflammation; stroke; multiple sclerosis; Huntington's disease; mitochondrial disorders; Fragile X syndrome; Angelman syndrome; hereditary ataxias; neuro-otological and eye movement disorders; neurodegenerative diseases of the retina amyotrophic lateral sclerosis; tardive dyskinesias; hyperkinetic disorders; attention deficit hyperactivity disorder and attention deficit disorders; restless leg syndrome; Tourette's syndrome; schizophrenia; autism spectrum disorders; tuberous sclerosis; Rett syndrome; cerebral palsy; disorders of the reward system including eating disorders such as anorexia nervosa (“AN”) and bulimia nervosa (“BN”); and binge eating disorder (“BED”), trichotillomania, dermotillomania, nail biting; migraine; fibromyalgia; and peripheral neuropathy of any etiology, and combinations thereof.

36. A method of treating a behavioral problem comprising administering a therapeutically effective amount of one or more compounds of any one of claims 1 to 24 to a non-human subject in need thereof.

37. The method of claim 36, wherein the non-human subject is a canine or feline suffering from neurological diseases, behavioral problems, trainability problems and/or a combination thereof.

38. The method of claim 37, wherein and the neurological diseases, behavioral problems, trainability problems include, but are not limited to, anxiety, fear and stress, sleep disturbances, cognitive dysfunction, aggression, and/or a combination thereof.

39. A method of treating a disease, disorder or condition by activation of a serotonin receptor comprising administering a therapeutically effective amount of one or more compounds of any one of claims 1 to 24 in combination with another known agent useful for treatment of a disease, disorder or condition by activation of a serotonin receptor to a subject in need thereof.

40. The method of any one of claims 29 to 39, comprising a decreased or lower risk of the subject experiencing or having serotonin syndrome.

41. A pharmaceutical composition comprising a compound of any one of claims 1 to 24 and an additional therapeutic agent.

42. The composition of claim 41, wherein the additional therapeutic agent is a psychoactive drug.

43. The composition of claim 42, wherein the additional therapeutic agent is a psychoactive drug that modifies release of serotonin or activates serotonin receptors.
